(19) United States
(12) Patent Application Publication
(10) Pub. No.: US 2025/0257133 A1
(43) Pub. Date: Aug. 14, 2025

(54) Anti-CD28 Antibodies and Methods of Use Thereof

(71) Applicant: ADAGENE PTE. LTD., Singapore (SG)

(72) Inventors: Peter Peizhi LUO, Suzhou (CN); Fangyong DU, Suzhou (CN); Yan LI, Suzhou (CN); Guizhong LIU, Suzhou (CN)

(73) Assignee: ADAGENE PTE. LTD., Singapore (SG)

(21) Appl. No.: 18/855,066

(22) PCT Filed: Apr. 10, 2023
(86) PCT No.: PCT/CN2023/087277

§ 371 Date: Oct. 8, 2024

(30) Foreign Application Priority Data
Apr. 8, 2022 (WO) PCT/CN2022/085838

(51) Int. Cl. C07K 16/28; A61K 39/00; A61K 47/68; A61P 35/00; C07K 16/30; C07K 16/32

(52) U.S. Cl. CPC C07K 16/2818; A61K 47/6879; A61P 35/00; C07K 16/2809; C07K 16/2827; C07K 16/30; C07K 16/32; A61K 2039/507; C07K 2317/31; C07K 2317/33; C07K 2317/34; C07K 2317/526; C07K 2317/55; C07K 2317/565; C07K 2317/622; C07K 2317/71; C07K 2317/92; C07K 2319/50

(57) ABSTRACT

The present disclosure relates to anti-CD28 antibodies that bind to human CD28 and antigen-binding fragments thereof, compositions comprising same, and uses thereof in delaying and/or preventing tumor growth.

### CROSS-REFERENCE TO RELATED APPLICATION

This application claims priority from PCT/CN2022/085838, filed Apr. 8, 2023, whose disclosure is incorporated by reference herein in its entirety.

### SEQUENCE LISTING

The instant application contains a Sequence Listing that has been submitted electronically in XML format and is hereby incorporated by reference in its entirety. The electronic copy of the Sequence Listing, created on Apr. 6, 2023 and named 123687_W0003_SL. xml, is 488, 908 bytes in size.

### BACKGROUND OF THE INVENTION

CD28 is a costimulatory signaling molecule on T cells and plays a key role in their activation, proliferation, and survival. Activation of the CD28 costimulatory signal has the potential to enhance anti-tumor T cell immunity. However, therapies targeting CD28 for systemic T cell activation have caused deleterious cytokine storm and multiorgan failure (Suntharalingam et al., N Engl J Med. (2006) 355 (10): 1018-28). Thus, there is a need for improved CD28-targeting therapies that avoid serious autoimmune adverse events associated with systemic CD28 activation.

### SUMMARY OF THE INVENTION

The present disclosure provides novel binding molecules (e.g., antibodies) targeting a tumor-associated antigen (e.g., B7-H3, HER2, TROP2, or PD-L1) and CD28, as well as pharmaceutical compositions comprising one or more of these binding molecules, and use of these binding molecules and pharmaceutical compositions for treating cancer. Compared to currently available cancer treatments, including antibody treatments, it is contemplated that the binding molecules of the present disclosure may provide a superior clinical response.

In some aspects, the present disclosure provides an antigen-binding protein such as an antibody or an antigen-binding fragment thereof, comprising an CD28-binding portion, wherein the CD28-binding portion binds human CD28 and is cross-reactive with cynomolgus monkey and mouse CD28. In some embodiments, the CD28-binding portion binds to a CD28 epitope comprising amino acid residues 51-122 of human CD28 (SEQ ID NO: 1). In particular embodiments, the CD28 epitope comprises amino acid residues 51, 52, 54, 55, 98-101, 110-111, 113-114, and 118-122 of SEQ ID NO: 1.

In some embodiments, the present disclosure provides an antigen-binding protein such as an antibody or an antigen-binding fragment thereof comprising a CD28-binding portion that binds human CD28, wherein the CD28-binding portion comprises an antibody heavy chain variable domain (V_{H}) and an antibody light chain variable domain (V_{L}), and wherein the V_{H} and V_{L} comprises heavy chain complementarity-determining regions (CDRs) 1-3 and light chain CDR1-3 set forth in SEQ ID NOs: 5-10, respectively, SEQ ID NOs: 15, 6, 16, 17-19, respectively, SEQ ID NOs: 24, 6, 25, 26-28, respectively, SEQ ID NOs: 33, 6, 35-38, respectively, SEQ ID NOs: 43, 6, 44, 45, 9, and 46, respectively, SEQ ID NOs: 33, 51-53, 300, and 10, respectively, SEQ ID NOs: 24, 58, 59, 60, 300, and 61, respectively, SEQ ID NOs: 66-69, 300 and 70, respectively, SEQ ID NOs: 24, 6, 75, 76, 18, and 28, respectively, SEQ ID NOs: 24, 58, 81, 82, 27, and 83, respectively, SEQ ID NOs: 88-91, 300, and 70, respectively, SEQ ID NOs: 24, 96-98, 9, and 70, respectively, SEQ ID NOs: 103-106, 18, and 83, respectively, SEQ ID NOs: 111, 6, 112, 113, 18, and 114, respectively, SEQ ID NOs: 15, 6, 119, 120, 9, and 121, respectively, SEQ ID NOs: 126, 67, 127, 128, 18, and 129, respectively, SEQ ID NOs: 134, 6, 135, 136, 27, and 83, respectively, SEQ ID NOs: 43, 58, 141, 142, 300, and 143, respectively, SEQ ID NOs: 148, 6, 149, 150, 300, and 83, respectively, SEQ ID NOs: 15, 155, 16, 156, 27, and 70, respectively, or SEQ ID NOs: 161, 6, 162, 163, 300, and 164.

In some embodiments, the CD28-binding portion comprises V_{H} and V_{L} set forth in SEQ ID NOs: 11 and 12, respectively, SEQ ID NOs: 20 and 21, respectively, SEQ ID NOs: 29 and 30, respectively, SEQ ID NOs: 39 and 40, respectively, SEQ ID NOs: 47 and 48, respectively, SEQ ID NOs: 54 and 55, respectively, SEQ ID NOs: 62 and 63, respectively, SEQ ID NOs: 71 and 72, respectively, SEQ ID NOs: 77 and 78, respectively, SEQ ID NOs: 84 and 85, respectively, SEQ ID NOs: 92 and 93, respectively, SEQ ID NOs: 99 and 100, respectively, SEQ ID NOs: 107 and 108, respectively, SEQ ID NOs: 115 and 116, respectively, SEQ ID NOs: 122 and 123, respectively, SEQ ID NOs: 130 and 131, respectively, SEQ ID NOs: 137 and 138 respectively, SEQ ID NOs: 144 and 145, respectively, SEQ ID NOs: 151 and 152, respectively, SEQ ID NOs: 157 and 158, respectively, SEQ ID NOs: 165 and 166, respectively, SEQ ID NOs: 362 and 363, respectively, SEQ ID NOs: 364 and 365, respectively, SEQ ID NOs: 366 and 367, respectively, SEQ ID NOs: 368 and 369, respectively, SEQ ID NOs: 370 and 371, respectively, SEQ ID NOs: 372 and 373, respectively, SEQ ID NOs: 374 and 375, respectively, SEQ ID NOs: 376 and 12, respectively, SEQ ID NOs: 377 and 378, respectively, SEQ ID NOs: 379 and 380, respectively, SEQ ID NOs: 381 and 12, respectively, SEQ ID NOs: 382 and 12, respectively, SEQ ID NOs: 383 and 12, respectively, SEQ ID NOs: 384 and 12, respectively, SEQ ID NOs: 385 and 12, respectively, or SEQ ID NOs: 386 and 12, respectively.

In some embodiments, the CD28-binding protein comprises a heavy chain (HC) and a light chain (LC) set forth in SEQ ID NOs: 13 and 14, respectively, SEQ ID NOs: 22 and 23, respectively, SEQ ID NOs: 31 and 32, respectively, SEQ ID NOs: 41 and 42, respectively, SEQ ID NOs: 49 and 50, respectively, SEQ ID NOs: 56 and 57, respectively, SEQ ID NOs: 64 and 65, respectively SEQ ID NOs: 73 and 74, respectively, SEQ ID NOs: 79 and 80, respectively, SEQ ID NOs: 86 and 87, respectively, SEQ ID NOs: 94 and 95, respectively, SEQ ID NOs: 101 and 102, respectively, SEQ ID NOs: 109 and 110, respectively, SEQ ID NOs: 117 and 118, respectively, SEQ ID NOs: 124 and 125, respectively, SEQ ID NOs: 141 and 142, respectively, SEQ ID NOs: 132 and 133, respectively, SEQ ID NOs: 139 and 140, respectively, SEQ ID NOs: 146 and 147, respectively, SEQ ID NOs: 153 and 154, respectively, SEQ ID NOs: 159 and 160, respectively, or SEQ ID NOs: 167 and 168, respectively.

In some aspects, the present disclosure also provides pharmaceutical compositions comprising an antigen-binding protein such as an antibody or an antigen-binding fragment thereof herein and a pharmaceutically acceptable carrier; nucleic acid molecule (s) encoding the antigen-binding protein; expression vector (s) comprising the nucleic acid molecule (s); and a host cell comprising the vector (s), wherein the host cell may be a prokaryotic cell or an eukaryotic cell such as a mammalian cell.

In some aspects, the present disclosure also provides a method of producing the antigen-binding protein such as an antibody or an antigen-binding fragment thereof herein, comprising culturing the host cell under conditions that allow expression of the antigen-binding protein, and isolating the antigen-binding protein from the culture.

In some aspects, the present disclosure also provides a method of treating cancer in a patient in need thereof, comprising administering to the patient a therapeutically effective amount of the antigen-binding protein, such as an antibody or an antigen-binding fragment, herein. In some embodiments, the method further comprises administering to the patient another anti-cancer therapeutic. In further embodiments, the additional anti-cancer therapeutic is a bispecific antibody (bsAb) targeting CD3 and a tumor-associated antigen (TAA). In particular embodiments, the TAA is B7-H3, HER2, or TROP2. In some embodiments, the additional anti-cancer therapeutics is an immune checkpoint inhibitor, optionally an anti-PD-1, anti-CTLA-4, or anti-PD-L1 antibody.

Also provided herein are the present antigen-binding proteins such as antibodies or antigen-binding fragments thereof, or pharmaceutical compositions, for use in treating a cancer in a patient in need thereof; use of the present antigen-binding proteins such as antibodies or antigen-binding fragments thereof for the manufacture of a medicament for treating a cancer in a patient in need thereof; and articles of manufacture (e.g., kits) comprising one or more dosing units of the present antigen-binding proteins such as antibodies or or fragments thereof.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE DRAWINGS

FIG. 1 is a panel of graphs showing a binding affinity assay of anti-CD28 antibodies to recombinant CD28 (human and mouse) protein.

FIG. 2 is a graph showing binding of anti-CD28 IgGs to human CD3+ T cells.

FIG. 3 is a panel of graphs showing ligand blocking assays of IgGs for human CD28-CD80 (top right and top left graphs) and CTLA-4 CD80 pairs (bottom graph).

FIG. 4A is a graph showing a T cell proliferation assay showing that IgGs does not induce systemic T cell activation in comparison to CD28 superagonist TAC2386 (TGN1412).

FIG. 4B is a graph showing a T cell activation co-stimulation assay-proliferation by CellTiter-Glo® (CTG) readout.

FIG. 4C is a graph showing a T cell activation co-stimulation assay for IFN-γ release.

FIG. 5A is a graph showing a T cell activation and proliferation assay-proliferation by CTG readout using anti-CD28 test antibodies with OKT3.

FIG. 5B is a graph that shows a T cell activation and proliferation assay for IL-2 release.

FIG. 6 is a graph showing binding of anti-HER2 antibodies and bispecific antibodies (BsAbs) to SK-OV3 cells. Also shown is a table containing plotted values.

FIG. 7 is a graph showing a Jurkat-NFκB Luciferase reporter gene assay measuring NFκB signaling stimulatory effects in terms of maximum signal and EC₅₀ values of anti-HER2×CD28 BsAb (TY27566 or TY27807) in combination with a fixed concentration of anti-HER2×CD3 BsAb (TY25238), and anti-HER2×CD3 BsAb in combination with a fixed concentration of anti-HER2×CD28 BsAb. Also shown is a table containing plotted values.

FIG. 8 is a graph showing a killing assay to MCF-7 cells of CD28 BsAb and CD3 BsAb with the same or different HER2 epitope and a table containing plotted values.

FIG. 9 is a panel of graphs showing an assay to measure concentration-dependent binding activity of anti-TROP2 bispecific antibodies on tumor cell lines.

FIG. 10A is a graph showing a Jurkat-NFκB Luciferase reporter assay measuring NFκB signaling stimulatory effects in terms of maximum signal and EC₅₀ values of bispecific antibodies on H292 cells. Also shown is a table containing plotted values.

FIG. 10B is a graph showing a Jurkat-NFκB Luciferase reporter assay measuring NFκB signaling stimulatory effects in terms of maximum signal and EC₅₀ values of a bispecific antibodies on H292 cells. Also shown is a table containing plotted values.

FIG. 11 is a panel of flow cytometry plots showing the co-expression of PD-L1 and B7H3 on MDA-MB-231 cells.

FIG. 12 is a graph showing a binding assay of B7H3 IgG and B7H3×CD28 bsAb to MDA-MB-231 cells. Also shown is a table containing plotted values.

FIGS. 13A and 13B are graphs showing a one-way MLR assay to test the activity of B7H3×CD28 bsAb in combination with anti-PD-1 or anti-PD-L1 blocking mAbs on primary human T cell activation, as measured by IL-2 secretion (FIG. 13A) and IFN-γ secretion (FIG. 13B).

FIG. 14 is a graph showing an in vitro assay that measures tumor killing activity of anti-CD3-based, or anti-CD28-based HER2-targeted bsAbs or their combinations on the MCF-7 tumor cell line. Also shown is a table containing plotted values.

FIG. 15 is a graph showing an in vitro assay that measures tumor killing activity of anti-CD3-based, or anti-CD28-based HER2-targeted bsAbs or their combinations on the EMT6-HER2 tumor cell line. Also shown is a table containing plotted values.

FIG. 16 is panel of graphs showing an assay to measure systemic cytokine release of IL-6 and IFN-γ (top left and right graphs, respectively), and to measure CD3+ T cells percentage of total CD45+ T cells (bottom graph), in WT mice treated with TCEs.

FIG. 17 shows an in vivo efficacy study and graph of HER2×CD3 bsAb and B7H3×CD28 bsAb mono or in combination in SK-OV3+PBMC xenograft tumor model.

FIG. 18 is a panel of graphs showing an in vivo efficacy study of B7H3×CD28 or HER2×CD28 bsAb in EMT6-HER2 model.

FIG. 19 is a graph showing the ELISA measurement of masking efficiency of anti-CD28 activatable antibodies binding to recombinant human CD28.

FIG. 20 is a graph showing the ELISA measurement of masking efficiency of anti-CD28 activatable antibodies binding to recombinant human CD28.

FIG. 21 (SEQ ID NOs: 359-361) is table showing the different binding residues from human and mouse CD28.

FIG. 22A is a graph showing binding of anti-PD-L1×CD28 bsAbs TY29815 and TY30413 to human CD28.

FIG. 22B is a graph showing binding of anti-PD-L1×CD28 bsAbs TY29815, TY30406, TY30410, and TY30413 to mouse CD28.

FIG. 23A is a graph showing binding of anti-HER2×CD28 bsAbs TY27566, TY28652, TY28653, TY28654, TY28655, TY28656, TY28657, TY28658, TY28659, TY28660, and TY28661 to human CD28.

FIG. 23B is a graph showing binding of anti-HER2×CD28 bsAbs TY29109, TY29306, TY29307, TY29308, TY29309, TY29310, TY29311, TY29312, TY29313, TY29314, and TY29315 to human CD28.

FIG. 24A is a graph showing binding of anti-B7H3×CD28 bsAbs TY29021, TY30120, TY30121, and TY30123 to human CD28.

FIG. 24B is a graph showing binding of anti-B7H3×CD28 bsAbs TY29021, TY30120, TY30121, and TY30123 to mouse CD28.

FIG. 25 is a graph showing mouse T-cell binding assay measuring T-cell binding effects in terms of maximum signal, EC₅₀, and AUC values of anti-B7H3×CD28 bsAbs TY29021, TY30120, TY30121, and TY30123. Also shown is a table containing plotted values.

### DETAILED DESCRIPTION

### I. Definitions

Before describing the present disclosure in detail, it is to be understood that this present disclosure is not limited to particular compositions or biological systems, which can, of course, vary. It is also to be understood that the terminology used herein is for the purpose of describing particular embodiments only, and is not intended to be limiting.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure described herein include “comprising,” “consisting,” and “consisting essentially of” aspects and embodiments.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “antibody” encompasses various antibody structures, including but not limited to monoclonal antibodies (including full length monoclonal antibodies), polyclonal antibodies, masked antibodies (e.g., activatable or non-activatable antibodies), multi-specific antibodies (e.g., bispecific antibodies, including masked bispecific antibodies), and antibody fragments (e.g., a single-chain variable fragment or scFv) so long as they exhibit the desired biological activity (e.g., the ability to bind a target antigen with desired specificity and affinity).

The term “antibody” encompasses various antibody structures, including but not limited to monoclonal antibodies (including full length monoclonal antibodies), polyclonal antibodies, masked antibodies (e.g., activatable or non-activatable antibodies), and multi-specific antibodies (e.g., bispecific antibodies). The term “antibody” also includes, but is not limited to, chimeric antibodies, humanized antibodies, and fully human antibodies.

In some embodiments, the term “antibody” refers to an antigen-binding protein (i.e., immunoglobulin) having a basic four-polypeptide chain structure consisting of two identical heavy (H) chains and two identical light (L) chains. Each L chain is linked to an H chain by one covalent disulfide bond, while the two H chains are linked to each other by one or more disulfide bonds depending on the H chain isotype. Each heavy chain has, at the N-terminus, a variable region (also known as variable domain) (abbreviated herein as V_{H}) followed by a constant region. The heavy chain constant region is comprised of three domains, C_{H}1, C_{H}2 and C_{H}3. Each light chain has, at the N-terminus, a variable region (also known as variable domain) (abbreviated herein as V_{L}) followed by a constant region at its other end. The light chain constant region is comprised of one domain, C_{L}. The V_{L} is aligned with the V_{H} and the C_{L} is aligned with the first constant domain of the heavy chain (C_{H}1). The pairing of a V_{H} and V_{L} together forms a single antigen-binding site.

The V_{H} and V_{L} can be further subdivided into complementarity-determining regions (CDRs) and framework regions (FRs). CDRs are of highest sequence variability and/or involved in antigen recognition. CDRs and FRs intersperse in the order of FR1, CDR1, FR2, CDR2, FR3, CDR3, and FR4. CDRs also comprise “specificity determining residues,” or “SDRs,” which are residues that contact the antigen. SDRs are contained within regions of the CDRs called abbreviated-CDRs, or a-CDRs. Exemplary a-CDRs (a-LCDR1, a-LCDR2, a-LCDR3, a-HCDR1, a-HCDR2, and a-HCDR3) occur at amino acid residues 31-34, 50-55, 89-96 of the light chain, and 31-35, 50-58, and 95-102 of the heavy chain, respectively. See Almagro and Fransson, Front Biosci. (2008) 13: 1619-33). Unless otherwise indicated, residues in the variable domain are numbered herein according to Kabat et al., J Biol Chem. (1977) 252: 6609-16; Kabat et al., U.S. Dept. of Health and Human Services, “Sequences of proteins of immunological interest” (1991).

Table 1 below provides exemplary CDR definitions according to various algorithms known in the art.

TABLE 1
CDR Definitions

| | Kabat¹ | Chothia² | MacCallum³ | IMGT⁴ | AHo⁵ |
|---|---|---|---|---|---|
| HCDR1 | 31-35 | 26-32 | 30-35 | 27-38 | 25-40 |
| HCDR2 | 50-65 | 53-55 | 47-58 | 56-65 | 58-77 |
| HCDR3 | 95-102 | 96-101 | 93-101 | 105-117 | 109-137 |
| LCDR1 | 24-34 | 26-32 | 30-36 | 27-38 | 25-40 |
| LCDR2 | 50-56 | 50-52 | 46-55 | 56-65 | 58-77 |
| LCDR3 | 89-97 | 91-96 | 89-96 | 105-117 | 109-137 |

¹Residue numbering follows the nomenclature of Kabat et al., supra.
²Residue numbering follows the nomenclature Chothia et al., J. Mol. Biol. (1987) 196: 901-917; Al-Lazikani B. et al., J. Mol. Biol. (1997) 273: 927-948.
³Residue numbering follows the nomenclature of MacCallum et al., J. Mol. Biol. (1996) 262: 732-745; Abhinandan amd Martin, Mol. Immunol. (2008) 45: 3832-3839.
⁴Residue numbering follows the nomenclature of Lefranc M. P. et al., Dev. Comp. Immunol. (2003) 27: 55-77; and Honegger and Plückthun, J. Mol. Biol. (2001) 309: 657-670.
⁵Residue numbering follows the nomenclature of Honegger and Plückthun, J. Mol. Biol. (2001) 309: 657-670.

The L chain from any vertebrate species can be assigned to one of two clearly distinct types, called kappa and lambda, based on the amino acid sequences of their constant domains. Depending on the amino acid sequence of the constant domain of their heavy chains (C_{H}), antibodies can be assigned to different classes or isotypes. There are five classes of antibodies: IgA, IgD, IgE, IgG, and IgM, having heavy chains designated α (alpha), δ (delta), ε (epsilon), γ (gamma), and μ(mu), respectively. The IgG class of antibody can be further classified into four subclasses IgG₁, IgG₂, IgG₃, and IgG₄ by the gamma heavy chains, Y1-Y4, respectively.

The term “antigen-binding fragment” or “antigen-binding portion,” used herein interchangeably, refers to parts of an antibody that retain the ability to bind to the antigen of the antibody. Examples of “antigen-binding fragments” of an antibody include, but are not limited to, (i) a Fab fragment, a monovalent fragment consisting of the V_{L}, V_{H}, C_{L} and C_{H1} domains, obtainable by papain digestion; (ii) a F(ab′)₂ fragment, a bivalent fragment comprising two Fab fragments linked by a disulfide bridge at the hinge region, obtainable by pepsin digestion; (iii) a Fv fragment consisting of the V_{L} and V_{H} domains of a single arm of an antibody, (iv) a single chain Fv (scFv) fragment comprising the V_{H} and V_{L} domains of an antibody that are fused to each other; and (v) a single chain Fab (scFab) fragment comprising a single polypeptide comprising the V_{L}, V_{H}, C_{L} and C_{H1} domains.

The term “masked antibody” refers to an antibody (including a multi-specific antibody), or an antigen-binding fragment thereof, comprising a masking peptide that interferes with, obstructs, reduces the ability of, prevents, inhibits, or competes with the antigen binding domain of the antibody, for binding to its target. A masked antibody may be generated by linking a masking peptide to the antigen-binding domain of an antibody. In some embodiments, a masked antibody, or an antigen-binding fragment thereof, exhibits a first binding affinity to a target when in an inactivated state (e.g., inhibited or masked by a masking peptide), and exhibits a second binding affinity to the target in an activated state (e.g., uninhibited or unmasked by the masking peptide (e.g., the masking peptide is cleaved from the antibody)), where the second binding affinity is greater than the first binding affinity. A masked antibody may be generated by linking a masking peptide comprising an activatable component (e.g., a cleavable site within a linkage unit, or “LU”) to the antigen binding domain of an antibody. In some embodiments, the masked antibody, or an masked antigen-binding fragment thereof, is a multi-specific antibody comprising a binding domain that is specific for a T-cell surface molecule (e.g., CD28, CD3) and a binding domain that is specific for tumor cell surface antigen (e.g., HER2, B7H3, TROP2, etc.). In some embodiments, the masked antibody is bivalent and has a single mask on one of the two binding domains. In some embodiments, the masked antibody is bivalent and has a mask on each of the two binding domains. For example, for a single-masked antibody, one of the binding domains of the antibody is masked by a fused or conjugated masking peptide. For a bispecific antibody, one or both binding domains may be masked by specific but different masking peptides. An unactivated bispecific antibody that targets both cancer cells and T cells may have the binding sites of both binding domains masked to inhibit (or minimize) binding to antigen-expressing cancer cells and T cells. However, in an activated state, the masks are cleaved off to allow binding of the antibody to both the tumor antigen and the T-cell surface molecule (e.g., CD28) in the tumor microenvironment (TME). In this instance, the activated bispecific antibody selectively engages T cells to kill target tumor antigen-expressing cancer cells.

A “masking peptide” refers to a peptide which inhibits binding of an antigen binding domain to its target antigen, and typically comprises, from N terminus to C terminus, a masking unit (MU) and a linkage unit (LU). The C terminus of the masking peptide is typically linked to the N terminus of the V_{H} or the V_{L} of the antigen-binding domain. In some embodiments, the masking peptide, or a portion thereof, interferes with or inhibits binding of the antigen binding domain to its target so efficiently that binding of the antigen-binding domain to its target is extremely low and/or below the limit of detection (e.g., binding cannot be detected in an ELISA or flow cytometry assay). The masked antibodies or polypeptides described herein may comprise one or more linkers, e.g., within the LU, disposed between MU and LU, LU and V_{H} or V_{L}, or V_{H} and hinge region of an Fc.

The LU of the masking peptide may comprise at least one cleavable site. A cleavage site generally includes an amino acid sequence that is cleavable, for example, serves as the substrate for an enzyme and/or a cysteine-cysteine pair capable of forming a reducible disulfide bond. As such, when the terms “cleavage,” “cleavable,” “cleaved” and the like are used in connection with a cleavage site, the terms encompass enzymatic cleavage, e.g., by a protease, as well as disruption of a disulfide bond between a cysteine-cysteine pair via reduction of the disulfide bond that can result from exposure to a reducing agent. The amino acid sequence of the cleavage site may overlap with or be included within the MU. Masked antibodies or masked polypeptides may comprise a cleavage site configured to mediate activation of the antibody or the polypeptide. For example, when the cleavage site of an activatable antibody is intact (e.g., uncleaved by a corresponding enzyme, and/or containing an unreduced cysteine-cysteine disulfide bond), the masking peptide, or a portion thereof, may interfere with or inhibit binding of the antigen binding domain to its target. In some embodiments, the LU of the masking peptide does not comprise a cleavable site.

The term “masking efficiency” refers to the efficiency with which the masking peptide inhibits binding of the antigen binding domain to the target antigen. Masking efficiency may be measured as the difference in or the ratio of the binding affinity of a masked antibody or masked polypeptide comprising an antigen binding domain and the binding affinity of an unmasked antibody or unmasked polypeptide comprising an antigen binding domain (e.g., the masking peptide is cleaved from the antibody). For example, the masking efficiency may be measured by dividing the EC₅₀ or K_{D} of a masked antibody for binding a target antigen in its inactivated (e.g., inhibited, masked, and/or uncleaved) state, relative to the EC₅₀ or K_{D} of the unmasked antibody to bind to the target antigen in its activated (e.g., uninhibited, unmasked, and/or cleaved) state, or relative to EC₅₀ or K_{D} of the parental antibody (e.g., not linked to a masking peptide) to bind to the target antigen. The EC₅₀ values may be measured in an ELISA assay, or a Jurkat NFAT reporter assay, for example, as described in U.S. Pat. App. Pub. No. US2021/0207126 A1. The K_{D} values may be measured by, for example, using surface plasmon resonance.

The term “epitope” refers to a part of an antigen to which an antibody (or antigen-binding fragment thereof) binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope can include various numbers of amino acids in a unique spatial conformation. Methods of determining spatial conformation of epitopes include, for example, x-ray crystallography, 2-dimensional nuclear magnetic resonance, deuterium and hydrogen exchange in combination with mass spectrometry, or site-directed mutagenesis, or all methods used in combination with computational modeling of antigen and its complex structure with its binding antibody and its variants (see e.g., Epitope Mapping Protocols in Methods in Molecular Biology, Vol. 66, G. E. Morris, Ed. (1996)). Once a desired epitope of an antigen is determined, antibodies to that epitope can be generated, e.g., using the techniques described herein. The generation and characterization of antibodies may also elucidate information about desirable epitopes. From this information, it is then possible to competitively screen antibodies for binding to the same epitope. An approach to achieve this is to conduct cross-competition studies to find antibodies that competitively bind with one another, i.e., the antibodies compete for binding to the antigen. A high throughput process for “binning” antibodies based upon their cross-competition is described in PCT Publication No. WO 03/48731.

The term “germline” refers to the nucleotide sequences of the antibody genes and gene segments as they are passed from parents to offspring via the germ cells. The germline sequence is distinguished from the nucleotide sequences encoding antibodies in mature B cells which have been altered by recombination and hypermutation events during the course of B cell maturation.

The term “glycosylation sites” refers to amino acid residues which are recognized by a eukaryotic cell as locations for the attachment of sugar residues. The amino acids where carbohydrate, such as oligosaccharide, is attached are typically asparagine (N-linkage), serine (O-linkage), and threonine (O-linkage) residues. The specific site of attachment is typically signaled by a sequence of amino acids, referred to herein as a “glycosylation site sequence”. The glycosylation site sequence for N-linked glycosylation is: -Asn-X-Ser- or -Asn-X-Thr-, where X may be any of the conventional amino acids, other than proline. The terms “N-linked” and “O-linked” refer to the chemical group that serves as the attachment site between the sugar molecule and the amino acid residue. N-linked sugars are attached through an amino group; O-linked sugars are attached through a hydroxyl group. The term “glycan occupancy” refers to the existence of a carbohydrate moiety linked to a glycosylation site (i.e., the glycan site is occupied). Where there are at least two potential glycosylation sites on a polypeptide, either none (0-glycan site occupancy), one (1-glycan site occupancy) or both (2-glycan site occupancy) sites can be occupied by a carbohydrate moiety.

The term “host cell” refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; human cells (e.g., HEK293F cells, HEK293T cells; or human tissues or hybridoma cells, yeast cells, insect cells (e.g., S2 cells), bacterial cells (e.g., E. coli cells) and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.”

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “humanized antibody” refers to a chimeric antibody that contains amino acid residues derived from human antibody sequences. A humanized antibody may contain some or all of the CDRs from a non-human animal or synthetic antibody while the framework and constant regions of the antibody contain amino acid residues derived from human antibody sequences.

The term “exemplary antibody” refers to any one of the antibodies described herein. These antibodies may be in any class (e.g., IgA, IgD, IgE, IgG, and IgM). Thus, each antibody identified above encompasses antibodies in all five classes that have the same amino acid sequences for the V_{L} and V_{H} regions. Further, the antibodies in the IgG class may be in any subclass (e.g., IgG₁ IgG₂, IgG₃, and IgG₄). Thus, each antibody identified above in the IgG subclass encompasses antibodies in all four subclasses that have the same amino acid sequences for the V_{L} and V_{H} regions. The amino acid sequences of the heavy chain constant regions of human antibodies in the five classes, as well as in the four IgG subclasses, are known in the art.

An “isolated” antibody or binding molecule is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see e.g., Flatman et al., J. Chromatogr. B 848: 79-87 (2007). The term “k_{d}” refers to the association rate constant of a particular antibody-antigen interaction, whereas the term “k_{d}” refers to the dissociation rate constant of a particular antibody-antigen interaction.

The term “K_{D}” refers to the equilibrium dissociation constant of a particular antibody-antigen interaction. It is obtained from the ratio of k_{d} to k_{d} (i.e., k_{d}/k_{d}) and is expressed as a molar concentration (M). K_{D} is used as a measure for the affinity of an antibody's binding to its binding partner. The smaller the K_{D}, the more tightly bound the antibody is, or the higher the affinity between antibody and the antigen. For example, an antibody with a nanomolar (nM) dissociation constant binds more tightly to a particular antigen than an antibody with a micromolar (μM) dissociation constant. K_{D} values for antibodies can be determined using methods well established in the art. One method for determining the K_{D} of an antibody is by using an ELISA. For example, an assay procedure using an ELISA.

The term “mammal” refers to any animal species of the Mammalia class. Examples of mammals include: humans; laboratory animals such as rats, mice, hamsters, rabbits, non-human primates, and guinea pigs; domestic animals such as cats, dogs, cattle, sheep, goats, horses, and pigs; and captive wild animals such as lions, tigers, elephants, and the like.

The term “prevent” or “preventing,” with reference to a certain disease condition in a mammal, refers to preventing or delaying the onset of the disease, or preventing the manifestation of clinical or subclinical symptoms thereof.

As used herein, “sequence identity” between two polypeptide sequences indicates the percentage of amino acids that are identical between the sequences. The amino acid sequence identity of polypeptides can be determined conventionally using known computer programs such as Bestfit, FASTA, or BLAST (see e.g., Pearson, Methods Enzymol. (1990) 183: 63-98; Pearson, Methods Mol. Biol. (2000) 132: 185-219; Altschul et al., J. Mol. Biol. (1990) 215: 403-10; Altschul et al., Nucleic Acids Res. (1997) 25: 3389-3402). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, fragments, or variants thereof disclosed herein.

As used herein, the term “binds,” “binds to,” “specifically binds” “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding. For example, a masked anti-CD28 antibody described herein is said to selectively bind to human CD28 if it binds to human CD28 at an EC₅₀ that is below 10 percent of the EC₅₀ at which it binds to different antigen in an in vitro assay.

The term “treat,” “treating,” or “treatment,” with reference to a certain disease condition in a mammal, refers causing a desirable or beneficial effect in the mammal having the disease condition. The desirable or beneficial effect may include reduced frequency or severity of one or more symptoms of the disease (i.e., tumor growth and/or metastasis, or other effect mediated by the numbers and/or activity of immune cells, and the like), or arrest or inhibition of further development of the disease, condition, or disorder. In the context of treating cancer in a mammal, the desirable or beneficial effect may include inhibition of further growth or spread of cancer cells, death of cancer cells, inhibition of reoccurrence of cancer, reduction of pain associated with the cancer, or improved survival of the mammal. The effect can be either subjective or objective. For example, if the mammal is human, the human may note improved vigor or vitality or decreased pain as subjective symptoms of improvement or response to therapy. Alternatively, the clinician may notice a decrease in tumor size or tumor burden based on physical exam, laboratory parameters, tumor markers or radiographic findings. Some laboratory signs that the clinician may observe for response to treatment include normalization of tests, such as white blood cell count, red blood cell count, platelet count, erythrocyte sedimentation rate, and various enzyme levels. Additionally, the clinician may observe a decrease in a detectable tumor marker. Alternatively, other tests can be used to evaluate objective improvement, such as sonograms, nuclear magnetic resonance testing and positron emissions testing.

The term “vector” refers to a nucleic acid molecule capable of transporting a foreign nucleic acid molecule. The foreign nucleic acid molecule is linked to the vector nucleic acid molecule by a recombinant technique, such as ligation or recombination. This allows the foreign nucleic acid molecule to be multiplied, selected, further manipulated or expressed in a host cell or organism. A vector can be a plasmid, phage, transposon, cosmid, chromosome, virus, or virion. One type of vectors can be integrated into the genome of a host cell upon introduction into the host cell, and thereby are replicated along with the host genome (e.g., non-episomal mammalian vectors). Another type of vector is capable of autonomous replication in a host cell into which it is introduced (e.g., bacterial vectors having a bacterial origin of replication and episomal mammalian vectors). Another specific type of vector capable of directing the expression of expressible foreign nucleic acids to which they are operatively linked is commonly referred to as “expression vectors.” Expression vectors generally have control sequences that drive expression of the expressible foreign nucleic acids. Simpler vectors, known as “transcription vectors,” are only capable of being transcribed but not translated: they can be replicated in a target cell but not expressed. The term “vector” encompasses all types of vectors regardless of their function. Vectors capable of directing the expression of expressible nucleic acids to which they are operatively linked are commonly referred to “expression vectors.” Other examples of “vectors” may include display vectors (e.g., vectors that direct expression and display of an encoded polypeptide on the surface of a virus or cell (such as a bacterial cell, yeast cell, insect cell, and/or mammalian cell).

As used herein, a “subject”, “patient”, or “individual” may refer to a human or a non-human animal. A “non-human animal” may refer to any animal not classified as a human, such as domestic, farm, or zoo animals, sports, pet animals (such as dogs, horses, cats, cows, etc.), as well as animals used in research. Research animals may refer without limitation to nematodes, arthropods, vertebrates, mammals, frogs, rodents (e.g., mice or rats), fish (e.g., zebrafish or pufferfish), birds (e.g., chickens), dogs, cats, and non-human primates (e.g., rhesus monkeys, cynomolgus monkeys, chimpanzees, etc.). In some embodiments, the subject, patient, or individual is a human.

An “effective amount” refers to at least an amount effective, at dosages and for periods of time necessary, to achieve one or more desired or indicated effects, including a therapeutic or prophylactic result. An effective amount can be provided in one or more administrations. For purposes of the present disclosure, an effective amount of antibody, drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition (e.g., an effective amount as administered as a monotherapy or combination therapy). Thus, an “effective amount” may be considered in the context of administering one or more therapeutic agents, and a single agent may be considered to be given in an effective amount if, in conjunction with one or more other agents, a desirable result may be or is achieved.

All references cited herein, including patent applications and publications, are hereby incorporated by reference in their entirety.

### II. Antibodies

Certain aspects of the present disclosure relate to monospecific antibodies (e.g., traditional, non-masked monospecific antibodies), multi-specific antibodies (e.g., non-masked multi-specific antibodies), masked antibodies (e.g., activatable monospecific or multi-specific antibodies), antigen-binding fragments thereof, or derivatives of such antibodies.

### A. Fc Regions and C_{H}3 Domains

In some embodiments, the antibody (e.g., multi-specific antibody) described herein comprises one or more antibody constant regions, such as human heavy chain constant regions and/or human light chain constant regions. In some embodiments, the human heavy chain constant region is of an isotype selected from IgA, IgG, and IgD. In some embodiments, the human light chain constant region is of an isotype selected from K and k. In some embodiments, the antibody comprises a human IgG constant region. In some embodiments, the antibody comprises a human IgG₄ heavy chain constant region. In some embodiments, the antibody comprises a human IgG₁ heavy chain constant region. In some such embodiments, the antibody comprises an S228P mutation in the human IgG₄ constant region.

Whether or not effector function is desirable may depend on the particular method of treatment intended for an antibody. In some embodiments, when effector function is desirable, an antibody comprising a human IgG₁ heavy chain constant region or a human IgG₃ heavy chain constant region is selected. In some embodiments, when effector function is not desirable, an antibody comprising a human IgG₄ or IgG₂ heavy chain constant region is selected. In some embodiments, the antibody comprises a human IgG₁ heavy chain constant region comprising one or more mutations that reduces effector function. In some embodiments, the antibody comprises an IgG₁ heavy chain constant region comprising an N297A substitution.

The multi-specific antibodies (including the activatable multi-specific antibodies) described herein may comprise C_{H}3 domains having one or more engineered disulfide bonds, one or more engineered (e.g., rearranged or inversed) salt bridges, or a combination thereof. Unless stated otherwise, all amino acid residue numbering herein is based on Eu numbering, and the amino acid substitutions are relative to the wildtype (or naturally occurring) sequence at the corresponding amino acid positions in a wild type (or naturally occurring) C_{H}3 domain sequence. It is appreciated that the mutations or substitutions described herein are applicable to all IgG subclasses and allotypes. IgG allotypes have been described, for example, in Jefferis and Lefranc, mAbs (2009) 1: 4, 1-7, which is incorporated herein by reference in its entirety. In some embodiments, the amino acid mutations or substitutions described herein are relative to a wildtype C_{H}3 domain sequence of an IgG1, such as IgG1 allotype G1m, 1 (a), 2 (x), 3 (f) or 17 (z). In some embodiments, the amino acid mutations or substitutions described herein are relative to a wildtype C_{H}3 domain sequence of an IgG₄. For example, a D356K substitution relative to a wildtype C_{H}3 domain of one human IgG₁ allotype (Uniprot ID P01857) is equivalent to an E356K substitution relative to a wildtype C_{H}3 domain of a second human IgG₁ allotype, or a wildtype C_{H}3 domain of a human IgG₄. Exemplary C_{H}3 domain mutations are shown in Tables 2 and 3. In some embodiments, the amino acid mutations or substitutions described herein are relative to a wildtype Fc region sequence, e.g., an IgG₁ Fc region or an IgG₄ Fc region. C_{H}3 sequences with the mutations are described in WO 2021/148006, the disclosure of which is incorporated herein by reference in its entirety. In the tables below and elsewhere in the specification, the apostrophes in the C_{H}3 mutation annotations denote residues in the second C_{H}3 domain. For example, in N390C-S400′C, the S400C mutation is in the second C_{H}3 domain.

TABLE 2
Fc mutations

| Designs | Mutations (first C_{H}3 domain-second C_{H}3 domain) |
|---|---|
| Disulfide | N390C-S400′C |
| bond | S400C-N390′C |
| | K392C-V397′C |
| | V397C-K392′C |
| | K392C-S400′C |
| | S400C-K392′C |
| Charge | E357K:T411K-L351′D:K370′D |
| designs | E357K:S364K-L351′D:K370′D |
| | D356K:E357K:S364K-L351′D:K370′D:K439′D |
| Charge + | E357K:S364K:N390C-L351′D:K370′D:S400′C |
| disulfide | E357K:S364K:S400C-L351′D:K370′D:N390′C |
| bond designs | D356K:E357K:S364K:N390C- |
| | L351′D:K370′D:S400′C:K439′D |
| | D356K:E357K:S364K:S400C- |
| | L351′D:K370′D:N390′C:K439′D |

TABLE 3
Fc mutations ID

| Mutation ID | Mutations (first C_{H}3 domain-second C_{H}3 domain) |
|---|---|
| TRF01 | T366S, L368A, Y407V, Y349C-T366′W, S354′C |
| TRF02 | T350V, L351Y, F405A, Y407V-T350′V, T366′L, K392′L, T394′W |
| TRF03 | K196Q, S228P, F296Y, E356K, R409K, H435R, L445P-K196′Q, S228′P; |
| | F296′Y, R409′K, K439′E, L445′P |
| TYM01 | T366S, L368A, Y407V, N390C-T366′W, S400′C |
| TYM02 | T366S, L368A, Y407V, S400C-T366′W, N390′C |
| TYM03 | Y349C, L368V, Y407V-S354′C, T366′W |
| TYM04 | L368V, Y407V-T366′W |
| TYM05 | L368V, Y407V, N390C-T366′W, S400′C |
| TYM06 | L368V, Y407V, S400C-T366′W, N390′C |
| TYM07 | E357K:T411K-L351′D:K370′D |
| TYM08 | E357K:S364K-L351′D:K370′D |
| TYM09 | D or E356K:E357K:S364K-L351′D:K370′D:K439′D |
| TYM10 | E357K:S364K:N390C-L351′D:K370′D:S400′C |
| TYM11 | E357K:S364K:S400C-L351′D:K370′D:N390′C |
| TYM12 | D or E356K:E357K:S364K:N390C-L351′D:K370′D:S400′C:K439′D |
| TYM13 | D or E356K:E357K:S364K:S400C-L351′D:K370′D:N390′C:K439′D |

In some embodiments, the multi-specific antibody (e.g., the activatable multi-specific antibody) comprises an engineered disulfide bond between C390 in a first C_{H}3 domain and C400 in a second C_{H}3 domain, between C392 in a first C_{H}3 domain and C397 in a second C_{H}3 domain, or between C392 in a first C_{H}3 domain and C400 in a second C_{H}3 domain. In some embodiments, the multi-specific antibody (e.g., the activatable multi-specific antibody) comprises a rearranged salt-bridge network as compared to wildtype C_{H}3 domains, e.g., among positions 357 and 411 in a first C_{H}3 domain and positions 351 and 370 in a second C_{H}3 domain (e.g., E357K: T411K-L351′D: K370′D), or among positions 357 and 364 in a first C_{H}3 domain and positions 351 and 370 in a second C_{H}3 domain (e.g., E357K: S364K-L351′D: K370′D). In some embodiments, the multi-specific antibody comprises an inversed salt bridge as compared to wildtype C_{H}3 domains between position 356 in a first C_{H}3 domain and position 439 in a second C_{H}3 domain (e.g., D356-K439′). The antibodies having C_{H}3 mutations may have high yield, superior stability (e.g., resistance to aggregation and precipitation at high temperature or due to freeze-thaw cycles), and potent activity.

In some embodiments, the multi-specific antibody (e.g., the activatable multi-specific antibody) comprises C_{H}3 domains having one or more engineered residues, which promote heterodimer formation as described herein. Heteromultimers comprising multiple heterodimers formed by a first polypeptide comprising a first engineered C_{H}3 domain and a second polypeptide comprising a second engineered C_{H}3 domain are also contemplated herein.

In some embodiments, the multi-specific antibody (e.g., the activatable multi-specific antibody) comprises: i) a first C_{H}3 domain comprising a cysteine (C) residue at position 390 and a second C_{H}3 domain comprising a cysteine residue at position 400, or a first C_{H}3 domain comprising a cysteine residue at position 400 and a second C_{H}3 domain comprising a cysteine residue at position 390; or ii) a first C_{H}3 domain comprising a cysteine residue at position 392 and a second C_{H}3 domain comprising a cysteine residue at position 397, or a first C_{H}3 domain comprising a cysteine residue at position 397 and a second C_{H}3 domain comprising a cysteine residue at position 392; or iii) a first C_{H}3 domain comprising a cysteine residue at position 392 and a second C_{H}3 domain comprising a cysteine residue at position 400, or a first C_{H}3 domain comprising a cysteine residue at position 400 and a second C_{H}3 domain comprising a cysteine residue at position 392; and wherein the amino acid residue numbering is based on Eu numbering.

In some embodiments, the multi-specific antibody (e.g., the activatable multi-specific antibody) comprises: a first polypeptide comprising a first C_{H}3 domain and a second polypeptide comprising a second C_{H}3 domain, wherein: i) the first C_{H}3 domain further comprises a positively charged residue at position 357 and the second C_{H}3 domain further comprises a negatively charged residue at position 351, or the first C_{H}3 domain further comprises a negatively charged residue at position 351 and the second C_{H}3 domain further comprises a positively charged residue at position 357; or ii) the first C_{H}3 domain further comprises a positively charged residue at position 411 and the second C_{H}3 domain further comprises a negatively charged residue at position 370, or the first C_{H}3 domain further comprises a negatively charged residue at position 370 and the second C_{H}3 domain further comprises a positively charged residue at position 411; or iii) the first C_{H}3 domain further comprises a positively charged residue at position 364 and the second C_{H}3 domain further comprises a negatively charged residue at position 370, or the first C_{H}3 domain further comprises a negatively charged residue at position 370 and the second C_{H}3 domain further comprises a positively charged residue at position 364; or a combination of i) and ii), or a combination or i) and iii), wherein the amino acid residue numbering is based on Eu numbering.

In some embodiments, the first C_{H}3 domain further comprises a positively charged residue at position 356 and the second C_{H}3 domain further comprises a negatively charged residue at position 439, or the first C_{H}3 domain further comprises a negatively charged residue at position 439 and the second C_{H}3 domain further comprises a positively charged residue at position 356, and wherein the amino acid residue numbering is based on Eu numbering.

In some embodiments, the multi-specific antibody (e.g., the activatable multi-specific antibody) comprises: a first polypeptide comprising a first C_{H}3 domain and a second polypeptide comprising a second C_{H}3 domain, wherein: i) the first C_{H}3 domain comprises a cysteine (C) residue at position 390 and the second C_{H}3 domain comprises a cysteine residue at position 400, or the first C_{H}3 domain comprises a cysteine residue at position 400 and the second C_{H}3 domain comprises a cysteine residue at position 390; or ii) the first C_{H}3 domain comprises a cysteine residue at position 392 and the second C_{H}3 domain comprises a cysteine residue at position 397, or the first C_{H}3 domain comprises a cysteine residue at position 397 and the second C_{H}3 domain comprises a cysteine residue at position 392; or iii) the first C_{H}3 domain comprises a cysteine residue at position 392 and the second C_{H}3 domain comprises a cysteine residue at position 400, or the first C_{H}3 domain comprises a cysteine residue at position 400 and the second C_{H}3 domain comprises a cysteine residue at position 392; and wherein: a) the first C_{H}3 domain further comprises a positively charged residue at position 357 and the second C_{H}3 domain further comprises a negatively charged residue at position 351, or the first C_{H}3 domain further comprises a negatively charged residue at position 351 and the second C_{H}3 domain further comprises a positively charged residue at position 357; or b) the first C_{H}3 domain further comprises a positively charged residue at position 411 and the second C_{H}3 domain further comprises a negatively charged residue at position 370, or the first C_{H}3 domain further comprises a negatively charged residue at position 370 and the second C_{H}3 domain further comprises a positively charged residue at position 411; or c) the first C_{H}3 domain further comprises a positively charged residue at position 364 and the second C_{H}3 domain further comprises a negatively charged residue at position 370, or the first C_{H}3 domain further comprises a negatively charged residue at position 370 and the second C_{H}3 domain further comprises a positively charged residue at position 364; or a combination of a) and b), or a combination of a) and c); wherein the amino acid residue numbering is based on Eu numbering.

In some embodiments, the first C_{H}3 domain further comprises a positively charged residue at position 356 and the second C_{H}3 domain further comprises a negatively charged residue at position 439, or first C_{H}3 domain further comprises a negatively charged residue at position 439 and the second C_{H}3 domain further comprises a positively charged residue at position 356, and wherein the amino acid residue numbering is based on Eu numbering.

The C_{H}3 domains may be derived from any naturally occurring immunoglobulin molecules. In some embodiments, the C_{H}3 domains are derived from an IgG1 molecule, an IgG₂ molecule, an IgG₃ molecule, or an IgG₄ molecule. In some embodiments, the C_{H}3 domains are human C_{H}3 domains. In some embodiments, the C_{H}3 domains are derived from human IgG₁ molecules.

In some embodiments, the multi-specific antibody (e.g., the activatable multi-specific antibody) comprises: a first polypeptide comprising a first C_{H}3 domain and a second polypeptide comprising a second C_{H}3 domain, wherein: i) the first C_{H}3 domain comprises N390C substitution and the second C_{H}3 domain comprises S400C substitution, or the first C_{H}3 domain comprises S400C substitution and the second C_{H}3 domain comprises N390C substitution; or ii) the first C_{H}3 domain comprises K392C substitution and the second C_{H}3 domain comprises V397C substitution, or the first C_{H}3 domain comprises V397C substitution and the second C_{H}3 domain comprises K392C substitution; or iii) the first C_{H}3 domain comprises K392C substitution and the second C_{H}3 domain comprises S400C substitution, or the first C_{H}3 domain comprises S400C substitution and the second C_{H}3 domain comprises K392C substitution.

In some embodiments, the multi-specific antibody (e.g., the activatable multi-specific antibody) comprises: a first polypeptide comprising a first C_{H}3 domain and a second polypeptide comprising a second C_{H}3 domain, wherein: i) the first C_{H}3 domain comprises E357K and T411K substitutions and the second C_{H}3 domain comprises L351D and K370D substitutions, or the first C_{H}3 domain comprises L351D and K370D substitutions and the second C_{H}3 domain comprises E357K and T411K substitutions; or ii) the first C_{H}3 domain comprises E357K and S364K substitutions and the second C_{H}3 domain comprises L351D and K370D substitutions, or the first C_{H}3 domain comprises L351D and K370D substitutions and the second C_{H}3 domain comprises E357K and S364K substitutions; or iii) the first C_{H}3 domain comprises D356K, E357K, and S364K substitutions and the second C_{H}3 domain comprises L351D, K370D, and K439D substitutions, or the first C_{H}3 domain comprises L351D, K370D, and K439D substitutions and the second C_{H}3 domain comprises D356K, E357K and S364K substitutions.

In some embodiments, the multi-specific antibody (e.g., the activatable multi-specific antibody) comprises: a first polypeptide comprising a first C_{H}3 domain and a second polypeptide comprising a second C_{H}3 domain, wherein the first C_{H}3 domain comprises E357K, S364K, and N390C substitutions and the second C_{H}3 domain comprises L351D, K370D, and S400C substitutions, or the first C_{H}3 domain comprises L351D, K370D, and S400C substitutions and the second C_{H}3 domain comprises E357K, S364K, and N390C substitutions.

In some embodiments, the multi-specific antibody (e.g., the activatable multi-specific antibody) comprises: a first polypeptide comprising a first C_{H}3 domain and a second polypeptide comprising a second C_{H}3 domain, wherein the first C_{H}3 domain comprises E357K, S364K, and S400C substitutions and the second C_{H}3 domain comprises L351D, K370D, and N390C substitutions, or the first C_{H}3 domain comprises L351D, K370D, and N390C substitutions and the second C_{H}3 domain comprises E357K, S364K, and S400C substitutions.

In some embodiments, the multi-specific antibody (e.g., the activatable multi-specific antibody) comprises: a first polypeptide comprising a first C_{H}3 domain and a second polypeptide comprising a second C_{H}3 domain, wherein the first C_{H}3 domain comprises D356K, E357K, S364K, and S400C substitutions and the second C_{H}3 domain comprises L351D, K370D, N390C, and K439D substitutions, or the first C_{H}3 domain comprises L351D, K370D, N390C, and K439D substitutions and the second C_{H}3 domain comprises D356K, E357K, S364K, and S400C substitutions.

In some embodiments, the multi-specific antibody (e.g., the activatable multi-specific antibody) comprises: a first polypeptide comprising a first C_{H}3 domain and a second polypeptide comprising a second C_{H}3 domain, wherein the first C_{H}3 domain comprises D356K, E357K, S364K, and N390C substitutions and the second C_{H}3 domain comprises L351D, K370D, K439D, and S400C substitutions, or the first C_{H}3 domain comprises L351D, K370D, K439D, and S400C substitutions and the second C_{H}3 domain comprises D356K, E357K, S364K, and N390C substitutions.

In some embodiments, the multi-specific antibody (e.g., the activatable multi-specific antibody) comprises an IgG Fc region that comprises the engineered C_{H}3 domains. The Fc region may be derived from any suitable Fc subclasses, including, but not limited to, IgG₁, IgG₂, IgG₃, and IgG₄ subclasses.

### B. Cysteine Mutations

In some embodiments, the multi-specific antibodies described herein (e.g., the activatable multi-specific antibodies described herein) comprise a first polypeptide comprising a first C_{H}3 domain and a second polypeptide comprising a second C_{H}3 domain, wherein the first C_{H}3 domain comprises a first engineered cysteine residue and the second C_{H}3 domain comprises a second engineered cysteine residue, wherein the first engineered cysteine residue and the second cysteine residue form a disulfide bond.

In some embodiments, the first C_{H}3 domain comprises a C at position 390 and the second C_{H}3 domain comprises a C at position 400, or the first C_{H}3 domain comprises a C at position 400 and the second C_{H}3 domain comprises a C at position 390. In some embodiments, the first C_{H}3 domain comprises N390C substitution and the second C_{H}3 domain comprises S400C substitution, or the first C_{H}3 domain comprises S400C substitution and the second C_{H}3 domain comprises N390C substitution.

In some embodiments, the first C_{H}3 domain comprises a C at position 392 and the second C_{H}3 domain comprises a C at position 397, or the first C_{H}3 domain comprises a C at position 397 and the second C_{H}3 domain comprises a C at position 392. In some embodiments, the first C_{H}3 domain comprises K392C substitution and the second C_{H}3 domain comprises V397C substitution, or the first C_{H}3 domain comprises V397C substitution and the second C_{H}3 domain comprises K392C substitution.

In some embodiments, the first C_{H}3 domain comprises a C at position 392 and the second C_{H}3 domain comprises a C at position 400, or the first C_{H}3 domain comprises a C at position 400 and the second C_{H}3 domain comprises a C at position 392. In some embodiments, the first C_{H}3 domain comprises K392C substitution and the second C_{H}3 domain comprises S400C substitution, or the first C_{H}3 domain comprises S400C substitution and the second C_{H}3 domain comprises K392C substitution.

### C. Salt Bridge Mutations

In some embodiments, the multi-specific antibodies described herein (e.g., the activatable multi-specific antibodies described herein) comprise a first polypeptide comprising a first C_{H}3 domain and a second polypeptide comprising a second C_{H}3 domain, wherein the first C_{H}3 domain comprises an engineered positively charged residue and the second C_{H}3 domain comprises an engineered negatively charged residue, wherein the engineered positively charged residue and the engineered negatively charged residue form a salt bridge. The engineered salt bridge may introduce new salt bridges between the C_{H}3 domains, rearrange a salt-bridge network among two or more amino acid residues, or reverse the charges on the amino acid residues forming the salt bridge (i.e., “inverse” a salt bridge) with respect to wildtype C_{H}3 domains. In some embodiments, the engineered positively charged residue substitutes a negatively charged residue in a wildtype C_{H}3 domain with a positively charged residue. In some embodiments, the engineered negatively charged residue substitutes a positively charged residue in a wildtype C_{H}3 domain with a negatively charged residue. The rearranged and inversed salt bridges may result in changes in the isoelectric points (PI) of the heterodimer and the homodimer comprising the engineered C_{H}3 domains, thereby allowing better separation of the heterodimer from the homodimer in a purification process.

In some embodiments, the first C_{H}3 domain comprises a positively charged residue at position 357 and the second C_{H}3 domain comprises a negatively charged residue at position 351, or the first C_{H}3 domain comprises a negatively charged residue at position 351 and the second C_{H}3 domain comprises a positively charged residue at position 357. In some embodiments, the first C_{H}3 domain comprises a K at position 357 and the second C_{H}3 domain comprises a D at position 351, or the first C_{H}3 domain comprises a D at position 351 and the second C_{H}3 domain comprises a K at position 357. In some embodiments, the first C_{H}3 domain comprises a K at position 357 and the second C_{H}3 domain comprises an E at position 351, or the first C_{H}3 domain comprises an E at position 351 and the second C_{H}3 domain comprises a K at position 357. In some embodiments, the first C_{H}3 domain comprises an R at position 357 and the second C_{H}3 domain comprises a D at position 351, or the first C_{H}3 domain comprises a D at position 351 and the second C_{H}3 domain comprises an R at position 357. In some embodiments, the first C_{H}3 domain comprises an R at position 357 and the second C_{H}3 domain comprises an E at position 351, or the first C_{H}3 domain comprises an E at position 351 and the second C_{H}3 domain comprises an R at position 357. In some embodiments, the first C_{H}3 domain comprises E357K substitution and the second C_{H}3 domain comprises L351D substitution, or the first C_{H}3 domain comprises L351D substitution and the second C_{H}3 domain comprises E357K substitution.

In some embodiments, the first C_{H}3 domain comprises a positively charged residue at position 411 and the second C_{H}3 domain comprises a negatively charged residue at position 370, or the first C_{H}3 domain comprises a negatively charged residue at position 370 and the second C_{H}3 domain comprises a positively charged residue at position 411. In some embodiments, the first C_{H}3 domain comprises a K at position 411 and the second C_{H}3 domain comprises a D at position 370, or the first C_{H}3 domain comprises a D at position 370 and the second C_{H}3 domain comprises a K at position 411. In some embodiments, the first C_{H}3 domain comprises a K at position 411 and the second C_{H}3 domain comprises an E at position 370, or the first C_{H}3 domain comprises an E at position 370 and the second C_{H}3 domain comprises a K at position 411. In some embodiments, the first C_{H}3 domain comprises an R at position 411 and the second C_{H}3 domain comprises a D at position 370, or the first C_{H}3 domain comprises a D at position 370 and the second C_{H}3 domain comprises an R at position 411. In some embodiments, the first C_{H}3 domain comprises an R at position 411 and the second C_{H}3 domain comprises an E at position 370, or the first C_{H}3 domain comprises an E at position 370 and the second C_{H}3 domain comprises an R at position 411. In some embodiments, the first C_{H}3 domain comprises T411K substitution and the second C_{H}3 domain comprises K370D substitution, or the first C_{H}3 domain comprises K370D substitution and the second C_{H}3 domain comprises T411K substitution.

In some embodiments, the first C_{H}3 domain comprises a positively charged residue at position 364 and the second C_{H}3 domain comprises a negatively charged residue at position 370, or the first C_{H}3 domain comprises a negatively charged residue at position 370 and the second C_{H}3 domain comprises a positively charged residue at position 364. In some embodiments, the first C_{H}3 domain comprises a K at position 364 and the second C_{H}3 domain comprises a D at position 370, or the first C_{H}3 domain comprises a D at position 370 and the second C_{H}3 domain comprises a K at position 364. In some embodiments, the first C_{H}3 domain comprises a K at position 364 and the second C_{H}3 domain comprises an E at position 370, or the first C_{H}3 domain comprises an E at position 370 and the second C_{H}3 domain comprises a K at position 364. In some embodiments, the first C_{H}3 domain comprises an R at position 364 and the second C_{H}3 domain comprises a D at position 370, or the first C_{H}3 domain comprises a D at position 370 and the second C_{H}3 domain comprises an R at position 364. In some embodiments, the first C_{H}3 domain comprises an R at position 364 and the second C_{H}3 domain comprises an E at position 370, or the first C_{H}3 domain comprises an E at position 370 and the second C_{H}3 domain comprises an R at position 364. In some embodiments, the first C_{H}3 domain comprises S364K substitution and the second C_{H}3 domain comprises K370D substitution, or the first C_{H}3 domain comprises K370D substitution and the second C_{H}3 domain comprises S364K substitution.

In some embodiments, the first C_{H}3 domain comprises a positively charged residue at position 356 and the second C_{H}3 domain comprises a negatively charged residue at position 439, or the first C_{H}3 domain comprises a negatively charged residue at position 439 and the second C_{H}3 domain comprises a positively charged residue at position 356. In some embodiments, the first C_{H}3 domain comprises a K at position 356 and the second C_{H}3 domain comprises a D at position 439, or the first C_{H}3 domain comprises a D at position 439 and the second C_{H}3 domain comprises a K at position 356. In some embodiments, the first C_{H}3 domain comprises a K at position 356 and the second C_{H}3 domain comprises an E at position 439, or the first C_{H}3 domain comprises an E at position 439 and the second C_{H}3 domain comprises a K at position 356. In some embodiments, the first C_{H}3 domain comprises an R at position 356 and the second C_{H}3 domain comprises a D at position 439, or the first C_{H}3 domain comprises a D at position 439 and the second C_{H}3 domain comprises an R at position 356. In some embodiments, the first C_{H}3 domain comprises an R at position 356 and the second C_{H}3 domain comprises an E at position 439, or the first C_{H}3 domain comprises an E at position 439 and the second C_{H}3 domain comprises an R at position 356. In some embodiments, the first C_{H}3 domain comprises D356K substitution and the second C_{H}3 domain comprises K439D substitution, or the first C_{H}3 domain comprises K439D substitution and the second C_{H}3 domain comprises D356K substitution.

Any of the engineered salt bridges described herein may be combined with each other. In some embodiments, the first C_{H}3 domain comprises a positively charged residue at position 357 and a positively charged residue at position 411, and the second C_{H}3 domain comprises a negatively charged residue at position 351 and a negatively charged residue at position 370, or the first C_{H}3 domain comprises a negatively charged residue at position 351 and a negatively charged residue at position 370, and the second C_{H}3 domain comprises a positively charged residue at position 357 and a positively charged residue at position 411. In some embodiments, the first C_{H}3 domain comprises E357K and T411K substitutions, and the second C_{H}3 domain comprises L351D and K370D substitutions, or the first C_{H}3 domain comprises L351D and K370D substitutions, and the second C_{H}3 domain comprises E357K and T411K substitutions.

In some embodiments, the first C_{H}3 domain comprises a positively charged residue at position 357 and a positively charged residue at position 364, and the second C_{H}3 domain comprises a negatively charged residue at position 351 and a negatively charged residue at position 370, or the first C_{H}3 domain comprises a negatively charged residue at position 351 and a negatively charged residue at position 370, and the second C_{H}3 domain comprises a positively charged residue at position 357 and a positively charged residue at position 364. In some embodiments, the first C_{H}3 domain comprises E357K and S364K substitutions, and the second C_{H}3 domain comprises L351D and K370D substitutions, or the first C_{H}3 domain comprises L351D and K370D substitutions, and the second C_{H}3 domain comprises E357K and S364K substitutions.

In some embodiments, the first C_{H}3 domain comprises a positively charged residue at position 356, a positively charged residue at position 357, and a positively charged residue at position 364 and the second C_{H}3 domain comprises a negatively charged residue at position 351, a negatively charged residue at position 370, and a negatively charged residue at position 439, or the first C_{H}3 domain comprises a negatively charged residue at position 351, a negatively charged residue at position 370, and a negatively charged residue at position 439 and the second C_{H}3 domain comprises a positively charged residue at position 356, a positively charged residue at position 357, and a positively charged residue at position 364. In some embodiments, the first C_{H}3 domain comprises D356K, E357K, and S364K substitutions and the second C_{H}3 domain comprises L351D, K370D, and K439D substitutions, or the first C_{H}3 domain comprises L351D, K370D, and K439D substitutions and the second C_{H}3 domain comprises D356K, E357K, and S364K substitutions.

### D. Other Mutations

The C_{H}3 domains or the Fc regions described herein may further comprise engineered disulfide bonds and/or salt bridges listed in Table 4 below.

TABLE 4
Exemplary Fc Mutations

| Mutation(s) in first | |
|---|---|
| polypeptide chain | Mutation(s) in second polypeptide chain |
| F405L | K409R |
| S364H, F405A | Y349T, T394F |
| S364K, E357Q | L368D, K370S |
| T366W | T366S, L368A, Y407V |
| S354C, T366W | Y349C, T366S, L368A, Y407V |
| T350, L351, F405, Y407 | T350, T366, K392, T394 |
| T350 is T350V, T350I, T350L | T350 is T350V, T350I, T350L or T350M |
| or T350M | T366 is T366L, T366I, T366V or T366M |
| L351 is L351Y | K392 is K392F, K392L or K392M |
| F450 is F450A, F450V, F450T | T394 is T394W |
| or F450S | |
| Y407 is Y407V, Y407A or | |
| Y407I | |
| D399K, E356K | K409D, K392D |
| D221E, P228E, L368E | D221R, P228R, K409R |
| C223E, E225E, P228E, L368E | C223R, E225R, P228R, K409R |
| H435R | None |
| K196Q, S228P, F296Y, E356K, | K196Q, S228P, F296Y, R409K, K439E, L445P |
| R409K, H435R, L445P | |
| K409W | D399V/F40ST |
| K360E | Q347R |
| Y349C/K360E/K409W | Q347R/S354C/D399V/F405T |
| K360E/K409W | Q347R/D399V/F405T |
| Y349S/K409W | E357W/D399V/F405T |
| Y349S/S354C/K409W | Y349C/E357W/D399V/F405T |
| T366K | L351D |
| Y349E or D and L368E | L351D |
| Y349C/T366W | D356C/T366S/L368A/Y407V/F405K |
| Y349C/T366W/F405K | D356C/T366S/L368A/Y407V |
| Y349C/T366W/K409E | D356C/T366S/L368A/Y407V/F405K |
| Y349C/T366W/K409A | D356C/T366S/L368A/Y407V/F405K |
| S364K | L368D |
| S364K | K370S |
| F405K | K409F |
| F405R | K409F |
| S364K/K409F | L368D/F405R |
| S364K/K409F | K370S/F405R |
| S364K/K409W | K370S/F405R |
| K370E or E356K and K409R | E357K and K409R or K370E |
| S354I/S364L/Y407V | Q347V/Y349I/T350K/L351Q/T366W/L368I/Y407V |
| Y349I | S354I/E357Q/M/G/S364L/T366S/L368A/Y407V |
| P395K/P396K/V397K | T394D/P395D/P396D |
| F405E/Y407E/K409E | F405K/Y407K |
| M428S/N434S/Y436H | H435R |

In some embodiments, the first C_{H}3 domain further comprises a C at position 392 and the second C_{H}3 domain comprises a C at position 399, or the first C_{H}3 domain comprises a C at position 399 and the second C_{H}3 domain comprises a C at position 392. In some embodiments, the first C_{H}3 domain further comprises K392C substitution and the second C_{H}3 domain further comprises D399C substitution, or the first C_{H}3 domain further comprises D399C substitution and the second C_{H}3 domain further comprises K392C substitution.

In some embodiments, the first C_{H}3 domain further comprises a C at position 394 and the second C_{H}3 domain comprises a C at position 354, or the first C_{H}3 domain comprises a C at position 354 and the second C_{H}3 domain comprises a C at position 394. In some embodiments, the first C_{H}3 domain further comprises Y394C substitution and the second C_{H}3 domain further comprises S354C substitution, or the first C_{H}3 domain further comprises S354C substitution and the second C_{H}3 domain further comprises Y394C substitution.

In some embodiments, the first C_{H}3 domain further comprises a C at position 356 and the second C_{H}3 domain comprises a C at position 349, or the first C_{H}3 domain comprises a C at position 349 and the second C_{H}3 domain comprises a C at position 356. In some embodiments, the first C_{H}3 domain further comprises D356C substitution and the second C_{H}3 domain further comprises Y349C substitution, or the first C_{H}3 domain further comprises Y349C substitution and the second C_{H}3 domain further comprises D356C substitution.

In some embodiments, the first C_{H}3 domain further comprises K392D and K409D substitutions and the second C_{H}3 domain further comprises D356K and D399K substitutions, or the first C_{H}3 domain further comprises D356K and D399K substitutions and the second C_{H}3 domain further comprises K392D and K409D substitutions.

In some embodiments, the first C_{H}3 domain further comprises L368D and K370S substitutions and the second C_{H}3 domain further comprises E357Q and S364K substitutions, or the first C_{H}3 domain further comprises E357Q and S364K substitutions and the second C_{H}3 domain further comprises L368D and K370S substitutions.

In some embodiments, the first C_{H}3 domain further comprises L351K and T366K substitutions and the second C_{H}3 domain further comprises L351D and L368E substitutions, or the first C_{H}3 domain further comprises L351D and L368E substitutions and the second C_{H}3 domain further comprises L351K and T366K substitutions.

In some embodiments, the first C_{H}3 domain further comprises P395K, P396K, and V397K substitutions and the second C_{H}3 domain comprises T394D, P395D, and P396D substitutions, or the first C_{H}3 domain further comprises T394D, P395D, and P396D substitutions and the second C_{H}3 domain further comprises P395K, P396K, and V397K substitutions.

In some embodiments, the first C_{H}3 domain further comprises F405E, Y407E, and K409E substitutions and the second C_{H}3 domain comprises F405K and Y407K substitutions, or the first C_{H}3 domain further comprises F405K and Y407K substitutions and the second C_{H}3 domain further comprises F405E, Y407E and K409E substitutions.

In some embodiments, the first C_{H}3 domain further comprises T336S, L368A, and Y407V substitutions and the second C_{H}3 domain further comprises T366W substitution, or the first C_{H}3 domain further comprises T366W substitution and the second C_{H}3 domain further comprises T336S, L368A, and Y407V substitutions.

In some embodiments, the first C_{H}3 domain comprises L368V and Y407V substitutions and the second C_{H}3 domain comprises T366W substitution, or the first C_{H}3 domain comprises T366W substitution and the second C_{H}3 domain comprises L368V and Y407V substitutions.

### III. CD28-Binding Molecules

The present disclosure provides isolated binding molecules that bind to human CD28, including anti-CD28 antibodies and anti-CD28 antigen-binding fragments thereof. In some embodiments, the binding molecules include antibodies described with reference to epitope binding and antibodies described with reference to specific amino acid sequences of complementarity determining regions (CDR), variable regions (V_{L}, V_{H}), and IgG (e.g., IgG₄) light and heavy chains.

In some embodiments, the antibodies or the antigen-binding fragments thereof bind to one or more amino acid residues within amino acid residues 34-108 of SEQ ID NO: 1. In some embodiments, the antibodies or antigen-binding fragments bind to one or more amino acid residues within amino acid residues 51-122 of SEQ ID NO: 1. In some embodiments, the antibodies or antigen-binding fragments bind to one or more amino acid residues selected from the group consisting of amino acid residues 51, 52, 54, 55, 98-101, 110-111, 113-114, and 118-122 of SEQ ID NO: 1. Methods of measuring an antibody or antigen-binding fragment's ability to bind a target antigen may be carried out using any method known in the art, including for example, by surface plasmon resonance, an ELISA, isothermal titration calorimetry, a filter binding assay, an EMSA, etc. In some embodiments, the ability of the antibody or antigen-binding fragment to bind a target antigen is measured by ELISA or Octet® RED96 (see, e.g., Example 3 below).

In some embodiments, the antibodies or antigen-binding fragments bind to human CD28 with a K_{D} of about 500 nM or less (e.g., about 500 nM or less, about 400 nM or less, about 300 nM or less, about 200 nM or less, about 150 nM or less, about 100 nM or less, about 90 nM or less, about 80 nM or less, about 75 nM or less, about 70 nM or less, about 60 nM or less, about 50 nM or less, about 40 nM or less, about 30 nM or less, about 25 nM or less, about 20 nM or less, about 10 nM or less, about 1 nM or less, about 0.1 nM or less, etc.) In some embodiments, the antibodies or antigen-binding fragments bind to human CD28 with a K_{D} of about 100 nM or less. In some embodiments, the antibodies or antigen-binding fragments bind to human CD28 with a K_{D} of about 50 nM or less. Methods of measuring the K_{D} of an antibody or antigen-binding fragment may be carried out using any method known in the art, including for example, by surface plasmon resonance, an ELISA, isothermal titration calorimetry, a filter binding assay, an EMSA, etc. In some embodiments, the K_{D} is measured by Octet® RED96 Systems (See, e.g., Example 3 below).

### A. Anti-CD28 Antibodies

In some embodiments, the present disclosure provides an isolated monoclonal antibody that binds to human CD28 at an epitope within amino acid residues 33-37, 80-83, 92-96, and 100-104 of SEQ ID NO: 1. In particular embodiments, the present disclosure provides an isolated antibody that binds to human CD28 at an epitope represented by amino acid residues 33, 34, 36 and 37, 80-83, 92 and 93, 95 and 96, and 100-104 of SEQ ID NO: 1. The antibody, in some embodiments, binds human CD28 with a K_{D} of 10 nM or less as measured by Octet® RED96 Systems. In certain embodiments, in addition to binding human epitopes, the antibody disclosed herein is cross-reactive (exhibits cross-species binding features) with at least one non-human species selected from the list consisting of cynomolgus monkey, mouse, rat and dog. In certain embodiments, the antibody disclosed herein has the advantage of cross-species binding to mouse, humans and monkeys, whereas the benchmark controls TAC2386 and TAC2387 disclosed herein do not have this range of species cross-reactivity. In particular embodiments, the benchmark controls TAC2386 and TAC2387 bind human epitopes but not mouse epitopes (see Table 7 herein). The species cross reactivity of the antibody disclosed herein also provides the added advantage of being able to use a mouse to model the antibody's safety, activity, and function. Hence, compared to TAC2386 and TAC2387 disclosed herein, it is easier to do animal modeling with the antibody disclosed herein.

In particular embodiments, the isolated anti-CD28 monoclonal antibody comprises a HCDR1 of SEQ ID NO: 5, HCDR2 of SEQ ID NO: 6, and HCDR3 of SEQ ID NO:7, and a LCDR1 of SEQ ID NO: 8, a LCDR2 of SEQ ID NO: 9 and a LCDR3 of SEQ ID NO:10. In particular embodiments, the isolated monoclonal antibody comprises heavy chain variable region of a SEQ ID NO: 11 and light chain variable region of SEQ ID NO: 12. In particular embodiments, the isolated monoclonal antibody comprises a heavy chain of SEQ ID NO: 13 and light chain of SEQ ID NO: 14.

The CD28 antibodies described herein can be in any class, such as IgG, IgM, IgE, IgA, or IgD. It is preferred that the anti-CD28 antibodies are in the IgG class, such as IgG₁, IgG₂, IgG₃, or IgG₄ subclass. An anti-CD28 antibody can be converted from one class or subclass to another class or subclass using methods known in the art. An exemplary method for producing an antibody in a desired class or subclass comprises the steps of isolating a nucleic acid encoding a heavy chain of an anti-CD28 antibody and a nucleic acid encoding a light chain of a CD28 antibody, isolating the sequence encoding the V_{H} region, ligating the V_{H} sequence to a sequence encoding a heavy chain constant region of the desired class or subclass, expressing the light chain gene and the heavy chain construct in a cell, and collecting the CD28 antibody.

The anti-CD28 antibodies described herein can be in any class, such as IgG, IgM, IgE, IgA, or IgD. It is preferred that the anti-CD28 antibodies are in the IgG class, such as IgG₁, IgG₂, IgG₃, or IgG₄ subclass. An anti-CD28 antibody can be converted from one class or subclass to another class or subclass using methods known in the art. An exemplary method for producing an antibody in a desired class or subclass comprises the steps of isolating a nucleic acid encoding a heavy chain of an anti-CD28 antibody and a nucleic acid encoding a light chain of an anti-CD28 antibody, isolating the sequence encoding the V_{H} region, ligating the V_{H} sequence to a sequence encoding a heavy chain constant region of the desired class or subclass, expressing the light chain gene and the heavy chain construct in a cell, and collecting the CD28 antibody.

Further, the antibodies provided by the present disclosure can be monoclonal or polyclonal, but preferably monoclonal.

Antibodies of the present disclosure can be produced by techniques known in the art, including conventional monoclonal antibody methodology e.g., the standard somatic cell hybridization technique (see e.g., Kohler and Milstein, Nature (1975) 256: 495), viral or oncogenic transformation of B lymphocytes, or recombinant antibody technologies as described in detail herein below.

Hybridoma production is a very well-established procedure. The common animal system for preparing hybridomas is the murine system. Immunization protocols and techniques for isolation of immunized splenocytes for fusion are known in the art. Fusion partners (e.g., murine myeloma cells) and fusion procedures are also known. One well-known method that may be used for making human CD28 antibodies provided by the present disclosure involves the use of a XenoMouse™ animal system. XenoMouse™ mice are engineered mouse strains that comprise large fragments of human immunoglobulin heavy chain and light chain loci and are deficient in mouse antibody production. See, e.g., Green et al., Nature Genetics (1994) 7: 13-21 and WO2003/040170. The animal is immunized with an CD28 antigen. The CD28 antigen is isolated and/or purified CD28, preferably CD28. It may be a fragment of CD28, such as the extracellular domain of CD28, particularly a CD28 extracellular domain fragment comprising amino acid resides 33, 34, 36 and 37, 80-83, 92 and 93, 95 and 96, and 100-104 of SEQ ID NO: 1. Immunization of animals can be carried out by any method known in the art. See, e.g., Harlow and Lane, Antibodies: A Laboratory Manual, New York: Cold Spring Harbor Press, 1990. Methods for immunizing non-human animals such as mice, rats, sheep, goats, pigs, cattle and horses are well known in the art. See, e.g., Harlow and Lane, supra, and U.S. Pat. No. 5,994,619. The CD28 antigen may be administered with an adjuvant to stimulate the immune response. Exemplary adjuvants include complete or incomplete Freund's adjuvant, RIBI (muramyl dipeptides) or ISCOM (immune-stimulating complexes). After immunization of an animal with a CD28 antigen, antibody-producing immortalized cell lines are prepared from cells isolated from the immunized animal. After immunization, the animal is sacrificed and lymph node and/or splenic B cells are immortalized. Methods of immortalizing cells include, but are not limited to, transferring them with oncogenes, inflecting them with the oncogenic virus cultivating them under conditions that select for immortalized cells, subjecting them to carcinogenic or mutating compounds, fusing them with an immortalized cell, e.g., a myeloma cell, and inactivating a tumor suppressor gene. See, e.g., Harlow and Lane, supra. If fusion with myeloma cells is used, the myeloma cells preferably do not secrete immunoglobulin polypeptides (anon-secretory cell line). Immortalized cells are screened using CD28, a portion thereof, or a cell expressing CD28. CD28 antibody-producing cells, e.g., hybridomas, are selected, cloned and further screened for desirable characteristics, including robust growth, high antibody production and desirable antibody characteristics, as discussed further below. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

Antibodies of the disclosure can also be prepared using phage display or yeast display methods. Such display methods for isolating human antibodies are established in the art, such as Knappik, et al., “Fully Synthetic Human Combinatorial Antibody Libraries (HuCAL) Based on Modular Consensus Frameworks and CDRs Randomized with Trinucleotides. “J. Mol. Biol. (2000) 296, 57-86; and Feldhaus, et al, “Flow-cytometric isolation of human antibodies from a non-immune Saccharomyces cerevisiae surface display library” Nat Biotechnol (2003) 21: 163-170.

### B. Antigen Binding Fragments

In some other aspects, the present disclosure provides antigen-binding fragments of any of the CD28 antibodies provided by the present disclosure.

The antigen-binding fragment may comprise any sequences of the antibody. In some embodiments, the antigen-binding fragment comprises the amino acid sequence of: (1) a light chain of an anti-CD28 antibody; (2) a heavy chain of a CD28 antibody; (3) a variable region from the light chain of an anti-CD28 antibody; (4) a variable region from the heavy chain of a CD28 antibody; (5) one or more CDRs (two, three, four, five, or six CDRs) of an anti-CD28 antibody; or (6) three CDRs from the light chain and three CDRs from the heavy chain of an anti-CD28 antibody.

In some other particular embodiments, the antigen-binding fragments of an anti-CD28 antibody include: (i) a Fab fragment, which is a monovalent fragment consisting of the V_{L}, V_{H}, C_{L} and C_{H}1 domains; (ii) a F (ab′) 2 fragment, which is a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody; (v) a dAb fragment (Ward et al., Nature (1989) 341: 544-546), which consists of a V_{H} domain; (vi) an isolated CDR, and (vii) single chain antibody (scFv), which is a polypeptide comprising a V_{L} region of an antibody linked to a V_{H} region of an antibody. Bird et al., Science (1988) 242: 423-426, and Huston et al., Proc. Natl. Acad. Sci. USA (1988) 85: 5879-5883.

In some embodiments, the anti-CD28 antibody or antibody fragment disclosed herein comprises a V_{H} region that is at least 65%, at least 75%, at least 85%, at least 90%, at least 95%, at least 96%, at least 97%, at least 98%, or at least 99% identical to an amino acid sequence as set forth in any of SEQ ID NOs: 11, 20, 29, 39, 47, 54, 62, 71, 77, 84, 92, 99, 107, 115, 122, 130, 137, 144, 151, 157, and 165. In some embodiments, the anti-CD28 antibody or antibody fragment disclosed herein comprises a V_{L} region that is at least 65%, at least 75%, at least 85%, at least 90%, at least 95%, at least 96%, at least 97%, at least 98%, or at least 99% identical to an amino acid sequence as set forth in any of SEQ ID NOs: 12, 21, 30, 40, 48, 55, 63, 72, 78, 85, 93, 100, 108, 116, 123, 131, 138, 145, 152, 158 and 166. In some embodiments, the anti-CD28 antibody or antibody fragment disclosed herein comprises an HCDR1 amino acid sequence that is at least 65%, at least 75%, at least 85%, at least 90%, at least 95%, at least 96%, at least 97%, at least 98%, or at least 99% identical to an amino acid sequence as set forth in any of SEQ ID NOs: 5, 15, 24, 33, 43, 66, 88, 103, 111, 126, 134, 148, and 161. In some embodiments, the anti-CD28 antibody or antibody fragment disclosed herein comprises an HCDR2 amino acid sequence that is at least 65%, at least 75%, at least 85%, at least 90%, at least 95%, at least 96%, at least 97%, at least 98%, or at least 99% identical to an amino acid sequence as set forth in any of SEQ ID NOs: 6, 51, 58, 67, 89, 96, 104, and 155. In some embodiments, the anti-CD28 antibody or antibody fragment disclosed herein comprises an HCDR3 amino acid sequence that is at least 65%, at least 75%, at least 85%, at least 90%, at least 95%, at least 96%, at least 97%, at least 98%, or at least 99% identical to an amino acid sequence as set forth in any of SEQ ID NOs: 7, 16, 25, 35, 44, 52, 59, 81, 90, 97, 105, 112, 119, 127, 135, 141, 149, and 162. In some embodiments, the anti-CD28 antibody or antibody fragment disclosed herein comprises an LCDR1 amino acid sequence that is at least 65%, at least 75%, at least 85%, at least 90%, at least 95%, at least 96%, at least 97%, at least 98%, or at least 99% identical to an amino acid sequence as set forth in any of SEQ ID NOs: 8, 17, 26, 36, 45, 53, 60, 76, 82, 91, 98, 106, 113, 120, 128, 136, 142, 150, 156, and 163. In some embodiments, the anti-CD28 antibody or antibody fragment disclosed herein comprises an LCDR2 amino acid sequence that is at least 65%, at least 75%, at least 85%, at least 90%, at least 95%, at least 96%, at least 97%, at least 98%, or at least 99% identical to an amino acid sequence as set forth in any of SEQ ID NOs: 9, 18, 27, 37, and 300. In some embodiments, the anti-CD28 antibody or antibody fragment disclosed herein comprises an LCDR3 amino acid sequence that is at least 65%, at least 75%, at least 85%, at least 90%, at least 95%, at least 96%, at least 97%, at least 98%, or at least 99% identical to an amino acid sequence as set forth in any of SEQ ID NOs: 10, 19, 28, 38, 46, 61, 70, 83, 114, 121, 129, 143, and 164.

In some embodiments, the antibody disclosed herein comprises a heavy chain that is at least 65%, at least 75%, at least 85%, at least 90%, at least 95%, at least 96%, at least 97%, at least 98%, or at least 99% identical to an amino acid sequence as set forth in any of SEQ ID NOs: 13, 22, 31, 41, 49, 56, 64, 73, 79, 86, 94, 101, 109, 117, 124, 141, 132, 139, 146, 153, 159, and 167.

In some embodiments, the antibody disclosed herein comprises a light chain that is at least 65%, at least 75%, at least 85%, at least 90%, at least 95%, at least 96%, at least 97%, at least 98%, or at least 99% identical to an amino acid sequence as set forth in any of SEQ ID NOs: 14, 23, 32, 42, 50, 57, 65, 74, 80, 87, 95, 102, 110, 118, 125, 142, 133, 140, 147, 154, 160, and 168.

### Iv. Multi-Specific Antibodies

Also provided are multi-specific antibodies that correspond to the activatable multi-specific antibodies or masked multi-specific antibodies described herein. One aspect of the present application provides multi-specific antibodies (including activatable bispecific T cell engager (TCE) molecules) that are capable of binding to both T cells and target cells such as tumor cells. Because of their on-target off-tumor effects, traditional TCE molecules are associated with high cytotoxicity, including toxicity to the central nervous system (CNS) and cytokine storms. Therefore, there is a need for antibodies capable of binding a T cell and a target cell such as a tumor cell with enhanced specificity and reduced side effects.

In some embodiments, the multi-specific antibody is bispecific (bsAb). In some embodiments, the multi-specific antibody is trispecific (tsAb).

In particular embodiments, a bispecific antibody of the present disclosure is specific for CD28 on the surface of T cells. In some embodiments, the multi-specific antibody is a tumor-associated antigen (TAA) ×CD28 bispecific antibody that specifically binds to the TAA and CD28. In some embodiments, the antibody of the present disclosure is an IgG antibody, e.g., comprises an IgG Fc region (e.g., a human IgG Fc region).

In some embodiments, the multi-specific antibody binds to CD28 on the surface of T cells. In some embodiments, the multi-specific antibody is a tumor-associated antigen (TAA) ×CD28 bispecific antibody that specifically binds to the TAA and CD28. In some embodiments, the multi-specific antibody does not comprise any masking moiety or cleavable moiety. In some embodiments, the multi-specific antibody is obtained upon cleavage of the cleavable moiety or cleavable moieties.

In some embodiments, the multi-specific antibody binds to CD3 on the surface of T cells. In some embodiments, the multi-specific antibody is a tumor-associated antigen (TAA) ×CD3 bispecific antibody that specifically binds to the TAA and CD3. In some embodiments, the multi-specific antibody specifically binds CD3 with a weak affinity, e.g., an EC₅₀ of at least 10 nM (e.g., at least 100 nM) as determined by an ELISA assay, and/or a Kd of at least 50 nM. In some embodiments, the multi-specific antibody does not comprise any masking moiety or cleavable moiety. In some embodiments, the multi-specific antibody is obtained upon cleavage of the cleavable moiety or cleavable moieties.

In some embodiments, provided is a multi-specific antibody comprising: a) a first antigen-binding fragment comprising a VH1 and a V_{L} 1 of an antibody that specifically binds a target antigen (e.g., a tumor antigen, such as B7-H3, HER2, or TROP2); and b) a second antigen-binding fragment comprising a VH2 and a V_{L} 2 of an anti-CD3 antibody that specifically binds CD3, wherein the first and/or second antigen-binding fragment is fused to a first and/or second masking peptide (MP1/MP2). In some embodiments, provided is a multi-specific antibody comprising: a) a first antigen-binding fragment comprising a VH1 and a V_{L} 1 of an antibody that specifically binds a target antigen (e.g., a tumor antigen, such as B7-H3, HER2, or TROP2); and b) a second antigen-binding fragment comprising a VH2 and a V_{L} 2 of an anti-CD28 antibody that specifically binds CD28, wherein the first and/or second antigen-binding fragment is fused to a first and/or second masking peptide (MP1/MP2).

In some embodiments, the first antigen-binding fragment is selected from the group consisting of a Fab, a Fv, a scFab and a scFv. In some embodiments, the first antigen-binding fragment is a Fab. In some embodiments, the second antigen-binding fragment is selected from the group consisting of a Fab, a Fv, a scFab and a scFv. In some embodiments, the second antigen-binding fragment is a scFv comprising, from N-terminus to C-terminus, V_{L} 2, an optional linker, and VH2. In some embodiments, the first antigen-binding fragment is a Fab and the second antigen-binding fragment is a Fab. In some embodiments, the first antigen-binding fragment is a Fab and the second antigen-binding fragment is a scFv.

The antibodies comprising engineered C_{H}3 domains with disulfide bonds and/or salt bridges described herein may further comprise one or more knob-into-hole residues. “Knob-into-hole” or “KIH” refers to an approach known in the art for making bispecific antibodies also known as the “protuberance-into-cavity” approach (see, e.g., U.S. Pat. No. 5,731,168). In this approach, two immunoglobulin polypeptides (e.g., heavy chain polypeptides) each comprise an interface. An interface of one immunoglobulin polypeptide interacts with a corresponding interface on the other immunoglobulin polypeptide, thereby allowing the two immunoglobulin polypeptides to associate. These interfaces may be engineered such that a “knob” or “protuberance” (these terms may be used interchangeably herein) located in the interface of one immunoglobulin polypeptide corresponds with a “hole” or “cavity” (these terms may be used interchangeably herein) located in the interface of the other immunoglobulin polypeptide. In some embodiments, the hole is of identical or similar size to the knob and suitably positioned such that when the two interfaces interact, the knob of one interface is positionable in the corresponding hole of the other interface. Without wishing to be bound to theory, this is thought to stabilize the heteromultimer and favor formation of the heteromultimer over other species, for example homomultimers. In some embodiments, the KIH approach is used in combination with the engineered disulfide bonds and/or salt bridges described herein to promote the heteromultimerization of two different immunoglobulin polypeptides, creating a bispecific antibody comprising two immunoglobulin polypeptides with binding specificities for different epitopes. In some embodiments, the C_{H}3 domains of the activatable multi-specific antibodies described herein do not comprise KIH residues.

In some embodiments, there is provided a bispecific antibody targeting CD28 and a tumor antigen (e.g., B7-H3, HER2, TROP2 or PD-L1), comprising a first polypeptide, a second polypeptide, and a third polypeptide, wherein:
(i) the first polypeptide comprises a structure represented by the formula: V_{H}1-C_{H}1-hinge-C_{H}2-first CH3;
(ii) the second polypeptide comprises a structure represented by the formula: scFv-hinge-C_{H}2-second CH3; and
(iii) the third polypeptide comprises a structure represented by the formula: V_{L}1-C_{L};
wherein:
V_{L}1 is a first immunoglobulin light chain variable domain;
V_{H}1 is a first immunoglobulin heavy chain variable domain;
scFv is a single-chain variable fragment comprising a second immunoglobulin light chain variable domain (V_{L}2) and a second immunoglobulin heavy chain variable domain (V_{H}2);
C_{L} is an immunoglobulin light chain constant domain;
C_{H}1 is an immunoglobulin heavy chain constant domain 1;
C_{H}2 is an immunoglobulin heavy chain constant domain 2; and
hinge is an immunoglobulin hinge region connecting the C_{H}1 and C_{H}2 domains; wherein V_{L}1 and VH1 associate to form a first Fv that specifically binds the tumor antigen (e.g., B7-H3, HER2, TROP2 or PD-L1); and wherein the scFv specifically binds CD28. In some embodiments, the scFv binds CD28 with half-maximal binding at a concentration of antibody (EC₅₀) that ranges from 0.1 nM to 1000 nM as determined by ELISA (e.g., as described in Example 3). In some embodiments, the scFv binds CD28 with half-maximal binding at a concentration of antibody (EC₅₀) that is at less than 10 nM (e.g., between 1 nM and 0.1 μM) as determined by an Octet® RED96 assay (e.g., as described in Example 3). In particular embodiments, the scFv binds CD28 with a dissociation constant (Kd) of less than 10 nM.

In some embodiments, there is provided a bispecific antibody targeting CD28 and a tumor antigen (e.g., B7-H3, HER2, or TROP2), comprising a first polypeptide, a second polypeptide, a third polypeptide and a fourth polypeptide, wherein:
(i) the first polypeptide comprises a structure represented by the formula: V_{H}1-C_{H}1-hinge-C_{H}2-first CH3;
(ii) the second polypeptide comprises a structure represented by the formula: V_{H}2-C_{H}1-hinge-C_{H}2-second CH3;
(iii) the third polypeptide comprises a structure represented by the formula: V_{L}1-C_{L}; and
(iv) the fourth polypeptide comprises a structure represented by the formula: V_{L}2-C_{L};
wherein:
V_{L}1 is a first immunoglobulin light chain variable domain;
V_{H}1 is a first immunoglobulin heavy chain variable domain;
V_{L}2 is a second immunoglobulin light chain variable domain;
V_{H} 2 is a second immunoglobulin heavy chain variable domain;
C_{L} is an immunoglobulin light chain constant domain;
C_{H}1 is an immunoglobulin heavy chain constant domain 1;
C_{H}2 is an immunoglobulin heavy chain constant domain 2; and
hinge is an immunoglobulin hinge region connecting the C_{H}1 and C_{H}2 domains; wherein V_{L}1 and V_{H} 1 associate to form a first Fv that specifically binds the tumor antigen (e.g., B7-H3, HER2, TROP2 or PD-L1); and wherein V_{L}2 and V_{H} 2 associate to form a second Fv that specifically binds CD28. In some embodiments, the scFv binds CD28 with half-maximal binding at a concentration of antibody (EC₅₀) that is range from 0.1 nM to 1000 nM as determined by ELISA assay (e.g., as described in Example 3). In some embodiments, the second Fv binds CD28 with half-maximal binding at a concentration of antibody (EC₅₀) that is less than 10 nM (e.g., between 1 nM and 0.1 μM) as determined by an Octet® RED96 assay (e.g., as described in Example 3). In particular embodiments, the second Fv binds CD28 with a dissociation constant (Kd) of less than 10 nM.

In some embodiments, provided a multi-specific antibody comprising:
a) a first antigen-binding fragment comprising a V_{H} 1 and a V_{L}1 of an antibody that specifically binds a target antigen (e.g., a tumor antigen, such as B7-H3, HER2, or TROP2); and
b) a second antigen-binding fragment comprising a V_{H} 2 and a V_{L}2 of an anti-CD28 antibody that specifically binds CD28, wherein the second antigen-binding fragment is fused to a first masking peptide (MP1).

In some embodiments, there is provided a multi-specific antibody comprising:
a) a first antigen-binding fragment comprising a V_{H} 1 and a V_{L}1 of an antibody that specifically binds a target antigen (e.g., a tumor antigen, such as B7-H3, HER2, or TROP2), wherein the first antigen-binding fragment is fused to a first masking peptide (MP1); and
b) a second antigen-binding fragment comprising a V_{H}2 and a V_{L}2 of an anti-CD28 antibody that specifically binds CD28, wherein the second antigen-binding fragment is fused to a second masking peptide (MP2).

In some embodiments, the first antigen-binding fragment is selected from the group consisting of a Fab, a Fv, a scFab and a scFv. In some embodiments, the first antigen-binding fragment is a Fab. In some embodiments, the second antigen-binding fragment is selected from the group consisting of a Fab, a Fv, a scFab and a scFv. In some embodiments, the second antigen-binding fragment is a scFv comprising, from N-terminus to C-terminus, V_{L}2, an optional linker, and V_{H}2.

In some embodiments, provided a multi-specific antibody comprising a first polypeptide, a second polypeptide, and a third polypeptide, wherein:
(i) the first polypeptide comprises a structure represented by the formula: V_{H}1-C_{H}1-hinge-C_{H}2-first C_{H}3;
(ii) the second polypeptide comprises a structure represented by the formula: MP1-V_{L}2-V_{H} 2-hinge-C_{H}2-second C_{H}3; and
(iii) the third polypeptide comprises a structure represented by the formula: V_{L}1-C_{L};
wherein:
V_{L}1 is a first immunoglobulin light chain variable domain;
V_{H}1 is a first immunoglobulin heavy chain variable domain;
V_{L}2 is a second immunoglobulin light chain variable domain;
V_{H}2 is a second immunoglobulin heavy chain variable domain;
C_{L} is an immunoglobulin light chain constant domain;
C_{H}1 is an immunoglobulin heavy chain constant domain 1;
C_{H}2 is an immunoglobulin heavy chain constant domain 2;
first C_{H}3 is a first immunoglobulin heavy chain constant domain 3;
second C_{H}3 is a second immunoglobulin heavy chain constant domain 3;
hinge is an immunoglobulin hinge region connecting the C_{H}1 and C_{H}2 domains;
MP1 is a first masking peptide; MP1 comprises, from N-terminus to C-terminus, an N-terminal unit (NU), a masking unit (MU), and a linkage unit (LU); the LU of the masking peptide may not comprise a cleavage site, or comprise at least one cleavage site.

In some embodiments, there is provided a multi-specific antibody comprising a first polypeptide, a second polypeptide, and a third polypeptide, wherein:
(i) the first polypeptide comprises a structure represented by the formula: V_{H}1-C_{H}1-hinge-C_{H}2-first C_{H}3);
(ii) the second polypeptide comprises a structure represented by the formula: MP2-V_{L}2-V_{H}2-hinge-C_{H}2-second C_{H}3; and
(iii) the third polypeptide comprises a structure represented by the formula: MP1-V_{L}1-C_{L};
wherein:
V_{L}1 is a first immunoglobulin light chain variable domain;
V_{H}1 is a first immunoglobulin heavy chain variable domain;
V_{L}2 is a second immunoglobulin light chain variable domain;
V_{H}2 is a second immunoglobulin heavy chain variable domain;
C_{L} is an immunoglobulin light chain constant domain;
C_{H}1 is an immunoglobulin heavy chain constant domain 1;
C_{H}2 is an immunoglobulin heavy chain constant domain 2;
first C_{H}3 is a first immunoglobulin heavy chain constant domain 3;
second C_{H}3 is a second immunoglobulin heavy chain constant domain 3;
hinge is an immunoglobulin hinge region connecting the C_{H}1 and C_{H}2 domains;
MP1 is a masking peptide; MP1 comprises, from N-terminus to C-terminus, an N-terminal unit (NU), a masking unit (MU) and a linkage unit (LU); the LU of the masking peptide may comprise non, at least one or more cleavage site.
MP2 is a masking peptide; MP2 comprises, from N-terminus to C-terminus, an N-terminal unit (NU), a masking unit (MU) and a linkage unit (LU); the LU of the masking peptide may not comprise a cleavage site, or comprise at least one cleavage site.

In some embodiments, the bispecific antibody binds to a first and second target, where the first target is CD28, and the bispecific antibody comprises an HCDR1 comprising an amino acid sequence selected from the group consisting of SEQ ID NOs: 5, 15, 24, 33, 43, 66, 88, 103, 111, 126, 134, 148, and 161; an HCDR2 comprising an amino acid sequence selected from the group consisting of SEQ ID NOs: 6, 51, 58, 67, 89, 96, 104, and 155; and an HCDR3 comprising an amino acid sequence selected from SEQ ID NOs: 7, 16, 25, 35, 44, 52, 59, 81, 90, 97, 105, 112, 119, 127, 135, 141, 149, and 162; and an LCDR1 comprising an amino acid sequence selected from the group consisting of SEQ ID NOs: 8, 17, 26, 36, 45, 53, 60, 76, 82, 91, 98, 106, 113, 120, 128, 136, 142, 150, 156, and 163; an LCDR2 comprising an amino acid sequence selected from the group consisting of SEQ ID NOs: 9, 18, 27, 37, and 300; and an LCDR3 comprising an amino acid sequence selected from the group consisting of SEQ ID NOs: 10, 19, 28, 38, 46, 61, 70, 83, 114, 121, 129, 143, and 164. In some embodiments, the bispecific antibody binds to a first and second target, where the first target is human CD28, and where the bispecific antibody comprises a heavy chain variable region comprising an amino acid sequence selected from the group consisting of SEQ ID NOs: 11, 20, 29, 39, 47, 54, 62, 71, 77, 84, 92, 99, 107, 115, 122, 130, 137, 144, 151, 157, and 165, and a light chain variable region comprising an amino acid sequence selected from the group consisting of SEQ ID NOs: 12, 21, 30, 40, 48, 55, 63, 72, 78, 85, 93, 100, 108, 116, 123, 131, 138, 145, 152, 158 and 166.

In some embodiments, the bispecific antibody binds to a first and second target, wherein the first target is CD28 and the second target is B7H3, wherein the bispecific antibody comprises a CD28-binding portion comprising an amino acid sequence selected from the group consisting of SEQ ID NOs: 11, 12, and 171, and where the bispecific antibody comprises a heavy chain comprising an amino acid sequence set forth in SEQ ID NO: 176, and a light chain comprising an amino acid sequence set forth in SEQ ID NO: 175. In particular embodiments, the bispecific antibody binds to a first and second target, wherein the first target is CD28 and the second target is B7H3, wherein the bispecific antibody comprises a heavy chain comprising an amino acid sequence set forth in SEQ ID NO: 171, a second heavy chain comprising an amino acid sequence set forth in SEQ ID NO: 1176, and a light chain comprising an amino acid sequence set forth in SEQ ID NO: 175.

In some embodiments, the bispecific antibody binds to a first and second target, wherein the first target is CD28 and the second target is HER2, wherein the bispecific antibody comprises a CD28-binding portion comprising an amino acid sequence set forth in SEQ ID NO: 172, and where the bispecific antibody comprises a heavy chain comprising an amino acid set forth in SEQ ID NO: 170, and a light chain comprising an amino acid sequence set forth in SEQ ID NO: 169. In particular embodiments, the bispecific antibody binds to a first and second target, wherein the first target is CD28 and the second target is HER2, wherein the bispecific antibody comprises a heavy chain comprising an amino acid sequence set forth in SEQ ID NO: 172, a second heavy chain comprising an amino acid sequence set forth in SEQ ID NO: 170, and a light chain comprising an amino acid sequence set forth in SEQ ID NO: 169.

In some embodiments, the bispecific antibody binds to a first and second target, wherein the first target is CD28 and the second target is HER2, wherein the bispecific antibody comprises a CD28-binding portion comprising an amino acid sequence set forth in SEQ ID NO: 11, 12, 171, a heavy chain comprising an amino acid set forth in SEQ ID NO: 170, and a light chain comprising an amino acid sequence set forth in SEQ ID NO: 169. In particular embodiments, the bispecific antibody binds to a first and second target, wherein the first target is CD28 and the second target is HER2, wherein the bispecific antibody comprises a heavy chain comprising an amino acid sequence set forth in SEQ ID NO: 171, a second heavy chain comprising an amino acid sequence set forth in SEQ ID NO: 170, and a light chain comprising an amino acid sequence set forth in SEQ ID NO: 169.

In some embodiments, the bispecific antibody binds to a first and second target, wherein the first target is human CD28 and the second target is TROP2, wherein the bispecific antibody comprises a CD28-binding portion comprising an amino acid sequence selected from the group consisting of SEQ ID NOs: 13, 14, and 171, and where the bispecific antibody comprises a heavy chain comprising an amino acid sequence set forth in SEQ ID NO: 174, and a light chain comprising an amino acid sequence set forth in SEQ ID NO: 173. In particular embodiments, the bispecific antibody binds to a first and second target, wherein the first target is CD28 and the second target is TROP2, wherein the bispecific antibody comprises a heavy chain comprising an amino acid sequence set forth in SEQ ID NO: 171, a second heavy chain comprising an amino acid sequence set forth in SEQ ID NO: 174, and a light chain comprising an amino acid sequence set forth in SEQ ID NO: 173.

In particular embodiments, the bispecific antibody binds to a first and second target, where the first target is CD3 and the second target is B7-H3, and where the bispecific antibody comprises a heavy chain comprising an amino acid sequence set forth in SEQ ID NO: 177 or an scFv fusion polypeptide set forth in SEQ ID NO: 299, a heavy chain comprising an amino acid sequence set forth in SEQ ID NO: 176, and a light chain comprising an amino acid sequence set forth in SEQ ID NO: 175.

In particular embodiments, the bispecific antibody binds to a first and second target, where the first target is CD3 and the second target is TROP2.

In some embodiments, the bispecific antibody binds to a first and second target, where the first target is human CD28, and where the bispecific antibody comprises a heavy chain amino acid sequence selected from the group consisting of SEQ ID NOs: 13, 22, 31, 41, 49, 56, 64, 73, 79, 86, 94, 101, 109, 117, 124, 141, 132, 139, 146, 153, 159, and 167, and a light chain amino acid sequence selected from the group consisting of SEQ ID NOs: 14, 23, 32, 42, 50, 57, 65, 74, 80, 87, 95, 102, 110, 118, 125, 142, 133, 140, 147, 154, 160, and 168.

### V. Masked Antibodies

In some embodiments, the present disclosure provides a masked antibody which may be a masked monoclonal antibody to a specific target or a multi-specific (e.g., bispecific) antibody. In some embodiments, the masked antibody provided herein comprises a full-length antibody light chain, e.g., a kappa or lambda light chain. Additionally or alternatively, in some embodiments, the antibody comprises a full-length antibody heavy chain. The antibody heavy chain may be in any class, such as IgG, IgM, IgE, IgA, or IgD. In some embodiments, the antibody heavy chain is in the IgG class, such as IgG₁, IgG₂, IgG₃, or IgG₄ subclass. An antibody heavy chain described herein may be converted from one class or subclass to another class or subclass using methods known in the art. In some embodiments, the masked antibody is or comprises a full-length antibody that comprises an Fc region, e.g., a human Fc region or a variant thereof. In some embodiments, the human Fc region is a human IgG₁ Fc region, a human IgG₂ Fc region, a human IgG₄ Fc region, or a variant of any one of the preceding. In some embodiments, the variant Fc region comprises one or more amino acid substitutions, insertions, or deletions relative to the wild type human Fc region from which the variant is derived. In some embodiments, the masked antibody comprises a variant of a human IgG₁ Fc region. In some embodiments, the IgG₁ Fc variant comprises one or more amino acid substitutions that increases the affinity of the Fc variant for FcγRIIb. In some embodiments, the variant of the human IgG₁ Fc region comprises substitution (s) selected from the group consisting of: G236D; L328F; S239D; S267E; G236D and S267E; S239D and S267E; S267E and L328S; and S267E and L328F, wherein amino acid numbering is according to the EU index (see, e.g., Edelman et al., Proc Natl Acad Sci USA (1969) 63: 78-85). The preceding substitutions are described in Chu et al. Mol Immunol. (2008) 45 (15): 3926-33. Additionally or alternatively, in some embodiments, the variant of the human IgG₁ Fc region comprises substitution (s) selected from the group consisting of: E233D and P238D; G237D and P238D; H268D and P238D; P271G and P238D; A330R and P238D; E233D, P238D, and A330R; E233D, P231G, P238D. and A330R; G237D, H268D, P238D, and P271G; G237D, P238D, P271G, and A330R; E233D, H268D, P238D, P271G, and A330R; G237D, H268D, P238D, P271G, and A330R; and E233D, G237D, P238D, H268D, P271G and A330R, wherein amino acid numbering is according to the EU index. The preceding substitutions are described in Mimoto et al. Protein Eng Des Sel. (2013) 26 (10): 589-98. Additionally or alternatively, in some embodiments, the variant of the human IgG₁ Fc region comprises an S2657A substitution (see Buschor et al. Int Arch Allergy Immunol. (2014) 163 (3): 206-14), wherein amino acid numbering is according to the EU index. Additionally or alternatively, in some embodiments, the variant of the human IgG₁ Fc region comprises a T437R and/or a K248E substitution (see Zhang et al. MAbs. (2017) 9 (7): 1129-1142), wherein amino acid numbering is according to the EU index. In some embodiments, the masked antibody comprises a variant of a human IgG₄ Fc region. In some embodiments, the IgG₄ Fc variant comprises one or more amino acid substitutions that increases the affinity of the Fc variant for FcγRIIb. In some embodiments, the variant of the human IgG₄ Fc region comprises substitution (s) selected from the group consisting of: G236D; L328F; S239D; S267E; G236D and S267E; S239D and S267E; S267E and L328S; and S267E and L328F, wherein amino acid numbering is according to the EU index. Additionally or alternatively, in some embodiments, the variant of the human IgG₄ Fc region comprises substitution (s) selected from the group consisting of: E233D and P238D; G237D and P238D; H268D and P238D; P271G and P238D; A330R and P238D; E233D, P238D, and A330R; E233D, P231G, P238D. and A330R; G237D, H268D, P238D, and P271G; G237D, P238D, P271G, and A330R; E233D, H268D, P238D, P271G, and A330R; G237D, H268D, P238D, P271G, and A330R; and E233D, G237D, P238D, H268D, P271G and A330R, wherein amino acid numbering is according to the EU index. Additionally or alternatively, in some embodiments, the variant of the human IgG₄ Fc region comprises an S2657A substitution, wherein amino acid numbering is according to the EU index. Additionally or alternatively, in some embodiments, the variant of the human IgG₁ Fc region comprises a T437R and/or a K248E substitution wherein amino acid numbering is according to the EU index.

In some embodiments, the masked antibodies disclosed herein further comprise a human IgG₁ domain or a variant thereof that comprises one or more substitution mutation (s). In some embodiments the IgG₁ variant comprises substitution (s) selected from the group consisting of: G236D; L328F; S239D; S267E; G236D and S267E; S239D and S267E; S267E and L328S; and S267E and L328F; E233D and P238D; G237D and P238D; H268D and P238D; P271G and P238D; A330R and P238D; E233D, P238D, and A330R; E233D, P231G, P238D. and A330R; G237D, H268D, P238D, and P271G; G237D, P238D, P271G, and A330R; E233D, H268D, P238D, P271G, and A330R; G237D, H268D, P238D, P271G, and A330R; and E233D, G237D, P238D, H268D, P271G and A330R; S2657A; T437R; K248E; and T437R and K248E, wherein amino acid numbering is according to the EU index. In some embodiments, the masked antibodies disclosed herein further comprise a human IgG₄ domain or a variant thereof that comprises one or more substitution mutation (s). In some embodiments the IgG₄ variant comprises substitution (s) selected from the group consisting of: G236D; L328F; S239D; S267E; G236D and S267E; S239D and S267E; S267E and L328S; and S267E and L328F; E233D and P238D; G237D and P238D; H268D and P238D; P271G and P238D; A330R and P238D; E233D, P238D, and A330R; E233D, P231G, P238D. and A330R; G237D, H268D, P238D, and P271G; G237D, P238D, P271G, and A330R; E233D, H268D, P238D, P271G, and A330R; G237D, H268D, P238D, P271G, and A330R; and E233D, G237D, P238D, H268D, P271G and A330R; S2657A; T437R; K248E; and T437R and K248E, wherein amino acid numbering is according to the EU index.

In some embodiments, the term “masked antibody” refers to an antibody fragment, e.g., a masked antigen-binding fragment of a masked anti-CD28 antibody. In some embodiments, the antibody fragment is or comprises a Fab, an Fab′, a Fab′-SH, a F (ab′) 2, an Fv, an scFv (see Bird et al. (1988) Science 242: 423-426 and Huston et al. (1988) Proc. Natl. Acad. Sci. USA 85: 5879-5883), an (scFv) 2, a linear antibody, a single-chain antibody, a minibody, or a diabody.

In some embodiments, a masked anti-CD28 antibody described herein cross-reacts with CD28 from different species, thus permitting the masked anti-CD28 antibody to be used in both preclinical and clinical studies. In some embodiments, a masked anti-CD28 antibody described herein binds to two or more of human CD28, cynomolgus CD28, murine (mouse) CD28, and/or rat CD28 following activation (i.e., after activation of the masked antibody via cleavage, e.g., protease cleavage). In some embodiments, a masked anti-CD28 antibody binds human CD28, cynomolgus CD28, murine (mouse) CD28, and a rat CD28 following activation (i.e., after activation of the masked antibody via cleavage, e.g., protease cleavage).

In some embodiments, masked anti-CD28 antibodies described herein are context-dependent (e.g., are activated (are only capable of binding their targets) in certain contexts (such as in the protease-rich tumor microenvironment)). In some embodiments, the masked anti-CD28 antibodies described herein provide improved safety over more traditional, non-masked antibodies (e.g., show reduced toxicity, do not induce significant alterations to the weights of many organs, do not alter liver histopathology, hematology, and/or blood biochemistry, etc.). In some embodiments, masked anti-CD28 antibodies described herein exhibit pharmacokinetic properties that are similar to those of traditional, non-masked anti-CD28 antibodies (e.g., have similar in vivo half-lives). In some embodiments, masked anti-CD28 antibodies described herein exhibit improved pharmacokinetic properties as compared to more traditional, non-masked anti-CD28 antibodies (e.g., have longer in vivo half-lives).

In some embodiments, the antibody heavy chain variable region (V_{H}) and the antibody light chain variable region (V_{L}) of a masked anti-CD28 antibody described herein form an antigen binding domain (ABD) that binds hCD28. In some embodiments, the masking unit (MU) of a masked anti-CD28 antibody described herein binds to the ABD of the and reduces or inhibits binding of the masked anti-CD28 antibody to hCD28, as compared to the binding of a corresponding anti-CD28 antibody lacking the MU to hCD28 and/or as compared to the binding of the ABD to hCD28.

In some embodiments, the masking unit (MU) has a masking efficiency of at least about 2.0 (e.g., at least about 2.0, at least about 3.0, at least about 4.0, at least about 5.0, at least about 6.0, at least about 7.0, at least about 8.0, at least about 9.0, at least about 10, at least about 25, at least about 50, at least about 75, at least about 100, at least about 150, at least about 200, at least about 300, at least about 400, at least about 500, at least about 600, at least about 700, at least about 800, at least about 900, at least about 1,000, at least about 1,100, at least about 1, 200, at least about 1, 300, at least about 1, 400, at least about 1,500, etc., including any range in between these values) prior to removing the MU from a masked antibody provided herein. For example, and in some embodiments, the masking efficiency of a masked anti-CD28 antibody is measured as the difference in affinity of the masked anti-CD28 antibody comprising the masking unit (MU) for binding to hCD28 (i.e., before activation of the masked antibody) relative to the affinity of an anti-CD28 antibody lacking the MU for binding to hCD28. In a further example, masking efficiency is measured as the difference in affinity for hCD28 of a masked anti-CD28 antibody comprising a MU (i.e., before activation of the masked antibody via cleavage, e.g., protease cleavage) relative to the affinity for hCD28 of the unmasked anti-CD28 antibody (i.e., after activation of the masked antibody via cleavage, e.g., protease cleavage). In some embodiments, the masking efficiency is measured by dividing the EC₅₀ for target-binding of a masked antibody comprising an MU (i.e., before activation) by the EC₅₀ of a corresponding antibody specific for the same target that lacks the masking peptide or masking unit. In some embodiments, the EC₅₀ is measured by ELISA. In some embodiments, the masking unit (MU) of the masked antibody binds to the ABD, and prevents the masked polypeptide from binding to its target. In particular embodiments, the target is CD28. In other embodiments, the target is CD3, B7-H3, HER2, or TROP2.

In some embodiments, the affinity of a masked antibody of the present disclosure increases by at least about 2-fold (e.g., at least about 2-fold, at least about 2.5-fold, at least about 3, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 25-fold, at least about 50-fold, at least about 75-fold, at least about 100-fold, at least about 250-fold, at least about 500-fold, at least about 750-fold, or at least about 1000-fold, or more, including any range in between the preceding values) when the masking unit is removed from the antibody (e.g., after activation by treatment with one or more proteases that cleave within the linkage unit) as compared to a corresponding antibody specific for the same target but without the masking peptide or masking unit. In some embodiments, the EC₅₀ of a masked antibody described herein decreases by at least about 2-fold (e.g., at least about 2-fold, at least about 2.5-fold, at least about 3, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 25-fold, at least about 50-fold, at least about 75-fold, at least about 100-fold, at least about 250-fold, at least about 500-fold, at least about 750-fold, or at least about 1000-fold, or more, including any range in between the preceding values) after activation by treatment with one or more proteases that cleave within the linkage unit (e.g., as measured by an ELISA or FACS assay).

In some embodiments, when the masking unit is bound to the ABD of a masked antibody described herein, the K_{D} of the antibody for its target is about 2 (e.g., about 2, about 2.5, about 3, about 3.5 about 4, about 4.5, about 5, about 5.5, about 6, about 6.5, about 7, about 7.5, about 8, about 8.5, about 9, about 9.5, about 10, about 25, about 50, about 75, about 100, about 250, about 500, about 750, or about 1000 or more, including any range in between the preceding values) times greater than the K_{D} of the antibody when the masking unit of the masked anti-CD28 antibody is removed from the ABD (such as after protease treatment to cleave within the linkage unit). In some embodiments, when the masking unit is bound to the ABD of a masked antibody described herein, the K_{D} of the antibody for its target is about 2 (e.g., about 2, about 2.5, about 3, about 3.5 about 4, about 4.5, about 5, about 5.5, about 6, about 6.5, about 7, about 7.5, about 8, about 8.5, about 9, about 9.5, about 10, about 25, about 50, about 75, about 100, about 250, about 500, about 750, or about 1000 or more, including any range in between the preceding values) times greater than the K_{D} of a corresponding antibody that is specific to the same target but lacks a masking peptide or masking unit.

In some embodiments, the masking unit sterically hinders binding of the masked binding polypeptide to its target and/or allosterically hinders binding of the masked binding polypeptide to its target.

In some embodiments, the dissociation constant of the masking unit for the ABD of a masked antibody (e.g., anti-CD28) described herein is greater than the dissociation constant of the masked antibody for its target (e.g., hCD28; when the masked antibody is in active form, such as after protease treatment). In some embodiments, the dissociation constant of the masking unit for the ABD of a masked antibody (e.g., anti-CD28) described herein is about 2 (e.g., about 2, about 2.5, about 3, about 3.5 about 4, about 4.5, about 5, about 5.5, about 6, about 6.5, about 7, about 7.5, about 8, about 8.5, about 9, about 9.5, about 10, about 25, about 50, about 75, about 100, about 250, about 500, about 750, or about 1000 or more, including any range in between the preceding values) times greater than the dissociation constant of the masked antibody for its target (e.g., hCD28; when the masked antibody is in active form, such as after protease treatment). In some embodiments, the dissociation constant of the masking unit for the ABD of a masked antibody (e.g., anti-CD28) described herein is about equal to the dissociation constant of the masked antibody for its target (e.g., hCD28; when the masked antibody is in active form, such as after protease treatment). In some embodiments, the masking unit (MU) binds to the ABD of a masked antibody (e.g., anti-CD28) described herein and prevents the antibody from binding to its target (e.g., hCD28) only when the masked antibody has not been activated (e.g., by treatment with one or more proteases that cleave within the linkage unit). In some embodiments, activation induces cleavage of the polypeptide within the cleavage site. In some embodiments, activation induces conformation changes in the polypeptide (e.g., displacement of the masking unit (MU)), leading to the masking peptide no longer preventing the polypeptide from binding to its target.

### A. Single-Masked Antibodies

In some embodiments, provided is a masked monoclonal antibody comprising a masking peptide (MP) and an antibody that binds CD28, wherein the antibody comprises a heavy chain variable regions (V_{H}) and a light chain variable region (V_{L}), wherein the MP is linked to an N-terminus of the V_{L}, wherein the MP comprises, from N-terminus to C-terminus, a masking unit (MU), and a linkage unit (LU), wherein the MP comprises an amino acid sequence selected from the group consisting of SEQ ID NOs: 215-248, and wherein the antibody V_{H} region comprises a HCDR1 amino acid sequence selected from the group consisting of SEQ ID NOs: 5, 15, 24, 33, 43, 66, 88, 103, 111, 126, 134, 148, and 161; a HCDR2 amino acid sequence selected from the group consisting of SEQ ID NOs: 6, 51, 58, 67, 89, 96, 104, and 155; and a HCDR3 amino acid sequence selected from the group consisting of SEQ ID NOs: 7, 16, 25, 35, 44, 52, 59, 81, 90, 97, 105, 112, 119, 127, 135, 141, 149, and 162; and the antibody V_{L} region comprises a LCDR1 amino acid sequence selected from the group consisting of SEQ ID NOs: 8, 17, 26, 36, 45, 53, 60, 76, 82, 91, 98, 106, 113, 120, 128, 136, 142, 150, 156, and 163; a LCDR2 amino acid sequence selected from the group consisting of SEQ ID NOs: 9, 18, 27, 37, and 300; and a LCDR3 amino acid sequence selected from the group consisting of SEQ ID NOs: 10, 19, 28, 38, 46, 61, 70, 83, 114, 121, 129, 143, and 164.

In some embodiments, provided is a masked monoclonal antibody comprising a masking peptide (MP) and an antibody that binds CD28, wherein the antibody comprises a heavy chain variable regions (V_{H}) and a light chain variable region (V_{L}), wherein the MP is linked to an N-terminus of the V_{L}, wherein the MP comprises, from N-terminus to C-terminus, a masking unit (MU), and a linkage unit (LU), wherein the MP comprises an amino acid sequence selected from the group consisting of SEQ ID NOs: 215-248, and wherein the antibody heavy chain variable region comprises an amino acid sequence selected from the group consisting of SEQ ID NOs: 11, 20, 29, 39, 47, 54, 62, 71, 77, 84, 92, 99, 107, 115, 122, 130, 137, 144, 151, 157, and 165, and the light chain variable region comprises an amino acid sequence selected from the group consisting of SEQ ID NOs: 12, 21, 30, 40, 48, 55, 63, 72, 78, 85, 93, 100, 108, 116, 123, 131, 138, 145, 152, 158 and 166. In some embodiments, provided is a masked monoclonal antibody comprising a masking peptide (MP) and an antibody that binds human CD28, wherein the antibody comprises a heavy chain and a light chain, wherein the MP is linked to an N-terminus of the LC, wherein the MP comprises, from N-terminus to C-terminus, a masking unit (MU), and a linkage unit (LU), wherein the MP comprises an amino acid sequence selected from the group consisting of SEQ ID NOs: 215-248, wherein the antibody HC comprises an amino acid sequence selected from group consisting of SEQ ID NOs: 13, 22, 31, 41, 49, 56, 64, 73, 79, 86, 94, 101, 109, 117, 124, 141, 132, 139, 146, 153, 159, and 167, and wherein the antibody LC comprises the amino acid sequences selected from group consisting of SEQ ID NOs: 14, 23, 32, 42, 50, 57, 65, 74, 80, 87, 95, 102, 110, 118, 125, 142, 133, 140, 147, 154, 160, and 168.

### B. Single-Masked Multi-Specific Antibodies

In some embodiments, also provided is a masked bispecific monoclonal antibody specific for a first and second target, where the first target is CD28, wherein the antibody comprises a masking peptide (MP) and a CD28-binding portion, wherein the antibody comprises a heavy chain variable region (V_{H}) and a light chain variable region (V_{L}), wherein the MP is linked to an N-terminus of the V_{L}, wherein the MP comprises, from N-terminus to C-terminus, a masking unit (MU), and a linkage unit (LU), wherein the MP comprises an amino acid sequence selected from the group consisting of SEQ ID NOs: 215-248, wherein the CD28-binding portion comprises a V_{H} region comprising a HCDR1 of SEQ ID NO: 5, HCDR2 of SEQ ID NO: 6, and HCDR3 of SEQ ID NO: 7, and a V_{L} region comprising a LCDR1 of SEQ ID NO: 8, a LCDR2 of SEQ ID NO: 9 and a LCDR3 of SEQ ID NO: 10, and wherein the second target is a B7-H3, HER2, or TROP2 protein.

In some embodiments, also provided is a masked bispecific monoclonal antibody specific for a first and second target, where the first target is CD28, wherein the antibody comprises a masking peptide (MP) and a CD28-binding portion, wherein the antibody comprises a heavy chain variable region (V_{H}) and a light chain variable region (V_{L}), wherein the MP is linked to an N-terminus of the V_{L}, wherein the MP comprises, from N-terminus to C-terminus, a masking unit (MU), and a linkage unit (LU), wherein the MP comprises an amino acid sequence selected from the group consisting of SEQ ID NOs: 215-248, and wherein the CD28-binding portion comprises a heavy chain variable region comprising an amino acid sequence selected from the group consisting of SEQ ID NOs: 11, 20, 29, 39, 47, 54, 62, 71, 77, 84, 92, 99, 107, 115, 122, 130, 137, 144, 151, 157, and 165; and a light chain variable region comprising an amino acid sequence selected from the group consisting of SEQ ID NOs: 12, 21, 30, 40, 48, 55, 63, 72, 78, 85, 93, 100, 108, 116, 123, 131, 138, 145, 152, 158 and 166, and wherein the second target is a B7-H3, HER2, or TROP2 protein.

### C. Double-Masked Multi-Specific Antibodies

In particular embodiments, also provided is a masked bispecific monoclonal antibody specific for a first and second target, where the first target is CD28 and the second target is a tumor associated antigen selected from HER2, B7-H3, and TROP2, wherein the antibody comprises two masking peptides (MP).

### VI. Masking Peptides

In some embodiments, the MP further comprises a N-terminal unit. In some embodiments, the N-terminal unit is between about 1 and 10 amino acids in length. In some embodiments, the N-terminal unit comprises SEQ ID NO: 210. In some embodiments, the LU comprises at least a first cleavage site (CS₁) (e.g., a first protease cleavage site). In some embodiments, the LU further comprises a second cleavage site (CS₂). In some embodiments, the first and/or second cleavage site are a protease cleavage site. In some embodiments, the first and second cleavage sites are the same. In some embodiments, the first and second cleavage sites are different. Any suitable protease cleavage site recognized and/or cleaved by any protease (e.g., a protease that is known to be co-localized with a target of a polypeptide comprising the cleavage site) known in the art may be used, including, for example, a protease cleavage site recognized and/or cleaved by urokinase-type plasminogen activator (uPA); matrix metalloproteinases (e.g., MMP-1, MMP-2, MMP-3, MMP-7, MMP-8, MMP-9, MMP-10, MMP-11, MMP-12, MMP-13, MMP-14, MMP-15, MMP-16, MMP-17, MMP-19, MMP-20, MMP-23, MMP-24, MMP-26, and/or MMP-27); Tobacco Etch Virus (TEV) protease; plasmin; Thrombin; PSA; PSMA; ADAMS/ADAMTS (e.g., ADAM 8, ADAM 9, ADAM10, ADAM12, ADAM15, ADAM17/TACE, ADAMDEC1, ADAMTS1, ADAMTS4, and/or ADAMTS5); caspases (e.g., Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, and/or Caspase-14); aspartate proteases (e.g., RACE and/or Renin); aspartic cathepsins (e.g., Cathepsin D and/or Cathepsin E); cysteine cathepsins (e.g., Cathepsin B, Cathepsin C, Cathepsin K, Cathepsin L, Cathepsin S, Cathepsin V/L2, and/or Cathepsin X/Z/P); cysteine proteinases (e.g., Cruzipain, Legumain, and/or Otubain-2); KLKs (e.g., KLK4, KLK5, KLK6, KLK7, KLK8, KLK10, KLK11, KLK13, and/or KLK14); metalloproteinases (e.g., Meprin, Neprilysin, PSMA, and/or BMP-1); serine proteases (e.g., activated protein C, cathepsin A, cathepsin G, chymase, and/or coagulation factor proteases (such as FVIIa, FIXa, FXa, FXIa, FXIIa)); elastase; granzyme B; guanidinobenzoatase; HtrA1; human neutrophil elastase; lactoferrin; marapsin; NS3/4A; PACE4; tPA; tryptase; type II transmembrane serine proteases (TTSPs) (e.g., DESC1, DPP-4, FAP, hepsin, matriptase-2, MT-SP1/matriptase, TMPRSS2, TMPRSS3 and/or TMPRSS4); etc. In some embodiments, the first protease cleavage site is a cleavage site for a protease selected from uPA, MMP-1, MMP-2, MMP-3, MMP-8, MMP-9, MMP-14, TEV protease, plasmin, thrombin, Factor X, PSA, PSMA, cathepsin D, cathepsin K, cathepsin S, ADAM10, ADAM12, ADAMTS, caspase-1, caspase-2, caspase-3, caspase-4, caspase-5, caspase-6, caspase-7, caspase-8, caspase-9, caspase-10, caspase-11, caspase-12, caspase-13, caspase-14, and TACE. In some embodiments, the first protease cleavage site is a cleavage site for a protease selected from uPA, MMP-2, MMP-9, and/or TEV protease.

In some embodiments, the LU further comprises a first linker (L₁). In some embodiments, the first linker (L₁) is C-terminal to the first cleavage site (CS₁) (e.g., a first protease cleavage site). In some embodiments, the LU comprises a structure, from N-terminus to C-terminus, of: (CS₁)-L₁. In some embodiments, the LU further comprises a second linker (L₂). In some embodiments, the L₂ is C-terminal to the second cleavage site. In some embodiments, the LU comprises a structure, from N-terminus to C-terminus, of: (CS₁)-L₁-(CS₂)-L₂. In some embodiments, L₁ and L₂ are any suitable linker (e.g., a flexible linker) known in the art, including, without limitation, e.g., glycine polymers (G) n, where n is an integer of at least 1 (e.g., at least one, at least 2, at least 3, at least 4, at least 5, at least 6, at least 7, at least 8, at least 9, at least 10, etc.); glycine-serine polymers (GS) n, where n is an integer of at least 1 (e.g., at least one, at least 2, at least 3, at least 4, at least 5, at least 6, at least 7, at least 8, at least 9, at least 10, etc.) such as SEQ ID NOs: 249-257; glycine-alanine polymers; alanine-serine polymers; and the like. Linker sequences may be of any length, such as from about 1 amino acid (e.g., glycine or serine) to about 20 amino acids (e.g., 20 amino acid glycine polymers or glycine-serine polymers), about 1 amino acid to about 15 amino acids, about 3 amino acids to about 12 amino acids, about 4 amino acids to about 10 amino acids, about 5 amino acids to about 9 amino acids, about 6 amino acids to about 8 amino acids, etc. In some embodiments, the linker is any of about 1, 2, 3, 4, 5, 6, 7, 8, 9, 10, 11, 12, 13, 14, 15, 16, 17, 18, 19, or 20 amino acids in length.

In some embodiments, the LU comprises an amino acid sequence selected from the group consisting of SEQ ID NO: 213 and 213. In some embodiments, the masking peptide (MP) comprises the structure, from N-terminus to C-terminus, of: (MU)-(LU), wherein LU comprises the structure (CS₁)-L, or (CS₁)-L₁-(CS₂)-L₂. In some embodiments, the masking peptide of the present disclosure comprises an amino acid sequence selected from the group consisting of SEQ ID NOs: 215-248.

In some embodiments, the masking peptide (MP) comprises an MU set forth in any one of SEQ ID NOs: 178-211 and an LU set forth in SEQ ID NO: 213 or 214. In some embodiments, the MP comprises a sequence set forth in any one of SEQ ID NOs: 215-248.

### VII. Antibody Production

Another aspect of the disclosure provides one or more isolated nucleic acid molecule (s) that comprises nucleotide sequence (s) encoding an amino acid sequence (s) of an anti-CD28 antibody described herein, including a masked anti-CD28 antibody. In some embodiments, provided herein is one or more isolated nucleic acid molecule (s) that comprises nucleotide sequence (s) encoding an amino acid sequence (s) of a multi-specific antibody described herein, including a masked multi-specific antibody. The amino acid sequence encoded by the nucleotide sequence may be any portion of an antibody described herein, such as a CDR, a sequence comprising one, two, or three CDRs, a variable region of a heavy chain, variable region of a light chain, or may be a full-length heavy chain or full-length light chain. A nucleic acid of the disclosure can be, for example, DNA or RNA, and may or may not contain intronic sequences. Typically, the nucleic acid is a cDNA molecule.

In some embodiments, the disclosure provides an isolated nucleic acid molecule that comprises or consists of a nucleotide sequence encoding an amino acid sequence of, e.g., a heavy chain variable region and/or a light chain variable region of an antibody described herein, or, e.g., a full-length heavy chain and/or full-length light chain of an antibody described herein.

Nucleic acids of the disclosure can be obtained using any suitable molecular biology techniques, e.g., PCR amplification or cDNA cloning techniques. For antibodies described herein obtained via library screening, the nucleic acid encoding the antibody can be recovered from the library.

The isolated DNA encoding the V_{H} region can be converted to a full-length heavy chain gene by operatively linking the V_{H}-encoding DNA to another DNA molecule encoding heavy chain constant regions (C_{H}1, C_{H}2 and C_{H}3). The sequences of human heavy chain constant region genes are known in the art (see e.g., Kabat et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG₁, IgG₂, IgG₃, IgG₄, IgA, IgE, IgM or IgD constant region, but most preferably is an IgG₄ or IgG₂ constant region without ADCC effect. The IgG₄ constant region sequence can be any of the various alleles or allotypes known to occur among different individuals. These allotypes represent naturally occurring amino acid substitution in the IgG₄ constant regions. For a Fab fragment heavy chain gene, the V_{H}-encoding DNA can be operatively linked to another DNA molecule encoding only the heavy chain C_{H}1 constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art (see e.g., Kabat et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

To create a scFv gene, the V_{H}- and V_{L}-encoding DNA fragments are operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} regions joined by the flexible linker (see e.g., Bird et al., Science (1988) 242: 423-426; Huston et al., Proc. Natl. Acad. Sci. USA (1988) 85: 5879-83; and McCafferty et al., Nature (1990) 348: 552-554).

The present disclosure further provides a vector that comprises one or more nucleic acid molecule (s) provided by the present disclosure. In some embodiments, the vector is an expression vector useful for the expression of an antibody described herein or an antigen binding fragment of such an antibody. In some embodiments, provided herein are vectors, wherein a first vector comprises a polynucleotide sequence encoding a heavy chain variable region as described herein, and a second vector comprises a polynucleotide sequence encoding a light chain variable region as described herein. In some embodiments, a single vector comprises polynucleotides encoding a heavy chain variable region as described herein and a light chain variable region as described herein.

To express a binding molecule of the disclosure, DNAs encoding partial or full-length light and heavy chains are inserted into expression vectors such that the DNA molecules are operatively linked to transcriptional and translational control sequences. In this context, the term “operatively linked” means that an antibody gene is ligated into a vector such that transcriptional and translational control sequences within the vector serve their intended function of regulating the transcription and translation of the DNA molecule. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by any suitable methods (e.g., ligation of complementary restriction sites on the antibody gene fragment and vector, or homologous recombination-based DNA ligation). The light and heavy chain variable regions of the antibodies described herein can be used to create full-length antibody genes of any antibody isotype and subclass by inserting them into expression vectors already encoding heavy chain constant and light chain constant regions of the desired isotype and subclass such that the V_{H} segment is operatively linked to the C_{H} segment (s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the expression vectors of the disclosure typically carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Examples of regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SR promoter system, which contains sequences from the SV40 early promoter and the long terminal repeat of human T cell leukemia virus type 1 (Takebe, Y. et al. Mol. Cell. Biol. (1988) 8: 466-72).

In addition to the antibody chain genes and regulatory sequences, the expression vectors may carry additional sequences, such as enhancer element (s), a transcription termination sequence (s), sequence (s) that regulate replication of the vector in host cells (e.g., origins of replication) and selectable marker gene (s). The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the light and heavy chains, the expression vector (s) encoding the heavy and light chains is transfected into a host cell by any suitable techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies described herein in either prokaryotic or eukaryotic host cells, expression of antibodies in eukaryotic cells, e.g., mammalian host cells, is most typical.

The present disclosure further provides a host cell containing nucleic acid molecule (s) or vector (s) provided by the present disclosure. The host cell can be virtually any cell for which expression vectors are available. It may be, for example, a higher eukaryotic host cell, such as a mammalian cell, a lower eukaryotic host cell, such as a yeast cell, and may be a prokaryotic cell, such as a bacterial cell. Introduction of the recombinant nucleic acid construct into the host cell can be effected by calcium phosphate transfection, DEAE, dextran mediated transfection, electroporation or phage infection.

Suitable prokaryotic hosts for transformation include E. coli, Bacillus subtilis, Salmonella typhimurium and various species within the genera Pseudomonas, Streptomyces, and Staphylococcus.

Mammalian host cells for expressing a binding molecule of the disclosure include, for example, Chinese Hamster Ovary (CHO) cells (including dhfr-CHO cells, described in Urlaub and Chasin, Proc. Natl. Acad. Sci. USA (1980) 77: 4216-20, used with a DHFR selectable marker, e.g., as described in Kaufman and Sharp, J. Mol. Biol. (1982) 159: 601-21, NSO myeloma cells, COS cells and Sp2 cells. In particular, for use with NSO myeloma or CHO cells, another expression system is the GS (glutamine synthetase) gene expression system disclosed in WO 87/04462, WO 89/01036, and EP338841.

An antibody (or antigen binding fragment thereof) of the present disclosure may be produced by any means known in the art. Exemplary techniques for antibody production are in U.S. Pat. No. 4,816,567; however, these exemplary techniques are provided for illustrative purposes only and are not intended to be limiting. When nucleic acid (s) or expression vector (s) encoding an antibody described herein are introduced into a host cell, the antibody is produced by culturing the host cell for a period of time sufficient to allow for expression of the antibody in the host cells or secretion of the antibody into the culture medium in which the host cells are grown. Thus, in some embodiments, provided is a method of producing an antibody described herein, which method comprises culturing a host cell comprising one or more nucleic acid (s) or vector (s) that encode the antibody (e.g., as provided above) under conditions suitable for expression of the antibody. In some embodiments, the method further comprises recovering the antibody from the host cell (or host cell culture medium). The antibody can be recovered from the culture medium using any suitable protein purification methods.

### VIII. Pharmaceutical Compositions

In some embodiments, the present disclosure provides a composition comprising one or more of the antibodies described herein. In some embodiments, the composition is a pharmaceutical composition comprising an antibody described herein and a pharmaceutically acceptable carrier. The compositions can be prepared by conventional methods known in the art.

The term “pharmaceutically acceptable carrier” refers to any inactive substance that is suitable for use in a formulation for the delivery of a polypeptide (e.g., an antibody described herein). A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride.

The compositions may be in any suitable forms, such as liquid, semi-solid, and solid dosage forms. Examples of liquid dosage forms include solution (e.g., injectable and infusible solutions), microemulsion, liposome, dispersion, or suspension. Examples of solid dosage forms include tablet, pill, capsule, microcapsule, and powder. A particular form of the composition suitable for delivering an antibody described herein is a sterile liquid, such as a solution, suspension, or dispersion, for injection or infusion. Sterile solutions can be prepared by incorporating the antibody in the required amount in an appropriate carrier, followed by sterilization microfiltration. Dispersions may be prepared by incorporating the antibody into a sterile vehicle that contains a basic dispersion medium and other carriers. In the case of sterile powders for the preparation of sterile liquid, methods of preparation include vacuum drying and freeze-drying (lyophilization) to yield a powder of the active ingredient plus any additional desired ingredient from a previously sterile-filtered solution thereof. The various dosage forms of the compositions can be prepared by conventional techniques known in the art.

The relative amount of an antibody included in the composition will vary depending upon a number of factors, such as the specific polypeptide and carriers used, dosage form, and desired release and pharmacodynamic characteristics. The amount of an antibody in a single dosage form will generally be that amount which produces a therapeutic effect, but may also be a lesser amount. Generally, this amount will range from about 0.01 percent to about 99 percent, from about 0.1 percent to about 70 percent, or from about 1 percent to about 30 percent relative to the total weight of the dosage form.

In addition to the antibody described herein, one or more additional therapeutic agents may be included in the composition. In some embodiments, the at least one additional therapeutic agent is selected from the group consisting of viral gene therapy, immune checkpoint inhibitors, target therapies, radiation therapies, and chemotherapies. In some embodiments, the at least one additional therapeutic agent is selected from the group consisting of pomalyst, revlimid, lenalidomide, pomalidomide, thalidomide, a DNA-alkylating platinum-containing derivative, cisplatin, 5-fluorouracil, cyclophosphamide, an anti-CTLA4 antibody, an anti-PD-1 antibody, an anti-PD-L1 antibody, an anti-CD20 antibody, an anti-CD40 antibody, an anti-DR5 antibody, an anti-CD1d antibody, an anti-TIM3 antibody, an anti-SLAMF7 antibody, an anti-KIR receptor antibody, an anti-OX40 antibody, an anti-HER2 antibody, an anti-ErbB-2 antibody, an anti-EGFR antibody, cetuximab, rituximab, trastuzumab, pembrolizumab, radiotherapy, single dose radiation, fractionated radiation, focal radiation, whole organ radiation, IL-12, IFNα, GM-CSF, a chimeric antigen receptor, adoptively transferred T cells, an anti-cancer vaccine, and an oncolytic virus. The suitable amount of the additional therapeutic agent to be included in the composition can be readily selected by a person skilled in the art, and will vary depending on a number of factors, such as the particular agent and carriers used, dosage form, and desired release and pharmacodynamic characteristics. The amount of the additional therapeutic agent included in a single dosage form will generally be that amount of the agent which produces a therapeutic effect, but may be a lesser amount as well.

The antibodies described herein may be further modified. In some embodiments, the antibodies are linked to an additional molecular entity. Examples of additional molecular entities include pharmaceutical agents, peptides or proteins, detection agent or labels, and antibodies.

In some embodiments, an antibody described herein is linked to a pharmaceutical agent. Examples of pharmaceutical agents include cytotoxic agents or other cancer therapeutic agents, and radioactive isotopes. Specific examples of cytotoxic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Examples of radioactive isotopes that can be conjugated to antibodies for use diagnostically or therapeutically include, but are not limited to, iodine³¹, indium¹¹¹, yttrium⁹⁰ and lutetium¹⁷⁷. Methods for linking a polypeptide to a pharmaceutical agent are known in the art, such as using various linker technologies. Examples of linker types include hydrazones, thioethers, esters, disulfides and peptide-containing linkers. For further discussion of linkers and methods for linking therapeutic agents to antibodies see e.g., Saito et al., Adv. Drug Deliv. Rev. (2003) 55: 199-15; Trail, et al., Cancer Immunol. Immunother. (2003) 52: 328-37; Payne, Cancer Cell (2003) 3:207-12; Allen, Nat. Rev. Cancer (2002) 2: 750-63; Pastan and Kreitman, Curr. Opin. Investig. Drugs (2002) 3: 1089-91; Senter and Springer Adv. Drug Deliv. Rev. (2001) 53: 247-64.

Any of the antibodies and/or compositions (e.g., pharmaceutical compositions) described herein may be used in the preparation of a medicament (e.g., a medicament for use in treating or delaying progression of cancer in a subject in need thereof).

### IX. Methods of Treatment

The antibodies and pharmaceutical compositions described herein are useful for therapeutic purposes, such as treating cancer or enhancing the efficacy of other cancer therap(ies). Thus, in some embodiments, the present disclosure provides methods of using the antibodies or pharmaceutical compositions described herein. In some embodiments, the present disclosure provides a method of treating cancer in a subject (e.g., a human subject), comprising administering to the subject an effective amount of an antibody described herein. In some embodiments, the cancer is breast cancer, liver cancer, or colorectal cancer, gastric cancer, ovarian cancer, lung cancer, pancreatic cancer, or kidney cancer.

In practicing the therapeutic methods, the masked anti-CD28 antibodies described herein may be administered alone, i.e., as monotherapy, or administered in combination with one or more additional therapeutic agents or therapies. Thus, in another aspect, the present disclosure provides a combination therapy, which comprises a binding molecule in combination with one or more additional therapies or therapeutic agents for separate, sequential or simultaneous administration. In some embodiments, the term “additional therapy” refers to a therapy which does not employ an antibody described herein as a therapeutic agent. In some embodiments, the term “additional therapeutic agent” refers to any therapeutic agent other than an antibody described herein. In some embodiments, the present disclosure provides a method of treating cancer in a subject (e.g., a human subject) that comprises administering to the subject an effective amount of an antibody described herein (e.g., an anti-CD28 antibody or multi-specific antibody that targets CD28 and one or more other targets) and an effective amount of an anti-PD-1 antibody. In some embodiments, the present disclosure provides a method of treating cancer in a subject (e.g., a human subject) that comprises administering to the subject an effective amount of an antibody described herein (e.g., an anti-CD28 antibody or multi-specific antibody that targets CD28 and one or more other targets) and an effective amount of an anti-CTLA4 antibody. In some embodiments, the anti-CTLA4 antibody is a masked anti-CTLA4 antibody.

### X. Kits and Articles of Manufacture

In some embodiments, provided is a kit comprising one or more antibodies described herein (e.g., an anti-CD28 antibody or multi-specific antibody that targets CD28 and one or more other targets). In some embodiments, the kit further comprises a package insert comprising instructions for use of the antibodies described herein. In some embodiments, the article of manufacture or kit comprises a container containing one or more of the masked antibodies or compositions described herein. In certain embodiments, the article of manufacture or kit comprises a container containing nucleic acid (s) encoding one (or more) of the masked antibodies described herein. In some embodiments, the kit includes a cell of cell line that produces an antibody described herein. In some embodiments, the kit includes one or more positive controls, for CD28 (e.g., human CD28, cynomolgus CD28, mouse CD28, rat CD28 or fragments of any of the preceding) or CD28⁺ cells. In some embodiments, the kit includes negative controls, for example a surface or solution that is substantially free of CD28, or a cell that does not express CD28.

In certain embodiments, the article of manufacture or kit comprises a container and a label or package insert on or associated with the container. Suitable containers include, for example, bottles, vials, syringes, IV solution bags, test tubes, etc. The containers may be formed from a variety of materials such as glass or plastic. The container holds an antibody described herein (or a composition comprising such an antibody), which is by itself or combined with another composition effective for treating, delaying progression of, and/or preventing cancer in a subject (e.g., a human subject). The container may have a sterile access port (for example the container may be an intravenous solution bag or a vial having a stopper pierceable by a hypodermic injection needle). In some embodiments, the label or package insert indicates that the composition is used for treating breast cancer, liver cancer, or colorectal cancer in a subject (e.g., a human subject).

Moreover, the article of manufacture or kit may comprise (a) a first container with a composition contained therein, wherein the composition comprises an antibody described herein (or immunologically active fragment thereof); and (b) a second container with a composition contained therein, wherein the composition comprises a further cytotoxic or otherwise therapeutic agent. In some embodiments, the second container contains a composition comprising an anti-PD-1 antibody, and the article of manufacture comprises a the label or package insert indicates that the antibody and the anti-PD-L1 are for use in the treatment of colon cancer in a subject (e.g., human subject) in need thereof, e.g., according to a method provided herein. In some embodiments, the second container contains a composition comprising an anti-CTLA4 antibody (e.g., a masked anti-CTLA4 antibody), and the article of manufacture comprises a label or package insert indicates that the antibody described herein and the anti-CTLA4 antibody (e.g., a masked anti-CTLA4 antibody) are for use in the treatment of colon cancer in a subject (e.g., human subject) in need thereof, e.g., according to a method provided herein.

Additionally, the article of manufacture may further comprise an additional container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The foregoing written description is considered to be sufficient to enable one skilled in the art to practice the present disclosure. The following Examples are offered for illustrative purposes only, and are not intended to limit the scope of the present disclosure in any way. Indeed, various modifications of the present disclosure in addition to those shown and described herein will become apparent to those skilled in the art from the foregoing description and fall within the scope of the appended claims.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All references cited herein, including patent applications, patent and non-patent publications, and UniProtKB/Swiss-Prot Accession numbers, are herein incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Any compound disclosed herein can be used in any of the treatment method here, wherein the individual to be treated is as defined anywhere herein. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Generation of Primary Fabs that Specifically Bind to Human CD28

Proprietary phagemid libraries were employed to pan against human CD28 antigens. A total of three or four rounds of panning were conducted. After the final round of panning, the culture supernatants of individual clones were tested by ELISA to identify those that specifically recognized human CD28 (i.e., primary hits). Clones were defined as positive when ELISA signals were at least twice that of background. The positive clones were picked to confirm sequence and the Fabs corresponding to the unique hits were expressed in in E. coli and affinity purified. Their affinities against human CD28 were measured by the Octet® RED96 Systems (ForteBio). Briefly, Dip and Read Anti-Human IgG Fc Capture (AHC) biosensors (ForteBio) were used to capture human or mouse CD28-Fc fusion proteins, and dipped into wells containing purified Fabs that were diluted 5 to 10-fold with ForteBio kinetic buffer (10 mM HEPES, 150 mM NaCl, 3 mM EDTA, 0.005% v/v Surfactant P20, pH 7.4). The acquired data were processed with Octet Data Analysis Software Version 7.1 (ForteBio), and kinetic data were fitted to a 1:1 Langmuir binding model.

The primary Fabs hits were further characterized for human and mouse CD28 species cross-reactivity by ELISA and, from these primary hits, a panel of 46 unique sequence Fabs were then converted into IgG₄ isotype mAbs with the core hinge mutation S241P (Kabat numbering scheme) for detailed biophysical and functional characterization (Table 5).

### Example 2: IgG₄ Conversion and Expression

The heavy chains and light chains of the engineered human anti-CD28 IgG₄ isotype mAbs listed in Table 5 were cloned into the mammalian expression vector pcDNA3.3 (ThermoFisher Scientific). Pairs of plasmids bearing one heavy and one light chain were transiently transfected into HEK293 cells following the manufacturer's instructions. After incubation, the supernatants were harvested, cleared by centrifugation and filtration, and the IgG₄ isotype mAbs were captured by MabSelect™ SuRe™ protein A affinity chromatography (GE Healthcare).

The mAbs were eluted and neutralized, and the eluate buffer exchanged into restore buffer (20 mM histidine, pH 5.5). Protein concentrations were determined by UV spectrophotometry, and IgG purity was analyzed under denaturing, reducing, and non-reducing conditions by SDS-PAGE or SEC-HPLC.

TABLE 5
List of Human Anti-CD28 IgG₄ Monoclonal Antibodies

| No. | IgG ID | Isotype/mutation |
|---|---|---|
| 1 | TY24853 | hIgG₄/S241P |
| 2 | TY24854 | hIgG₄/S241P |
| 3 | TY24855 | hIgG₄/S241P |
| 4 | TY24856 | hIgG₄/S241P |
| 5 | TY24857 | hIgG₄/S241P |
| 6 | TY24858 | hIgG₄/S241P |
| 7 | TY24859 | hIgG₄/S241P |
| 8 | TY24860 | hIgG₄/S241P |
| 9 | TY24861 | hIgG₄/S241P |
| 10 | TY24862 | hIgG₄/S241P |
| 11 | TY24863 | hIgG₄/S241P |
| 12 | TY24864 | hIgG₄/S241P |
| 13 | TY24865 | hIgG₄/S241P |
| 14 | TY24866 | hIgG₄/S241P |
| 15 | TY24867 | hIgG₄/S241P |
| 16 | TY24868 | hIgG₄/S241P |
| 17 | TY24869 | hIgG₄/S241P |
| 18 | TY24870 | hIgG₄/S241P |
| 19 | TY24871 | hIgG₄/S241P |
| 20 | TY24872 | hIgG₄/S241P |
| 21 | TY24873 | hIgG₄/S241P |
| 22 | TY24874 | hIgG₄/S241P |
| 23 | TY24875 | hIgG₄/S241P |
| 24 | TY24876 | hIgG₄/S241P |
| 25 | TY24877 | hIgG₄/S241P |
| 26 | TY24878 | hIgG₄/S241P |
| 27 | TY24879 | hIgG₄/S241P |
| 28 | TY24880 | hIgG₄/S241P |
| 29 | TY24881 | hIgG₄/S241P |
| 30 | TY24882 | hIgG₄/S241P |
| 31 | TY24883 | hIgG₄/S241P |
| 32 | TY24884 | hIgG₄/S241P |
| 33 | TY24885 | hIgG₄/S241P |
| 34 | TY24886 | hIgG₄/S241P |
| 35 | TY24887 | hIgG₄/S241P |
| 36 | TY24888 | hIgG₄/S241P |
| 37 | TY24889 | hIgG₄/S241P |
| 38 | TY24890 | hIgG₄/S241P |
| 39 | TY24891 | hIgG₄/S241P |
| 40 | TY24892 | hIgG₄/S241P |
| 41 | TY24772 | hIgG₄/S241P |
| 42 | TY24773 | hIgG₄/S241P |
| 43 | TY24774 | hIgG₄/S241P |
| 44 | TY24775 | hIgG₄/S241P |
| 45 | TY24776 | hIgG₄/S241P |
| 46 | TY24777 | hIgG₄/S241P |

### Example 3: Binding Characterization of Anti-CD28 Antibodies

The binding affinities of the panel of anti-CD28 mAbs to human, cynomolgus monkey, and mouse CD28 and human CTLA4 were measured by the Octet® RED96 Systems (ForteBio), ELISA, and CytoFlex flow cytometry (Beckman). The anti-CD28 antibody TAC2386 (also known as TGN1412 as described in Patent WO2006/050949A2) and TAC2387 (as described in Patent WO2019/246514A2) were used as a benchmark control.

### Binding Affinity to Human CD28 and CTLA4 by the Octet RED96 Systems

The Octet® RED96 Systems (ForteBio) was used to assess the binding kinetics of the panel of anti-CD28 mAbs to human CD28 and CTLA4. Briefly, the mAbs were diluted to 15 μg/mL in kinetic buffer (PBS supplemented with 0.02% Tween 20 and 0.1% BSA), and captured by Dip and Read AHC biosensors (ForteBio) in parallel. The sensors were then allowed to associate with His-tagged human CD28 and CTLA4 proteins (100 nM) for 300 seconds, and to dissociate in kinetic buffer for another 300 seconds. The association and dissociation curves were fitted to a 1:1 Langmuir binding model using the Octet® Data Analysis Software Version 7.1 (ForteBio).

As shown in Table 6, the panel of anti-CD28 test and benchmark mAbs exhibited high binding affinity (<10 nM) to human CD28, as measured by the Octet® RED96 Systems. In addition, the test mAbs TY24773, TY24853, TY24854, TY24860, TY24865, and TY24871 and the benchmark control mAbs exhibited no detectable affinity to human CTLA4.

TABLE 6
Binding Affinity to Human CD28 and CTLA4

| | Human CD28 | | | Human CTLA4 | | |
|---|---|---|---|---|---|---|
| | KD | Kon | Koff | KD | Kon | Koff |
| mAb ID | (nM) | (1/Ms) | (1/s) | (nM) | (1/Ms) | (1/s) |
| TAC2386 | <0.001 | 9.625E+04 | <1.0E−07 | ND | | |
| TAC2387 | 4.873 | 7.071E+04 | 3.446E−04 | | | |
| TY24773 | 8.285 | 3.848E+04 | 3.188E−04 | | | |
| TY24853 | 3.955 | 4.159E+04 | 1.645E−04 | | | |
| TY24854 | 5.434 | 6.488E+04 | 3.526E−04 | | | |
| TY24855 | 7.927 | 7.918E+04 | 6.277E−04 | 4.022 | 7.596E+05 | 3.055E−03 |
| TY24856 | 7.805 | 6.887E+04 | 5.376E−04 | 5.127 | 3.036E+05 | 1.557E−03 |
| TY24857 | 8.639 | 7.294E+04 | 6.301E−04 | 5.442 | 6.515E+05 | 3.546E−03 |
| TY24858 | 9.882 | 7.101E+04 | 7.017E−04 | 7.904 | 3.055E+05 | 2.415E−03 |
| TY24859 | 2.684 | 7.081E+04 | 1.900E−04 | 6.631 | 4.687E+05 | 3.108E−03 |
| TY24860 | 6.664 | 6.323E+04 | 4.213E−04 | ND | | |
| TY24864 | 4.413 | 9.418E+04 | 4.157E−04 | 5.501 | 5.168E+05 | 2.843E−03 |
| TY24865 | <0.001 | 5.958E+04 | <1.0E−07 | ND | | |
| TY24866 | <0.001 | 5.804E+04 | <1.0E−07 | 7.645 | 5.238E+05 | 4.004E−03 |
| TY24869 | 2.733 | 4.676E+04 | 1.278E−04 | 1.198 | 3.793B+05 | 4.544E−04 |
| TY24871 | 4.993 | 7.011E+04 | 3.501E−04 | ND | | |
| TY24876 | <0.001 | 3.251E+04 | <1.0E−07 | 4.280 | 1.366E+05 | 5.848E−04 |
| TY24877 | 7.191 | 2.686E+04 | 1.931E−04 | 13.20 | 2.353E+05 | 3.105E−03 |
| TY24878 | 4.214 | 9.747E+04 | 4.108E−04 | 0.9686 | 4.233E+05 | 4.100E−04 |
| TY24879 | <0.001 | 5.939E+04 | <1.0E−07 | 7.165 | 4.369E+05 | 3.130E−03 |
| TY24881 | 5.246 | 1.262E+05 | 6.618E−04 | 2.353 | 1.114E+06 | 2,622E−03 |
| TY24884 | 2.420 | 6.836E+04 | 1.655E−04 | 6.359 | 6.409E+05 | 4.076E−03 |
| TY24890 | 4.440 | 9.846E+04 | 4.371E−04 | 3.133 | 1.099E+06 | 3.444E−03 |

ND: Not detected

### Binding Affinity to Human and Mouse CD28 Using ELISA

Recombinant human and mouse CD28-Fc were diluted to 2 g/mL in PBS and coated on Nunc MaxiSorp™ high protein-binding capacity 96 well ELISA plates (ThermoFisher Scientific) at 4° C. overnight. Plates were blocked with PBS supplemented with 3% non-fat milk at 37° C. for 1 hour. After washing, 50 PL of 3-fold serial dilutions of a panel of anti-CD28 test mAbs were added to each well. After incubation at 37° C. for 1 hour, plates were washed four times, and 100 μL of a horseradish peroxidase (HRP)-conjugated anti-human IgG (Fab specific) (1:6000 dilution) secondary antibody was added to each well. Plates were incubated at 37° C. for 1 hour, washed four times, and then 50 μL of a TMB substrate (3, 3′, 5, 5′-tetramethylbenzidine) solution was added to each well, and the plate was incubated at room temperature. Absorbance at 450 nm was measured after the reactions were stopped by adding 50 μL of a sulfuric acid stop solution to each well. The EC₅₀ was evaluated by fitting the ELISA data using the asymmetrical sigmoidal (4-parameter logistic equation) model of GraphPad Prism version 7 for Windows, GraphPad Software, La Jolla California USA, www.graphpad.com.

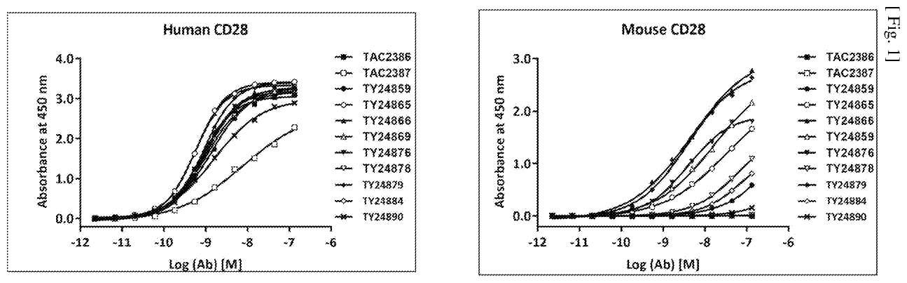

As shown in Table 7 and FIG. 1, the panel of anti-CD28 test mAbs exhibited similar affinities to human CD28 as the two benchmark controls. Moreover, the test mAbs except TY24890 bound to mouse CD28 while the two benchmark controls did not.

TABLE 7
Binding Affinity to Recombinant CD28 Measured by ELISA

EC₅₀ (nM)

| mAb ID | Human CD28 | Mouse CD28 |
|---|---|---|
| TAC2386 | 0.78 | ND |
| TAC2387 | >1.65 | ND |
| TY24859 | 1.34 | >44.44 |
| TY24865 | 0.58 | >4.94 |
| TY24866 | 0.57 | >1.65 |
| TY24869 | 0.87 | >14.81 |
| TY24876 | 1.02 | 3.93 |
| TY24878 | 1.06 | >14.81 |
| TY24879 | 0.9 | >1.65 |
| TY24884 | 1.02 | >44.44 |
| TY24890 | 1.55 | ND |

ND: Not detected

### Binding Activities of Anti-CD28 Antibodies on Jurkat Cells

Jurkat (clone E6-1) cells were seeded in 96-well plates at 1.0×10⁵ (50 μL/well) and incubated with serially diluted benchmark positive controls, an isotype negative control antibody, and a panel of anti-CD28 test mAbs (100, 20, 4, 0.8, 0.16, and 0.032 nM) for 30 minutes at 4° C. in 2% fetal bovine serum/Dulbecco's PBS (FBS/DPBS). Next, the cells were washed twice with DPBS and further incubated with a APC-anti-human IgG Fc secondary antibody (1 μg/mL, 100 μL/well, Biolegend) for 30 minutes at 4° C. Finally, the cells were washed twice with DPBS and suspended in FACS buffer for flow cytometry analysis. For analysis, the Mean Fluorescence Intensity (MFI) values versus concentrations were plotted using FlowJo 10 software (FlowJo LLC) and the data were further fitted with four-parameter non-linear regression to obtain EC₅₀ values by GraphPad Prism version 7 for Windows, GraphPad Software, La Jolla California USA, www.graphpad.com.

As shown in Table 8, except for TY24775 and TY24867, all other anti-CD28 test mAbs exhibited high binding activity (EC₅₀ values ranging from 0.38 to 10.42 nM) to CD28 molecules on Jurkat cells. In addition, the measured binding activities were comparable to the two benchmark controls.

TABLE 8
Binding Affinity to CD28 expressed on
Jurkat cells Measured by Flow Cytometry

| Ab ID | EC₅₀ (nM) | Ab ID | EC₅₀ (nM) | Ab ID | EC₅₀ (nM) |
|---|---|---|---|---|---|
| TAC2386 | 1.10 | TY24863 | 2.60 | TY24881 | 0.38 |
| TAC2387 | 0.81 | TY24864 | 0.54 | TY24882 | 2.24 |
| TY24772 | 0.74 | TY24865 | 0.46 | TY24883 | 5.92 |
| TY24773 | 0.71 | TY24866 | 0.70 | TY24884 | 1.09 |
| TY24774 | 3.35 | TY24867 | ND | TY24885 | 5.77 |
| TY24775 | ND | TY24868 | 2.08 | TY24886 | 10.42 |
| TY24776 | 1.83 | TY24869 | 1.07 | TY24887 | 3.17 |
| TY24777 | 1.50 | TY24870 | 3.95 | TY24888 | 0.58 |
| TY24853 | 0.43 | TY24871 | 0.89 | TY24889 | 2.32 |
| TY24854 | 0.60 | TY24872 | 1.07 | TY24890 | 0.59 |
| TY24855 | 0.61 | TY24873 | 2.08 | TY24891 | 0.62 |
| TY24856 | 0.76 | TY24874 | 1.65 | TY24892 | 1.30 |
| TY24857 | 0.75 | TY24875 | 1.72 | Isotype | ND |
| | | | | control | |
| TY24858 | 0.70 | TY24876 | 0.38 | | |
| TY24859 | 0.97 | TY24877 | 0.79 | | |
| TY24860 | 0.78 | TY24878 | 0.39 | | |
| TY24861 | 2.25 | TY24879 | 1.17 | | |
| TY24862 | 2.82 | TY24880 | 1.62 | | |

ND: Not detected

### In Vitro Binding of Anti-CD28 Antibodies to Human T Cells by Flow Cytometry

Human CD3⁺ T cells were isolated from cryopreserved peripheral blood mononuclear cells (PBMCs) using the EasySep™ Human Naïve Pan T Cell Isolation Kit (STEMCELL Technologies). The isolated human T cells were added in 96-well plate at 1.0×10⁵ cells/well and incubated with 100 nM of benchmark controls, an isotype negative control antibody, and a panel of anti-CD28 test mAbs for 30 minutes on ice in FACS buffer. Next, the cells were washed three times with PBS and further incubated with PE-labeled secondary antibody for 30 minutes on ice. Finally, the cells were washed three times with PBS and resuspended in FACS buffer for flow cytometry analysis. For analysis, the MFI values were calculated using FlowJo 10 software (FlowJo LLC) and the geometric mean MFI values versus mAb were plotted using GraphPad Prism version 7 for Windows, GraphPad Software, La Jolla California USA, www.graphpad.com.

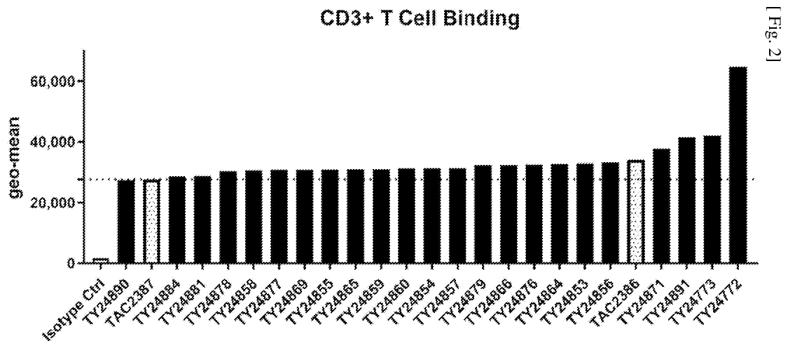

As shown in FIG. 2, compared with the benchmark controls TAC2386 and TAC2387, all of the test mAbs except TY24772 exhibited comparable MFI values. TY24772 binding was approximately twice that of the benchmark controls.

### Example 4: Ligand Competition Using Elisa

Antibodies were tested for their ability to block the binding of CD28 or CTLA4 to its natural ligand CD80 by ELISA.

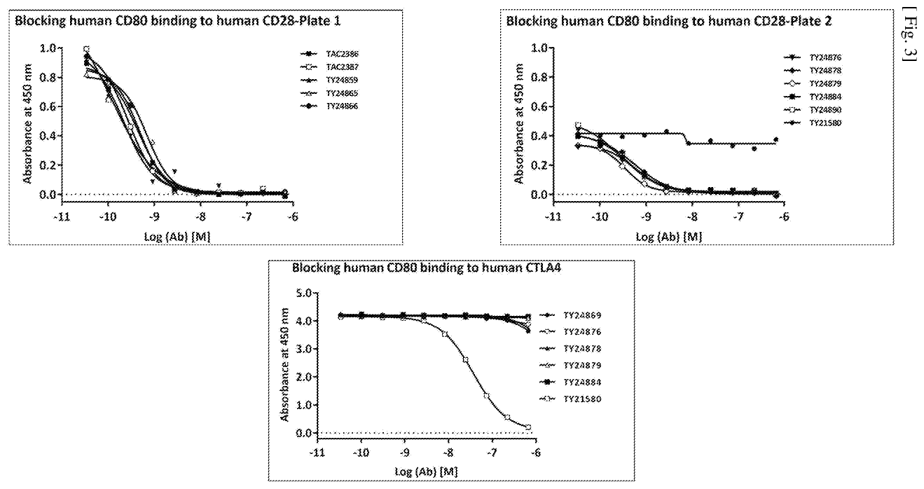

As shown in FIG. 3 and Table 9, the benchmark controls TAC2386 and TAC2387 and all of the tested anti-CD28 test mAbs blocked the binding of CD28 to CD80, while none blocked the binding of CTLA4 to CD80, compared with an anti-CTLA4 antibody TY21580 included as a control.

TABLE 9
IC₅₀ of Anti-CD28 or Anti-CTLA4 Antibodies
for Human CD28-CD80 and CTLA4-CD80 Pairs

IC₅₀ (nM)

| Sample ID | CD28/CD80 | CTLA4/CD80 |
|---|---|---|
| TAC2386 | 0.48 | — |
| TAC2387 | 0.12 | — |
| TY24859 | 0.42 | — |
| TY24865 | 0.68 | — |
| TY24866 | 0.27 | — |
| TY24890 | 0.21 | — |
| TY24869 | 0.55 | ND |
| TY24876 | 0.54 | ND |
| TY24878 | 0.35 | ND |
| TY24879 | 0.42 | ND |
| TY24884 | 0.28 | ND |
| TY21580 | ND | 37.24 |

### Example 5: Assessment of Superagonism in Dry-Coated Human T Cell Proliferation Assays

The levels of lymphocyte proliferation induced by the benchmark controls TAC2386 and TAC2387, an isotype negative control antibody, and a panel of anti-CD28 test mAbs were measured by the CellTiter-Glo® Luminescent Cell Viability Assay (Promega). In this assay, 100 nM of test mAbs at 50 μL per well were added to 96-well microplate in triplicate and air-dried directly onto the walls of the wells. Next, the microplate were washed twice with PBS. Cryopreserved PBMCs were restored and the cell densities were adjusted to 5.0×10⁵ cells/mL with 10% FBS/RPMI1640 and 1.0×10⁵ PBMCs (200 μL per well) were added to the pre-coated microplates. The cells were incubated at 37° C., 5% CO₂ for 72 hours and then the lymphocyte proliferation was assessed using the CellTiter-Glo® assay.

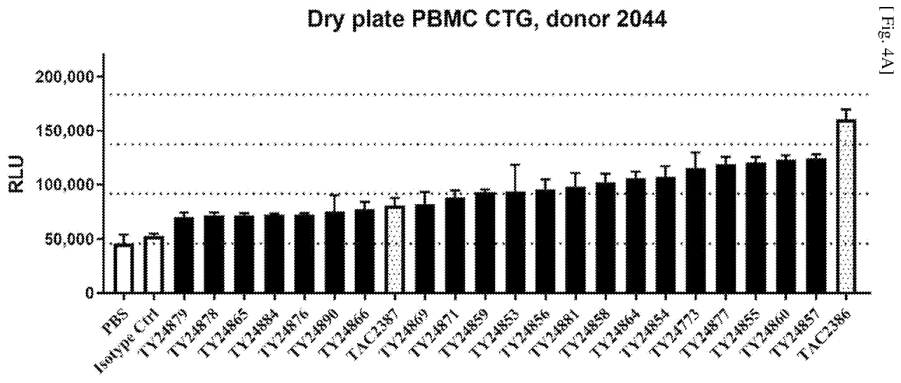

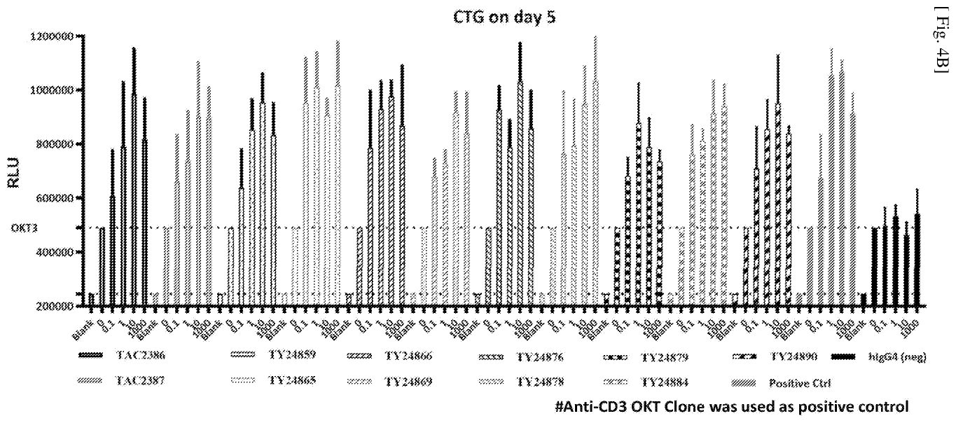

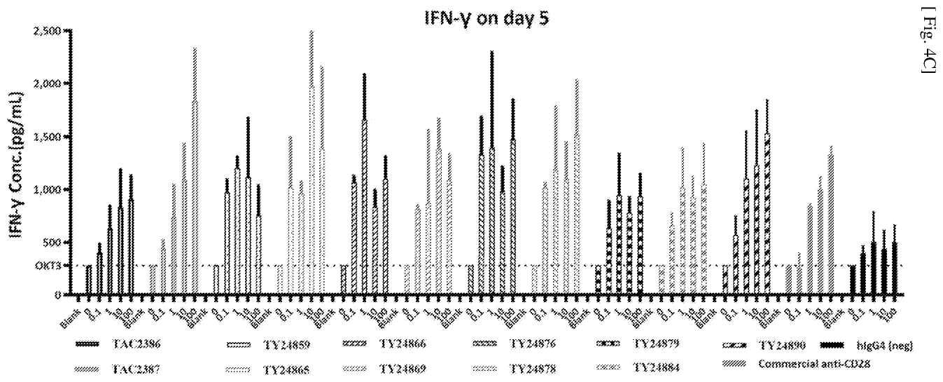

As shown in FIG. 4A, the level of lymphocyte proliferation induced by immobilized benchmark control TAC2386 (aknown CD28 superagonist) was significantly higher than that of all the test mAbs. Compared with TAC2386, the benchmark control TAC2387 and the anti-CD28 test mAbs including TAC2387, TY24865, TY24866, TY24876, TY24878, TY24879 and TY24884 exhibited weak super agonistic activity in vitro.

### Example 6: Effects of Anti-CD28 Antibodies on Human T Cell Activation in Vitro T Cell Activation Co-Stimulation Assay: IFN-γ Release

The biological activity of the anti-CD28 mAbs, as agonistic T cell co-stimulatory agents in activating human T cells in vitro was measured by IFN-γ cytokine secretion using ELISA. Ultra-LEAF™ Purified anti-human CD28 Antibody (Biolegend) was included as a positive control.

Human CD3⁺ T cells were isolated from cryopreserved PBMCs using the EasySep™ Human Naïve Pan T Cell Isolation Kit (STEMCELL Technologies). The isolated cells were cultured in 96-well tissue culture plates (1.0×10⁵ per well) pre-coated with a suboptimal concentration (10 nM, 50 μL per well) of anti-human CD3 antibody (OKT3), in the presence of serially diluted benchmark controls, an isotype negative control antibody, a commercial anti-human CD28 positive control antibody, and a panel of anti-CD28 test mAbs (0.1, 1, 10, and 100 nM). The cells were incubated at 37° C., 5% CO₂ for 120 hours and then cell supernatants were collected for IFN-γ cytokine analysis by ELISA, with T cell proliferation measured by CellTiter-Glo®.

As shown in FIGS. 4B and 4C, in comparison with the isotype control antibody, the anti-CD28 mAbs showed concentration-dependent biological activity including T cell proliferation and IFN-γ cytokine secretion. In general, the effects of anti-CD28 mAbs on human T cell activation were comparable to or more potent than the two benchmark control antibodies.

### T Cell Activation Co-Stimulation Assay: IL-2 Release

The biological activity of the anti-CD28 mAbs as agonistic T cell co-stimulatory agents in activating human T cells in vitro was measured by T cell proliferation with the CellTiter-Glo® Luminescent Cell Viability Assay (Promega) and IL-2 cytokine secretion by ELISA. Ultra-LEAF™ Purified anti-human CD28 Antibody (Biolegend) was included as a positive control.

Human T cells were isolated from fresh PBMCs from Asian donor using the EasySep™ Human Naïve Pan T Cell Isolation Kit (STEMCELL Technologies). The cells were cultured in 96-well tissue culture plates (1.0×10⁵ cells/well) pre-coated with a suboptimal concentration (5 nM) of anti-human CD3 antibody (OKT3), in the presence of serially diluted benchmark control TAC2387, an isotype negative control antibody, a commercial anti-human CD28 positive control antibody, and a panel of anti-CD28 test mAbs. The cells were incubated at 37° C., 5% CO, for 72 hours, and then the cell supernatants were collected for IL-2 cytokine analysis by ELISA and the level of T cell proliferation was measured using the CellTiter-Glo®ωassay.

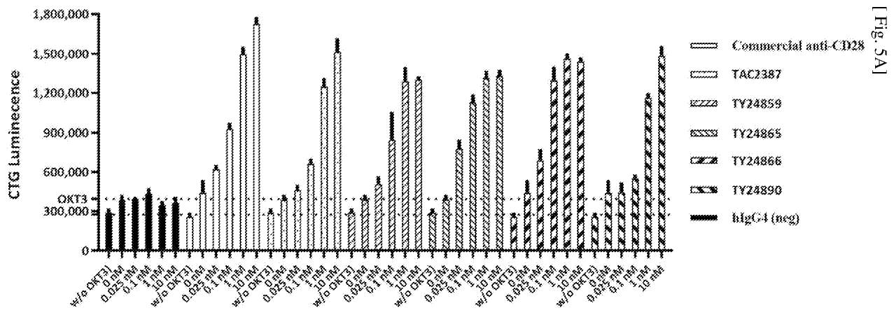

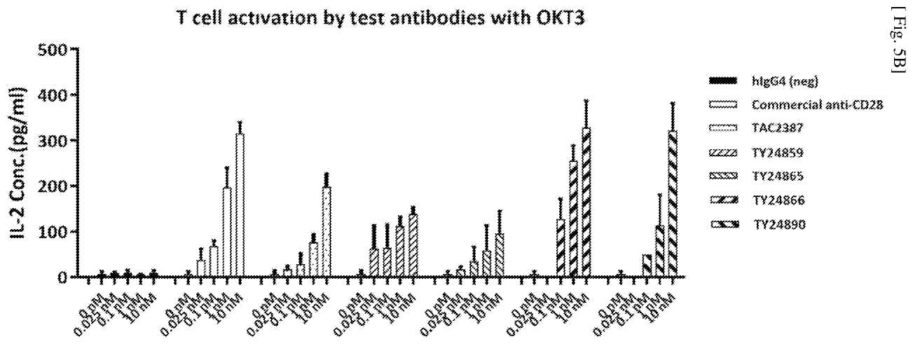

As shown in FIGS. 5A-B, in comparison with the isotype control antibody, the anti-CD28 antibodies showed concentration-dependent biological activity including T cell proliferation and IL-2 cytokine secretion. The effects of TY24859, TY24865, TY24866, and TY24890 on human T cell activation were comparable to the benchmark control TAC2387. The negative control groups without pre-coating anti-CD3 showed no detectable T cell proliferation and IL-2 cytokine secretion.

### Example 7: Anti-HER2×CD28 Bispecific Antibody Construction and Functional Characterization

### Generation of Anti-HER2×CD28 Bispecific Antibodies

A heterodimeric bispecific scaffold was engineered using the TYM13 Fc mutant (D or E356K: E357K: S364K: S400C L351′D: K370′D: N390′C: K439′D; according to Kabat numbering scheme for an IgG1 C_{H}3 domain). A light chain-heavy chain half antibody and a single-chain fragment variable (scFv)-Fc chain were combined to form a bispecific antibody (BsAb), with TYM13 mutations in the hetero-Fc domain.

Plasmids encoding the heavy chain, light chain, and scFv-Fc chain of BsAbs were transiently transfected into mammalian cells. Bispecific antibody-containing cell culture supernatants were harvested 7 days after transfection by centrifugation at 14000 g for 30 minutes and were filtered through a sterile filter (0.22 μm). Antibodies were purified by protein A affinity chromatography using MabSelect™ SuRe™ prepacked columns (GE Healthcare) and were subsequently buffer exchanged in 20 mM histidine (pH 5.5) buffer.

TY24865 (high affinity CD28) and TY24865 mutant (low affinity CD28) were selected for constructing CD28 BsAbs using this scaffold. The constructs are described in Table 10.

TABLE 10
Design of Anti-HER2xCD28 Bispecific Antibody

| BsAb ID | Fab Arm (Anti-HER2) | scFv Arm (Anti-CD28) |
|---|---|---|
| TY27566 | Perjeta ® | TY24865 |
| TY27881 | Perjeta ® | TY24865 mutant |
| TY27807 | Herceptin ® | TY24865 |

### Binding to SK-OV-3 Cells by FACS

The concentration-dependent binding activities of anti-HER2×CD28 BsAb (TY27566), anti-HER2×CD3 BsAb (TY25238, also described in PCT/CN2021/076626, which is incorporated by reference herein in its entirety) and anti-HER2 mAbs for Perjeta® (TAC2319) or Herceptin((TAC2320) were measured using flow cytometry. Perjeta® (TAC2319) binds to a different epitope of the HER2 dimerization domain than Herceptin® (TAC2320).

SK-OV-3 cells were cultured and added to 96-well plates at 8.0×10⁴ cells/well and incubated with serially diluted test BsAbs for 60 minutes at 4° C. in 2% FBS/RPMI1640 buffer. Next, the cells were washed twice with DPBS and further incubated with a secondary APC-anti-human IgG Fc antibody (1:400 dilution) for 30 minutes at 4° C. Finally, the cells were washed twice with DPBS and resuspended in FACS buffer for flow cytometry analysis. For analysis, the MFI values versus concentrations were plotted using FlowJo 10 software (FlowJo LLC) and the data were further fitted with four-parameter non-linear regression to obtain EC₅₀ values by GraphPad Prism version 7 for Windows, GraphPad Software, La Jolla California USA, www.graphpad.com. An isotype antibody was included as a negative control.

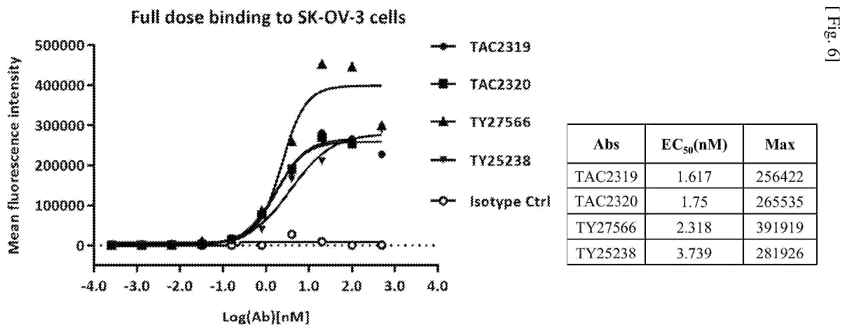

As shown in FIG. 6, anti-HER2×CD28 or anti-HER2×CD3 BsAbs showed very similar binding affinity to SK-OV-3 cells, compared with anti-HER2 mAbs for Perjeta® (TAC2319) or Herceptin® (TAC2320).

### Stimulatory Activities on T Cell Receptor and CD28 Receptor Signaling

The stimulatory activities on T Cell Receptor (TCR) and CD28 receptor signaling by the anti-HER2×CD3 BsAb, the anti-HER2×CD28 BsAb, or their combination were evaluated. In this assay, the simultaneous TCR and CD28 activation leads to enhanced transcriptional activity of NFkB, which in turn induces the production of the reporter gene. Isotype antibodies were included as negative controls.

Jurkat-NFkB-Nluc effector (E) reporter cells (5×10⁴ cells/well) were co-cultured with SK-OV-3 target (T) cells (1×10⁴ cells/well for a E:T=5:1), that endogenously express HER2 antigen. Serially diluted anti-HER2×CD3 BsAb or isotype control antibody with a fixed concentration of anti-HER2×CD28 BsAb (10 nM), or conversely serially diluted anti-HER2×CD28 BsAb or isotype control antibody with a fixed concentration of anti-HER2×CD3 BsAb (0.01 nM), were added to the reporter cell system to evaluate their combined effect in stimulating downstream luciferase activity. The co-cultured cells were incubated at 37° C., 5% CO₂ for 6 hours. Then, 100 μL of Nano-Glo Luciferase Assay System (Promega) reagent was added to the cells, and the cells were lysed for 10 minutes. Supernatants (100 L) were collected for luminescence measurements using a SpectraMax® i3× Multi-Mode Microplate Reader (Molecular Devices).

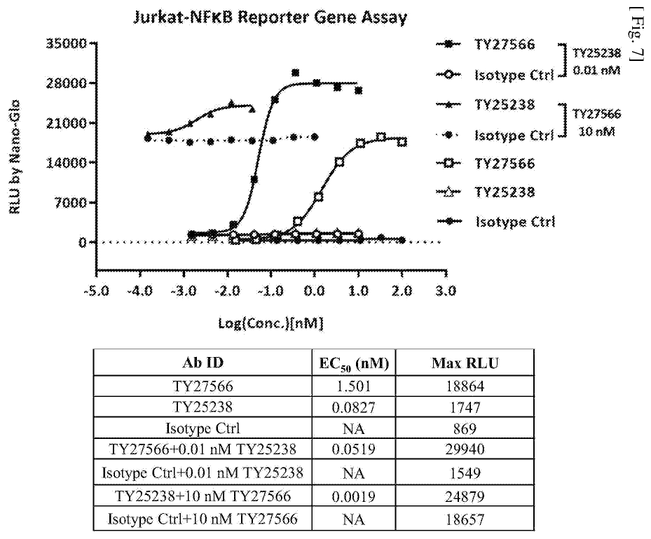

As shown in FIG. 7, anti-HER2×CD28 BsAb (TY27566) in combination with a fixed concentration of anti-HER2×CD3 BsAb (TY25238), and anti-HER2×CD3 BsAb in combination with a fixed concentration of anti-HER2×CD28 BsAb, exhibited synergistic or enhanced stimulatory effects in terms of maximum signal and EC₅₀ values.

Cytotoxicity of Anti-HER2×CD28 BsAb and Anti-HER2×CD3 BsAb with the Same or Different TAA Epitopes

The in vitro cytotoxicity activity of anti-HER2×CD3 BsAb (TY25238) alone or combined with anti-HER2×CD28 BsAbs (TY27566 or TY27807) on the MCF-7 tumor cell line was measured using a lactate dehydrogenase (LDH) release assay. TY25238 binds to a different tumor-associated antigen (TAA) epitope of the HER2 dimerization domain than TY27566 but to the same TAA epitope as TY27807.

Human T cells were isolated from cryopreserved PBMCs. Cultured MCF-7 tumor target cells (1×10⁴ cells/well) were incubated with serially diluted anti-CD3 BsAb FG14127 alone or combined with a fixed concentration (1 μg/mL) of two anti-CD28 BsAbs against different TAA epitopes, TY27566 or TY27807, for 30 minutes at 37° C. Then, isolated human T effector cells (2×10⁴ cells/well) were added and incubated at 37° C., 5% CO₂ for 72 hours (E:T=2:1). Cellular cytotoxicity based on LDH release into supernatants by killed MCF-7 target cells was quantified using the CytoTox 96® Non-Radioactive Cytotoxicity Assay (Promega).

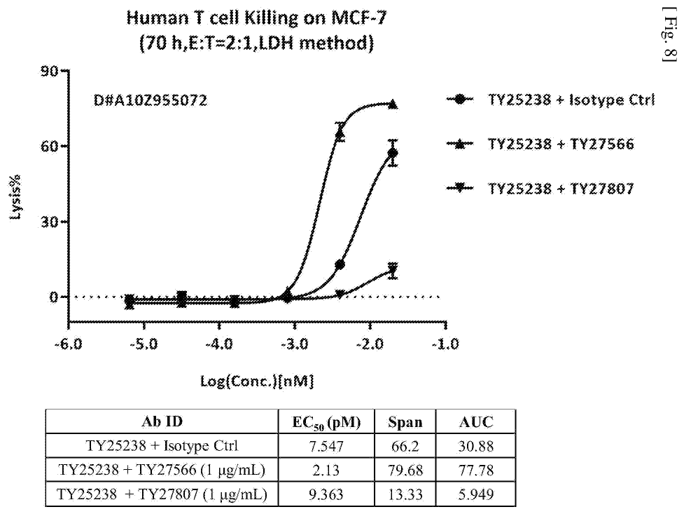

As shown in FIG. 8, as a single agent, anti-HER2×CD3 BsAb (TY25238) elicited potent concentration-dependent cytotoxicity on MCF-7 target cells. The combination of the anti-HER2×CD28 BsAb (TY27566) and anti-HER2×CD3 BsAb (TY25238) against different TAA epitopes further enhanced the in vitro tumor killing activity in terms of EC₅₀. However, compared to treatment with anti-HER2×CD3 BsAb (TY25238) alone, the addition of anti-HER2×CD28 BsAb (TY27807) with the same TAA epitope significantly decreased the MCF-7 tumor cell lysis in terms of EC₅₀ and maximum lysis. An isotype antibody was included as a negative control for the single agent assay.

### Example 8: Anti-TROP2×CD28 or Anti-TROP2×CD3 BsAb Construction and Functional Characterization

An anti-tumor-associated calcium signal transducer 2 (TROP2) ×CD28 or anti-TROP2×CD3 heterodimeric bispecific scaffold was designed using the TYM13 Fc mutant as described in Example 7. The constructs of anti-TROP2×CD28 BsAb are described in Table 11.

The constructs of anti-TROP2×CD3 BsAb are described in Table 12.

TABLE 11
Design of Anti-TROP2xCD28 Bispecific Antibody

| | Fab Arm (Anti- | |
|---|---|---|
| BsAb ID | TROP2) | scFv Arm (Anti-CD28) |
| TY27571 | TY25616 | TY24865 |

TABLE 12
Design of Anti-TROP2xCD3 Bispecific Antibody

| | Fab Arm (Anti- | |
|---|---|---|
| BsAb ID | TROP2) | scFv Arm (Anti-CD3) |
| TY25839 | TY25616 | TY24742 |

CD3- or CD28-Based Bispecific Binding to Tumor Cell Lines with High, Medium, or Low TROP2 Expression

The concentration-dependent binding activity of anti-TROP2 BsAbs on different tumor cell lines with high, medium, or low TROP2 expression, was measured using flow cytometry. Isotype antibodies were included as negative controls.

H292, NCI-N87, or HT29 cells were cultured and seeded in 96-well plates at 1.0×10⁵ cells/well and incubated with serially diluted test anti-TROP2 BsAbs for 30 minutes at 4° C. in 2% FBS/RPMI1640 buffer. Next, the cells were washed twice with DPBS and further incubated with secondary anti-human IgG antibodies (APC-anti-human IgG Fc antibody, 1:300 dilution for H292 cell; APC-F (ab′) 2 fragment goat anti-human IgG (H+L), 1:500 dilution for NCI-N87 and HT29 cells) for 30 minutes at 4° C. Finally, the cells were washed twice with DPBS and resuspended in FACS buffer for flow cytometry analysis. For analysis, the MFI values versus concentrations were plotted using FlowJo 10 software (FlowJo LLC) and the data were further fitted with four-parameter non-linear regression to obtain EC₅₀ values by GraphPad Prism version 7 for Windows.

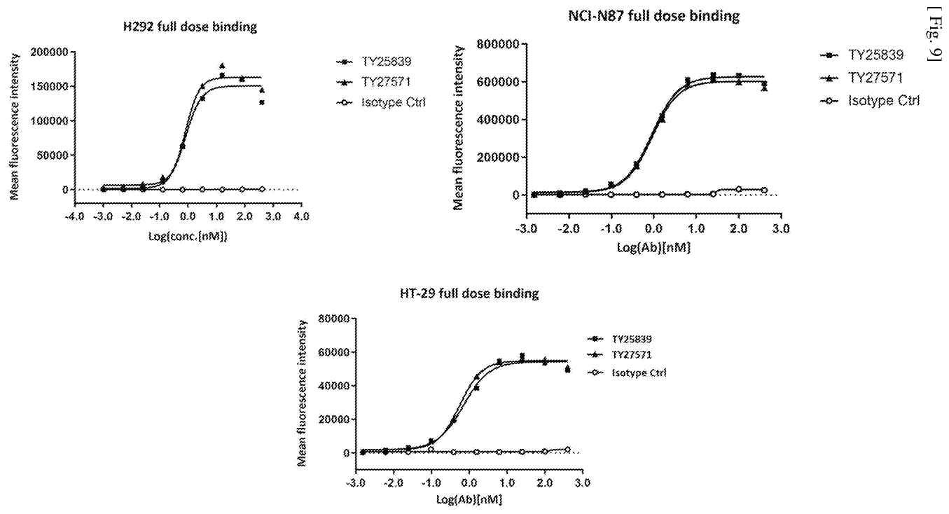

As shown in FIG. 9 and Table 13, the TROP2 arm binding activities were similar between TY25839 and TY27571 on three different tumor cell lines with high, medium, or low TROP2 expression.

TABLE 13
EC₅₀ Values of Anti-TROP2 Bispecific Antibodies on Tumor Cell Lines

| | H292 | | | NCI-N87 | | | HT-29 | | |
|---|---|---|---|---|---|---|---|---|---|
| BsAb | EC₅₀ | | | EC₅₀ | | | EC₅₀ | | |
| ID | (nM) | Span | AUC | (BM) | Span | AUC | (BM) | Span | AUC |
| TY25839 | 0.813 | 149008 | 418398 | 0.901 | 612873 | 1715321 | 0.693 | 52548 | 156975 |
| TY27571 | 0.822 | 156304 | 465058 | 0.911 | 589022 | 1644423 | 0.559 | 52992 | 162739 |

### Stimulatory Activities on TCR and CD28 Receptor Signaling

The stimulatory activities on TCR and CD28 receptor signaling by anti-TROP2×CD3 BsAbs, anti-TROP2×CD28 BsAbs or the combination were evaluated. The simultaneous TCR and CD28 activation leads to enhanced transcriptional activity of NFkB, which in turn induces the production of the reporter gene.

The Jurkat-NFkB-Nluc effector reporter cells (5×10⁴ cells/well) were co-cultured with H292 target cells (E:T=5:1). Serially diluted anti-TROP2×CD3 BsAbs with fixed concentration of anti-TROP2×CD28 BsAb (5 nM), or conversely serially diluted anti-TROP2×CD28 BsAbs with fixed concentration of anti-TROP2×CD3 BsAb (0.01 nM), were added to the reporter cell system to evaluate their combined activity in stimulating downstream luciferase activity. The co-cultured cells were incubated at 37° C., 5% CO₂ for 6 hours. Then, 100 μL of Nano-Glo Luciferase Assay System (Promega) reagent was added to the cells, and the cells were lysed for 10 minutes. Supernatants (100 μL) were collected for luminescence measurements using a SpectraMax i3x Multi-Mode Microplate Reader (Molecular Devices).

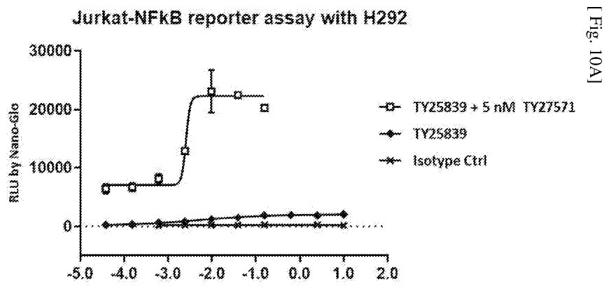

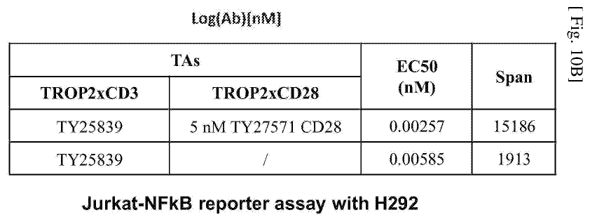

As shown in FIG. 10, single agent CD28-based BsAbs exhibited very weak reporter gene activity. However, the combination of anti-TROP2×CD3 and anti-TROP2×CD28 BsAbs exhibited synergistic or enhanced stimulatory effects in terms of maximal signals or EC₅₀ values.

### Example 9: B7H3×CD28 Bispecific Antibody Construction and Functional Characterization

A B7H3×CD28 BsAb was constructed. The constructs are described in Table 14.

The constructs of B7H3×CD3 BsAbs are described in Table 15.

TABLE 14
Design of B7H3xCD28 Bispecific Antibody

| | Fab Arm (Anti- | |
|---|---|---|
| BsAb ID | B7H3) | scFv Arm (Anti-CD28) |
| TY27556 | TY21601 | TY24865 |

TABLE 15
Design of B7H3xCD3 Bispecific Antibody

| | Fab Arm (Anti- | |
|---|---|---|
| BsAb ID | B7H3) | scFv Arm (Anti-CD3) |
| TY26999 | TY21601 | Medium affinity CD3 |

### Binding to MDA-MB-231 Cells by FACS

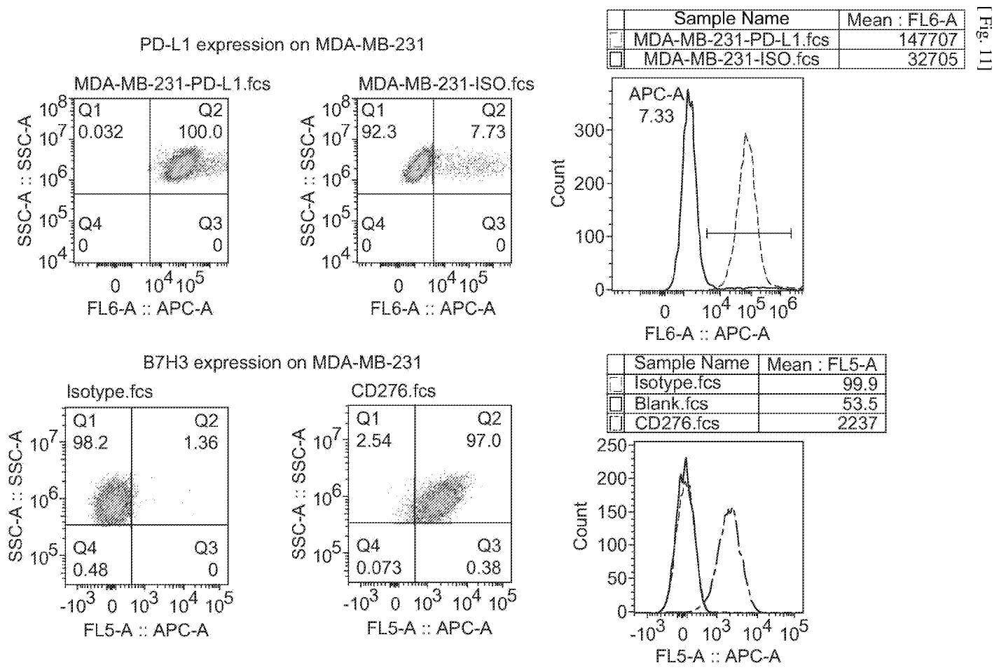

The concentration-dependent binding activity of anti-B7H3×CD28 BsAb TY27556 and its parental anti-B7H3 mAb TY21601 on MDA-MB-231 cells, was measured using flow cytometry (FIG. 11). An isotype antibody was included as a negative control.

MDA-MB-231 cells were cultured and seeded in 96-well plates at 1.0×10⁵ cells/well and incubated with serially diluted test BsAbs for 60 minutes at 4° C. in 2% FBS/RPMI1640 buffer. Next, the cells were washed twice with DPBS and further incubated with secondary APC-anti-human IgG Fe antibody (1:400 dilution) for 30 minutes at 4° C. Finally, the cells were washed twice with DPBS and resuspended in FACS buffer for flow cytometry analysis. For analysis, the MFI values versus concentrations were plotted using FlowJo 10 software (FlowJo LLC) and the data were further fitted with four-parameter non-linear regression to obtain EC₅₀ values by GraphPad Prism version 7 for Windows.

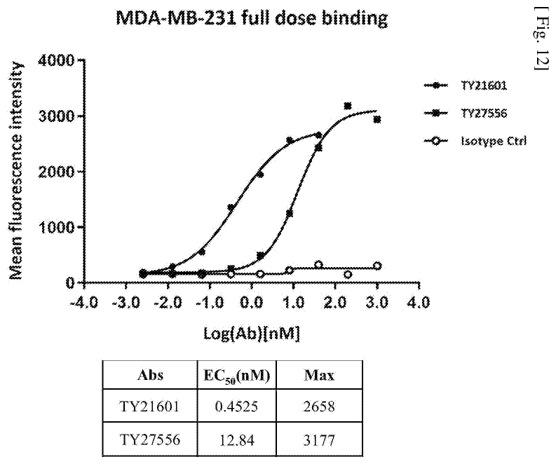

As shown in FIG. 12, the anti-B7H3 mAb TY21601 showed sub-nM (0.4525 nM) binding affinity for MDA-MB-231 target cells and the binding activity of the BsAb TY27556 to target cells was reduced by about 28-fold (12.84 nM).

### B7H3×CD28 Bispecific Antibody Enhanced the Ability of PD-1 or PD-L1 Blockade to Induce T Cell Activation in Vitro

The effect of combining anti-PD-1 blocking mAbs with the B7H3×CD28 bispecific Abs on primary human T cell activation in vitro, was measured with IFN-γ and IL-2 secretion by ELISA. A modified mixed lymphocyte reaction (MLR) to simulate physiological PD-L1 expression and TCR/CD3 stimulation was used here. To generate a one-way MLR assay, human T cells (1×10⁵ cells per well) from one healthy donor (D #XC11147W) were incubated with allogenic MDA-MB-231 cells (E:T=5:1) in the presence of different test antibodies (B7H3×CD28 or isotype control) alone or in combinations with anti-PD-1 (pembrolizumab, Keytruda®) or anti-PD-L1 (atezolizumab, Tecentriq®). The cells were then co-cultured at 37° C., 5% CO₂ in the incubator for 120 h. IL-2 (72 h) and IFN-γ (120 h) cytokine secretion into supernatants by activated T cells was quantified with Elisa kit.

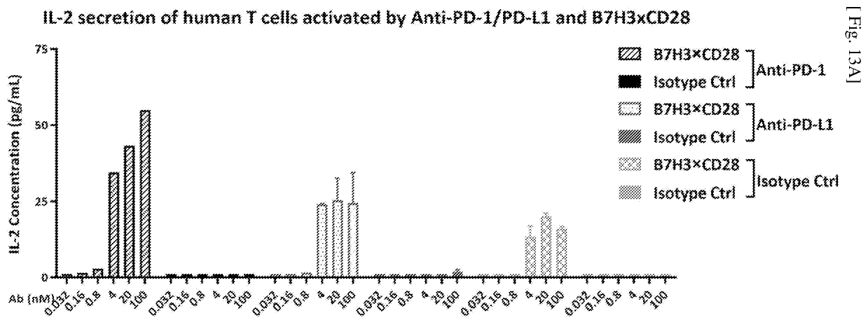

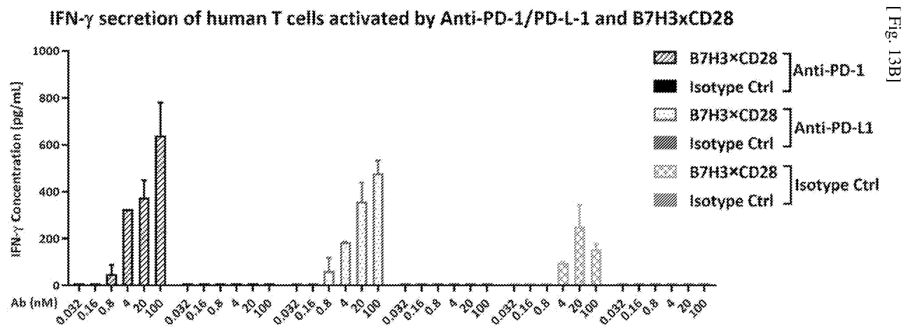

As shown in FIGS. 13A-13B, in this MLR assay, addition of 100 nM PD-1/PD-L1 mAbs or titrated B7H3×CD28 resulted in no or only slight cytokine release. However, B7H3×CD28 combination with 100 nM PD-1/PD-L1 mAbs markedly increased T cell activation, compared with monotherapy. These results demonstrate that the B7H3×CD28 bispecific can synergistically combine with PD-1/PD-L1 blockade to promote T cell activation in the presence of tumor cells, that endogenously express PD-L1 and B7H3.

### Co-Stimulatory Bispecific Antibodies Enhance in Vitro T Cell Cytotoxicity Against MCF-7 Cells Upon Bidirectional Binding

In vitro tumor cell killing activity of anti-CD3-based, or anti-CD28-based BsAbs or their combination on MCF-7 tumor cell line was measured using a LDH release cytotoxicity assay.

Human T cells were isolated from cryopreserved PBMCs. MCF-7 cells (1×10⁴ cells/well) were incubated with serially diluted anti-HER2×CD3 BsAb TY25238 or combined with fixed concentrations of the high affinity CD28 arm anti-HER2×CD28 BsAb TY27566, anti-CD28×B7H3 TY27556, or the low affinity CD28 arm anti-HER2×CD28 BsAb TY27881 (1 ug/mL or 10 μg/mL) for 30 minutes at 37° C., and the human T cells (2×10⁴ cells/well) were added and incubated at 37° C., 5% CO₂ for 72 hours (E:T=2:1). Cellular cytotoxicity based on LDH release into supernatants by killed MCF-7 target cells was quantified using the CytoTox 96® Non-Radioactive Cytotoxicity Assay (Promega).

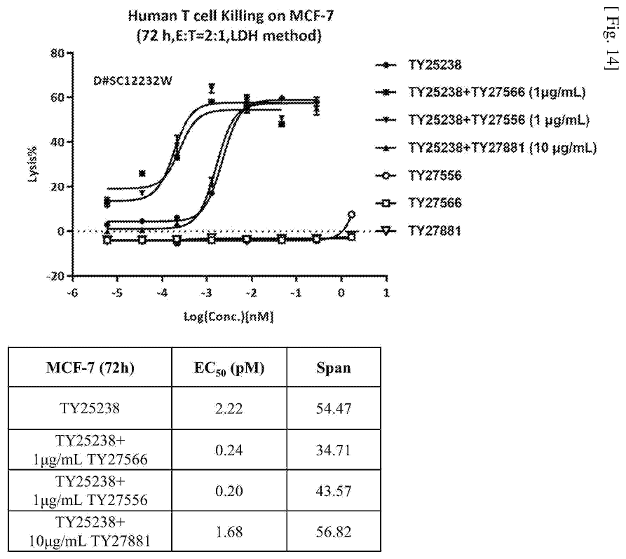

As shown in FIG. 14, as single agents, CD28-based BsAbs elicited no cytotoxicity on MCF-7 target cells. In contrast, as a single agent, the anti-CD3 BsAb TY25238 showed concentration-dependent effects and the highest cytotoxicity was elicited with BsAb directed against HER2 with 54.47% of MCF-7 cells lysed. The combination of anti-CD3 and anti-CD28 BsAbs further enhanced the in vitro tumor cell killing activity as manifested by EC₅₀ reduction. Compared to treatment with anti-CD3 BsAb TY25238 alone, the addition of anti-CD28 BsAb TY27566 with a high affinity CD28 arm considerably enhanced tumor cell lysis by about 10-fold (EC₅₀). However, when combined with anti-CD28 BsAb TY27881 with a low affinity CD28 arm, no enhanced cytotoxicity was observed.

### Co-Stimulatory Bispecific Antibodies Enhance in Vitro T Cell Cytotoxicity Against EMT-6-HER2 Cells Upon Bidirectional Binding

In vitro tumor cell killing activity of anti-CD3-based or anti-CD28-based BsAbs or their combination on EMT-6-HER2 tumor cell line was measured using a LDH release cytotoxicity assay.

The human T cells were isolated from fresh PBMCs using EasySep™ Human Naïve Pan T Cell Isolation Kit (STEMCELL Technologies). EMT-6-HER2 (5×10³ cells/well) target cells were incubated with serially diluted anti-HER2×CD3 BsAb (TY25238) or in combination with fixed concentrations of anti-B7H3×CD28 BsAb TY27556 (8 nM) for 30 minutes at 37° C., or conversely EMT-6-HER2 (5×10³ cells/well) cells were incubated with serially diluted anti-B7H3×CD3 BsAbs TY26999 or combination with fixed concentrations of anti-HER2×CD28 BsAb TY27566 (8 or 0.8 nM) for 30 minutes at 37° C. Next, isolated human T effector cells (2×10⁴ cells/well) were added and incubated at 37° C., 5% CO₂ for 72 hours (E:T=2:1). Cellular cytotoxicity based on LDH release into supernatants by killed EMT-6-HER2 target cells was quantified using the CytoTox 96® Non-Radioactive Cytotoxicity Assay (Promega). An isotype antibody was included as a negative control for the single agent assays.

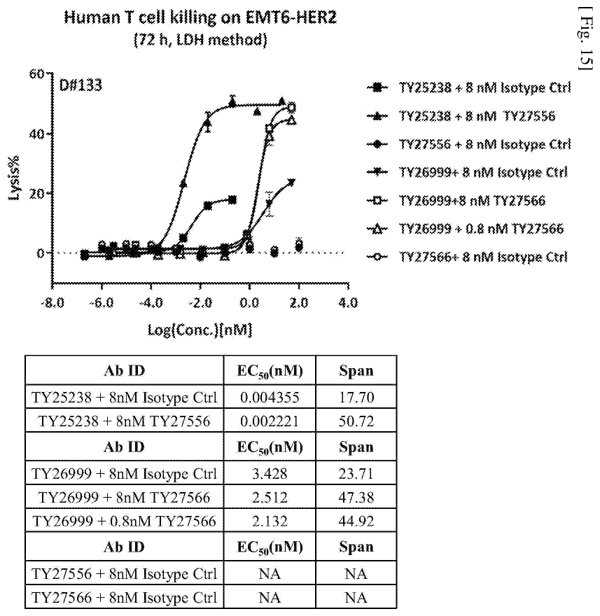

As shown in FIG. 15, as a single agent, TY27556 (anti-B7H3×CD28) or TY27566 (anti-HER2×CD28) exhibited no killing potency on EMT-6-HER2 cells. TY25238 in combination with TY27556 resulted in about 2-fold decrease of EC₅₀, and about 3-fold increase of maximal killing (from 17% to 50%), compared with TY25238.

TY26999 combined with TY27566 resulted in about 1.3-fold decrease of EC₅₀ and about 2-fold increase of maximal killing (from 23% to 47%), compared with TY26999.

In Vivo Systemic Cytokine Release Risk of Anti-CD3- or Anti-CD28-Based Bispecific Antibodies or their Combination

In vivo systemic cytokine release risk (IL-6 and IFN-γ) of anti-mouse CD3- or anti-CD28-based BsAbs alone or combined in BALB/c mouse model was measured using ELISA.

The BALB/c mice were randomly divided into four groups (3 mice per group), and were injected with an anti-mouse CD3 mAb (145-2C11 clone, 1 mg/kg), anti-B7H3×CD28 (TY27556, 2 mg/kg), anti-B7H3×CD3 (TY27042, in which CD3 arm was derived from mouse specific 145-2C11 clone, 2 mg/kg) or the combination (TY27556, 2 mg/kg with TY27042, 2 mg/kg), respectively. The mouse serum and whole blood were collected at various time points before and post injection (pre-dose, 3.5, and 24 hours). The systemic cytokine release risk was assessed with IL-6 and IFN-γ by ELISA. The percentage of total peripheral T cells that were CD3⁺ T cells was determined by flow cytometry at each time point.

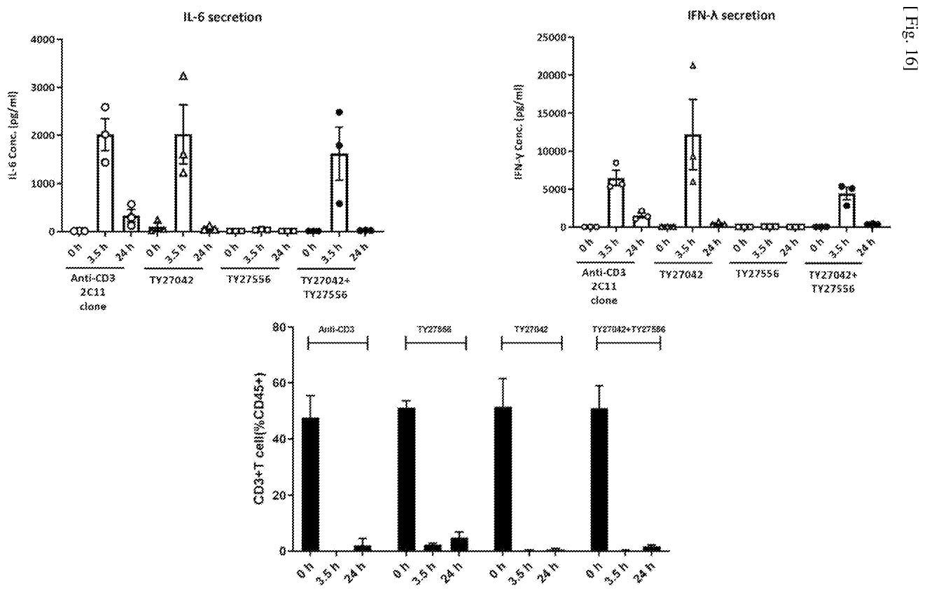

As shown in FIG. 16, mice administered anti-mCD3 or anti-B7H3×CD3 (TY27042) lead to a significant induction of cytokine release (IL-6 and IFN-γ) at 3.5 h after test antibody treatment. Mice treated with anti-B7H3×CD28 (TY27556) showed no detectable cytokine release after test antibody treatment. In addition, anti-B7H3×CD3 (TY27042) combined with anti-B7H3×CD28 (TY27556) showed no increased cytokine release risk above that found with the single agent anti-B7H3×CD3 (TY27042). The peripheral CD3⁺ T cells were sharply reduced to almost zero in the single agent anti-mouse CD3, anti-B7H3×CD3 (TY27042), and anti-B7H3×CD28 (TY27556) groups and in the anti-B7H3×CD3 (TY27042) combined with anti-B7H3×CD28 (TY27556) group. Only about 2% to 3% of CD3⁺ T cells remained in the peripheral blood at 3.5 hours post injection.

### Example 10: In Vivo Efficacy Studies

In Vivo Efficacy Study of B7H3×CD28 BsAb Monotherapy or Combination Therapy with HER2×CD3 BsAb in SK-OV3 Model

Immunodeficient M-NSG mice (n=8 per group, female, 7-8 weeks old) were transplanted with 5×10⁶ PBMC through i. p. injection. Seven days later, the mice were inoculated subcutaneously with 2×10⁶ SK-OV3 cells. Treatment began at Day 8 post tumor inoculation when the average tumor volume reached about 90 mm³. The mice were administered with hIgG₁ isotype control at 5 mg/kg, anti-HER2×CD3 bispecific double masked antibody at 0.2 mg/kg, anti-B7H3×CD28 bispecific antibody TY27556 at 5 mg/kg, or TY27151 at 0.2 mg/kg in combination with TY27556 at 5 mg/kg by i. p. injection. TY27151 was previously described in PCT/CN2021/076626. The mice were administered these Abs twice per week for a total of five doses. Tumor growth was monitored twice a week and reported as the mean tumor volume±s. e. m. over time.

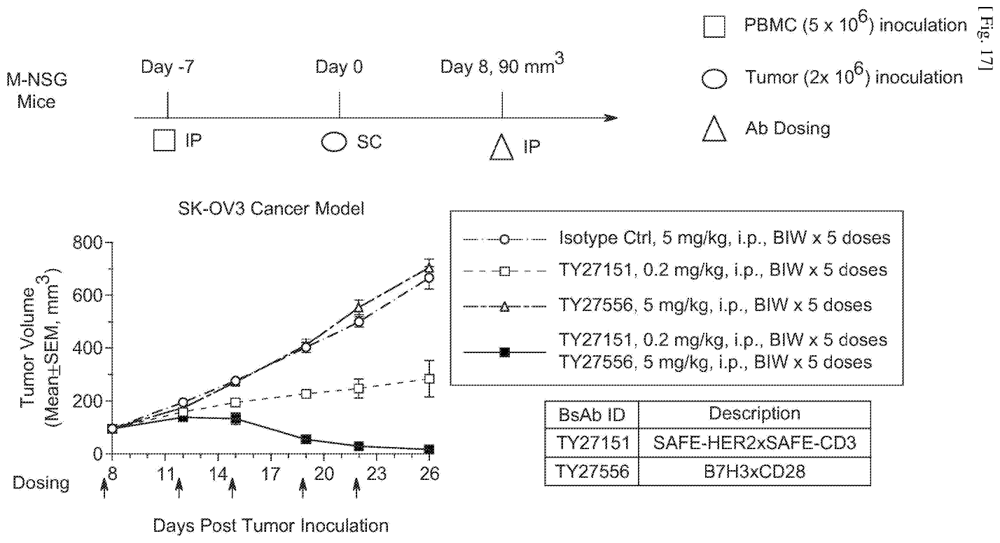

As shown in FIG. 17, the double masked anti-HER2×CD3 bispecific antibody TY27151 showed strong synergistic anti-tumor effect with the anti-B7H3×CD28 bispecific antibody TY27556 in this model.

### In Vivo Efficacy Study of CD28 BsAbs in EMT6-HER2 Murine Breast Cancer Syngeneic Model

BALB/c mice (n=5 per group, female, 8-9 weeks old) were inoculated subcutaneously with 5×10⁵ EMT-HER2 cells. Treatment began at Day 7 post tumor inoculation when the average tumor volume reached about 110 mm³. The mice were administered Vehicle, anti-B7H3×CD28 BsAb TY27556 at 0.5 mg/kg and 0.05 mg/kg, or anti-HER2×CD28 bispecific antibody TY27566 at 0.2 mg/kg by i. p. injection. The mice were administered these Abs twice per week for a total of four doses. Tumor growth was monitored twice a week and reported as the mean tumor volume±s. e. m. over time.

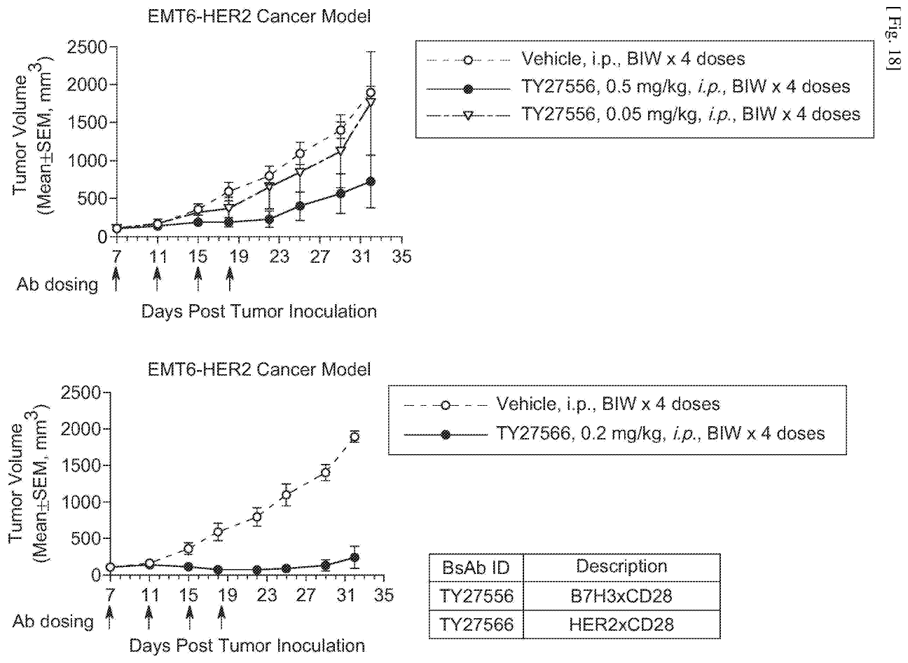

As shown in FIG. 18, anti-B7H3×CD28 bispecific antibody TY27556 showed dose dependent anti-tumor effect in this model. As shown in FIG. 18, anti-HER2×CD28 bispecific antibody TY27566 showed strong anti-tumor effect.

### Example 11: Methods of Identifying Self-Blocking Peptides for Masked Anti-CD28 Antibodies

### Generation of Masked Anti-CD28 Antibodies

A screening system has been designed and executed for efficient discovery of masking moieties that can effectively mask a non-masked parental anti-CD28 antibody with good developability. In this system, the target anti-CD28 scFv was first displayed on the surface of yeast and confirmed to be functional in binding to its CD28 antigen. Then masking peptides (MP) from an improved MP peptide library were directly fused to the N-terminus of the light chain of the target anti-CD28 scFv, and a yeast library was constructed that displayed the fusion protein on the yeast surface. The yeast library then underwent several rounds of FACS-based screening: 1) the yeast clones that had low binding to antigen were enriched, 2) the enriched yeast clones were treated with a protease to remove the N-terminal MP, and 3) the resulting clones that exhibited high binding to antigen were selected. After 5 to 6 rounds of sorting, the plasmids were extracted from these clones and the MP sequences were confirmed through DNA sequencing. The selected masked anti-CD28 antibody clones in scFv format, exhibited little binding to antigen in the presence of MP. However, binding to antigen was dramatically increased when the yeast cells were treated with Tobacco Etch Virus nuclear-inclusion-a endopeptidase (TEV) to remove the MP. The incorporation of the TEV recognition site as a cleavage site in the MP, combined with the application of TEV protease to verify the selected clones, significantly increased the success rate of MP selection.

To identify the MP sequences, the shuttle plasmids were extracted from the selected yeast clones using a plasmid extraction kit (Generay), and transformed into competent E. coli cells. The plasmids were prepared, and the regions encoding the MPs were sequenced and aligned. As anticipated, these sequences could be separated into several groups, indicating clear enrichment through rounds of sorting. The masking efficiency selected MPs are shown in the Table 16 below and the sequences of each MP is shown in the Sequences section below.

The masked anti-CD28 scFv proteins were converted into IgG₄ isotype mAbs. The masked IgG₄ mAbs were engineered to include a MP with a single invariant matrix metalloproteinase (MMP) cleavage site fused to the N-terminus of the light chain in the same manner as displayed on the yeast surface. The heavy and light chains were cloned into the mammalian expression vector pCDNA3.3 (Thermo Fisher Scientific) separately. The V_{H} and V_{L} sequences for the parental anti-CD28 antibody (TY24865) are listed in the Sequences section below.

Pairs of plasmids were transiently transfected into HEK293F cells. After six days, the supernatants were harvested, cleared by centrifugation and filtration, and IgGs were purified with standard protein A affinity chromatography (MabSelect SuRe, GE Healthcare). The IgGs were eluted and neutralized, and buffer exchanged into 20 mM histidine, pH 5.5 buffer. Protein concentrations were determined by UV-spectrophotometry, and IgG purity was analyzed under denaturing, reducing and non-reducing conditions by SDS-PAGE or SEC-HPLC. Importantly, the expression levels of the masked antibodies in HEK293 cells were similar to or lower than their parental antibody, and their purification yields after protein A resin were also similar, suggesting that the presence of the masking and cleavage peptides do not have a significantly negative impact on antibody expression in mammalian cells.

TABLE 16
Masking Peptides

| | ELISA Masking | | ELISA Masking | | ELISA Masking |
|---|---|---|---|---|---|
| IgG ID | Efficiency | IgG ID | Efficiency | IgG ID | Efficiency |
| TY26142 | 3062 | TY26152 | 1867 | TY26162 | ND |
| TY26143 | ND | TY26153 | 1825 | TY26163 | ND |
| TY26144 | ND | TY26154 | ND | TY26164 | ND |
| TY26145 | 1430 | TY26155 | 368 | TY26165 | ND |
| TY26146 | 2179 | TY26156 | 670 | TY26166 | ND |
| TY26147 | 4882 | TY26157 | ND | TY26167 | 1427 |
| TY26148 | ND | TY26158 | 887 | TY26168 | 2567 |
| TY26149 | 3841 | TY26159 | 468 | TY26169 | 1818 |
| TY26150 | 1027 | TY26160 | 1461 | TY26170 | ND |
| TY26151 | 2476 | TY26161 | 1484 | TY26171 | 406 |

### Measurement of Masking Efficiency

When measuring the masking efficiency through ELISA, recombinant human CD28-Fc was diluted to 2 g/mL in PBS and coated onto a MaxiSorp™ high protein-binding capacity 96 well ELISA plate (ThermoFisher Scientific) at 4° C. overnight. Plates were blocked with PBS supplemented with 3% non-fat milk at 37° C. for 1 hour. After washing, 100 μL of 3-fold serial dilutions of anti-CD28 test mAbs were added to each well. After incubation at 37° C. for 1 hour, plates were washed four times, and 100 μL horseradish peroxidase (HRP)-conjugated anti-human IgG (Fab specific) (1:6000 dilution) secondary antibody was added to each well. Plates were incubated at 37° C. for 1 hour, washed four times, and then 50 μL a TMB substrate (3, 3′, 5, 5′-tetramethylbenzidine) solution was added to each well, and the plate was incubated at room temperature. Absorbance at 450 nm was measured after the reactions were stopped with 50 μL sulfuric acid stop solution per well. The EC₅₀ was evaluated by fitting the ELISA data using the sigmoidal (four-parameter logistic equation) model of GraphPad Prism version 6 for Windows, GraphPad Software, La Jolla California USA, www.graphpad.com.

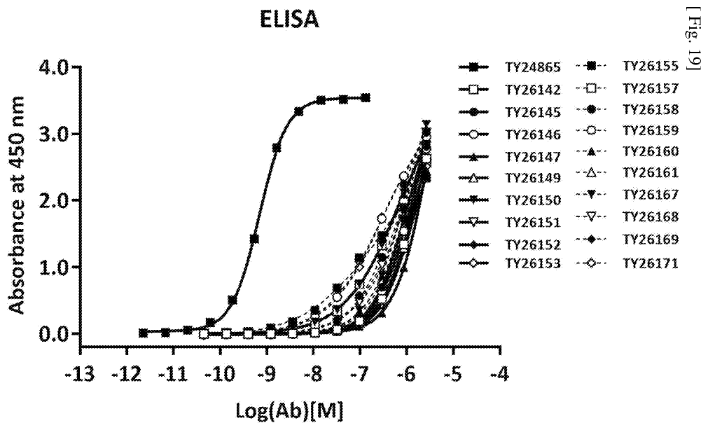

Masking efficiencies for selected masked anti-CD28 test mAbs were calculated by dividing the EC₅₀ for binding of the masked mAb by the EC₅₀ of the non-masked parental mAb (TY24865), and are listed in Table 17. As shown in FIG. 19, compared with the parental mAb, all of the activatable mAbs showed dramatically reduced binding to its antigen, and the calculated masking efficiency ranged from 368 to more than 4000. These results indicated that multiple MPs identified from an improved MP peptide library maintained their masking efficiency when expressed in mammalian cells, and as part of a full IgG molecule.

TABLE 17
SEC Purity and Masking Efficiencies
of Masked Anti-CD28 Antibodies

| | SEC Purity | | Masking |
|---|---|---|---|
| IgG ID | HMW (%) | LMW (%) | Efficiency |
| TY26142 | 2 | 0 | 3062 |
| TY26143 | >2.0 | 0 | ND |
| TY26144 | 24.6 | 0 | ND |
| TY26145 | 4.5 | 0 | 1430 |
| TY26146 | 3.6 | 0.2 | 2179 |
| TY26147 | 1.9 | 0 | 4882 |
| TY26148 | 1.9 | 0.1 | ND |
| TY26149 | 1.4 | 0.1 | 3841 |
| TY26150 | 1.9 | 0 | 1027 |
| TY26151 | 1.7 | 0 | 2476 |
| TY26152 | 2.1 | 0 | 1867 |
| TY26153 | 1.7 | 0 | 1825 |
| TY26154 | 14.2 | 0 | ND |
| TY26155 | 1.8 | 0 | 368 |
| TY26156 | 2.7 | 0 | 670 |

| | | |
|---|---|---|
| TY26157 | No main peak | ND |
| TY26158 | Low peak | 887 |

| | | | |
|---|---|---|---|
| TY26159 | 36.6 | 0 | 468 |
| TY26160 | 1.4 | 0 | 1461 |
| TY26161 | 1.8 | 0 | 1484 |
| TY26162 | 0.9 | 0 | ND |
| TY26163 | 1.1 | 0 | ND |
| TY26164 | 2.5 | 0 | ND |
| TY26165 | 2.8 | 0 | ND |
| TY26166 | 58.5 | 0 | ND |
| TY26167 | 2.2 | 0.3 | 1427 |
| TY26168 | 20.8 | 0 | 2567 |

| | | |
|---|---|---|
| TY26169 | Asymmetric main peak | 1818 |

| | | | |
|---|---|---|---|
| TY26170 | 3.9 | 0.8 | ND |
| TY26171 | 5.9 | 0 | 406 |

### Optimization of Selected Anti-CD28 Activatable Antibodies

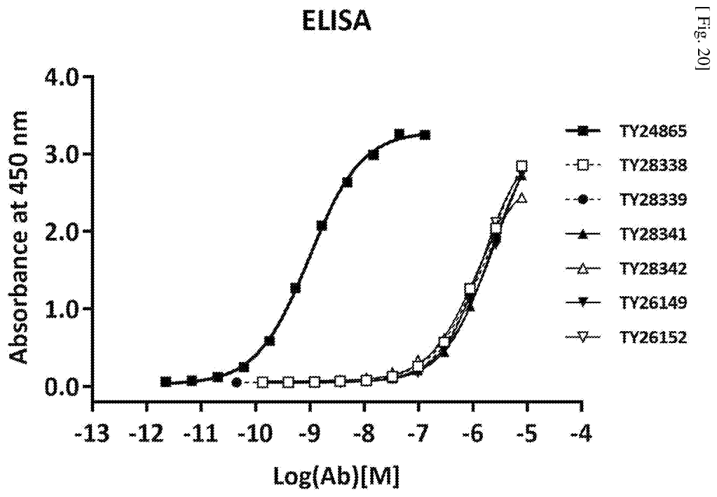

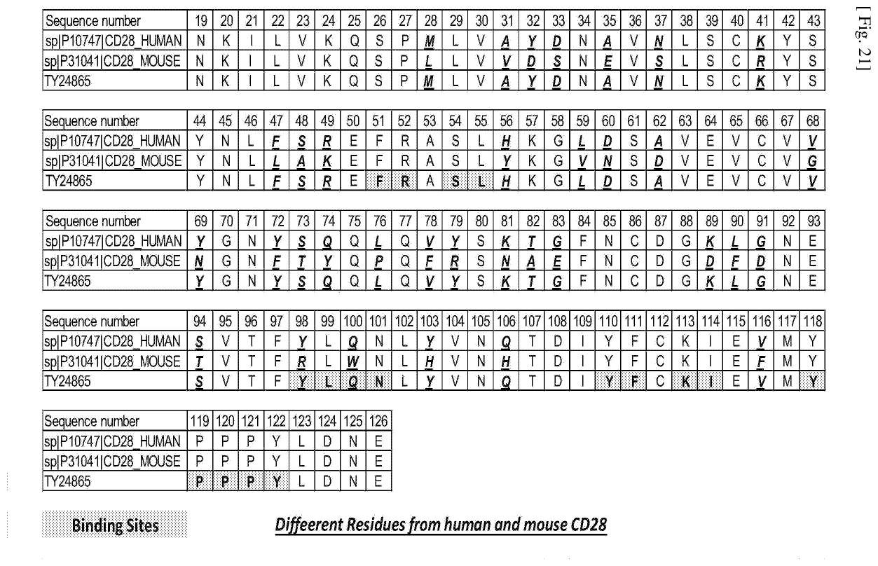

For the two lead masked anti-CD28 mAbs, TY26149 and TY26152 were modified on their MPs, including removing some N-terminal residues, and adding an “S” amino acid residue between residues “D” and “G” of the TY26149 sequence (bolded and underlined residues of TY26149 in Table 18). As shown in FIG. 20, the expression and masking efficiency of the new masked antibodies were not significantly influenced.

TABLE 18
Modification of Masking Peptide Sequences

| | | SEQ |
|---|---|---|
| | Masking + | ID |
| IgG ID | cleavage peptide sequences: | NO: |
| TY26149 | EVGSYAATYSDCYSDPYACHDGGGPLGLAGSGGS | 301 |
| TY28338 | EAATYSDCYSDPYACHDSGGGPLGLAGSGGS | 302 |
| TY28339 | EYSDCYSDPYACHDSGGGPLGLAGSGGS | 303 |
| TY26152 | EVGSYAAFSPACYTDPYSCHAGGGPLGLAGSGGS | 304 |
| TY28341 | EAAFSPACYTDPYSCHAGGGPLGLAGSGGS | 305 |
| TY28342 | ESPACYTDPYSCHAGGGPLGLAGSGGS | 306 |

### Example 12: Epitope Mapping

To determine the binding regions of the tested antibodies at amino acid residue level, a series of mutations (Table 19) were made at the extracellular domain of human CD28. These CD28 mutation plasmids were used to transfect HEK293F cells. The binding of antibodies to the human CD28 mutants were assessed by flow cytometry analysis. The results are summarized in Table 19, together with the cross-reactivity of these antibodies with human, monkey, and mouse CD28 in interesting differentiation. TY24865 is cross-reactive to human, monkey, and mouse CD28, while TAC2386 and TAC2387 do not bind to mouse CD28. The mutant constructs were meant to differentiate the epitopes by TY24865 from the reference antibodies by TAC2386 and TAC2387. It is clearly that TY24865 kept the binding ability to RE49AA, VY68AA, YS79AA and KT81AA, which indicated TY24865 does not bind to residues RE49, VY68, YS79 and KT81, and these residues are in un-conserved region of human and mouse CD28. While TY24865 lost the binding ability to FR51AA, SL54AA, YL98AA, QN100AA, YF110AA, KI113AA, YP118AA, PPP119AAA, PP120AA, PY121AA, and Y122A mutations, indicating that their binding epitopes are within these regions, e.g., amino acid residues 51, 52, 54, 55, 98-101, 110-111, 113-114, 118-122 of SEQ ID NO.: 1.

TABLE 19
Epitope Mapping

| Mutations | TY24865 | TAC2386 | TAC2387 |
|---|---|---|---|
| Hu_WT | + | + | + |
| Cyno_WT | + | + | + |
| Mouse_WT | + | − | − |
| Hu_RE49AA | + | + | − |
| Hu_FR51AA | − | − | − |
| Hu_SL54AA | − | − | − |
| Hu_VY68AA | + | + | − |
| Hu_Y69A | + | + | − |
| Hu_YS79AA | + | − | + |
| Hu_KT81AA | + | − | + |
| Hu_YL98AA | − | − | − |
| Hu_QN100AA | − | − | − |
| Hu_YF110AA | − | − | − |
| Hu_KI113AA | − | − | − |
| Hu_M117A | + | + | − |
| Hu_YP118AA | − | + | − |
| Hu_PPP119AAA | − | + | − |
| Hu_PP120AA | − | + | − |
| Hu_PY121AA | − | + | − |
| Hu Y122A | − | + | + |

### Example 13: TY24865 Variants

Two approaches were employed for generating variants of TY24865. Different point mutations were introduced into the YYYY sequences of TY24865 scFv and a new batch of TY24865 scFv variants were discovered through yeast-based maturation library screening. Anti-TAA×CD28 bispecific antibodies were constructed in Fab-scFv-Fc format and the CD28 affinities of these bsAbs were measured using ELISA.

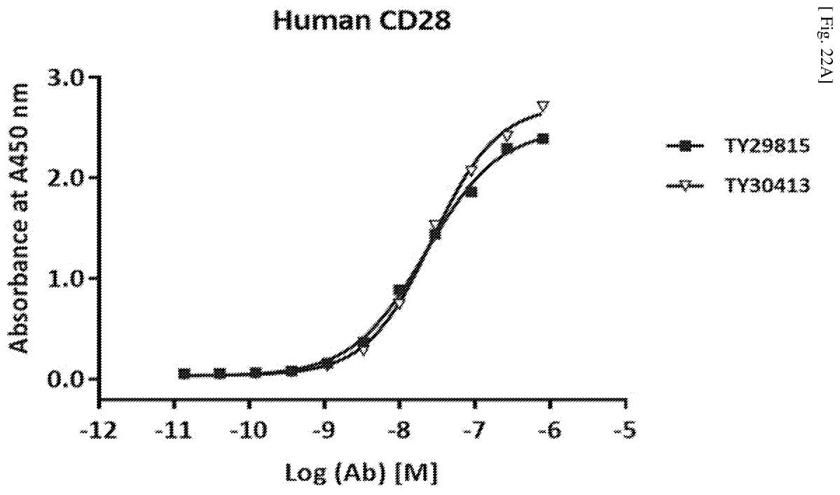

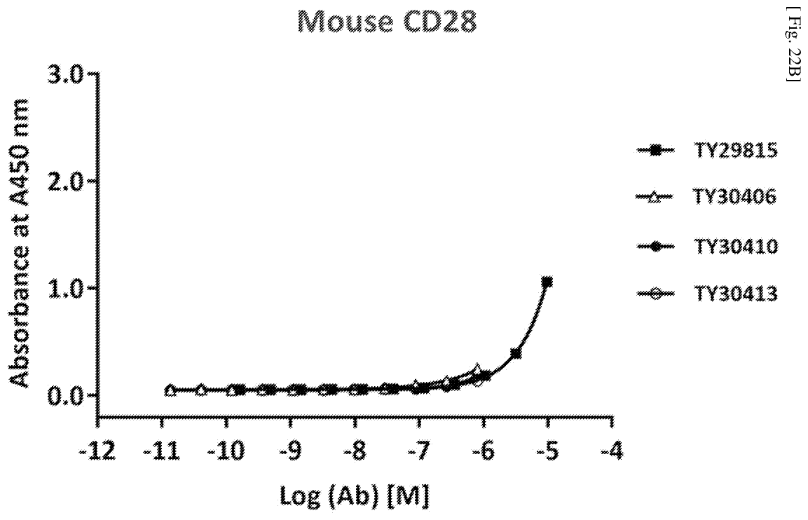

Anti-PD-L1×CD28 bispecific antibodies were constructed in Fab-scFv-Fc format with anti-PD-L1 TY21421 Fab (see WO 2019/185035) and TY24865 scFv (in VH-VL format) or its variants. The affinities of some of these bsAbs to CD28 are summarized in the Table 20 and FIGS. 22A and 22B; TY29815 is the wild type bsAb and TY30413 has similar affinity as TY29815, while the other bsAbs have higher or reduced affinity.

TABLE 20
Construction and Characterization of Anti-
PD-L1 × CD28 BsAbs with TY24865 Variants

| | | Titer | SEC purity | | Single point | ELISA |
|---|---|---|---|---|---|---|
| BsAb | CD28 scFv | (mg/L) | HMW(%) | LMW (%) | ELISA | EC₅₀ (nM) |
| TY29815 | VH-VL (YYYY) | 54.1 | 6.4 | 0.6 | | 22.26 |
| TY30406 | VH-VL (YDYY | 37.5 | 7.2 | 1.6 | Higher | |
| TY30410 | VH-VL (YYEY) | 23.2 | 9.5 | 1.7 | Reduced | |
| TY30413 | VH-VL (YYHY) | 60.2 | 2.8 | 0.5 | No change | 26.81 |

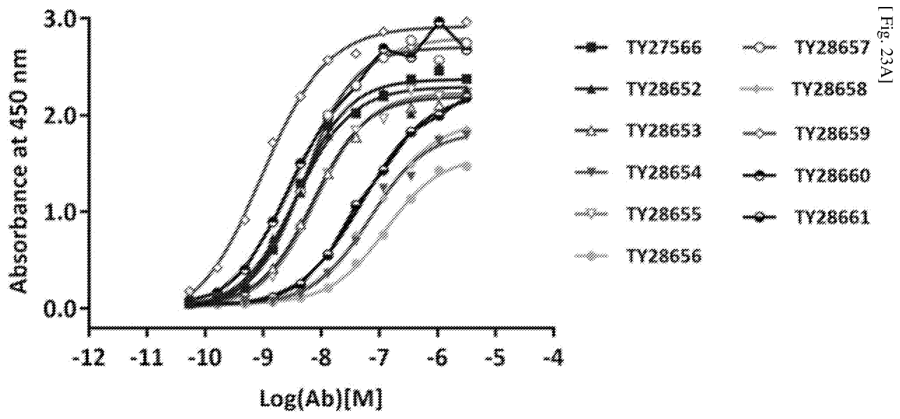

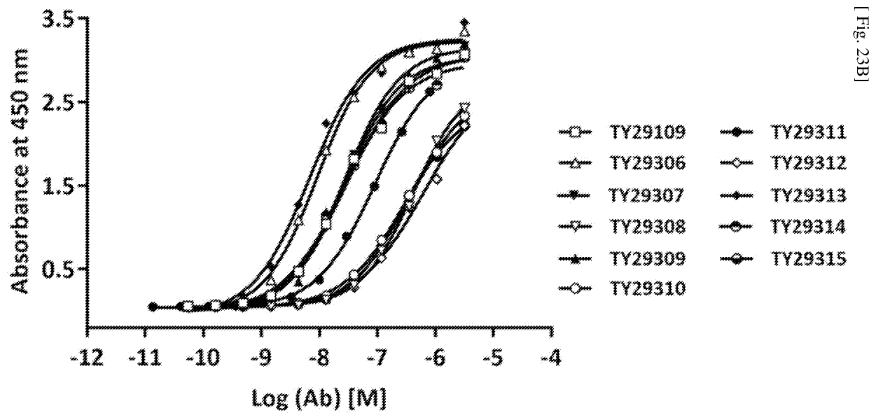

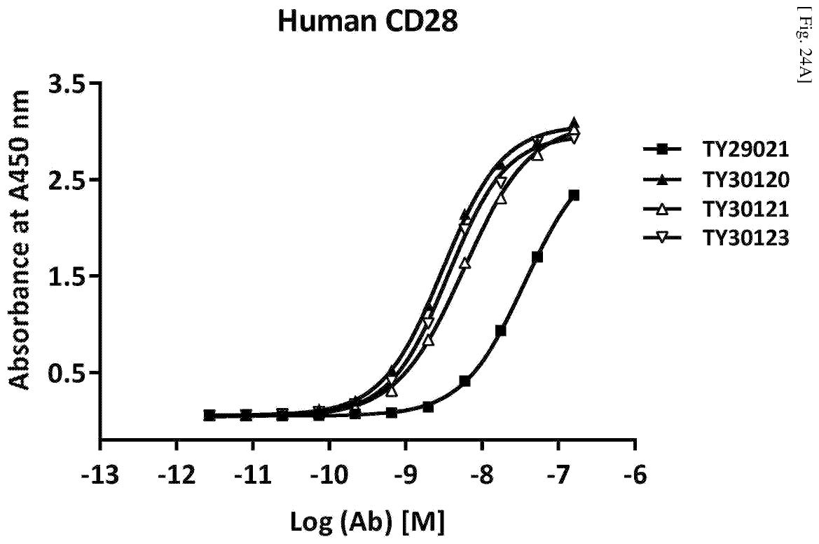

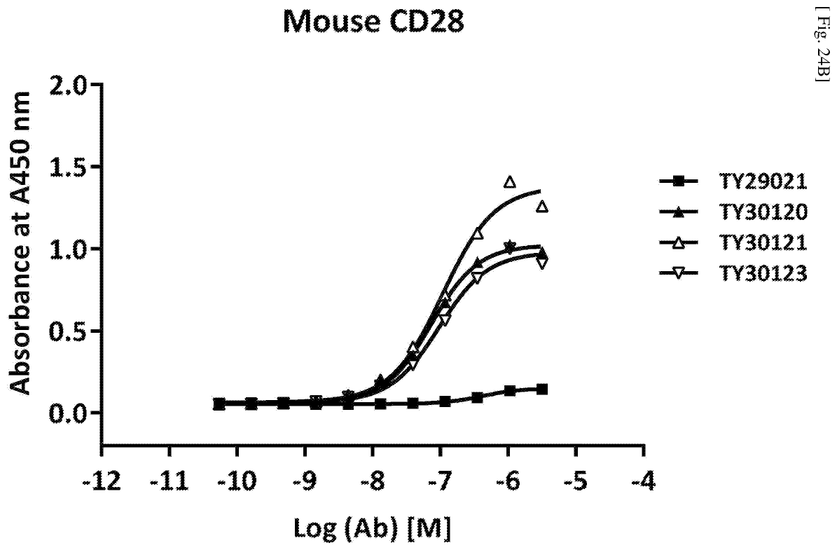

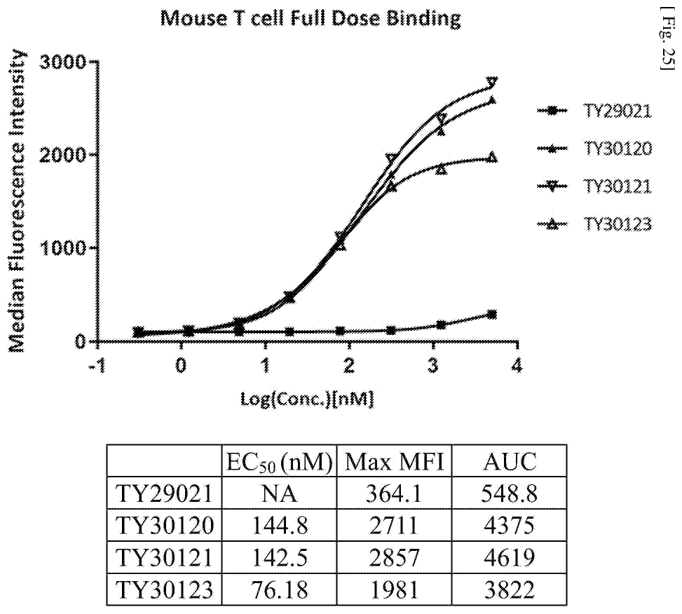

Anti-HER2×CD28 bsAbs or anti-B7-H3×CD28 bsAbs were constructed using TY24865 scFv variants in VH-VL or VL-VH formats. As shown in Table 21, these bsAbs show normal titers and SEC-purity. The CD28 affinities of these bsAbs were measured through ELISA and shown in FIGS. 23A and 23B. These scFv variants shows various affinities, ranging from 0.954 to 417 nM, and scFvs in VL-VH formats have higher affinity than those in VH-VL formats. Some variants in Table 22 and FIGS. 24A and 24B show high affinity to human and mouse, and these affinities were further confirmed in a mouse T-cell binding assay (FIG. 25).

TABLE 21
Construction and Characterization of Anti-
HER2xCD28 BsAbs with TY24865 Variants

| | CD28 scFv | Titer | SEC purity | | |
|---|---|---|---|---|---|
| BsAb ID | (VL-VH) | (mg/L) | HMW(%) | LMW (%) | EC₅₀ (nM) |
| TY27566 | WT | 61.7 | 4.4 | 0.0 | 3.89 |
| TY28652 | BC8227 | 60.5 | NA | NAA | 3.68 |
| TY28653 | BC8228 | 42.2 | 6.8 | 0.1 | 7.30 |
| TY28654 | BC8230 | 59.6 | 3 | 0 | 68.41 |
| TY28655 | BC8233 | 55.2 | 3.4 | 0.2 | 8.14 |
| TY28656 | BC8235 | 70.6 | 2.7 | 0 | 127.7 |
| TY28657 | BC8237 | 67.1 | 3.3 | 0 | 5.29 |
| TY28658 | BC8240 | 67.3 | 4.9 | 0 | 78.02 |
| TY28659 | BC8242 | 64 | 3.4 | 0.3 | 0.954 |
| TY28660 | BC8257 | 67.5 | 4.9 | 0.4 | 3.77 |
| TY28661 | BC8263 | 62.8 | 4.9 | 0 | 52.57 |
| | CD28 scFv | Titer | SEC purity | | |
| BsAb ID | (VH-VL) | (mg/L) | HMW(%) | LMW (%) | EC₅₀ (nM) |
| TY29109 | WT | 73.4 | 5.1 | 0.0 | 30.33 |
| TY29306 | BC8227 | 63.7 | 1.4 | 0.6 | 9.27 |
| TY29307 | BC8228 | 69.3 | 1.4 | 0 | 27.79 |
| TY29308 | BC8230 | 34.1 | 2.4 | 0 | 417.3 |
| TY29309 | BC8233 | 38.6 | 2 | 0 | 27.7 |
| TY29310 | BC8235 | 50.4 | 2.7 | 0 | 348.9 |
| TY29311 | BC8237 | 25.4 | 3 | 2.1 | 91.28 |
| TY29312 | BC8240 | 43.0 | 2.6 | 0 | 624.1 |
| TY29313 | BC8242 | 36.0 | 2.4 | 1.8 | 6.97 |
| TY29314 | BC8257 | 96.5 | 4 | 0.9 | 27.51 |
| TY29315 | BC8263 | 60.5 | 1.3 | 0 | 291.2 |

TABLE 22
Construction and Characterization of Anti-
B7-H3xCD28 BsAbs with TY24865 Variants

| | CD28 scFv | Human CD28 | Mouse CD28 |
|---|---|---|---|
| BsAb ID | (VH-VL) | EC₅₀ (nM) | EC₅₀ (nM) |
| TY29021 | WT | 36.39 | NM |
| TY30120 | BC10599 | 3.026 | 74.18 |
| TY30121 | BC10601 | 5.731 | 108.3 |
| TY30123 | BC10621 | 3.588 | 96.41 |

The above non-limiting examples are provided for illustrative purposes only in order to facilitate a more complete understanding of the disclosed subject matter. These examples should not be construed to limit any of the embodiments described in the present specification, including those pertaining to the antibodies, pharmaceutical compositions, or methods and uses for treating cancer, a neurodegenerative or an infectious disease.

### SEQUENCES

The tables below list sequences disclosed herein and SEQ ID NOs (SEQ) are shown in the left columns.

| SEQ | Description | Sequence |
|---|---|---|
| 1 | Human CD28 | MLRLLLALNLFPSIQVTGNKILVKQSPMLVAYDNAVNLSCKYSYNLF |
| | | SREFRASLHKGLDSAVEVCVVYGNYSQQLQVYSKTGFNCDGKLGNES |
| | | VTFYLQNLYVNQTDIYFCKIEVMYPPPYLDNEKSNGTIIHVKGKHLC |
| | | PSPLFPGPSKPFWVLVVVGGVLACYSLLVTVAFIIFWVRSKRSRLLH |
| | | SDYMNMTPRRPGPTRKHYQPYAPPRDFAAYRS |
| 2 | Macaca | MLRLLLALNLLPSIQVTGNKILVKQSPMLVAYDNAVNLSCKYSYNLF |
| | mulatta CD28 | SREFRASLHKGLDSAVEVCVVYGNYSQQLQVYSKTGFNCDGKLGNES |
| | | VTFYLQNLYVNQTDIYFCKIEVMYPPPYLDNEKSNGTIIHVKGKHLC |
| | | PSPLFPGPSKPFWALVVVGGVLACYSLLVTVAFCIFWMRSKRSRLLH |
| | | SDYMNMTPRRPGPTRKHYQPYAPPRDFAAYRS |
| 3 | Macaca | MLRLLLALNLLPSIQVTGNKILVKQSPMLVAYDNAVNLSCKYSYNLF |
| | fascicularis | SREFRASLHKGLDSAVEVCVVYGNYSQQLQVYSKTGFNCDGKLGNFS |
| | CD28 | VTFYLQNLYVNQTDIYFCKIEVMYPPPYLDNEKSNGTIIHVKGKHLC |
| | | PSPLFPGPSKPFWALVVVGGVLACYSLLVTVAFCIFWMRSKRSRLLH |
| | | SDYMNMTPRRPGPTRKHYQPYAPPRDFAAYRS |
| 4 | Mouse CD28 | MTLRLLFLALNFFSVQVTENKILVKQSPLLVVDSNEVSLSCRYSYNL |
| | | LAKEFRASLYKGVNSDVEVCVGNGNFTYQPQFRSNAEQNCDGDQDNE |
| | | TVTFRLWNLHVNHTDIYFCKIEFMYPPPYLDNERSNGTIIHIKEKHL |
| | | CHTQSSPKLFWALVVVAGVLFCYGLLVTVALCVIWTNSRRNRLLQSD |
| | | YMNMTPRRPGLTRKPYQPYAPARDFAAYRP |

Non-masked Anti-CD28 Antibodies
Anti-CD28 mAb (TY24865)

| | | |
|---|---|---|
| 5 | HCDR1 | TGGVGVS |
| 6 | HCDR2 | EIYHSGSTYYSPSLKS |
| 7 | HCDR3 | YEYYYYDFDY |
| 8 | LCDR1 | RASQSVDFHGISFLH |
| 9 | LCDR2 | AASSLQS |
| 10 | LCDR3 | QQSYRSPRT |
| 11 | VH | EVQLVESGGGLVQPGGSLRLSCAASGFSLSTGGVGVSWIRQAPGKGL |
| | | EWIGEIYHSGSTYYSPSLKSRVTISRDNSKNTLYLQLNSLRAEDTAV |
| | | YYCARYEYYYYDFDYWGQGTLVTVSS |
| 12 | VL | DIQLTQSPSSLSASVGDRVTITCRASQSVDFHGISFLHWYQQKPGKA |
| | | PKLLIYAASSLQSGVPSRFSGSGSGTDFTLTISSLQPEDFATYYCQQ |
| | | SYRSPRTFGQGTKVEIK |
| 13 | HC | EVQLVESGGGLVQPGGSLRLSCAASGFSLSTGGVGVSWIRQAPGKGL |
| | | EWIGEIYHSGSTYYSPSLKSRVTISRDNSKNTLYLQLNSLRAEDTAV |
| | | YYCARYEYYYYDFDYWGQGTLVTVSSASTKGPSVFPLAPCSRSTSES |
| | | TAALGCLVKDYFPEPVTVSWNSGALTSGVHTFPAVLQSSGLYSLSSV |
| | | VTVPSSSLGTKTYTCNVDHKPSNTKVDKRVESKYGPPCPPCPAPEFL |
| | | GGPSVFLFPPKPKDTLMISRTPEVTCVVVDVSQEDPEVQFNWYVDGV |
| | | EVHNAKTKPREEQENSTYRVVSVLTVLHQDWLNGKEYKCKVSNKGLP |
| | | SSIEKTISKAKGQPREPQVYTLPPSQEEMTKNQVSLTCLVKGFYPSD |
| | | IAVEWESNGQPENNYKTTPPVLDSDGSFFLYSRLTVDKSRWQEGNVF |
| | | SCSVMHEALHNHYTQKSLSLSLGK |
| 14 | | DIQLIQSPSSLSASVGDRVTITCRASQSVDFHGISFLHWYQQKPGKA |
| | | PKLLIYAASSLQSGVPSRFSGSGSGTDFTLTISSLQPEDFATYYCQQ |
| | | SYRSPRTFGQGTKVEIKRTVAAPSVFIFPPSDEQLKSGTASVVCLLN |
| | | NFYPREAKVQWKVDNALQSGNSQESVTEQDSKDSTYSLSSTLTLSKA |
| | | DYEKHKVYACEVTHQGLSSPVTKSFNRGEC |

Anti-CD28 mAb (TY24853)

| | | |
|---|---|---|
| 15 | HCDR1 | SGHHWS |
| 6 | HCDR2 | EIYHSGSTYYSPSLKS |
| 16 | HCDR3 | KDDYYAFDY |
| 17 | LCDR1 | RASESVDFYGISFLH |
| 18 | LCDR2 | AASTLQS |
| 19 | LCDR3 | QQSYRTPIT |
| 20 | VH | EVQLVESGGGLVQPGGSLRLSCAASGYSITSGHHWSWIRQAPGKGLE |
| | | WIGEIYHSGSTYYSPSLKSRVTISRDNSKNTLYLQINSIRAEDTAVY |
| | | YCARKDDYYAFDYWGQGTLVTVSS |
| 21 | VL | DIQLTQSPSSLSASVGDRVTITCRASESVDFYGISFLHWYQQKPGKA |
| | | PKLLIYAASTLQSGVPSRFSGSGSGTDFTLTISSLQPEDFATYYCQQ |
| | | SYRTPITFGQGTKVEIK |
| 22 | HC | EVQLVESGGGLVQPGGSLRLSCAASGYSITSGHHWSWIRQAPGKGLE |
| | | WIGEIYHSGSTYYSPSLKSRVTISRDNSKNTLYLQINSLRAEDTAVY |
| | | YCARKDDYYAFDYWGQGTLVTVSSASTKGPSVFPLAPCSRSTSESTA |
| | | ALGCLVKDYFPEPVTVSWNSGALTSGVHTFPAVLQSSGLYSLSSVVT |
| | | VPSSSLGTKTYTCNVDHKPSNTKVDKRVESKYGPPCPPCPAPEFLGG |
| | | PSVFLFPPKPKDTLMISRTPEVTCVVVDVSQEDPEVQFNWYVDGVEV |
| | | HNAKTKPREEQENSTYRVVSVLTVLHQDWLNGKEYKCKVSNKGLPSS |
| | | IEKTISKAKGQPREPQVYTLPPSQEEMTKNQVSLTCLVKGEYPSDIA |
| | | VEWESNGQPENNYKTTPPVLDSDGSFFLYSRLTVDKSRWQEGNVFSC |
| | | SVMHEALHNHYTQKSLSLSLGK |
| 23 | LC | DIQLTQSPSSLSASVGDRVTITCRASESVDFYGISFLHWYQQKPGKA |
| | | PKLLIYAASTLQSGVPSRFSGSGSGTDFTLTISSLQPEDFATYYCQQ |
| | | SYRTPITFGQGIKVEIKRTVAAPSVFIFPPSDEQLKSGTASVVCLLN |
| | | NFYPREAKVQWKVDNALQSGNSQESVTEQDSKDSTYSLSSTLTLSKA |
| | | DYEKHKVYACEVTHQGLSSPVTKSFNRGEC |

Anti-CD28 mAb (TY24854)

| | | |
|---|---|---|
| 24 | HCDR1 | SGYHWG |
| 6 | HCDR2 | EIYHSGSTYYSPSLKS |
| 25 | HCDR3 | RRGDGYFDF |
| 26 | LCDR1 | RASQSVDFLGISFLH |
| 27 | LCDR2 | DASNRAT |
| 28 | LCDR3 | QQSYRTPLT |
| 29 | VH | EVQLVESGGGLVQPGGSLRLSCAASGYSITSGYHWGWIRQAPGKGLE |
| | | WIGEIYHSGSTYYSPSLKSRVTISRDNSKNTLYLQLNSLRAEDTAVY |
| | | YCARRRGDGYFDFWGQGTLVTVSS |
| 30 | VL | DIQLTQSPSSLSASVGDRVTITCRASQSVDFLGISFLHWYQQKPGKA |
| | | PKLLIYDASNRATGIPSRFSGSGSGTDFTLTISSLQPEDFATYYCQQ |
| | | SYRTPLTEGQGTKVEIK |
| 31 | HC | EVQLVESGGGLVQPGGSLRLSCAASGYSITSGYHWGWIRQAPGKGLE |
| | | WIGEIYHSGSTYYSPSLKSRVTISRDNSKNTLYLQLNSLRAEDTAVY |
| | | YCARARGDGYFDFWGQGTLVTVSSASTKGPSVFPLAPCSRSTSESTA |
| | | ALGCLVKDYFPEPVTVSWNSGALTSGVHIFPAVLQSSGLYSLSSVVT |
| | | VPSSSLGTKTYTCNVDHKPSNTKVDKRVESKYGPPCPPCPAPEFLGG |
| | | PSVFLFPPKPKDSLMISRTPEVTCVVVDVSQEDPEVQFNWYVDGVEV |
| | | HNAKTKPREEQENSTYRVVSVLTVLHQDWLNGKEYKCKVSNKGLPSS |
| | | IEKTISKAKGQPREPQVYTLPPSQEEMTKNQVSLTCLVKGFYPSDIA |
| | | VEWESNGQPENNYKTTPPVLDSDGSFFLYSRLTVDKSRWQEGNVFSC |
| | | SVMHEALHNHYTQKSLSLSLGK |
| 32 | LC | DIQLTQSPSSLSASVGDRVTITCRASQSVDFLGISFLHWYQQKPGKA |
| | | PKLLIYDASNRATGIPSRFSGSGSGTDFTLTISSLQPEDFATYYCQQ |
| | | SYRTPLTFGQGTKVEIKRTVAAPSVFIFPPSDEQLKSGTASVVCLLN |
| | | NEYPREAKVQWKVDNALQSGNSQESVTEQDSKDSTYSLSSTLTLSKA |
| | | DYEKHKVYACEVCHQGLSSPVTKSFNRGEC |

Anti-CD28 mAb (TY24859)

| | | |
|---|---|---|
| 33 | HCDR1 | TGGVGVG |
| 6 | HCDR2 | EIYHSGSTYYSPSLLKS |
| 35 | HCDR3 | YGYSYYALDY |
| 36 | LCDR1 | SASSSVGYVQ |
| 37 | LCDR2 | DASNLET |
| 38 | LCDR3 | QQSYTSPRT |
| 39 | VH | EVQLVESGGGLVQPGGSLRLSCAASGFSLSTGGVGVGWIRQAPGKGL |
| | | EWIGEIYHSGSTYYSPSLKSRVTISRDNSKNTLYLQLNSLRAEDTAV |
| | | YYCARYGYSYYALDYWGQGTLVTVSS |
| 40 | VL | DIQLTQSPSSLSASVGDRVTITCSASSSVGYVQWYQQKPGKAPKLLI |
| | | YDASNLETGVPSRFSGSGSGTDFTLTISSLQPEDFATYYCQQSYTSP |
| | | RTFGQGTKVEIK |
| 41 | HC | EVQLVESGGGLVQPGGSLRLSCAASGFSLSTGGVGVGWIRQAPGKGL |
| | | EWIGEIYHSGSTYYSPSLKSRVTISRDNSKNTLYLQLNSLRAEDTAV |
| | | YYCARYGYSYYALDYWGQGTLVTVSSASTKGPSVFPLAPCSRSTSES |
| | | TAALGQLVKDYFPEPVTVSWNSGALTSGVHTFPAVLQSSGLYSLSSV |
| | | VTVPSSSLGTKTYTCNVDHKPSNTKVDKRVESKYGPPCPPCPAPEFL |
| | | GGPSVFLFPPKPKDTLMISRTPEVTCVVVDVSQEDPEVQFNWYVDGV |
| | | EVHNAKTKPREEQFNSTYRVVSVLTVLHQDWLNGKEYKCKVSNKGLP |
| | | SSIEKTISKAKGQPREPQVYTTPPSQEEMTKNQVSLTCLVKGEYPSD |
| | | IAVEWESNGQPENNYKTTPPVLDSDGSFFLYSRLTVDKSRWQEGNVF |
| | | SCSVMHEALHNHYTQKSLSLSLGK |
| 42 | LC | DIQLTQSPSSLSASVSDRVTITCSASSSVGYVQWYQQKPGKAPKLLI |
| | | YDASNLETGVPSRFSGSGSGTDFTLTISSLQPEDFATYYCQQSYTSP |
| | | RTFGQGTKVEIKRTVAAPSVFIFPPSDEQLKSGTASVVCLLNNFYPR |
| | | EAKVQWKVDNALQSGNSQESVTEQDSKDSTYSLSSTLILSKADYEKH |
| | | KVYACEVTHQGLSSPVTKSFNRGEC |

Anti-CD28 mAb (TY24860)

| | | |
|---|---|---|
| 43 | HCDR1 | TSGVGVS |
| 5 | HCDR2 | EIYHSGSTYYSPSLKS |
| 44 | HCDR3 | KGYYYYAFDY |
| 45 | LCDR1 | RASQDIGSFLA |
| 9 | LCDR2 | AASSLQS |
| 46 | LCDR3 | QQSYRSPPT |
| 47 | VH | EVQLVESGGGLVQPGGSLRLSCAASGFSLSTSGVGVSWIRQAPGKGL |
| | | EWIGEIYHSGSTYYSPSLKSRVTISRDNSKNTLYLQLNSLRAEDTAV |
| | | YYCARKGYYYYAFDYWGPGTLVTVSS |
| 48 | VL | DIQLTQSPSSLSASVGDRVTITCRASQDIGSFLAWYQQKPGKAPKLL |
| | | IYAASSLQSGVPSRFSGSGSGTDFTLTISSLQPEDFATYYCQQSYRS |
| | | PPTFGQGTKVEIK |
| 49 | HC | EVQLVESGGGLVQPGGSLRLSCAASGFSLSTSGVGVSWIRQAPGKGL |
| | | EWIGEIYHSGSTYYSPSLKSRVTISRDNSKNTLYLQLNSLRAEDTAV |
| | | YYCARKGYYYYAFDYWGPGTLVTVSSASTKGPSVFPLAPCSRSTSES |
| | | TAALGCLVKDYFPEPVTVSWNSGALTSGVHTFPAVLQSSGLYSLSSV |
| | | VTVPSSSLGTKTYTCNVDHKPSNTKVDKRVESKYGPPCPPCPAPEFL |
| | | GGPSVFLFPPKPKDTLMISRTPEVTCVVVDVSQEDPEVQFNWYVDGV |
| | | EVHNAKTKPREEQFNSTYRVVSVLTVLHQDWLNGKEYKCKVSNKGLP |
| | | SSIEKTISKAKGQPREPQVYTLPPSQEEMTKNQVSLTCLVKGFYPSD |
| | | IAVEWESNGQPENNYKTTPPVLDSDGSFFLYSRLTVDKSRWQEGNVF |
| | | SCSVMHEALHNHYTQKSLSLSLGK |
| 50 | LC | DIQLTQSPSSLSASVGDRVTITCRASQDIGSFLAWYQQKPGKAPKLL |
| | | IYAASSLQSGVPSRFSGSGSGTDFTLTISSLQPEDFATYYCQQSYRS |
| | | PPTFGQGTKVEIKRTVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVIKSFNRGEC |

Anti-CD28 mAb (TY24866)

| | | |
|---|---|---|
| 33 | HCDR1 | TGGVGVG |
| 51 | HCDR2 | EIYHSGSTYYPPSLKS |
| 52 | HCDR3 | YEYSSYGLDY |
| 53 | LCDR1 | RASQDIRKFLA |
| 300 | LCDR2 | DASSLES |
| 10 | LCDR3 | QQSYRSPRT |
| 54 | VH | EVQLVESGGGLVQPGSSLRLSCAASGFSLSTGGVGVGWIRQAPGKGL |
| | | EWIGETYHSGSTYYPPSLKSRVTISRDNSKNTLYLQLNSLRAEDTAV |
| | | YYCARYEYSSYGLDYWGQGTLVTVSS |
| 55 | VL | DIQLTQSPSSLSASVGDRVTITCRASQDIRKFLAWYQQKEGKAPKLL |
| | | IYDASSLESGVPSRFSGSGSGTDFTLTISSLQPEDFATYYCQQSYRS |
| | | PRTEGQGTKVEIK |
| 56 | HC | EVQLVESGGGLVQPGGSLRLSCAASGFSLSTGGVGVGWIRQAPGKGL |
| | | EWIGEIYISGSTYYPPSLKSRVTISRDNSKNTLYLQLNSLRAEDTAV |
| | | YYCARYEYSSYGLDYWGQGTLVTVSSASTKGPSVFPLAPCSRSTSES |
| | | TAALGCLVKDYFPEPVTVSWNSGALTSGVHTFPAVLQSSGLYSLSSV |
| | | VTVPSSSLGTKTYTCNVDHKPSNTKVDKRVESKYGPPCPPCPAPEFL |
| | | GGPSVFLFPPKPKDTLMISRTPEVTCVVVDVSQEDPEVQFNWYVDGV |
| | | EVANAKTKPREEQFNSTYRVVSVLTVLHQDWLNGKEYKCKVSNKGLP |
| | | SSIEKTISKAKGQPREPQVYTLPPSQEEMTKNQVSLTCLVKGEYPSD |
| | | IAVEWESNGQPENNYKTTPPVLDSDGSFFLYSRLTVDKSRWQEGNVF |
| | | SCSVMHEALHNHYTQKSLSLSLGK |
| 57 | LC | DIQLTQSPSSLSASVGDRVTITCRASQDIRKFLAWYQQKPGKAPKLL |
| | | TYDASSLESGVPSRFSGSGSGTDFTLTISSLQPEDEATYYCQQSYRS |
| | | PRFFGQGTKVEIKRTVAAPSVFIFPPSDEQLKSGTASVVCLLNNFYP |
| | | REAKVQWKVDNALQSSNSQESVTEQDSKDSTYSLSSTLTLSKADYEK |
| | | HKVYACEVTHQGLSSPVIKSFNRGEC |

Anti-CD28 mAb (TY24869)

| | | |
|---|---|---|
| 24 | HCDR1 | SGYHWG |
| 58 | HCDR2 | AIYHSGSTYYSPSLKS |
| 59 | HCDR3 | SGLVYYAFDY |
| 60 | LCDR1 | RASQGIGSYLA |
| 300 | LCDR2 | DASSLES |
| 61 | LCDR3 | QQYSTSPLT |
| 62 | VH | QVQLVQSGAEVKKPGSSVKVSCKASGYSISSGYHWGWIRQAPGQGLE |
| | | WIGAIYHSGSTYYSPSLKSRVTITADKSTSTAYMELSSLRSEDTAVY |
| | | YCARSGLVYYAFDYWGQGTLVTVSS |
| 63 | VL | DIQLTQSPSSLSASVGDRVTITCRASQGIGSYLAWYQQKPGKAPKLL |
| | | IYDASSLESGVPSRFSGSGSGTDFTLTISSLQPEDFATYYCQQYSTS |
| | | PLTFGQGTKVEIK |
| 64 | HC | QVQLVQSGAEVKKPGSSVKVSCKASGYSISSGYHWGWIRQAPGQGLE |
| | | WIGAIYHSGSTYYSPSLKSRVTITADKSISTAYMELSSLRSEDIAVY |
| | | YCARSGLVYYAFDYWSQGTLVTVSSASTKGPSVFPLAPCSRSTSEST |
| | | AALGCLVKDYFPEPVTVSWNSGALTSGVHTFPAVLQSSGLYSLSSVV |
| | | TVPSSSLGTKTYCCNVDHKPSNTKVDKRVESKYGPPCPPCPAPEFLG |
| | | GPSVFLFPPKPKDTLMISRTPEVTCVVVDVSQEDPEVQFNWYVDGVE |
| | | VHNAKTKPREEQENSTYRVVSVLTVLHQDWLNGKEYKCKVSNKGLPS |
| | | SIEKTISKAKGQPREPQVYTLPPSQEEMTKNQVSLTCLVKGFYPSDI |
| | | AVEWESNGQPENNYKTTPPVLDSDGSFFLYSRLTVDKSRWQEGNVFS |
| | | CSVMHEALHNHYCQKSLSLSLGK |
| 65 | LC | DIQLTQSPSSLSASVGDRVTITCRASQGIGSYLAWYQQKPGKAPKLL |
| | | IYDASSLESGVPSRFSGSGSGTDFTLTISSLQPEDFATYYCQQYSTS |
| | | PLTFGQGTKVEIKRIVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVTKSFNRGEC |

Anti-CD28 mAb (TY24871)

| | | |
|---|---|---|
| 66 | HCDR1 | SYAIH |
| 67 | HCDR2 | SIYHSGSTYYSPSLKS |
| 68 | HCDR3 | DVYGSYALDV |
| 69 | LCDR1 | RASQSVDFDGISFLH |
| 300 | LCDR2 | DASSLES |
| 70 | LCDR3 | QQSYRSPIT |
| 71 | VH | QVQLVQSGAEVKKPGSSVKVSCKASGFTFSSYAIHWVRQAPGQGLEW |
| | | IGSIYHSGSTYYSPSLKSRVTITADKSTSTAYMELSSLRSEDTAVYY |
| | | CARDVYGSYALDVWGQGTLVTVSS |
| 72 | VL | DIQLTQSPSSLSASVGDRVTITCRASQSVDFDGISFLHWYQQKPGKA |
| | | PKLLIYDASSLESGVPSRFSGSGSGTDFTLTISSLQPEDFATYYCQQ |
| | | SYRSPITFGQGTKVEIK |
| 73 | HC | QVQLVQSGAEVKKPGSSVKVSCKASGFTFSSYAIHWVRQAPGQGLEW |
| | | IGSIYHSGSTYYSPSLKSRVTITADKSTSTAYMELSSLRSEDTAVYY |
| | | CARDVYGSYALDVWGQGTLVTVSSASTKGPSVFPLAPCSRSTSESTA |
| | | ALGCLVKDYFPEPVTVSWNSGALTSGVHTFPAVLQSSGLYSLSSVVT |
| | | VPSSSLGTKTYTCNVDHKPSNTKVDKRVESKYGPPCPPCPAPEFLGG |
| | | PSVFLFPPKPKDTLMISRIPEVTCVVVDVSQEDPEVQFNWYVDGVEV |
| | | HNAKTKPREEQFNSTYRVVSVLTVLHQDWLNGKEYKCKVSNKGLPSS |
| | | IEKTISKAKGQPREPQVYTLPPSQEEMTKNQVSLTCLVKGFYPSDIA |
| | | VEWESNGQPENNYKTTPPVLDSDGSFFLYSRLTVDKSRWQEGNVFSC |
| | | SVMHEALHNHYTQKSLSLSLGK |
| 74 | LC | DIQLTQSPSSLSASVGDRVTITCRASQSVDFDGISFLHWYQQKPGKA |
| | | PKLLIYDASSLESGVPSRFSGSGSGTDFTLTISSLQPEDFATYYCQQ |
| | | SYRSPITFGQGTKVEIKRTVAAPSVFIFPPSDEQLKSGTASVVCLLN |
| | | NEYPREAKVQWKVDNALQSGNSQESVTEQDSKDSTYSLSSTLTLSKA |
| | | DYEKHKVYACEVTHQGLSSPVTKSFNRGEC |

Anti-CD28 mAb (TY24773)

| | | |
|---|---|---|
| 24 | HCDR1 | SGYHWG |
| 6 | HCDR2 | EIYHSGSTYYSPSLKS |
| 75 | HCDR3 | KDYYYALDY |
| 76 | LCDR1 | RASQSVDFYGKSFLII |
| 18 | LCDR2 | AASTLQS |
| 28 | LCDR3 | QQSYRIPLT |
| 77 | VH | EVQLVESGGGLVQPGGSLRLSCAASGYSISSGYHWGWIRQAPGKGLE |
| | | WIGEIYHSGSTYYSPSLKSRVTISRDNSKNTLYLQLNSLRAEDTAVY |
| | | YCARKDYYYALDYWGQGTLVTVSS |
| 78 | VL | DIQLTQSPSSLSASVGDRVTITCRASQSVDFYGKSFLHWYQQKPGKA |
| | | PKLLIYAASTLQSGVPSRFSGSGSGTDFTLTISSLQPEDFATYYCQQ |
| | | SYRTPLTFGQGTKVEIK |
| 79 | HC | EVQLVESGGGLVQPGGSLRLSCAASGYSISSGYHWGWIRQAPGKGLE |
| | | WIGEIYHSGSTYYSPSLKSRVTISRDNSKNTLYLQLNSLRAEDTAVY |
| | | YCARKDYYYALDYWGQGTLVTVSSASTKGPSVFPLAPCSRSTSESTA |
| | | ALGCLVKDYFPEPVTVSWNSGALTSGVHTFPAVLQSSGLYSLSSVVT |
| | | VPSSSLGTKTYTCNVDHKPSNTKVDKRVESKYGPPCPPCPAPEFLGG |
| | | PSVFLFPPKPKDTLMISRTPEVTCVVVDVSQEDPEVQFNWYVDGVEV |
| | | HNAKTKPREEQENSTYRVVSVLTVLHQDWLNGKEYKCKVSNKGLPSS |
| | | IEKTISKAKGQPREPQVYTLPPSQEEMTKNQVSLTCLVKGFYPSDIA |
| | | VEWESNGQPENNYKTTPPVLDSDGSFFLYSRLTVDKSRWQEGNVFSC |
| | | SVMHEALHNHYTQKSLSLSLGK |
| 80 | LC | DIQLTQSPSSLSASVGDRVTITCRASQSVDFYGKSFLHWYQQKPGKA |
| | | PKLLIYAASTLQSGVPSRFSGSGSGTDFTLTISSLQPEDFATYYCQQ |
| | | SYRTPLTFGQGTKVEIKRTVAAPSVFIFPPSDEQLKSGTASVVCLLN |
| | | NEYPREAKVQWKVDNALQSGNSQESVTEQDSKDSTYSLSSTLTLSKA |
| | | DYEKHKVYACEVTHQGLSSPVTKSFNRGEC |

Anti-CD28 mAb (TY24876)

| | | |
|---|---|---|
| 24 | HCDR1 | SGYHWG |
| 58 | HCDR2 | AIYHSGSTYYSPSLKS |
| 81 | HCDR3 | GPLSYYAFDY |
| 82 | LCDR1 | RASQSVSRYLA |
| 27 | LCDR2 | DASNRAT |
| 83 | LCDRS | QQYYRSPIT |
| 84 | VH | QVQLVQSGAEVKKPGSSVKVSCKASGYSITSGYHWGWIRQAPGQGLE |
| | | WIGAIYHSGSTYYSPSLKSRVTITADKSTSTAYMELSSLRSEDTAVY |
| | | YCARGPLSYYAFDYWGQGTLVTVSS |
| 85 | VL | DIQLTQSPSSLSASVGDRVTITCRASQSVSRYLAWYQQKPGKAPKLL |
| | | IYDASNRATGIPSRFSGSGSGTDFTLTISSLQPEDEATYYCQQYYRS |
| | | PITFGQGTKVEIK |
| 86 | HC | QVQLVQSGAEVKKPGSSVKVSCKASGYSITSGYHWGWIRQAPGQGLE |
| | | WIGAIYHSGSTYYSPSLKSRVTITADKSTSTAYMELSSLRSEDTAVY |
| | | YCARGPLSYYAFDYWGQGTLVTVSSASTKGPSVFPLAPCSRSTSEST |
| | | AALGCLVKDYFPEPVTVSWNSGALTSGVHTEPAVLQSSGLYSLSSVV |
| | | TVPSSSLGTKTYTCNVDHKPSNTKVDKRVESKYGPPCPPCPAPEFLG |
| | | GPSVFLFPPKPKDTLMISRTPEVTCVVVDVSQEDPEVQFNWYVDGVE |
| | | VHNAKTKPREEQFNSTYRVVSVLTVLHQDWLNGKEYKCKVSNKGLPS |
| | | SIEKTISKAKGQPREPQVYTLPPSQEEMTKNQVSLTCLVKGFYPSDI |
| | | AVEWESNGQPENNYKTTPPVLDSDGSFFLYSRLTVDKSRWQEGNVFS |
| | | CSVMHEALHNHYTQKSLSLSLGK |
| 87 | LC | DIQLTQSPSSLSASVGDRVTITCRASQSVSRYLAWYQQKPGKAPKLL |
| | | IYDASNRATGIPSRFSGSGSGTDFTLTISSLQPEDFATYYCQQYYRS |
| | | PITFGQGTKVEIKRIVAAPSVFIFPPSDEQLKSGTASVVQLLNNFYP |
| | | REAKVQWKVDNALQSGNSQESVTEQDSKDSTYSLSSTLTLSKADYEK |
| | | HKVYACEVTHQGLSSPVTKSFNRGEC |

Anti-CD28 mAb (TY24878)

| | | |
|---|---|---|
| 88 | HCDR1 | TGGVAVA |
| 89 | HCDR2 | QIYYSGSTYYNPSLKS |
| 90 | HCDR3 | SSAHYYFDV |
| 91 | LCDR1 | RASESVDFQGISFLD |
| 300 | LCDR2 | DASSLES |
| 70 | LCDR3 | QQSYRSPIT |
| 92 | VH | QVQLVQSGAEVKKPGSSVKVSCKASGFSLSTGGVAVAWIRQAPGQGL |
| | | EWIGQIYYSGSTYYNPSLKSRVTITADKSTSTAYMELSSLRSEDTAV |
| | | YYCARSSAHYYEDVWGQGTLVTVSS |
| 93 | VL | DIQLTQSPSSLSASVGDRVTITCRASESVDFQGISFLDWYQQKPGKA |
| | | PKLLIYDASSLESGVPSRFSGSGSGTDFTLTISSLQPEDFATYYCQQ |
| | | SYRSPITFGQGTKVEIK |
| 94 | HC | QVQLVQSGAEVKKPGSSVKVSCKASGFSLSIGGVAVAWIRQAPGQGL |
| | | EWIGQIYYSGSTYYNPSLKSRVTITADKSTSTAYMELSSLRSEDTAV |
| | | YYCARSSAHYYFDVWGQGTLVTVSSASTKGPSVFPLAPCSRSTSEST |
| | | AALGCLVKDYFPEPVTVSWNSGALTSGVHTFPAVLQSSGLYSLSSVV |
| | | TVPSSSLGTKIYTCNVDHKPSNTKVDKRVESKYGPPCPPCPAPEFLG |
| | | GPSVFLFPPKPKDTLMISRTPEVTCVVVDVSQEDPEVQFNWYVDGVE |
| | | VHNAKTKPREEQFNSTYRVVSVLTVLHQDWLNGKEYKCKVSNKGLPS |
| | | SIEKTISKAKGQPREPQVYTTPPSQEEMTKNQVSLTCLVKGFYPSDI |
| | | AVEWESNGQPENNYKTTPPVLDSDGSFFLYSRLTVDKSRWQEGNVFS |
| | | CSVMHEALHNHYTQKSLSLSLGK |
| 95 | LC | DIQLTQSPSSLSASVGDRVTITCRASESVDFQGISFLDWYQQKPGKA |
| | | PKLLIYDASSLESGVPSRFSGSGSGTDFTLTISSLQPEDFATYYCQQ |
| | | SYRSPITFGQGTKVEIKRTVAAPSVFIFPPSDEQLKSGTASVVCLLN |
| | | NEYPREAKVQWKVDNALQSGNSQESVTEQDSKDSTYSLSSTLTLSKA |
| | | DYEKHKVYACEVTHQGLSSPVTKSFNRGEC |

Anti-CD28 mAb (TY24879)

| | | |
|---|---|---|
| 24 | HCDR1 | SGYHWG |
| 96 | HCDR2 | SIYHSGSTYYNPSLKS |
| 97 | HCDR3 | SGLRYYAFDV |
| 98 | LCDR1 | AASQDVSTAVA |
| 9 | LCDR2 | AASSLQS |
| 70 | LCDR3 | QQSYRSPIT |
| 99 | VH | QVQLVQSGAEVKKPGSSVKVSCKASGYSISSGYHWGWIRQAPGQGLE |
| | | WIGSIYHSGSTYYNPSLKSRVTITADKSTSTAYMELSSLRSEDTAVY |
| | | YCARSGLRYYAFDVWGQGTLVTVSS |
| 100 | VL | DIQLTQSPSSLSASVGDRVTITCAASQDVSTAVAWYQQKPGKAPKLL |
| | | IYAASSLQSGVPSRFSGSGSGTDFTLTISSLQPEDFATYYCQQSYRS |
| | | PITFGQGTKVEIK |
| 101 | HC | QVQLVQSGAEVKKPGSSVKVSCKASGYSISSGYHWGWIRQAPGQGLE |
| | | WIGSIYHSGSTYYNPSLKSRVTITADKSTSTAYMELSSLRSEDTAVY |
| | | YCARSGLRYYAFDVWGQGTLVTVSSASTKGPSVFPLAPCSRSTSEST |
| | | AALGCLVKDYFPEPVTVSWNSGALTSGVHTFPAVLQSSGLYSLSSVV |
| | | TVPSSSLGTKTYCCNVDHKPSNTKVDKRVESKYGPPCPPCPAPEFLG |
| | | GPSVFLFPPKPKDTLMISRTPEVTCVVVDVSQEDPEVQFNWYVDGVE |
| | | VHNAKTKPREEQFNSTYRVVSVLTVLHQDWLNGKEYKCKVSNKGLPS |
| | | SIEKTISKAKGQPREPQVYTLPPSQEEMTKNQVSLTCLVKGFYPSDI |
| | | AVEWESNGQPENNYKTTPPVLDSDGSFFLYSRLTVDKSRWQEGNVFS |
| | | CSVMHEALHNHYTQKSLSLSLGK |
| 102 | LC | DIQLTQSPSSLSASVSDRVTITCAASQDVSTAVAWYQQKPGKAPKLL |
| | | TYAASSLQSGVPSRFSGSGSGTDFTLTISSLQPEDEATYYCQQSYRS |
| | | PITFGQGTKVEIKRTVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVTKSFNRGEC |

Anti-CD28 mAb (TY24884)

| | | |
|---|---|---|
| 103 | HCDR1 | GYAIH |
| 104 | HCDR2 | QIYHSGSTYYNPSLKS |
| 105 | HCDR3 | SGYGGYDFDY |
| 106 | LCDRI | RASQSVPRRYLA |
| 18 | LCDR2 | AASTLQS |
| 83 | LCDR3 | QQYYRSPIT |
| 107 | VH | QVQLVQSGAEVKKPGSSVKVSCKASGFTFSGYAIHWVRQAPGQGLEW |
| | | IGQIYHSGSTYYNPSLKSRVTITADKSTSTAYMELSSLRSEDTAVYY |
| | | CARSGYGGYDFDYWGQGTLVTVSS |
| 108 | VL | DIQLTQSPSSLSASVGDRVTITCRASQSVPRRYLAWYQQKPGKAPKL |
| | | LIYAASTLQSGVPSRFSGSGSGTDFTLTISSLQPEDFATYYCQQYYR |
| | | SPITFGQGTKVEIK |
| 109 | HC | QVQLVQSGAEVKKPGSSVKVSCKASGFTFSGYAIHWVRQAPSQGLEW |
| | | IGQIYHSGSTYYNPSLKSRVTITADKSTSTAYMELSSLRSEDTAVYY |
| | | CARSGYGGYDFDYNGQGTLVTVSSASTKGPSVFPLAPCSRSTSESTA |
| | | ALGCLVKDYFPEPVTVSWNSGALTSGVHTFPAVLQSSGLYSLSSVVT |
| | | VPSSSLGTKTYTCNVDHKPSNTKVFKRVESKYGPPCPPCPAPEFLGG |
| | | PSVFLFPPKPKDTLMISRTPEVTCVVVDVSQEDPEVQFNWYVDGVEV |
| | | HNAKTKPREEQENSTYRVVSVLTVLHQDWLNGKEYKCKVSNKGLPSS |
| | | IEKTISKAKGOPREPQVYTTPPSQEEMTKNQVSLTCLVKGEYPSDIA |
| | | VEWESNGQPENNYKTTPPVLDSDGSFFLYSRLTVDKSRWQEGNVFSC |
| | | SVMHEALHNHYTQKSLSLSLGK |
| 110 | LC | DIQLTQSPSSLSASVGDRVTITCRASQSVPRRYLAWYQQKPGKAPKL |
| | | LIYAASTLQSGVPSRFSGSGSGTDFTLTISSLQPEDFATYYCQQYYR |
| | | SPITFGQGTKVEIKRQVAAPSVFIFPPSDEQLKSGTASVVCLLNNFY |
| | | PREAKVQWKVDNALQSGNSQESVTEQDSKDSTYSLSSTLTLSKADYE |
| | | KHKVYACEVTHQGLSSPVTKSFNRGEG |

Anti-CD28 mAb (TY24890)

| | | |
|---|---|---|
| 111 | HCDR1 | SGHHWG |
| 6 | HCDR2 | EIYHSGSTYYSPSLKS |
| 112 | HCDR3 | RELGYAYFDY |
| 113 | LCDR1 | RASQDIRKYLA |
| 18 | LCDR2 | AASTLQS |
| 114 | LCDR3 | QQYYRTPLT |
| 115 | VH | EVQLVESGGGLVQPGGSLRLSCAASGYSISSGHHWGWIRQAPGKGLE |
| | | WIGEIYHSGSTYYSPSLKSRVTISRDNSKNTLYLQLNSLRAEDTAVY |
| | | YCARRELGYAYFDYWGQGTLVTVSS |
| 116 | VL | EIVLTQSPATLSLSPGERATLSCRASQDIRKYLAWYQQKPGQAPRLL |
| | | IYAASTLQSGVPARFSGSGSGTDFTLTISSLEPEDEAVYYCQQYYRT |
| | | PLTFGQGTKVEIK |
| 117 | HC | EVQLVESGGGLVQPGGSLRLSCAASGYSISSGHHWGWIRQAPGKGLE |
| | | WIGEIYHSGSTYYSPSLKSRVTISRDNSKNTLYLQLNSLRAEDTAVY |
| | | YCARRELGYAYFDYWGQGTLVTVSSASTKGPSVFPLAPCSRSTSEST |
| | | AALGQLVKDYFPEPVCVSWNSGALTSGVHTFPAVLQSSGLYSLSSVV |
| | | TVPSSSLGTKTYTCNVDHKPSNTKVDKRVESKYGPPCPPCPAPEFLG |
| | | GPSVFLFPPKPKDTLMISRTPEVTCVVVDVSQEDPEVQFNWYVDGVE |
| | | VHNAKTKPREEQENSTYRVVSVLTVLHQDWLNGKEYKCKVSNKGLPS |
| | | SIEKTISKAKGQPREPQVYTLPPSQEEMTKNQVSLTCLVKGFYPSDI |
| | | AVEWESNGQPENNYKTTPPVLDSDGSFFLYSRLTVDKSRWQEGNVFS |
| | | CSVMHEALHNHYTQKSLSLSLGK |
| 118 | LC | EIVLTQSPATLSLSPGERATLSCRASQDIRKYLAWYQQKPGQAPRLL |
| | | IYAASTLQSGVPARFSGSGSGTDFTLTISSLEPEDFAVYYCQQYYRT |
| | | PLTFGQGTKVEIKRTVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVTKSFNRGEC |

Anti-CD28 mAb (TY24856)

| | | |
|---|---|---|
| 15 | HCDR1 | SGHHWS |
| 6 | HCDR2 | EIYHSGSTYYSPSLKS |
| 119 | HCDR3 | RDFGASYFDY |
| 120 | LCDR1 | RASQGVGPWLA |
| 9 | LCDR2 | AASSLQS |
| 121 | LCDR3 | QQYYSYST |
| 122 | VH | EVQLVESGGGLVQPGGSLRLSCAASGYSISSGHHWSWIRQAPGKGLE |
| | | WIGEIYHSGSTYYSPSLKSRVTISRDNSKNTLYLQLNSLRAEDTAVY |
| | | YCARRDEGASYFDYWGQGTLVTVSS |
| 123 | VL | DIQLTQSPSSLSASVGDRVTITCRASQGVGPWLAWYQQKPGKAPKLL |
| | | IYAASSLQSGVPSRFSGSGSGTDFTLTISSLQPEDFATYYCQQYYSY |
| | | STFGQGTKVEIK |
| 124 | HC | EVQLVESGGGLVQPGGSLRLSCAASGYSISSGHHWSWIRQAPGKGLE |
| | | WIGEIYHSGSTYYSPSLKSRVTISRDNSKNTLYLQLNSLRAEDTAVY |
| | | YCARRDFGASYEDYWGQGTLVTVSSASTKGPSVFPLAPCSRSTSEST |
| | | AALGCLVKDYFPEPVTVSWNSGALTSGVHTEPAVLQSSGLYSLSSVV |
| | | TVPSSSLGTKTYTCNVDHKPSNTKVDKRVESKYGPPCPPCPAPEFLG |
| | | GPSVFLFPPKPKDTLMISRTPEVTCVVVDVSQEDPEVQFNWYVDGVE |
| | | VHNAKTKPREEQENSTYRVVSVLTVLHQDWLNGKEYKCKVSNKGLPS |
| | | SIEKTISKAKGQPREPQVYTLPPSQEEMTKNQVSLTCLVKGFYPSDI |
| | | AVEWESNGQPENNYKTTPPVLDSDGSFFLYSRLTVDKSRWQEGNVFS |
| | | CSVMHEALHNHYTQKSLSLSLGK |
| 125 | LC | DIQLTQSPSSLSASVGDRVTITCRASQGVGPWLAWYQQKPGKAPKLL |
| | | IYAASSLQSGVPSRFSGSGSGTDFTLTISSLQPEDFATYYCQQYYSY |
| | | STFGQGTKVEIKRTVAAPSVFIFPPSDEQLKSGTASVVCLLNNFYPR |
| | | EAKVQWKVDNALQSGNSQESVTEQDSKDSTYSLSSTLTLSKADYEKH |
| | | KVYACEVTHQGLSSPVTKSFNRGEC |

Anti-CD28 mAb (TY24881)

| | | |
|---|---|---|
| 126 | HCDR1 | TGGVGVT |
| 67 | HCDR2 | SIYHSGSTYYSPSLKS |
| 127 | HCDR3 | YDYGYYSFDV |
| 128 | LCDR1 | RASQSVDFDAFSELA |
| 18 | LCDR2 | AASTLQS |
| 129 | LCDR3 | QQSYSAST |
| 130 | VH | QVQLVQSGAEVKKPGSSVKVSCKASGFSLSTGGVGVTWIRQAPGQGL |
| | | EWIGSIYHSGSTYYSPSLKSRVTITADKSTSTAYMELSSLRSEDTAV |
| | | YYCARYDYGYYSFDVWGQGTLVTVSS |
| 131 | VL | DIQLTQSPSSLSASVGDRVTITCRASQSVDFDAFSELAWYQQKPGKA |
| | | PKLLIYAASTLQSGVPSRFSGSGSGTDFTLTISSLQPEDFATYYCQQ |
| | | SYSASTFGQGTKVEIK |
| 132 | HC | QVQLVQSGAEVKKPGSSVKVSCKASGFSLSTGGVGVTWIRQAPGQGL |
| | | EWIGSIYHSGSTYYSPSLKSRVTITADKSTSTAYMELSSLRSEDTAV |
| | | YYCARYDYGYYSFDVWGQGTLVTVSSASTKGPSVFPLAPCSRSTSES |
| | | TAALGCLVKDYFPEPVTVSWNSGALTSGVHTFPAVLQSSGLYSLSSV |
| | | VTVPSSSLGTKTYTCNVDHKPSNTKVDKRVESKYGPPCPPCPAPEFL |
| | | GGPSVFLFPPKPKDTLMISRTPEVTCVVVDVSQEDPEVQFNWYVDGV |
| | | EVHNAKTKPREEQFNSTYRVVSVLTVLHQDWLNGKEYKCKVSNKGLP |
| | | SSIEKTISKAKGQPREPQVYTTPPSQEEMTKNQVSLTCLVKGFYPSD |
| | | IAVEWESNGQPENNYKTTPPVLDSDGSFFLYSRLTVDKSRWQEGNVF |
| | | SCSVMHEALHNHYTQKSLSLSLGK |
| 133 | LC | DIQLTQSPSSLSASVGDRVTITCRASQSVDFDAFSFLAWYQQKPGKA |
| | | PKLLIYAASTLQSGVPSRFSGSGSGTDFTLTISSLQPEDFATYYCQQ |
| | | SYSASTEGQGTKVEIKRTVAAPSVFIFPPSDEQLKSGTASVVCLLNN |
| | | FYPREAKVQWKVDNALQSGNSQESVTEQDSKDSTYSLSSTLTLSKAD |
| | | YEKHKVYACEVTHQGLSSPVTKSFNRGEC |

Anti-CD28 mAb (TY24858)

| | | |
|---|---|---|
| 134 | HCDR1 | SGHHWT |
| 6 | HCDR2 | EIYHSGSTYYSPSLKS |
| 135 | HCDR3 | RDYGSSYFDY |
| 136 | LCDR1 | RASQSVSRSFLA |
| 27 | LCDR2 | DASNRAT |
| 83 | LCDR3 | QQYYRSPIT |
| 137 | VH | EVQLVESGGGLVQPGGSLRLSCAASGYSISSGHHWTWIRQAPGKGLE |
| | | WIGEIYHSGSTYYSPSLKSRVTISRDNSKNTLYLQLNSLRAEDTAVY |
| | | YCARRDYGSSYFDYWGQGTLVTVSS |
| 138 | VL | DIQLTQSPSSLSASVGDRVTITCRASQSVSRSFLAWYQQKPGKAPKL |
| | | LIYDASNRATGIPSRFSGSGSGTDFTLTISSLQPEDFATYYCQQYYR |
| | | SPITFGQGTKVEIK |
| 139 | HC | EVQLVESGGGLVQPGGSLRLSCAASGYSISSGHHWTWIRQAPGKGLE |
| | | WIGEIYHSGSTYYSPSLKSRVTISRDNSKNTLYLQLNSLRAEDTAVY |
| | | YCARRDYGSSYEDYWGQGTLVTVSSASTKGPSVFPLAPCSRSTSEST |
| | | AALGCLVKDYFPEPVTVSWNSGALTSGVHTFPAVLQSSGLYSLSSVV |
| | | TVPSSSLGTKTYICNVDHKPSNTKVDKRVESKYGPPCPPCPAPEFLG |
| | | GPSVFLFPPKPKDTLMISRTPEVTCVVVDVSQEDPEVQFNWYVDGVE |
| | | VHNAKTKPREEQFNSTYRVVSVLTVLHQDWLNGKEYKCKVSNKGLPS |
| | | SIEKTISKAKGQPREPQVYTLPPSQEEMTKNQVSLTCLVKGFYPSDI |
| | | AVEWESNGQPENNYKTTPPVLDSDGSFFLYSRLTVDKSRWQEGNVFS |
| | | CSVMHEALHNHYTQKSLSLSLGK |
| 140 | LC | DIQLTQSPSSLSASVGDRVTITCRASQSVSRSFLAWYQQKPGKAPKL |
| | | LIYDASNRATGIPSRFSGSGSGTDFTLTISSLQPEDFATYYCQQYYR |
| | | SPITFGQGTKVEIKRTVAAPSVFIFPPSDEQLKSGTASVVCLLNNFY |
| | | PREAKVQWKVDNALQSGNSQESVTEQDSKDSTYSLSSTLTLSKADYE |
| | | KHKVYACEVTHQGLSSPVTKSFNRGEC |

Anti-CD28 mAb (TY24864)

| | | |
|---|---|---|
| 43 | HCDR1 | TSGVGVS |
| 58 | HCDR2 | AIYHSGSTYYSPSLKS |
| 141 | HCDR3 | YGYSYYAFDY |
| 142 | LCDR1 | RASQTIGSFLN |
| 300 | LCDR2 | DASSLES |
| 143 | LCDR3 | QQSYSYQT |
| 144 | VH | EVQLVESGGGLVQPGGSLRLSCAASGFSLSTSGVGVSWIRQAPGKGL |
| | | EWIGAIYHSGSTYYSPSLKSRVTISRDNSKNTLYLQLNSLRAEDTAV |
| | | YYCARYGYSYYAFDYWGQGTLVTVSS |
| 145 | VL | DIQLTQSPSSLSASVGDRVTITCRASQTIGSFLNWYQQKPGKAPKLL |
| | | IYDASSLESGVESRFSGSGSGTDFTLTISSLQPEDFATYYCQQSYSY |
| | | QTFGQGTKVEIK |
| 146 | HC | EVQLVESGGGLVQPGGSLRLSCAASGFSLSTSGVGVSWIRQAPGKGL |
| | | EWIGAIYHSGSTYYSPSLKSRVTISRDNSKNTLYLQLNSLRAEDTAV |
| | | YYCARYGYSYYAFDYWGQGTLVTVSSASTKGPSVFPLAPCSRSTSES |
| | | TAALGCLVKDYFPEPVTVSWNSGALTSGVHTFPAVLQSSGLYSLSSV |
| | | VTVPSSSLGTKTYTCNVDHKPSNTKVDKRVESKYGPPCPPCPAPEFL |
| | | GGPSVFLFPPKPKDTLMISRTPEVTCVVVDVSQEDPEVQFNWYVDGV |
| | | EVHNAKTKPREEQFNSTYRVVSVLTVLHQDWLNGKEYKCKVSNKGLP |
| | | SSIEKTISKAKGQPREPQVYTLPPSQEEMTKNQVSLTCLVKGFYPSD |
| | | IAVEWESNGQPENNYKTTPPVLDSDGSFFLYSRLTVDKSRWQEGNVF |
| | | SCSVMHEALHNHYTQKSLSLSLGK |
| 147 | LC | DIQLTQSPSSLSASVGDRVTITCRASQTIGSFLNWYQQKPGKAPKLL |
| | | IYDASSLESGVPSRFSGSGSGTDFTLTISSLQPEDFATYYCQQSYSY |
| | | QTFGQGTKVEIKRTVAAPSVFIFPPSDEQLKSGTASVVCLLNNFYPR |
| | | EAKVQWKVDNALQSGNSQESVTEQDSKDSTYSLSSTLTLSKADYEKH |
| | | KVYACEVTHQGLSSPVTKSFNRGEC |

Anti-CD28 mAb (TY24877)

| | | |
|---|---|---|
| 148 | HCDR1 | NYGIH |
| 6 | HCDR2 | EIYHSGSTYYSPSLKS |
| 149 | HCDR3 | GPLSYYALDY |
| 150 | LCDR1 | PASQSIRRFLN |
| 300 | LCDR2 | DASSLES |
| 83 | LCDR3 | QQYYRSPIT |
| 151 | VH | QVQLVQSGAEVKKPGSSVKVSCKASGYTFTNYGIHWVRQAPGQGLEW |
| | | IGEIYHSGSTYYSPSLKSRVTITADKSTSTAYMELSSLRSEDTAVYY |
| | | CARGPLSYYALDYWGQGTLVTVSS |
| 152 | VL | DIQLTQSPSSLSASVGDRVTITCRASQSIRRFLNWYQQKPGKAPKLL |
| | | IYDASSLESGVPSRFSGSGSGTDFTLTISSLQPEDFATYYCQQYYRS |
| | | PITFGQGTKVEIK |
| 153 | HC | QVQLVQSGAEVKKPGSSVKVSCKASGYTFTNYGIHWVRQAPGQGLEW |
| | | IGEIYHSGSTYYSPSLKSRVTITADKSTSTAYMELSSLRSEDTAVYY |
| | | CARGPLSYYALDYWGQGTLVTVSSASTKGPSVFPLAPCSRSTSESTA |
| | | ALGCLVKDYFPEPVTVSWNSGALTSGVHTFPAVLQSSGLYSLSSVVT |
| | | VPSSSLGTKTYTCNVDHKPSNTKVDKRVESKYGPPCPPCPAPEFLGG |
| | | PSVFLFPPKPKDTLMISRTPEVTCVVVDVSQEDPEVQFNWYVDGVEV |
| | | HNAKTKPREEQENSTYRVVSVLTVLHQDWLNGKEYKCKVSNKGLPSS |
| | | IEKTISKAKGQPREPQVYTLPPSQEEMTKNQVSLTCLVKGFYPSDIA |
| | | VEWESNGQPENNYKTTPPVLDSDGSFFLYSRLTVDKSRWQEGNVFSC |
| | | SVMHEALHNHYTQKSLSLSLGK |
| 154 | LC | DIQLTQSPSSLSASVGDRVTITCRASQSIRRFLNWYQQKPGKAPKLL |
| | | IYDASSLESGVPSRFSGSGSGTDFTLTISSLQPEDFATYYCQQYYRS |
| | | PITFGQGTKVEIKRIVAAPSVFIFPPSDEQLKSGTASVVCLLNNFYP |
| | | REAKVQWKVDNALQSSNSQESVTEQDSKDSTYSLSSTLTLSKADYEK |
| | | HKVYACEVTHQGLSSPVTKSFNRGEC |

Anti-CD28 mAb (TY24855)

| | | |
|---|---|---|
| 15 | HCDR1 | SGHHWS |
| 155 | HCDR2 | DIYHSGSTYYNPSLKS |
| 16 | HCDR3 | KDDYYAFDY |
| 156 | LCDR1 | RAPQSVDFHGKSFLH |
| 27 | LCDR2 | DASNRAT |
| 70 | LCDR3 | QQSYRSPIT |
| 157 | VH | EVQLVESGGGLVQPGGSLRLSCAASGYSITSGHHWSWIRQAPGKGLE |
| | | WIGDIYHSGSTYYNPSLKSRVTISRDNSKNTLYLQLNSLRAEDTAVY |
| | | YCARKDDYYAFDYWGQGTLVTVSS |
| 158 | VL | DIQLTQSPSSLSASVSDRVTITCRAPQSVDFHGKSFLHWYQQKPGKA |
| | | PKLLIYDASNRATGIPSRFSGSGSGTDFTLTISSLQPEDFATYYCQQ |
| | | SYRSPITFGQGTKVEIK |
| 159 | HC | EVQLVESGGGLVQPGGSLRLSCAASGYSITSGHHWSWIRQAPGKGLE |
| | | WIGDIYHSGSTYYNPSLKSRVTISRDNSKNTLYLQLNSLRAEDTAVY |
| | | YCARKDDYYAFDYWGQGTLVTVSSASTKGPSVFPLAPCSRSTSESTA |
| | | ALGCLVKDYFPEPVTVSWNSGALTSGVHTFPAVLQSSGLYSLSSVVT |
| | | VPSSSLGTKTYTCNVDHKPSNTKVDKRVESKYGPPCPPCPAPEFLGG |
| | | PSVFLFPPKPKDTLMISRTPEVTCVVVDVSQEDPEVQFNWYVDGVEV |
| | | HNAKTKPREEQFNSTYRVVSVLTVLHQDWLNGKEYKCKVSNKGLPSS |
| | | IEKTISKAKGQPREPQVYTLPPSQEEMTKNQVSLTCLVKGFYPSDIA |
| | | VEWESNGQPENNYKTTPPVLDSDGSFFLYSRLTVDKSRWQEGNVFSC |
| | | SVMHEALHNHYTQKSLSLSLGK |
| 160 | LC | DIQLTQSPSSLSASVGDRVTITCRAPQSVDFHGKSFLHWYQQKPGKA |
| | | PKLLIYDASNRATGIPSRFSGSGSGTDFTLTISSLQPEDFATYYCQQ |
| | | SYRSPITFGQGTKVEIKRTVAAPSVFIFPPSDEQLKSGTASVVCLLN |
| | | NFYPREAKVQWKVDNALQSGNSQESVTEQDSKDSTYSLSSTLTLSKA |
| | | DYEKHKVYACEVHQGLSSPVTKSFNRGEC |

Anti-CD28 mAb (TY24857)

| | | |
|---|---|---|
| 161 | HCDR1 | TSGVAVG |
| 6 | HCDR2 | EIYHSGSTYYSPSLKS |
| 162 | HCDR3 | HGYHYYALDY |
| 163 | LCDR1 | PASQDVGTAVA |
| 300 | LCDR2 | DASSLES |
| 164 | LCDR3 | QQYYSSPPT |
| 165 | VH | EVQLVESGGGLVQPGSSLRLSCAASGFSLSTSGVAVGWIRQAPGKGL |
| | | EWIGEIYHSGSTYYSPSLKSRVTISRDNSKNTLYLQLNSLRAEDTAV |
| | | YYCARHGYHYYALDYWGQGTLVTVSS |
| 166 | VL | DIQLTQSPSSLSASVGDRVTITCPASQDVGTAVAWYQQKPGKAPKLL |
| | | IYDASSLESGVPSRFSGSGSGTDFTLTISSLQPEDFATYYCQQYYSS |
| | | PPTFGQGTKVEIK |
| 167 | HC | EVQLVESGGGLVQPGGSLRLSCAASGFSLSTSGVAVGWIRQAPGKGL |
| | | EWIGEIYHSGSTYYSPSLKSRVTISRDNSKNTLYLQLNSLRAEDTAV |
| | | YYCARHGYHYYALDYWGQGTLVTVSSASTKGPSVFPLAPCSRSTSES |
| | | TAALGCLVKDYFPEPVTVSWNSGALTSGVHTFPAVLQSSGLYSLSSV |
| | | VTVPSSSLGTKTYTCNVDHKPSNTKVDKRVESKYGPPCPPCPAPEFL |
| | | GGPSVFLFPPKPKDTLMISRTPEVTCVVVDVSQEDPEVQFNWYVDGV |
| | | EVHNAKTKPREEQFNSTYRVVSVLTVLHQDWLNGKEYKCKVSNKGLP |
| | | SSIEKTISKAKGQPREPQVYTTPPSQEEMTKNQVSLTCLVKGFYPSD |
| | | IAVEWESNGQPENNYKTTPPVLDSDGSFFLYSRLTVDKSRWQEGNVF |
| | | SCSVMHEALHNHYTQKSLSLSLGK |
| 168 | LC | DIQLTQSPSSLSASVGDRVTITCPASQDVGTAVAWYQQKPGKAPKLL |
| | | IYDASSLESGVPSRFSGSGSGTDFTLTISSLQPEDFATYYCQQYYSS |
| | | PPTFGQGTKVEIKRTVAAPSVFIFPPSDEQLKSGTASVVCLLNNFYP |
| | | REAKVQWKVDNALQSSNSQESVTEQDSKDSTYSLSSTLTLSKADYEK |
| | | HKVYACEVTHQGLSSPVTKSFNRGEC |

Non-masked Bispecific Antibodies
HER2xCD28 Bispecific Antibodies-(Parent-TY24865)
TY27566

| | | |
|---|---|---|
| 169 | LC | DIQMTQSPSSLSASVGDRVTITCKASQDVSIGVAWYQQKPGKAPKLL |
| | | IYSASYRYTGVPSRFSGSGSGTDFTLTISSLQPEDFATYYCQQYYIY |
| | | PYTFGQGTKVEIKRTVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVTKSFNRGEC |
| 170 | HC-A | EVQLVESGGGLVQPGGSLRLSCAASGFTFTDYTMDWVRQAPGKGLEW |
| | | VADVNPNSGGSIYNQRFKGRFTLSVDRSKNTLYLQMNSLRAEDTAVY |
| | | YCARNLGPSFYFDYWGQGTLVTVSSASTKGPSVFPLAPSSKSTSGGT |
| | | AALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVD |
| | | GVEVHNAKTKPREEQYASTYRVVSVLTVLHQDWLNGKEYKCKVSNKA |
| | | LPAPIEKTISKAKGQPREPQVYTLPPSRKKLTKNQVKLTCLVKGFYP |
| | | SDIAVEWESNGQPENNYKTTPPVLDCDGSFFLYSKLTVDKSRWQQGN |
| | | VFSCSVMHEALHNHYTQKSLSLSPGK |
| 171 | HG-B | DIQLTQSPSSLSASVGDRVTITCRASQSVDFHGISFLHWYQQKPGKA |
| | | PKLLIYAASSLQSGVPSRFSGSGSGTDFTLTISSLQPEDFATYYCQQ |
| | | SYRSPRTFGQGTKVEIKGGGGSGGGGSGGGGSGGGGSEVQLVESGGG |
| | | LVQPGGSLRLSCAASGFSLSTGGVGVSWIRQAPGKGLEWIGEIYHSG |
| | | STYYSPSLKSRVTISRDNSKNTLYLQLNSLRAEDTAVYYCARYEYYY |
| | | YDFDYWGQGTLVSVSSEPKSSDKTHTCPPCPAPELLGGPSVFLFPPK |
| | | PKDTLMISRTPEVTCVVVDVSHEDPEVKFNWYVDGVEVHNAKTKPRE |
| | | EQYASTYRVVSVLTVLHQDWLNGKEYKCKVSNKALPAPIEKTISKAK |
| | | GQPREPQVYTDPPSRDELTKNQVSLTCLVDGFYPSDIAVEWESNGQP |
| | | ENCYKTTPPVLDSDGSFFLYSKLTVDKSRWQQGNVFSCSVMHEALHN |
| | | HYTQDSLSLSPGK |

TY27881

| | | |
|---|---|---|
| 169 | LC | DIQMTQSPSSLSASVGDRVTITCKASQDVSIGVAWYQQKPGKAPKLL |
| | | IYSASYRYTGVPSRFSGSGSGTDFTLTISSLQPEDFATYYCQQYYIY |
| | | PYTFGQGTKVEIKRTVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVTKSFNRGEC |
| 170 | HC-A | EVQLVESGGGLVQPGGSLRLSCAASGFTFTDYTMDWVRQAPGKGLEW |
| | | VADVNPNSGGSIYNQRFKGRFTLSVDRSKNTLYLQMNSLRAEDTAVY |
| | | YCARNLGPSFYFDYWGQGTLVTVSSASTKGPSVFPLAPSSKSTSGGT |
| | | AALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVD |
| | | GVEVHNAKTKPREEQYASTYRVVSVLTVLHQDWLNGKEYKCKVSNKA |
| | | LPAPIEKTISKAKGQPREPQVYTLPPSRKKLTKNQVKLTCLVKGFYP |
| | | SDIAVEWESNGQPENNYKTTPPVLDCDGSFFLYSKLTVDKSRWQQGN |
| | | VFSCSVMHEALHNHYTQKSLSLSPGK |
| 172 | HC-B | DIQLTQSPSSLSASVGDRVTITCRASQSVDFYGKSFLHWYQQKPGKA |
| | | PKLLIYAASSLQSGVPSRFSGSGSGTDFTLTISSLQPEDFATYYCQQ |
| | | SYRSPLTFGQGTKVEIKGGGGSGGGGSGGGGSGGGGSEVQLVESGGG |
| | | LVQPGGSLRLSCAASGFSLSTSGVGVSWIRQAPGKGLEWIGEIYHSG |
| | | STYYSPSLKSRVTISRDNSKNTLYLQLNSLRVEDTAVYYCARDEYDY |
| | | YAFDYWGQGTLVTVSSEPKSSDKTHTCPPCPAPELLGGPSVFLFPPK |
| | | PKDTLMISRTPEVTCVVVDVSHEDPEVKFNWYVDGVEVHNAKTKPRE |
| | | EQYASTYRVVSVLTVLHQDWLNGKEYKCKVSNKALPAPIEKTISKAK |
| | | GQPREPQVYTDPPSRDELTKNQVSLTCLVDGFYPSDIAVENESNGQP |
| | | ENCYKTTPPVLDSDGSFFLYSKLTVDKSRWQQGNVFSCSVMHEALHN |
| | | HYTQDSLSLSPGK |

TROP2xCD28 Bispecific Antibodies
TY27571

| | | |
|---|---|---|
| 173 | LC | DIQLTQSPSSLSASVGDRVTITCRASQDVSIAVAWYQQKPGKAPKLL |
| | | IYSASYRYTGVPARFSGSGSGTDFTLTISSLQPEDFATYYCQQHYIT |
| | | PLTFGQGTKVEIKRTVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVTKSFNRGEC |
| 174 | HC-A | QVQLLQSGSELKKPGASVKVSCKASGYTFTNYGINWVRQAPGQGLEW |
| | | LGWIHTYTGEPTYTDDFKGRFVFSLDTSVSTAYLQISSLKADDTAVY |
| | | YCARGGFGSSYWYFDVWGQGTLVTVSSASTKGPSVFPLAPSSKSTSG |
| | | GTAALGCLVKDYFPEPVTVSWNSGALTSGVHTFPAVLQSSGLYSLSS |
| | | VVTVPSSSIGTQTYICNVNHKPSNTKVDKKVEPKSCDKTHTCPPCPA |
| | | PELLGGPSVFLFPPKPKDTLMISRTPEVTCVVVDVSHEDPEVKFNWY |
| | | VDGVEVHNAKTKPREEQYASTYRVVSVLTVLHQDWLNGKEYKCKVSN |
| | | KALPAPIEKTISKAKGQPREPQVYTLPPSRKKLTKNQVKLTCLVKGF |
| | | YPSDIAVEWESNGQPENNYKTTPPVLDCDGSFFLYSKLTVDKSRWQQ |
| | | GNVFSCSVMHEALHNHYTQKSLSLSPGK |
| 171 | HC-B | DIQLTQSPSSLSASVGDRVTITCRASQSVDFHGISFLHWYQQKPGKA |
| | | PKLLIYAASSLQSGVPSRFSGSGSGTDFTLTISSLQPEDFATYYCQQ |
| | | SYRSPRTFGQGTKVEIKGGGGSGGGGSGGGGSGGGGSEVQLVESGGG |
| | | LVQPGGSLRLSCAASGFSLSTGGVGVSWIRQAPGKGLEWIGEIYHSG |
| | | STYYSPSLKSRVTISRDNSKNTLYLQLNSLRAEDTAVYYCARYEYYY |
| | | YDFDYWGQGTLVTVSSEPKSSDKTHTCPPCPAPELLGGPSVFLFPPK |
| | | PKDTLMISRTPEVTCVVVDVSHEDPEVKFNWYVDGVEVHNAKTKPRE |
| | | EQYASTYRVVSVLTVLHQDWLNGKEYKCKVSNKALPAPIEKTISKAK |
| | | GQPREPQVYTDPPSRDELTKNQVSLTCLVDGFYPSDIAVEWESNGQP |
| | | ENCYKTTPPVLDSDGSFFLYSKLTVDKSRWQQGNVFSCSVMHEALHN |
| | | HYTQDSLSLSPGK |

B7H3xCD28 Bispecific Antibody
TY27556

| | | |
|---|---|---|
| 175 | LC | DIQLTQSPSSLSASVGDRVTITCRASQSIRKYLNWYQQKPGKAPKLL |
| | | IYAASTLQSGVPSRFSGSGSGTDFTLTISSLQPEDFATYFCSQGTHV |
| | | PFTEGQGTKVEIKRIVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVTKSFNRGEC |
| 176 | HC-A | EVQLVESGGGLVQPGGSLRLSCAASGFTFSGYGIHWVRQAPGKGLEW |
| | | IGWISPSGGDTKYAQKFQGRVTISRDNSKNTLYLQLNSLRAEDTAVY |
| | | YCARGGTVVYFDYWGQGTLVTVSSASTKGPSVFPLAPSSKSTSGGTA |
| | | ALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDG |
| | | VEVHNAKTKPREEQYASTYRVVSVLTVLHQDWLNGKEYKCKVSNKAL |
| | | PAPIEKTISKAKGQPREPQVYTLPPSRKKLTKNQVKLTCLVKGFYPS |
| | | DIAVEWESNGQPENNYKTTPPVLDCDGSFFLYSKLTVDKSRWQQGNV |
| | | FSCSVMHEALHNHYTQKSLSLSPGK |
| 171 | HC-B | DIQLTQSPSSLSASVGDRVTITCRASQSVDFHGISFLHWYQQKPGKA |
| | | PKLLIYAASSLQSGVPSRFSGSGSGTDFTLTISSLQPEDFATYYCQQ |
| | | SYRSPRTFGQGTKVEIKGGGGSGGGGSGGGGSGGGGSEVQLVESGGG |
| | | LVQPGGSLRLSCAASGFSLSTGGVGVSWIRQAPGKGLEWIGEIYHSG |
| | | STYYSPSLKSRVTISRDNSKNTLYLQLNSLRAEDTAVYYCARYEYYY |
| | | YDFDYWGQGTLVTVSSEPKSSDKTHTCPPCPAPELLGGPSVFLFPPK |
| | | PKDTLMISRTPEVTCVVVDVSHEDPEVKFNWYVDGVEVHNAKTKPRE |
| | | EQYASTYRVVSVLTVLHQDWLNGKEYKCKVSNKALPAPIEKTISKAK |
| | | GQPREPQVYTDPPSRDELTKNQVSLTCLVDGFYPSDIAVEWESNGQP |
| | | ENCYKTTPPVLDSDGSFFLYSKLTVDKSRWQQGNVFSCSVMHEALHN |
| | | HYTQDSLSLSPGK |

TY26999

| | | |
|---|---|---|
| 175 | LC | DIQLTQSPSSLSASVGDRVTITCRASQSIRKYLNWYQQKPGKAPKLL |
| | | IYAASTLQSGVPSRFSGSGSGTDFTLTISSLQPEDFATYFCSQGTHV |
| | | PFTFGQGTKVEIKRTVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVTKSFNRGEC |
| 176 | HC-A | EVQLVESGGGLVQPGGSLRLSCAASGFTFSGYGIHWVRQAPGKGLEW |
| | | IGWISPSGGDTKYAQKFQGRVTISRDNSKNTLYLQLNSLRAEDTAVY |
| | | YCARGGTVVYFDYWGQGTLVTVSSASTKGPSVFPLAPSSKSTSGGTA |
| | | ALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDG |
| | | VEVHNAKTKPREEQYASTYRVVSVLTVLHQDWLNGKEYKCKVSNKAL |
| | | PAPIEKTISKAKGQPREPQVYTLPPSRKKLTKNQVKLTCLVKGFYPS |
| | | DIAVEWESNGQPENNYKTTPPVLDCDGSFFLYSKLTVDKSRWQQGNV |
| | | FSCSVMHEALHNHYTQKSLSLSPGK |
| 177 | HC-B | QAVVTQEPSLTVSPGGTVTLTCGSSTGAVTTSNYPNWVQQKPGQAPR |
| | | GLIGGTNKRAPGVPARFSGSLLGGKAALTLSGAQPEDEAEYYCALWY |
| | | SNLWVFGGGTKLTVLGGGGSGGGGSGGGGSGGGGSEVQLVESGGGLV |
| | | QPGGSLRLSCAASGFTFNTYAINWVRQAPGKGLEWVGRIRSKYNNYA |
| | | TYYAESVKGRFTISRDDSKNTLYLQINSLRAEDTAVYYCVRHGNFGT |
| | | SYVSWEAYWGQGTLVTVSSEPKSSDKTHTCPPCPAPELLGGPSVFLF |
| | | PPKPKDTLMISRTPEVTCVVVDVSHEDPEVKFNWYVDGVEVHNAKTK |
| | | PREEQYASTYRVVSVLTVLHQDWLNGKEYKCKVSNKALPAPIEKTIS |
| | | KAKGQPREPQVYTDPPSRDELTKNQVSLTCLVDGEYPSDIAVEWESN |
| | | GQPENCYKTTPPVLDSDGSFFLYSKLTVDKSRWQQGNVFSCSVMHEA |
| | | LHNHYTQDSLSLSPGK |

Masking Unit (MU)

| SEQ ID | |
|---|---|
| NO: | Sequence |
| 178 | ASDLYVCAHDPYFCHS |
| 179 | PNVEDTCYVDPYSCTI |
| 180 | YAAAPSCPPANYYCAD |
| 181 | HHADPLCFAQHSACNT |
| 182 | YDTHDYCLIHYHSCDT |
| 183 | ADFAPYCYADPYYCHD |
| 184 | DIASDSCYYAPNHCDT |
| 185 | AATYSDCYSDPYACHD |
| 186 | PTYDHDCAVYNNFCNA |
| 187 | HNHALTCYTDPYACHS |
| 188 | AAFSPACYTDPYSCHA |
| 189 | TPHPDACEVDPYHCHT |
| 190 | APSDAYCYADPYACHT |
| 191 | AVDDDPCYTDPYQCTA |
| 192 | THSDAVCAPDHAYCLY |
| 193 | PYIYVDCYAPPFFCAY |
| 194 | YECAEGVPIVLCIG |
| 195 | DYCEGPPAFFYCAK |
| 196 | FPCEPLADPFLCRO |
| 197 | LVCEDVPEPAACRA |
| 198 | ALCEFYAFPYFCAR |
| 199 | IVCKYFDDPELCRR |
| 200 | YECPVRVPLAVCLR |
| 201 | PSCPDYVPFPVCAQ |
| 202 | VACAKAFAYVYCLV |
| 203 | VLCGDSVPFLLCEP |
| 204 | ILCPEGVPVVFCVL |
| 205 | SDCGLGVPLLVCLK |
| 206 | TDCGIPFPVDDCLS |
| 207 | HIPDAYCAADHHYCYY |
| 208 | AATYSDCYSDPYACHD |
| 209 | YSDCYSDPYACHD |
| 210 | AAFSPACYTDPYSCHA |
| 211 | SPACYTDPYSCHA |

Masking peptide components

| SEQ ID NO: | Description | Sequence |
|---|---|---|
| 212 | N-terminal Unit | EVGSY |
| 213 | Linkage Unit (LU) | GGGPLGLAGSGGS |
| 214 | Linkage Unit (LU) | SGGGPLGLAGSGGS |

| SEQ | | | | | | |
|---|---|---|---|---|---|---|
| ID | Sample | Masking + Cleavage | SEQ ID NO | | | |
| NO: | ID Name | Peptide Sequences: | LC | HC | MU | LU |
| 215 | TY26142 | EVGSYASDLYVCAHDPYF | 14 | 13 | 171 | 213 |
| | | CHSGGGPLGLAGSGGS | | | | |

| SEQ | | | | | | |
|---|---|---|---|---|---|---|
| ID | Sample | Masking + Cleavage | | | | |
| NQ: | ID Name | Peptide Sequences: | SEQ ID NQ | | | |
| 216 | TY26143 | EVGSYPNVEDTCYVDPYSCTI | 14 | 13 | 172 | 213 |
| | | GGGPLGLAGSGGS | | | | |
| 227 | TY26144 | EVGSYYAAAPSCPPANYYCAD | 14 | 13 | 173 | 213 |
| | | GGGPLGLAGSGGS | | | | |
| 218 | TY26145 | EVGSYHHADPLCFAQHSACNT | 14 | 13 | 174 | 213 |
| | | GGGPLGLAGSGGS | | | | |
| 219 | TY26146 | EVGSYYDTHDYCLIHYHSCDT | 14 | 13 | 175 | 213 |
| | | GGGPLGLAGSGGS | | | | |
| 220 | TY26147 | EVGSYADFAPYCYADPYYCHD | 14 | 13 | 176 | 213 |
| | | GGGPLGLAGSGGS | | | | |
| 221 | TY26148 | EVGSYDIASDSCYYHPNECDT | 14 | 13 | 177 | 213 |
| | | GGGPLGLAGSGGS | | | | |
| 222 | TY26149 | EVGSYAATYSDCYSDPYACHD | 14 | 13 | 178 | 213 |
| | | GGGPLGLAGSGGS | | | | |
| 223 | TY26150 | EVGSYPTYDHDCAVYNNFCNA | 14 | 13 | 179 | 213 |
| | | GGGPLGLAGSGGS | | | | |
| 224 | TY26151 | EVGSYHNHALTCYTDPYACHS | 14 | 13 | 180 | 213 |
| | | GGGPLGLAGSGGS | | | | |
| 225 | TY26152 | EVGSYAAFSPACYTDPYSCHA | 14 | 13 | 181 | 213 |
| | | GGGPLGLAGSGGS | | | | |
| 226 | TY26153 | EVGSYTPHPDACFVDPYHCHT | 14 | 13 | 182 | 213 |
| | | GGGPLGLAGSGGS | | | | |
| 227 | TY26154 | EVGSYAPSDAYCYADPYACHT | 14 | 13 | 183 | 213 |
| | | GGGPLGLAGSGGS | | | | |
| 228 | TY26155 | EVGSYAVDDDPCYTDPYQCTA | 14 | 13 | 184 | 213 |
| | | GGGPLGLAGSGGS | | | | |
| 229 | TY26156 | EVGSYTHSDAVCAPDHAYCLY | 14 | 13 | 185 | 213 |
| | | GGGPLGLAGSGGS | | | | |
| 230 | TY26157 | EVGSYPYIYVDCYAPPFFCAY | 14 | 13 | 186 | 213 |
| | | GGGPLGLAGSGGS | | | | |
| 231 | TY26158 | EVGSYYFCAEGVPIVLCIGGG | 14 | 13 | 187 | 213 |
| | | GPLGLAGSGGS | | | | |
| 232 | TY26159 | EVGSYDYCEGPPAFFYCAKGG | 14 | 13 | 188 | 213 |
| | | GPLGLAGSGGS | | | | |
| 233 | TY26160 | EVGSYFPCEPLADPFLCRQGG | 14 | 13 | 189 | 213 |
| | | GPLGLAGSGGS | | | | |
| 234 | TY26161 | EVGSYLVCEDVPFPAACRAGG | 14 | 13 | 190 | 213 |
| | | GPLGLAGSGGS | | | | |
| 235 | TY26162 | EVGSYALCEFYAFPYFCARGG | 14 | 13 | 191 | 213 |
| | | GPLGLAGSGGS | | | | |
| 236 | TY26163 | EVGSYIVCKYFDDPFLCRRGG | 14 | 13 | 192 | 213 |
| | | GPLGLAGSGGS | | | | |
| 237 | TY26164 | EVGSYYFCPVRVPLAVCLRGG | 14 | 13 | 193 | 213 |
| | | GPLGLAGSGGS | | | | |
| 238 | TY26165 | EVGSYPSCPDYVPEPVCAQGG | 14 | 13 | 194 | 213 |
| | | GPLGLAGSGGS | | | | |

| SEQ | | | | | | |
|---|---|---|---|---|---|---|
| ID | Sample | Masking + Cleavage | | | | |
| NO: | ID Name | Peptide Sequences; | SEQ ID NO | | | |
| 239 | TY26166 | EVGSYVACAKAFAYVYCLV | 14 | 13 | 195 | 213 |
| | | GGGPLGLAGSGGS | | | | |
| 240 | TY26167 | EVGSYVLCGDSVPFLLCEP | 14 | 13 | 196 | 213 |
| | | GGGPLGLAGSGGS | | | | |
| 241 | TY26168 | EVGSYILCPEGVPVVFCVL | 14 | 13 | 197 | 213 |
| | | GGGPLGLAGSGGS | | | | |
| 242 | TY26169 | EVGSYSDCGLGVPLLVCLK | 14 | 13 | 198 | 213 |
| | | GGGPLGLAGSGGS | | | | |
| 243 | TY26170 | EVGSYTDCGIPFPVDDCLS | 14 | 13 | 199 | 213 |
| | | GGGPLGLAGSGGS | | | | |
| 244 | TY26171 | EVGSYHIPDHYCAADHHYC | 14 | 13 | 200 | 213 |
| | | YYGGGPLGLAGSGGS | | | | |
| 245 | TY28338 | EAATYSDCYSDPYACHDSG | 14 | 13 | 201 | 214 |
| | | GGPLGLAGSGGS | | | | |
| 246 | TY28339 | EYSDCYSDPYACHDSGGGP | 14 | 13 | 202 | 214 |
| | | LGLAGSGGS | | | | |
| 247 | TY28341 | EAAFSPACYTDPYSCHAGG | 14 | 13 | 203 | 213 |
| | | GPLGLAGSGGS | | | | |
| 248 | TY28342 | ESPACYTDPYSCHAGGGPL | 14 | 13 | 204 | 213 |
| | | GLAGSGGS | | | | |

| | Flexible |
|---|---|
| SEQ | Linkers |
| 249 | GGGGS |
| 250 | GGGGT |
| 251 | SGGS |
| 252 | GGSG |
| 253 | GGSGG |
| 254 | GSGSG |
| 255 | GSGGG |
| 256 | GGGSG |
| 257 | GSSSG |

masked Bispecific Antibodies
HER2 × CD28 Bispecific Antibodies - (Parent - TY24865)
TY27566

| | | |
|---|---|---|
| 258 | LCDR1 | KASQDVSIGVA |
| 259 | LCDR2 | SASYRYT |
| 260 | LCDR3 | QQYYIYPYT |
| 261 | VL | DIQMTQSPSSLSASVGDRVIITCKASQDVSIGVAWYQQKEGKAPKLLIYSA |
| | | SYRYTGVPSRFSGSGSGTDFTLTISSLQPEDFATYYCQQYYTYPYTFGQGT |
| | | KVEIK |
| 262 | HCDR1-A | DYTMD |
| 263 | HCDR2-A | DVNPNSGGSIYNQREKG |
| 264 | HCDR3-A | NLGPSFYFDY |
| 265 | VH-A | EVQLVESGGGLVQPGGSLRLSCAASGETFEDYIMDWVRQAPGKGLEWVADV |
| | | NPNSGGSIYNQRFKGRFTLSVDRSKNTLYLQMNSLRAEDTAVYYCARNLGP |
| | | SFYFDYWGQGTLVTVSS |
| 5 | HCDR1-B | TGGVGVS |
| 6 | HCDR2-B | EIYHSGSTYYSPSLKS |
| 7 | HCDR3-B | YEYYYYDEDY |
| 8 | LCDR1-B | RASQSVDFHGISELH |
| 9 | LCDR2-33 | AASSLQS |
| 10 | LCDR3-B | QQSYRSPRT |
| 266 | SCFV-B | DIQLTQSPSSLSASVGDRVTITCRASQSVDFEGISFLHWYQQKPGKAPKLL |
| | | IYAASSLQSGVPSRFSGSGSGEDEILTISSLQPEDFATYYCQQSYRSPRTF |
| | | GQGTKVEIKGGGGSGGGGSGGGGSGGGGSEVQLVESGGGLVQPGGSLRLSC |
| | | AASGFSLSTGGVGVSWIRQAPGKGLEWIGEIYHSGSTYYSPSLKSRVTISR |
| | | DNSKNILYLQLNSLRAEDTAVYYCARYEYYYYDEDYWGQGTEVTVSS |

TY27807

| | | |
|---|---|---|
| 267 | LCDR1 | RASQDVNTAVA |
| 268 | LCDR2 | SASFLYS |
| 269 | LCDR3 | QQHYTTPPT |
| 270 | VL | DIQMTQSPSSLSASVGDRVTITCRASQDVNTAVAWYQQKPGKAPKLLIYSA |
| | | SFLYSGVPSRESGSRSGIDFQLTISSLQPEDFATYYCQQHYTTPPTFGQGT |
| | | KVEIK |
| 271 | LC | DIQMTQSPSSLSASVGDRVTITCRASQDVNTAVAWYQQKPGKAPKLLIYSA |
| | | SFLYSGVPSRESGSRSGIDEELTISSLQPEDFATYYCQQHYTTPPTFGQGT |
| | | KVEIKRIVAAPSVEIFPPSDEQLKSGTASVVCLINNFYPREAKVQWKVDNA |
| | | LQSGNSQESVTEQDSKDSTYSLSSTLTLSKADYEKHKVYACEVTHQGLSSP |
| | | VTKSENRGEC |
| 272 | HCDR1-A | DTYIH |
| 273 | HCDR2-A | RIYPINGYTRYADSVKG |
| 274 | HCDR3-A | WGGDGFYAMDY |
| 275 | VH-A | EVQLVESGGGLVQPGGSLRLSCAASGENIKDTYIHWVRQAPGKGLEWVARI |
| | | YPTNGYTRYADSVKGRETISADTSKNTAYLQMNSLRAEDTAVYYCSRWGGD |
| | | GFYAMDYWGQGTLVTVSS |
| 276 | HC-A | EVQLVESGGGLVQPGGSLRLSCAASGENIKDTYIHWVRQAPGKGLEWVARI |
| | | YPINGYTRYADSVKGRETISADTSKNTAYLQMNSLRAEDTAVYYCSRWGGD |
| | | GTYAMDYWGQGTLVTVSSASTKGPSVIPLAPSSKSTSGGTAALGCLVKDYF |
| | | PEPVTVSWNSGALTSGVHTFPAVLQSSGLYSLSSVVIVESSSIGTQTYICN |
| | | VNHKPSNTKVDKKVEPKSCDKTHTCPPCPAPELLGGPSVFLFPPKPKDTLM |
| | | ISRTPEVIGVVVDVSHEDPEVKENWYVDGVEVHNAKTKPREEQYASTYRVV |
| | | SVLTVLHQDWLNGKEYKCKVSNKALPAPIEKTISKAKGQPREPQVYTLPPS |
| | | RKKLIKNQVKLTCLVKGFYPSDIAVEWESNGQPENNYKITPPVLDCDGSEE |
| | | LYSKLTVDKSRWQQGNVFSCSVMHEALHNHYTQKSLSLSPGK |
| 5 | HCDR1-B | TGGVGVS |
| 6 | HCDR2-B | EIYHSGSTYYSPSLKS |
| 7 | HCDR3-B | YEYYYYDEDY |
| 8 | LCDR1-B. | PASQSVDEHGISFLH |
| 9 | LCDR2-B | AASSLQS |
| 10 | LCDR3-B | QQSYRSPRT |
| 266 | SCFv-B | DIQLTQSPSSLSASVGDRVTITCRASQSVDFHGISELHWYQQKPGKAPKLL |
| | | IYAASSLQSGVPSRFSGSGSGEDFTLTISSLQPEDEATYYCQQSYRSPRTE |
| | | GQGIKVEIKGGGGSGGGGSGGGGSGGGGSEVQLVESGGGLVQPGGSLRLSC |
| | | AASGFSLSTGGVGVSWIRQAPGKGLEWIGETYHSGSTYYSPSLKSRVTISR |
| | | DNSKNTLYLQLNSLRAEDTAVYYCARYEYYYYDFDYWGQGTLVTVSS |
| 171 | HC-B | DIQLTQSPSSLSASVGDRVTITCRASQSVDFHGISELHWYQQKPGKAPKLL |
| | | IYAASSLQSGVPSRESGSGSGEDFTLTISSLQPEDEATYYCQQSYRSPRTE |
| | | GQGIKVEIKGGGGSGGGGSGGGGSGGGGSEVQLVESGGGLVQPGGSLRLSC |
| | | AASGESLSTGGVGVSWIRQAPGKGLEWIGEIYHSGSTYYSPSLKSRVTISR |
| | | DNSKNTLYLQLNSLRAEDTAVYYCARYEYYYYDFDYWGQGTLVTVSSEPKS |
| | | SDKIHTCPPCPAPELLGGPSVFLFPPKPKDTLMISRTPEVTCVVVDVSHED |
| | | PEVKFNWYVDGVEVHNAKIKPREEQYASTYRVVSVLTVLHQDWLNGKEYKC |
| | | KVSNKALPAPIEKTISKAKGQPREPQVYTDPPSRDELTKNQVSLTCLVDGF |
| | | YPSDIAVEWESNGQPENCYKTTPPVLDSDGSFFLYSKLTVDKSRWQQGNVE |
| | | SCSVMHEALHNHYTQDSLSLSPGK |

TROP2 × CD28 Bispecific Antibodies
TY27571

| | | |
|---|---|---|
| 277 | LCDR1-A | RASQDVSIAVA |
| 259 | LCDR2-A | SASYRYT |
| 278 | LCDR3-A | QQHYITPLT |
| 279 | VL-A | DIQLTQSPSSLSASVGDRVTITCRASQDVSIAVAWYQQKPGKAPKLLIYSA |
| | | SYRYTGVPARFSGSGSGIDFTLTISSLQPEDFATYYCQQHYITPLQFGQGT |
| | | KVEIK |
| 280 | HCDR1-A | NYGIN |
| 281 | HCDR2-A | WIHTYTGEPTYTDDEKG |
| 282 | HCDR3-A | GGFGSSYWYFDV |
| 283 | VH-A | QVQLLQSGSELKKPGASVKVSCKASGYTFENYGINWVRQAPGQGLEWLGWI |
| | | HTYTGEPTYTDDFKGREVESLDTSVSTAYLQISSLKADDEAVYYCARGGEG |
| | | SSYWYFDVWGQGTLVTVSS |
| 5 | HCDR1-B | TGGVGVS |
| 6 | HCDR2-B | EIYHSGSTYYSPSLKS |
| 7 | HCDR3-B | YEYYYYDEDY |
| 8 | LCDR1-B | RASQSVDFHGISFLH |
| 9 | LCDR2-B | AASSLQS |
| 10 | LCDR3-B | QQSYRSPRT |
| 266 | SCEV-B | DIQLTQSPSSLSASVGDRVTITCRASQSVDFHGISELHWYQQKPGKAPKLL |
| | | IYAASSLQSGVPSRFSGSGSGTDFTLTISSLQPEDFATYYCQQSYRSPRTE |
| | | GQGTKVEIKGGGGSGGGGSGGGGSGGGGSEVQLVESGGGLVQPGGSLRLSC |
| | | AASGFSLSTGGVGVSWIRQAPGKGLEWIGEIYHSGSTYYSPSLKSRVTLSR |
| | | DNSKNTLYLQLNSLRAEDTAVYYCARYEYYYYDEDYWGQGELVQVSS |

B7H3 × CD28 Bispecific Antibody
TY27556

| | | |
|---|---|---|
| 287 | LCDR1-A | RASQSIRKYLN |
| 18 | LCDR2-A | AASTLQS |
| 288 | LCDR3-A | QGTHVPET |
| 289 | VL-A | DIQLTQSPSSLSASVGDRVTITCRASQSIRKYLNWYQQKPGKAPKLLIYAA |
| | | STLQSGVPSRESGSGSGTDETLTISSLQPEDFATYECSQGTHVPFCFGQGT |
| | | KVEIK |
| 290 | HCDR1-A | GYGIH |
| 291 | HCDR2-A | WISPSGGDTKYAQKFQG |
| 292 | HCDR3-A | GGTVVYFDY |
| 293 | VH-A | EVQLVESGGGLVQPGGSLRLSCAASGFTFSGYGIHWVRQAPGKGLEWIGWI |
| | | SPSGGDTKYAQKFQGRVTISRDNSKNILYLQINSIRAEDTAVYYCARGGTV |
| | | VYFDYWGQGTLVTVSS |
| 5 | HCDR1-B | TGGVGVS |
| 6 | HCDR2-B | EIYHSGSTYYSPSLKS |
| 7 | HCDR3-B | YEYYYYDEDY |
| 8 | LCDR1-B | RASQSVDEHGISELI |
| 9 | LCDR2-B. | AASSLQS |
| 10 | LCDR3-B | QQSYRSPRT |
| 266 | SCEV-B | DIQLTQSPSSLSASVGDRVTITCRASQSVDFHGISFLHWYQQKPGKAPKLL |
| | | IYAASSLQSGVPSRFSGSGSGTDFTLTISSLQPEDFATYYCQQSYRSPRTF |
| | | GQGTKVEIKGGGGSGGGGSGGGGSGGGGSEVQLVESGGGLVQPGGSLRLSC |
| | | AASGFSLSTGGVGVSWIRQAPGKGLEWIGEIYHSGSTYYSPSLKSRVTISR |
| | | DNSKNTLYLQLNSLRAEDTAVYYCARYEYYYYDFDYWGQGELVTVSS |

B7H3 × CD3 Bispecific Antibody
TY26999

| | | |
|---|---|---|
| 287 | LCDR1-A | RASQSIRKYLN |
| 18 | LCDR2-A | AASTLQS |
| 288 | LCDR3-A | QGTHVPFT |
| 289 | VL-A | DIQLTQSPSSLSASVGDRVTITCRASQSIRKYLNWYQQKPGKAPKLLIYAA |
| | | STLQSGVPSRFSGSGSGTCFTLTISSLQPEDFATYFCSQGTHVPETFGQGT |
| | | KVEIK |
| 290 | HCDR1-A | GYGIH |
| 291 | HCDR2-A | WISPSGGDTKYAQKFQG |
| 292 | HCDR3-A | GGTVVYFDY |
| 293 | VH-A | EVQLVESGGGLVQPGGSLRLSCAASGFTFSGYGIHWVRQAPGKGLEWIGWI |
| | | SPSGGDTKYAQKFQGRVTISRDNSKNTLYLQINSLRAEDTAVYYCARGGTV |
| | | VYFDYWGQGTLVTVSS |
| 5 | HCDR1-B | TYAIN |
| 294 | HCDR2-B | RIRSKYNNYATYYAESVKG |
| 295 | HCDR3-B | HGNFGTSYVSWFAY |
| 296 | LCDR1-B | GSSTGAVITSNYPN |
| 297 | LCDR2-B | GINKRAP |
| 298 | LCDR3-B | ALWYSNLWV |
| 299 | SCFV-B | QAVVTQEPSLIVSPGGTVILTCGSSTGAVTTSNYPNWVQQKPGQAPRGLIG |
| | | GINKRAPGVPARFSGSLLGGKAALTLSGAQPEDEAEYYCALWYSNLWVEGG |
| | | GTKLTVLGGGGSGGGGSGGGGSGGGGSEVQLVESGGGLVQPGGSLRLSCAA |
| | | SGFTENTYAINWVRQAPGKGLEWVGRIRSKYNNYATYYAESVKGRFTISRD |
| | | DSKNILYLQINSLRAEDTAVYYCVRHGNEGTSYVSWFAYWGQGELVTVSS |

| SEQ | | |
|---|---|---|
| ID | | Masking + cleavage |
| NO: | IgG ID | peptide sequences: |
| 301 | TY26149 | EVGSYAATYSDCYSDPYACHDGGGPLGLAGSGGS |
| 302 | TY28338 | EAATYSDCYSDPYACHDSGGGPLGLAGSGGS |
| 303 | TY28339 | EYSDCYSDPYACHDSGGGPLGLAGSGGS |
| 304 | TY26152 | EVGSYAAFSPACYTDPYSCHAGGGPLGLAGSGGS |
| 305 | TY28341 | EAAFSPACYTDPYSCHAGGGPLGLAGSGGS |
| 306 | TY28342 | ESPACYTDPYSCHAGGGPLGLAGSGGS |

TY24865 Variants

| | | |
|---|---|---|
| SEQ ID | | |
| NO: | Description | Sequence |

TY28652

| | | |
|---|---|---|
| 5 | HCDR1 | TGGVGVS |
| 96 | HCDR2 | SIYHSGSTYYNPSLKS |
| 307 | UICDR3 | YEYDYYAFDY |
| 76 | LCDR1 | RASQSVDFYGKSFLH |
| 308 | LCDR2 | AASSLES |
| 46 | LCDR3 | QQSYRSPPT |
| 362 | VH | EVQLVESGGGLVQPGGSLRLSCAASGFSLSTGGVGVSWIRQAPGK |
| | | GLEWIGSIYHSGSTYYNPSLKSRVTISRDNSKNTLYLQLNSERAE |
| | | DTAVYYCARYEYDYYAFDYWGQGTLVTVSS |
| 363 | | DIQLTQSPSSLSASVGDRVTITCRASQSVDFYGKSFLHWYQQKPG |
| | | KAPKLLIYAASSLESGVPSRESGSGSGIDETLTISSLQPEDFATY |
| | | YCQQSYRSPPTEGQGTKVEIK |

TY28653

| | | |
|---|---|---|
| 5 | HCDR1 | TGGVGVS |
| 96 | HCDR2 | SIYHSGSTYYNPSLKS |
| 309 | HCDR3 | YEYGYYALDY |
| 310 | LCDR1 | RASQSVDFYGISFLH |
| 9 | LCDR2 | AASSLQS |
| 311 | LCDR3 | QQSYRTPPT |
| 364 | VH | EVQLVESGGGLVQPGGSLRLSCAASGESLSTGGVGVSWIRQAPGK |
| | | GLEWIGSIYHSGSTYYNPSLKSRVTISRDNSKNTLYLQLNSLRAE |
| | | DTAVYYCARYEYGYYALDYWGQGTLVTVSS |
| 365 | VL | DIQLTQSPSSLSASVGDRVTITCRASQSVDFYGISFLHWYQQKPG |
| | | KAPKLLIYAASSLQSGVPSRFSGSGSGTDFTLTISSLQPEDFATY |
| | | YCQQSYRIPPTFGQGTKVEIK |

TY28654

| | | |
|---|---|---|
| 43 | HCDR1 | TSGVGVS |
| 96 | HCDR2 | SIYHSGSTYYNPSLKS |
| 312 | HCDR3 | YEYGYYAFDY |
| 76 | LCDR1 | RASQSVDFYGKSFLH |
| 9 | LCDR2 | AASSLQS |
| 28 | LCDR3 | QQSYRTPLT |
| 366 | VH | EVQLVESGGGLVQPGGSLRLSCAASGFSISTSGVGVSWIRQAPGK |
| | | GLEWIGSIYHSGSTYYNPSLKSRVTISRDNSKNTLYLQLNSLRAE |
| | | DTAVYYCARYEYGYYAFDYWGQGTLVTVSS |
| 367 | VL | DIQLTQSPSSLSASVGDRVTITCRASQSVDFYGKSFLHWYQQKPG |
| | | KAPKLLIYAASSLQSGVPSRFSGSGSGTDFTLTISSLQPEDFATY |
| | | YCQQSYRTPLTFGQGTKVEIK |

TY28655

| | | |
|---|---|---|
| 43 | HCDR1 | TSGVGVS |
| 96 | HCDR2 | SIYHSGSTYYNPSLKS |
| 307 | HCDR3 | YEYDYYAFDY |
| 310 | LCDR1 | RASQSVDFYGISFLH |
| 9 | LCDR2 | AASSLQS |
| 313 | LCDR3 | QQSYRTPRT |
| 368 | VH | EVQLVESGGGLVQPGGSLRLSCAASGESLSTSGVGVSWIRQAPGK |
| | | GLEWIGSIYHSGSTYYNPSLKSRVTISRDNSKNTLYLQINSLRAE |
| | | DTAVYYCARYEYDYYAEDYWGQGTLVTVSS |
| 369 | VL | DIQLTQSPSSLSASVGDRVTITCRASQSVDFYGISFLHWYQQKPG |
| | | KAPKLLIYAASSLQSGVPSRFSGSGSGTDETLTISSLQPEDFATY |
| | | YCQQSYRIPRTFGQGTKVEIK |

TY28656

| | | |
|---|---|---|
| 314 | HCDR1 | TSGVGVG |
| 315 | HCDR2 | EIYHSGSTNYNPSLKS |
| 309 | HCDR3 | YEYGYYALDY |
| 76 | LCDR1 | RASQSVDFYGKSFLH |
| 9 | LCDR2 | AASSLQS |
| 313 | LCDR3 | QQSYRTPRT |
| 370 | VH | EVQLVESGGGLVQPGGSLRLSCAASGFSLSTSGVGVGWIRQAPGK |
| | | GLEWIGEIYHSGSTNYNPSLKSRVTISRDNSKNTLYLQINSLRAE |
| | | DTAVYYCARYEYGYYALDYWGQGTLVTVSS |
| 371 | VL | DIQLTQSPSSLSASVGDRVTITCRASQSVDFYGKSFLHWYQQKPG. |
| | | KAPKLLIYAASSLQSGVPSRESGSGSGTDETLTISSLQPEDFATY |
| | | YCQQSYRTPRTFGQGTKVEIK |

TY28657

| | | |
|---|---|---|
| 33 | HCDR1 | TGGVGVG |
| 316 | HCDR2 | EIYHSGSTYYNPSLKS |
| 312 | HCDR3 | YEYGYYAFDY |
| 8 | LCDR1 | RASQSVDFHGISFLH |
| 308 | LCDR2 | AASSLES |
| 313 | LCDR3 | QQSYRIPRT |
| 372 | VH | EVQLVESGGGLVQPGGSLRLSCAASGFSLSTGGVGVGWIRQAPGK |
| | | GLEWIGEIYUSGSTYYNPSLKSRVTISRDNSKNILYLQINSLRAE |
| | | DTAVYYCARYEYGYYAFDYWGQGTLVTVSS |
| 373 | VL | DIQLTQSPSSLSASVGDRVTITCRASQSVDFHGISFLHWYQQKPG |
| | | KAPKLLIYAASSLESGVPSRFSGSGSGIDETLTISSLQPEDFATY |
| | | YCQQSYRTPRIFGQGTKVEIK |

TY28658

| | | |
|---|---|---|
| 314 | HCDR1 | TSGVGVG |
| 96 | HICDR2 | SIYHSGSTYYNPSLKS |
| 317 | HCDR3 | YEYDYYDEDY |
| 76 | LCDR1 | RASQSVDFYGKSFLH |
| 308 | LCDR2 | AASSLES |
| 318 | LCDR3 | QQSYRSPLT |
| 374 | VH | EVQLVESGGGLVQPGGSLRLSCAASGESLSTSGVGVGWIRQAPGK |
| | | GLEWIGSIYHSGSTYYNPSLKSRVTISRDNSKNTLYLQLNSLRAE |
| | | DTAVYYCARYEYDYYDEDYWGQGTLVTVSS |
| 375 | VI | DIQLTQSPSSLSASVGDRVTITCRASQSVDFYGKSELHWYQQKPG |
| | | KAPKLLIYAASSLESGVPSRFSGSGSGTDETLTISSLQPEDFATY |
| | | YCQQSYRSPLTFGQGTKVEIK |

TY28659

| | | |
|---|---|---|
| 43 | HCDR1 | TSGVGVS |
| 316 | HCDR2 | EIYHSGSTYYNPSLKS |
| 312 | HCDR3 | YEYGYYAFDY |
| 8 | LCDR1 | RASQSVDFHGISFELH |
| 9 | LCDR2 | AASSLQS |
| 10 | LCDR3 | QQSYRSPRT |
| 376 | VH | EVQLVESGGGLVQPGGSLRLSCAASGESLSTSGVGVSWIRQAPGK |
| | | GLEWIGEIYHSGSTYYNPSLKSRVTISRDNSKNILYLQLNSIRAE |
| | | DTAVYYCARYEYGYYAFDYWGQGTLVTVSS |
| 12 | VL | DIQLTQSPSSLSASVGDRVTITCRASQSVDEHGISFLHWYQQKPG |
| | | KAPKLLIYAASSLQSGVPSRFSGSGSGTDETLTISSLQPEDFATY |
| | | YCQQSYRSPRIFGQGTKVEIK |

TY28660

| | | |
|---|---|---|
| 43 | HCDR1 | TSGVGVS |
| 6 | HCDR2 | EIYHSGSTYYSPSLKS |
| 319 | HCDR3 | YEYYSYAFDY |
| 320 | LCDR1 | RASQSVDEHGKSFLH |
| 9 | LCDR2 | AASSLQS |
| 10 | LCDR3 | QQSYRSPRT |
| 377 | VH | EVQLVESGGGLVQPGGSLRLSCAASGESLSTSGVGVSWIRQAPGK |
| | | GLEWIGEIYHSGSTYYSPSLKSRVTISRDNSKNTLYLQLNSLRAE |
| | | DTAVYYCARYEYYSYAFDYWGQGTLVTVSS |
| 378 | VL | DIQLTQSPSSLSASVGDRVTITCRASQSVDFHGKSFLHWYQQKPG |
| | | KAPKLLIYAASSLQSGVPSRFSGSGSGTDETLTISSLQPEDFATY |
| | | YCQQSYRSPRTEGQGTKVEIK |

TY28661

| | | |
|---|---|---|
| 5 | HCDR1 | TGGVGVS |
| 67 | HCDR2 | SIYHSGSTYYSPSLKS |
| 307 | HCDR3 | YEYDYYAFDY |
| 310 | LCDR1 | RASQSVDFYGISFLH |
| 308 | LCDR2 | AASSLES |
| 28 | LGDR3 | QQSYRIPLT |
| 379 | VH | EVQLVESGGGLVQPGGSLRLSCAASGFSLSTGGVGVSWIRQAPGK |
| | | GLEWIGSIYHSGSTYYSPSLKSRVTISRDNSKNILYLQINSLRAE |
| | | DTAVYYCARYEYDYYAFDYWGQGTLVTVSS |
| 380 | VL | DIQLTQSPSSLSASVGDRVTITCRASQSVDFYGISFLHWYQQKPG |
| | | KAPKLLIYAASSLESGVPSRFSGSGSGIDETLTISSLQPEDFATY |
| | | YCQQSYRTPLTFGQGTKVEIK |

TY30120

| | | |
|---|---|---|
| 43 | HCDR1 | TSGVGVS |
| 6 | HCDR2 | EIYHSGSTYYSPSLKS |
| 321 | HCDR3 | YGYYYYDEDY |
| 8 | LCDR1 | RASQSVDEHGISFLH |
| 9 | LCDR2 | AASSLQS |
| 10 | LCDR3 | QQSYRSPRT |
| 381 | VH | EVQLVESGGGLVQPGGSLRLSCAASGESLSTSGVGVSWIRQAPGK |
| | | GLEWIGEIYHSGSTYYSPSLKSRVTISRDNSKNTLYLQLNSLRAE |
| | | DTAVYYCARYGYYYYDEDYWGQGTLVTVSS |
| 12 | VL | DIQLTQSPSSLSASVGDRVTITCRASQSVDEHGISFLHWYQQKPG |
| | | KAPKLLIYAASSLQSGVPSRFSGSGSGIDFTLTISSLQPEDFATY |
| | | YCQQSYRSPRTFGQGTKVEIK |

TY30121

| | | |
|---|---|---|
| 314 | HCDR1 | TSGVGVG |
| 316 | HCDR2 | EIYHSGSTYYNPSLKS |
| 322 | HCDR3 | YKYDYYDEDY |
| 8 | LCDR1 | RASQSVDFHGISELH |
| 9 | LCDR2 | AASSLQS |
| 10 | LCDR3 | QQSYRSPRT |
| 382 | VH | EVQLVESGGGLVQPGGSLRLSCAASGESLSTSGVGVGWIRQAPGK |
| | | GLEWIGEIYHSGSTYYNPSLKSRVTISRDNSKNTLYLQLNSIRAE |
| | | DTAVYYCARYKYDYYDEDYWGQGTLVTVSS |
| 12 | VL | DIQLTQSPSSLSASVGDRVTITCRASQSVDEHGISFLHWYQQKPG |
| | | KAPKLLIYAASSLQSGVPSRFSGSGSGIDETLTISSLQPEDFATY |
| | | YCQQSYRSPRIFGQGTKVEIK |

TY30123

| | | |
|---|---|---|
| 33 | HCDR1 | TGGVGVG |
| 6 | HCDR2 | EIYHSGSTYYSPSLKS |
| 321 | HCDR3 | YGYYYYDEDY |
| 8 | LCDR1 | RASQSVDERGISFLH |
| 9 | LCDR2 | AASSLQS |
| 10 | LCDR3 | QQSYRSPRT |
| 383 | VH | EVQLVESGGGLVQPGGSLRLSCAASGESLSIGGVGVGWIRQAPGK |
| | | GLEWIGEIYHSGSTYYSPSLKSRVTISRDNSKNTLYLQLNSLRAE |
| | | DTAVYYCARYGYYYYDEDYWGQGTLVTVSS |
| 12 | VL | DIQLTQSPSSLSASVGDRVTITCRASQSVDFHGISFLHWYQQKPG |
| | | KAPKLLIYAASSLQSGVPSRFSGSGSGTDETLTISSLQPEDFATY |
| | | YCQQSYRSPRIEGQGTKVEIK |

TY30406

| | | |
|---|---|---|
| 5 | HCDR1 | TGGVGVS |
| 6 | HCDR2 | EIYHSGSTYYSPSLKS |
| 317 | HCDR3 | YEYDYYDEDY |
| 8 | LCDR1 | RASQSVDFHGISFLH |
| 9 | LCDR2 | AASSLQS |
| 10 | LCDR3 | QQSYRSPRT |
| 384 | VH | EVQLVESGGGLVQPGGSLRISCAASGFSLSIGGVGVSWIRQAPGK |
| | | GLEWIGEIYHSGSTYYSPSLKSRVTISRDNSKNTLYLQINSLRAE |
| | | DTAVYYCARYEYDYYDEDYWGQGTLVTVSS |
| 12 | VI | DIQLTQSPSSLSASVGDRVTITCRASQSVDFHGISELHWYQQKPG |
| | | KAPKLLIYAASSLQSGVPSRFSGSGSGTDFTLTISSLQPEDFATY |
| | | YCQQSYRSPRIFGQGTKVEIK |

TY30410

| | | |
|---|---|---|
| 5 | HCDR1 | TGGVGVS |
| 6 | HCDR2 | EIYHSGSTYYSPSLKS |
| 323 | HCDR3 | YEYYEYDEDY |
| 8 | LCDR1 | RASQSVDFHGISFLH |
| 9 | LCDR2 | AASSLQS |
| 10 | LCDR3 | QQSYRSPRT |
| 385 | VH | EVQLVESGGGLVQPGGSLRLSCAASGESLSTGGVGVSWIRQAPGK |
| | | GLEWIGEIYHSGSTYYSPSLKSRVTISRDNSKNTLYLQLNSLRAE |
| | | DIAVYYCARYEYYEYDEDYWGQGTLVTVSS |
| 12 | VL | DIQLTQSPSSLSASVGDRVTITCRASQSVDEHGISFLHWYQQKPG |
| | | KAPKLLIYAASSLQSGVPSRFSGSGSGTDFTLTISSLQPEDFATY |
| | | YCQQSYRSPRTEGQGTKVEIK |

TY30413

| | | |
|---|---|---|
| 5 | HCDR1 | TGGVGVS |
| 6 | HCDR2 | EIYHSGSTYYSPSLKS |
| 324 | HCDR3 | YEYYHYDEDY |
| 8 | LCDR1 | RASQSVDFHGISFLH |
| 9 | LCDR2 | AASSLQS |
| 10 | LCDR3 | QQSYRSPRT |
| 386 | VH | EVQLVESGGGLVQPGGSLRLSCAASGFSLSTGGVGVSWIRQAPGK |
| | | GLEWIGEIYHSGSTYYSPSLKSRVTISRDNSKNTLYLQLNSLRAE |
| | | DTAVYYCARYEYYHYDEDYWGQGTLVTVSS |
| 12 | VL | DIQLTQSPSSLSASVGDRVTITCRASQSVDFHGISFLHWYQQKPG |
| | | KAPKLLIYAASSLQSGVPSRFSGSGSGTDFTLTISSLQPEDFATY |
| | | YCQQSYRSPRTFGQGTKVEIK |

| SEQ ID | PD-L1xCD28 Bispecific Antibodies (VH-VL) | |
|---|---|---|
| NO: | TY29815 (WT) | |
| 325 | LC | DIQLTQSPSSLSASVGDRVTITCRASQSIPSFLNWYQQKPGKAPKLLIYAA |
| | | SSLQSGVPSRFSGSGSGIDFTLTISSLQPEDFATYYCQHYISWPRQFTEGQ |
| | | GTKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGLS |
| | | SPVIKSENRGEC* |
| 326 | HC_A | EVQLVESGGGLVQPGGSLRLSCAASGYSISSGYYWGWIRQAPGKGLEWIGI |
| | | IYPSGGGINYAQKFQGRVTISRDNSKNTLYLQLNSLRAEDTAVYYCARGGG |
| | | LGFDYWGQGTLVTVSSASTKGPSVFPLAPSSKSTSGGTAALGCLVKDYFRE |
| | | PVTVSWNSGALTSGVHTFPAVLQSSGLYSISSVVTVPSSSLGTQTYICNVN |
| | | HKPSNTKVDKKVEPKSCDKTHTCPPCPAPELLGGPSVFLFPPKPKDTLMIS |
| | | RTPEVTCVVVDVSHEDPEVKINWYVDGVEVHNAKTKPREEQYASTYRVVSV |
| | | LTVLHQDWLNGKEYKCKVSNKALPAPIEKTISKAKGQPREPQVYTLPPSRK |
| | | KLTKNQVKLTCLVKGFYPSDIAVEWESNGQPENNYKTTPPVLDQDGSEFLY |
| | | SKLTVDKSRWQQGNVESCSVMHEALHNHYTQKSLSLSPGK* |
| 327 | HC_B | EVQLVESGGGLVQPGGSLRLSCAASGFSLSTGGVGVSWIRQAPGKGLEWIG |
| | | EIYHSGSTYYSPSLKSRVJISRDNSKNTLYLQLNSLRAEDTAVYYCARYEY |
| | | YYYDFDYWGQGTLVTVSSGGGGSGGGGSGGGGSGGGGSDIQLTQSPSSLSA |
| | | SVGDRVTITCRASQSVDEHGISELHWYQQKPGKAPKLLIYAASSLQSGVPS |
| | | RESGSGSGTDFTLQISSLQPEDFATYYCQQSYRSPRTFGQGTKVEIKEPKS |
| | | SDKTHTCPPCPAPELLGGPSVFLFPPKPKDTLMISRTPEVTCVVVDVSHED |
| | | PEVKFNWYVDGVEVHNAKQKPREEQYASTYRVVSVLTVLHQDWINGKEYKC |
| | | KVSNKALPAPIEKTISKAKGQPREPQVYTDPPSRDELTKNQVSLTCLVDGF |
| | | YPSDIAVEWESNGQPENCYKTTPPVLDSDGSEFLYSKLEVDKSRWQQGNVT |
| | | SCSVMHEALHNHYQQDSLSLSPGK* |

TY30406

| | | |
|---|---|---|
| 325 | LC | DIQLTQSPSSLSASVGDRVTITCRASQSIPSELNWYQQKPGKAPKLLIYAA |
| | | SSLQSGVPSRESGSGSGTDETLT-SSLQPEDEATYYCQHYISWPRQFTEGQ |
| | | GIKVEIKRTVAAPSVFIEPPSDEQLKSGTASVVCLINNFYPREAKVQWKVD |
| | | NALQSGNSQESVTEQDSKDSTYSLSSTLELSKADYEKHKVYACEVTHQGLS |
| | | SPVTKSENRGEC* |
| 326 | HC_A | EVQLVESGGGLVQPGGSURLSCAASGYSISSGYYWGWIRQAPGKGLEWIGI |
| | | IYPSGGGTNYAQKFQGRVTISRDNSKNTLYLQLNSLRAEDTAVYYCARGGG |
| | | LGFDYWGQGTLVTVSSASTKGPSVFPLAPSSKSTSGGTAALGQLVKDYFPE |
| | | PVTVSWNSGALTSGVHTFPAVLQSSGLYSLSSVVTVPSSSLGTQTYICNVN |
| | | HKPSNTKVDKKVEPKSCEKTHTCPPCPAPELLGGPSVFLFPPKPKDTLMIS |
| | | RTPEVTCVVVDVSHEDPEVKFNWYVDGVEVHNAKTKPREEQYASTYRVVSV |
| | | LTVLHQDWLNGKEYKCKVSNKALPAPIEKTISKAKGQPREPQVYTLPPSRK |
| | | KLTKNQVKLTCLVKGFYPSDIAVEWESNGQPENNYKTTPPVLDCDGSFFLY |
| | | SKLTVDKSRWQQGNVFSCSVMHEALIINHYTQKSLSLSPGK* |
| 328 | HC_B | EVQLVESGGGLVQPGGSLRLSCAASGFSLSTGGVGVSWIRQAPGKGLEWIG |
| | | EIYHSGSTYYSPSLKSRVJISRDNSKNILYLQLNSLRAEDTAVYYCARYEY |
| | | DYYDFDYWGQGTLVIVSSGGGGSGGGGSGGGGSGGGGSDIQLTQSPSSLSA |
| | | SVGDRVTITCRASQSVDEHGISELHWYQQKPGKAPKLLIYAASSLQSGVPS |
| | | RESGSGSGIDFTLCISSLQPEDFATYYCQQSYRSPRTFGQGTKVEIKEPKS |
| | | SDKTHTCPPCPAPELLGGPSVFLFPPKPKDTLMISRTPEVTCVVVDVSHED |
| | | PEVKENWYVDGVEVHNAKIKPREEQYASTYRVVSVLTVLHQDWLNGKEYKC |
| | | KVSNKALPAPIEKTISKAKGQPREPQVYQDPPSRDELTKNQVSLTCLVDGF |
| | | YPSDIAVEWESNGQPENCYKTTPPVLDSDGSEFLYSKLEVDKSRWQQGNVE |
| | | SCSVMHEALHNHYTQDSLSLSPGK* |

TY30410

| | | |
|---|---|---|
| 325 | LC | DIQLTQSPSSLSASVGDRVTITCRASQS-PSELNWYQQKPGKAPKLLIYAA |
| | | SSLQSGVPSRFSGSGSGTDETLTISSLQPEDFATYYCQHYISWPRQFTEGQ |
| | | GTKVEIKRTVAAPSVFIEPPSDEQLKSGTASVVCLINNFYPREAKVQWKVD |
| | | NALQSGNSQESVTEQDSKDSTYSLSSTLQLSKADYEKHKVYACEVTHQGLS |
| | | SPVTKSENRGEC* |
| 326 | HC_A | EVQLVESGGGLVQPGGSLRISCAASGYSISSGYYWGWIRQAPGKGLEWIGI |
| | | IYPSGGGTNYAQKFQGRVTISRDNSKNTLYLQLNSLRAEDTAVYYCARGGG |
| | | LGEDYWGQGTLVTVSSASTKGPSVFPLAPSSKSTSGGTAALGQLVKDYFPE |
| | | PVTVSWNSGALTSGVHTFPAVLQSSGLYSLSSVVTVPSSSIGTQTYICNVN |
| | | HKPSNTKVDKKVEPKSCDKTHTCPPCPAPELLGGPSVFLFPPKPKDTLMIS |
| | | RTPEVTCVVVDVSHEDPEVKENWYVDGVEVHNAKTKPREEQYASTYRVVSV |
| | | LTVLHQDWINGKEYKCKVSNKALPAPIEKTISKAKGQPREPQVYTLPPSRK |
| | | KLTKNQVKLTCLVKGEYPSDIAVEWESNGQPENNYKTTPPVLDCDGSFFLY |
| | | SKLTVDKSRWQQGNVFSCSVMHEALHNHYTQKSLSLSPGK* |
| 329 | HC_B | EVQLVESGGGLVQPGGSIRLSCAASGESLSIGGVGVSWIRQAPGKGLEWIG |
| | | EIYHSGSTYYSPSLKSRVTISRDNSKNTLYLQLNSLRAEDTAVYYCARYEY |
| | | YEYDEDYWGQGTLVTVSSGGGGSGGGGSGGGGSGGGGSDIQLTQSPSSLSA |
| | | SVGDRVTITCRASQSVDEHGISFLHWYQQKPGKAPKLLIYAASSLQSGVPS |
| | | RFSGSGSGTDFTLTISSLQPEDFATYYCQQSYRSPRTFGQGTKVEIKEPKS |
| | | SDKTHTCPPCPAPELLGGPSVFLEPPKPKDTIMISRTPEVTCVVVDVSHED |
| | | PEVKENWYVDGVEVHNAKIKPREEQYASTYRVVSVLIVLHQDWLNGKEYKC |
| | | KVSNKALPAPIEKTISKAKGQPREPQVYTDPPSRDELTKNQVSLICLVDGE |
| | | YPSDIAVEWESNGQPENCYKTTPPVLDSDGSFFLYSKLTVEKSRWQQGNVE |
| | | SCSVMHEALHNHYTQDSLSLSPGK* |

TY30413

| | | |
|---|---|---|
| 325 | LC | DIQLTQSPSSLSASVGDRVTITCRASQSIPSFLNWYQQKPGKAPKLLIYAA |
| | | SSLQSGVPSRESGSGSGTDFTLTISSLQPEDFATYYCQHYISWPRQFTFGQ |
| | | GTKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGLS |
| | | SPVIKSENRGEC* |
| 326 | HC_A | EVQLVESGGGLVQPGGSIRLSCAASGYSISSGYYWGWIRQAPGKGLEWIGI |
| | | IYPSGGGINYAQKTQGRVTISRDNSKNTLYLQLNSLRAEDTAVYYCARGGG |
| | | LGFDYWGQGTLVTVSSASTKGPSVFPLAPSSKSTSGGTAALGCLVKDYFPE |
| | | PVTVSWNSGALTSGVHTFPAVLQSSGLYSLSSVVTVPSSSIGTQTYICNVN |
| | | HKPSNTKVDKKVEPKSCDKTHTCPPCPAPELLGGPSVELFPPKPKDTLMIS |
| | | RTPEVTCVVVDVSHEDPEVKENWYVDGVEVHNAKTKPREEQYASTYRVVSV |
| | | LTVLHQDWINGKEYKCKVSNKALPAPIEKTISKAKGQPREPQVYTLPPSRK |
| | | KLIKNQVKLTCLVKGFYPSDIAVEWESNGQPENNYKTTPPVLDCDGSFFLY |
| | | SKLTVDKSRWQQGNVFSCSVMHEALHNHYTQKSLSLSPGK* |
| 330 | HC_B | EVQLVESGGGLVQPGGSLRLSCAASGFSLSTGGVGVSWIRQAPGKGLEWIG |
| | | EIYHSGSTYYSPSLKSRVTISRQNSKNTLYLQLNSERAEDTAVYYCARYEY |
| | | YHYDFDYWGQGTLVTVSSGGGGSGGGGSGGGGSGGGGSDIQLTQSPSSLSA |
| | | SVGDRVTITCRASQSVDFHGISFLHWYQQKPGKAPKLLIYAASSLQSGVPS |
| | | RFSGSGSGTDFTITISSLQPEDFATYYCQQSYRSPRIEGQGTKVEIKEPKS |
| | | SDKTHTCPPCPAPELLGGPSVFLFPPKPKDTIMISRIPEVTCVVVDVSHED |
| | | PEVKFNWYVDGVEVHNAKTKPREEQYASTYRVVSVLIVLHQDWINGKEYKC |
| | | KVSNKALPAPIEKTISKAKGQPREPQVYTDPPSRDELTKNQVSLICLVDGF |
| | | YPSDIAVEWESNGQPENCYKTTPPVLDSDGSFFLYSKLTVDKSRWQQGNVE |
| | | SCSVMHEALUNHYTQDSLSLSPGK* |

HER2xCD28 Bispecific Antibodies (VL-VH)
TY27566 (WT)

| | | |
|---|---|---|
| 331 | LC | DIQMTQSPSSLSASVGDRVTITCKASQDVSIGVAWYQQKPGKAPKLLIYSA |
| | | SYRYTGVPSRFSGSGSGTDETLTISSLQPEDFATYYCQQYYTYPYTEGQGT |
| | | KVEIKRTVAAPSVEIFPPSDEQLKSGIASVVCLLNNFYPREAKVQWKVDNA |
| | | LQSGNSQESVTEQDSKDSTYSLSSTLILSKADYEKHKVYACEVTHQGLSSP |
| | | VTKSFNRGEC* |
| 332 | HC_A | EVQLVESGGGLVQPGGSLRLSCAASGFTFTDYTMDWVRQAPGKGLEWVADV |
| | | NPNSGGSIYNQRFKGRFTLSVDRSKNTLYLQMNSLRAEDTAVYYCARNLGP |
| | | SFYFDYWGQGILVTVSSASTKGPSVFPLAPSSKSTSGGTAALGCLVKDYFP |
| | | EPVTVSWNSGALTSGVHTFPAVLQSSGLYSLSSVVTVPSSSIGTQTYICNV |
| | | NHKPSNTKVDKKVEPKSCDKTHTCPPCPAPELLGGPSVFLFPPKPKDTLMI |
| | | SRTPEVTCVVVDVSHEDPEVKENWYVDGVEVHNAKTKPREEQYASTYRVVS |
| | | VLTVLHQDWLNGKEYKCKVSNKALPAPIEKTISKAKGQPREPQVYTLPPSR |
| | | KKLTKNQVKLTCLVKGFYPSDIAVEWESNGQPENNYKTTPPVLDCDGSFFL |
| | | YSKLTVDKSRWQQGNVFSCSVMHEALHNHYTQKSLSLSPGK* |
| 333 | HC_B | DIQLTQSPSSLSASVGDRVTITCRASQSVDERGISFLHWYQQKPGKAPKLL |
| | | IYAASSLQSGVPSRESGSGSGIDFILTISSLQPEDFATYYCQQSYRSPRTE |
| | | GQGTKVEIKGGGGSGGGGSGGGGSGGGGSEVQLVESGGGLVQPGGSLRLSC |
| | | AASGESLSIGGVGVSWIRQAPGKGLEWIGEIYHSGSTYYSPSLKSRVTISR |
| | | DNSKNTLYLQLNSLRAEDTAVYYCARYEYYYYDFDYWGQGTLVTVSSEPKS |
| | | SDKTHTCPPCPAPELLGGPSVFLFPPKPKDTLMISRTPEVTCVVVDVSHED |
| | | PEVKENWYVDGVEVHNAKTKPREEQYASTYRVVSVLTVLHQDWINGKEYKC |
| | | KVSNKALPAPIEKTISKAKGQPREPQVYIDPPSRDELTKNQVSLICLVDGF |
| | | YPSDIAVEWESNGQPENCYKTTPPVLDSDGSFFLYSKLTVDKSRWQQGNVE |
| | | SCSVMHEALHNHYTQDSLSLSPGK* |

TY28652

| | | |
|---|---|---|
| 331 | LC | DIQMTQSPSSLSASVGDRVTITCKASQDVSIGVAWYQQKPGKAPKLLIYSA |
| | | SYRYTGVPSRFSGSGSGTDFILTISSLQPEDFATYYCQQYYIYPYTEGQGT |
| | | KVEIKRTVAAPSVFIFPPSDEQLKSGTASVVCLINNFYPREAKVQWKVDNA |
| | | LQSGNSQESVTEQDSKDSTYSLSSTLTLSKADYEKHKVYACEVTHQGLSSP |
| | | VIKSENRGEC* |
| 332 | HC_A | EVQLVESGGGLVQPGGSLRLSCAASGFIFTDYTMDWVRQAPGKGLEWVADV |
| | | NPNSGGSIYNQRFKGRFTLSVDRSKNTLYLQMNSLRAEDTAVYYCARNIGP |
| | | SFYFDYWGQGTLVTVSSASTKGPSVFPLAPSSKSTSGGTAALGCLVKDYFP |
| | | EPVTVSWNSGALTSGVHTEPAVLQSSGLYSLSSVVIVPSSSLGTQTYICNV |
| | | NHKPSNTKVDKKVEPKSCDKTHICPPCPAPELLGGPSVELFPPKPKDTIMI |
| | | SRTPEVTCVVVDVSHEDPEVKENNYVDGVEVHNAKTKPREEQYASTYRVVS |
| | | VLTVLHQDWLNGKEYKCKVSNKALPAPIEKTISKAKGQPREPQVYTLPPSR |
| | | KKLIKNQVKLTCLVKGFYPSDIAVEWESNGQPENNYKITPPVLDCDGSFFL |
| | | YSKLTVDKSRWQQGNVFSCSVMHEALHNHYTQKSLSLSPGK* |
| 334 | HC_B | DIQLTQSPSSLSASVGDRVTITCRASQSVDFYGKSFLHWYQQKPGKAPKLL |
| | | IYAASSLESGVPSRFSGSGSGTDFTLTISSLQPEDFATYYCQQSYRSPPTE |
| | | GQGTKVEIKGGGGSGGGGSGGGGSGGGGSEVQLVESGGGLVQPGGSLRLSC |
| | | AASGFSLSTGGVGVSWIRQAPGKGLEWIGSIYHSGSTYYNPSLKSRVTISR |
| | | DNSKNTLYLQLNSLRAEDTAVYYCARYEYDYYAFDYWGQGTLVTVSSEPKS |
| | | SDKTHTCPPCPAPELLGGPSVFLFPPKPKDTLMISRTPEVTCVVVDVSHED |
| | | PEVKFNWYVDGVEVHNAKTKPREEQYASTYRVVSVLTVLHQDWINGKEYKC |
| | | KVSNKALPAPIEKTISKAKGQPREPQVYTDPPSRDELTKNQVSLICLVDGF |
| | | YPSDIAVEWESNGQPENCYKTIPPVLDSDGSEFLYSKLTVDKSRWQQGNVF |
| | | SCSVMHEALHNHYTQDSLSLSPGK* |

TY28653

| | | |
|---|---|---|
| 331 | LC | DIQMTQSPSSLSASVGDRVTITCKASQDVSIGVAWYQQKPGKAPKLLIYSA |
| | | SYRYTGVPSRFSGSGSGTDFTLTISSLQPEDFATYYCQQYYTYPYTFGQGT |
| | | KVEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VIKSFNRGEC* |
| 332 | HC_A | EVQLVESGGGLVQPGGSLRLSCAASGFTFTDYTMDWVRQAPGKGLEWVADV |
| | | NPNSGGSIYNQRFKGRFTLSVDRSKNTLYLQMNSLRAEDTAVYYCARNLGP |
| | | SFYFDYWGQGTLVTVSSASTKGPSVFPLAPSSKSTSGGTAALGCLVKDYEP |
| | | EPVTVSWNSGALTSGVHTTPAVLQSSGLYSLSSVVTVPSSSIGTQTYICNV |
| | | NHKPSNTKVDKKVEPKSCDKTHTCPPCPAPELLGGPSVFLFPPKPKDTLMI |
| | | SRTPEVTCVVVDVSHEDPEVKENWYVDGVEVHNAKIKPREEQYASTYRVVS |
| | | VLTVLHQDWLNGKEYKCKVSNKALPAPIEKTISKAKGQPREPQVYTLPPSR |
| | | KKLTKNQVKLTCLVKGFYPSDIAVEWESNGQPENNYKTTPPVLDCDGSFFL |
| | | YSKLTVDKSRWQQGNVFSCSVMHEALHNHYTQKSLSLSPGK* |
| 335 | HC_B | DIQLTQSPSSLSASVGDRVTITCRASQSVDFYGISELAWYQQKPGKAPKLL |
| | | IYAASSLQSGVPSRFSGSGSGTDFTLTISSLQPEDFATYYCQQSYRIPPTF |
| | | GQGTKVEIKGGGGSGGGGSGGGGSGGGGSEVQLVESGGGLVQPGGSLRLSC |
| | | AASGFSLSTGGVGVSWIRQAPGKGLEWIGSIYHSGSTYYNPSLKSRVTISR |
| | | DNSKNTLYLQLNSLRAEDTAVYYCARYEYGYYALDYWGQGTLVTVSSEPKS |
| | | SDKTHTCPPCPAPELLGGPSVFLEPPKPKDTLMISRIPEVTCVVVDVSHED |
| | | PEVKENWYVDGVEVHNAKTKPREEQYASTYRVVSVLIVLHQDWINGKEYKC |
| | | KVSNKALPAPIEKTISKAKGQPREPQVYTDPPSRDELIKNQVSLTCLVDGF |
| | | YPSDIAVEWESNGQPENCYKTIPPVLDSDGSFFLYSKLTVDKSRWQQGNVE |
| | | SCSVMHEALHNHYTQDSLSLSPGK* |

TY28654

| | | |
|---|---|---|
| 331 | LC | DIQMTQSPSSLSASVGDRVTITCKASQDVSIGVAWYQQKPGKAPKLLIYSA |
| | | SYRYTGVPSRFSGSGSGTDFILTISSLQPEDFATYYCQQYYTYPYTFGQGT |
| | | KVEIKRIVAAPSVFIFPPSDEQLKSGTASVVCLINNFYPREAKVQWKVDNA |
| | | LQSGNSQESVTEQDSKDSTYSLSSTLTLSKADYEKHKVYACEVTHQGLSSP |
| | | VIKSENRGEC* |
| 332 | HC_A | EVQLVESGGGLVQPGGSLRLSCAASGFTFTDYTMDWVRQAPGKGLEWVADV |
| | | NPNSGGSIYNQRFKGRFTLSVDRSKNTLYLQMNSLRAEDTAVYYCARNIGP |
| | | SFYEDYWGQGTLVTVSSASTKGPSVFPLAPSSKSTSGGIAALGCLVKDYFP |
| | | EPVTVSWNSGALTSGVHTEPAVLQSSGLYSLSSVVTVPSSSIGTQTYICNV |
| | | NHKPSNTKVDKKVEPKSCDKTHTCPPCPAPELLGGPSVELFPPKPKDTLMI |
| | | SRTPEVICVVVDVSHEDPEVKENWYVDGVEVHNAKTKPREEQYASTYRVVS |
| | | VLTVLHQDWLNGKEYKCKVSNKALPAPIEKTISKAKGQPREPQVYTIPPSR |
| | | KKLTKNQVKLTCLVKGFYPSDIAVEWESNGQPENNYKITPPVLDCDGSFFL |
| | | YSKLTVDKSRWQQGNVFSCSVMHEALHNHYTQKSLSLSPGK* |
| 336 | HC_B | DIQLTQSPSSLSASVGDRVTITCRASQSVDFYGKSFLHWYQQKPGKAPKLL |
| | | IYAASSLQSGVESRESGSGSGIDFTLTISSLQPEDEATYYCQQSYRTPITE |
| | | GQGTKVEIKGGGSGGGGGSGGGGSGGGGSEVQLVESGGGLVQPGGSLRLSC |
| | | AASGFSLSTSGVGVSWIRQAPGKGLEWIGSIYHSGSTYYNPSLKSRVTISR |
| | | DNSKNTLYLQLNSLRAEDTAVYYCARYEYGYYAFDYWGQGTLVTVSSEPKS |
| | | SDKTHTCPPCPAPELLGGPSVFLFPPKPKDTIMISRTPEVTCVVVDVSHED |
| | | PEVKENWYVDGVEVHNAKTKPREEQYASTYRVVSVLIVLHQDWINGKEYKC |
| | | KVSNKALPAPIEKTISKAKGQPREPQVYIDPPSRDELTKNQVSLTCLVDGE |
| | | YPSDIAVEWESNGQPENCYKTTPPVLDSDGSFFLYSKLTVDKSRWQQGNVF |
| | | SCSVMHEALHNHYTQDSLSLSPGK* |

TY28655

| | | |
|---|---|---|
| 331 | LC | DIQMTQSPSSLSASVGDRVTITCKASQDVSIGVAWYQQKPGKAPKLLIYSA |
| | | SYRYTGVPSRESGSGSGTDFTLTISSLQPEDFATYYCQQYYTYPYTEGQGT |
| | | KVEIKRIVAAPSVEIFPPSDEQLKSGTASVVCLLNNFYPREAKVQWKVDNA |
| | | LQSGNSQESVTEQDSKDSTYSLSSTLTLSKADYEKHKVYACEVIHQGLSSP |
| | | VIKSENRGEC* |
| 332 | HC_A | EVQLVESGGGLVQPGGSLRLSCAASGFTFTDYTMDWVRQAPGKGLEWVADV |
| | | NPNSGGSIYNQRFKGRFTLSVDRSKNTLYLQMNSLRAEDTAVYYCARNLGP |
| | | SFYFDYWGQGTLVTVSSASTKGPSVEPLAPSSKSTSGGTAALGCLVKDYFP |
| | | EPVTVSWNSGALTSGVHIFPAVLQSSGLYSLSSVVTVPSSSLGIQTYICNV |
| | | NHKPSNIKVDKKVEPKSCDKTHTCPPCPAPELLGGPSVFLFPPKPKDTLMI |
| | | SRTPEVICVVVDVSHEDPEVKENWYVDGVEVHNAKTKPREEQYASTYRVVS |
| | | VLTVLHQDWLNGKEYKCKVSNKALPAPIEKTISKAKGQPREPQVYTLPPSR |
| | | KKLTKNQVKLTCLVKGFYPSDIAVEWESNGQPENNYKTTPPVLDCDGSEFL |
| | | YSKLTVDKSRWQQGNVESCSVMHEALHNHYTQKSLSLSPGK* |
| 337 | HC_B | DIQLTQSPSSLSASVGDRVTITCRASQSVDFYGISELHWYQQKPGKAPKIL |
| | | IYAASSLQSGVPSRFSGSGSGTDFTLTISSLQPEDFATYYCQQSYRTPRTE |
| | | GQGTKVEIKGGGGSGGGGSGGGGSGGGGSEVQLVESGGGLVQPGGSLRLSC |
| | | AASGFSLSTSGVGVSWIRQAPGKGLEWIGSIYHSGSTYYNPSLKSRVTISR |
| | | DNSKNTLYLQLNSLRAEDTAVYYCARYEYDYYAFDYWGQGTLVTVSSEPKS |
| | | SDKTHTCPPCPAPELLGGPSVFLFPPKPKDTLMISRTPEVTCVVVDVSHED |
| | | PEVKFNWYVDGVEVHNAKTKPREEQYASTYRVVSVLTVLHQDWLNGKEYKC |
| | | KVSNKALPAPIEKTISKAKGQPREPQVYTDPPSRDELTKNQVSLTCLVDGF |
| | | YPSDIAVEWESNGQPENCYKITPPVLDSDGSFFLYSKLTVDKSRWQQGNVE |
| | | SCSVMHEALHNHYTQDSLSLSPGK* |

TY28656

| | | |
|---|---|---|
| 331 | LC | DIQMTQSPSSLSASVGDRVTITCKASQDVSIGVAWYQQKPGKAPKLLIYSA |
| | | SYRYTGVPSRESGSGSGIDFTLTISSLQPEDFATYYCQQYYTYPYTFGQGT |
| | | KVEIKRTVAAPSVFIFPPSDEQLKSGTASVVCLLNNFYPREAKVQWKVDNA |
| | | LQSGNSQESVTEQDSKDSTYSLSSTLILSKADYEKHKVYACEVTHQGLSSP |
| | | VTKSENRGEC* |
| 332 | HC_A | EVQLVESGGGLVQPGGSLRLSCAASGETFTDYTMDWVRQAPGKGLEWVADV |
| | | NPNSGGSIYNQRFKGRETLSVDRSKNTLYLQMNSLRAEDTAVYYCARNLGP |
| | | SFYEDYWGQGTLVTVSSASTKGPSVIPLAPSSKSTSGGTAALGCLVKDYFP |
| | | EPVTVSWNSGALTSGVHTFPAVLQSSGLYSLSSVVIVESSSIGTQTYICNV |
| | | NHKPSNTKVDKKVEPKSCDKTHTCPPCPAPELLGGPSVELEPPKPKDTLMI |
| | | SRIPEVTCVVVDVSHEDPEVKENWYVDGVEVHNAKIKPREEQYASTYRVVS |
| | | VLIVLHQDWLNGKEYKCKVSNKALPAPIEKTISKAKGQPREPQVYTLPPSR |
| | | KKLTKNQVKLTCLVKGEYPSDIAVEWESNGQPENNYKTTPPVLDCDGSFEL |
| | | YSKLTVDKSRWQQGNVFSCSVMHEALHNHYTQKSLSLSPGK* |
| 338 | HC_B | DIQLTQSPSSLSASVGDRVTITCRASQSVDFYGKSFLHWYQQKPGKAPKLL |
| | | IYAASSLQSGVPSRFSGSGSGTDFTLTISSLQPEDFATYYCQQSYRTPRTF |
| | | GQGTKVEIKGGGGSGGGGSGGGGSGGGGSEVQLVESGGGLVQPGGSLRLSC |
| | | AASGESLSTSGVGVGWIRQAPGKGLEWIGEIYHSGSTNYNPSLKSRVTISR |
| | | DNSKNTLYLQLNSLRAEDTAVYYCARYEYGYYALDYWGQGTLVTVSSEPKS |
| | | SDKTHTCPPCPAPELLGGPSVFLEPPKPKDTLMISRIPEVTCVVVDVSHED |
| | | PEVKENWYVDGVEVHNAKIKPREEQYASTYRVVSVLIVLHQDWLNGKEYKC |
| | | KVSNKALPAPIEKTISKAKGQPREPQVYTDPPSRDELIKNQVSLTCLVDGF |
| | | YPSDIAVEWESNGQPENCYKTTPPVLDSDGSTELYSKLTVDKSRWQQGNVE |
| | | SCSVMHEALHNHYTQDSLSLSPGK* |

TY28657

| | | |
|---|---|---|
| 331 | LC | DIQMTQSPSSLSASVGDRVTITCKASQDVSIGVAWYQQKPGKAPKLLIYSA |
| | | SYRYTGVPSRFSGSGSGIDFTLTISSLQPEDFATYYCQQYYTYPYTEGQGT |
| | | KVEIKRTVAAPSVFIFPPSDEQLKSGTASVVCLLNNFYPREAKVQWKVDNA |
| | | LQSGNSQESVTEQDSKDSTYSISSTLILSKADYEKHKVYACEVTHQGLSSP |
| | | VIKSENRGEC* |
| 332 | HC_A | EVQLVESGGGLVQPGGSERLSCAASGETFTDYTMDWVRQAPGKGLEWVADV |
| | | NPNSGGSIYNQRFKGRETLSVDRSKNTLYLQMNSLRAEDTAVYYCARNLGP |
| | | SFYEDYWGQGTLVTVSSASTKGPSVFPLAPSSKSTSGGTAALGCLVKDYFP |
| | | EPVTVSWNSGALISGVHTFPAVLQSSGLYSLSSVVTVPSSSLGTQTYICNV |
| | | NHKPSNTKVDKKVEPKSCDKTHICPPCPAPELLGGPSVFLFPPKPKDTLMI |
| | | SRIPEVTCVVVDVSHEDPEVKENWYVDGVEVHNAKIKPREEQYASTYRVVS |
| | | VLIVLHQDWINGKEYKCKVSNKALPAPIEKTISKAKGQPREPQVYTIPPSR |
| | | KKLIKNQVKLTCLVKGEYPSDIAVEWESNGQPENNYKITPPVLDCDGSFEL |
| | | YSKLTVDKSRWQQGNVFSCSVMHEALHNHYTQKSLSLSPGK* |
| 339 | HC_B | DIQLTQSPSSLSASVGDRVTITCRASQSVDEHGISFLHWYQQKPGKAPKLL |
| | | IYAASSLESGVPSRFSGSGSGIDFTLTISSLQPEDFATYYCQQSYRTPRTF |
| | | GQGTKVEIKGGGGSGGGGSGGGGSGGGGSEVQLVESGGGLVQPGGSLRLSC |
| | | AASGFSLSTGGVGVGWIRQAPGKGLEWIGEIYHSGSTYYNPSLKSRVTISR |
| | | DNSKNTLYLQLNSLRAEDTAVYYCARYEYGYYAFDYWGQGTLVTVSSEPKS |
| | | SDKTHTCPPCPAPELLGGPSVFLFPPKPKDTIMISRTPEVTCVVVDVSHED |
| | | PEVKFNWYVDGVEVHNAKTKPREEQYASTYRVVSVLIVLHQDWLNGKEYKC |
| | | KVSNKALPAPIEKTISKAKGQPREPQVYTDPPSRDELTKNQVSLTCLVDGE |
| | | YPSDIAVEWESNGQPENCYKTIPPVLDSDGSFFLYSKLTVDKSRWQQGNVF |
| | | SCSVMHEALHNHYTQDSLSLSPGK* |

TY28658

| | | |
|---|---|---|
| 331 | LC | DIQMTQSPSSLSASVGDRVTITCKASQDVSIGVAWYQQKPGKAPKLLIYSA |
| | | SYRYTGVPSRESGSGSGTDFTLTISSLQPEDFATYYCQQYYTYPYTEGQGT |
| | | KVEIKRIVAAPSVFIFPPSDEQLKSGIASVVCLINNFYPREAKVQWKVDNA |
| | | LQSGNSQESVTEQDSKDSTYSLSSTLTLSKADYEKHKVYACEVTHQGLSSP |
| | | VIKSENRGEC* |
| 332 | HC_A | EVQLVESGGGLVQPGSSLRISCAASGFTFTDYTMDWVRQAPGKGLEWVADV |
| | | NPNSGGSIYNQRFKGRFTLSVDRSKNTLYLQMNSLRAEDTAVYYCARNLGP |
| | | SFYFDYWGQGTLVTVSSASTKGPSVFPLAPSSKSTSGGTAALGCLVKDYFP |
| | | EPVTVSWNSGALTSGVHTEPAVLQSSGLYSLSSVVTVPSSSIGTQTYICNV |
| | | NHKPSNTKVDKKVEPKSCDKTHTCPPCPAPELLGGPSVFLPPPKPKDTLMI |
| | | SRIPEVTCVVVDVSHEDPEVKENWYVDGVEVHNAKIKPREEQYASTYRVVS |
| | | VLTVLHQDWLNGKEYKCKVSNKALPAPIEKTISKAKGQPREPQVYTLPPSR |
| | | KKLTKNQVKLTCLVKGFYPSDIAVEWESNGQPENNYKTTPPVLDCDGSFFL |
| | | YSKLIVDKSRWQQGNVESCSVMHEALHNHYTQKSLSLSPGK* |
| 340 | HC_B | DIQLIQSPSSLSASVGDRVTITCRASQSVDFYGKSFLHWYQQKPGKAPKLL |
| | | IYAASSLESGVPSRFSGSGSGTDFTLTISSLQPEDFATYYCQQSYRSPLTF |
| | | GQGIKVEIKGGGGSGGGGSGGGGSGGGGSEVQLVESGGGLVQPGGSLRLSC |
| | | AASGFSLSTSGVGVGWIRQAPGKGLEWIGSIYHSGSTYYNPSLKSRVTISR |
| | | DNSKNTLYLQINSLRAEDTAVYYCARYEYDYYDFDYWGQGTLVTVSSEPKS |
| | | SDKTHTCPPCPAPELLGGPSVELFPPKPKDILMISRTPEVTCVVVDVSHED |
| | | PEVKENWYVDGVEVHNAKTKPREEQYASTYRVVSVLTVLHQDWINGKEYKC |
| | | KVSNKALPAPIEKTISKAKGQPREPQVYTDPPSRDELIKNQVSLTCLVDGE |

TY28659

| | | |
|---|---|---|
| 331 | LC | DIQMTQSPSSLSASVGDRVTITCKASQDVSIGVAWYQQKPGKAPKLLIYSA |
| | | SYRYTGVPSRESGSGSGTDETLIISSLQPEDFATYYCQQYYIYPYTEGQGT |
| | | KVEIKRTVAAPSVFIFPPSDEQLKSGTASVVCLLNNFYPREAKVQWKVDNA |
| | | LQSGNSQESVTEQDSKDSTYSISSTLTLSKADYEKHKVYACEVTHQGLSSP |
| | | VIKSENRGEC* |
| 332 | HC_A | EVQLVESGGGLVQPGGSLRLSCAASGETETDYTMDWVRQAPGKGLEWVADV |
| | | NPNSGGSIYNQRFKGRFTLSVDRSKNTLYLQMNSLRAEDTAVYYCARNIGP |
| | | SFYFDYWGQGTLVTVSSASTKGPSVFPLAPSSKSTSGGTAALGCLVKDYFP |
| | | EPVTVSWNSGALTSGVHTEPAVLQSSGLYSLSSVVTVPSSSIGTQTYICNV |
| | | NIKPSNTKVDKKVEPKSCDKTHTCPPCPAPELLGGPSVFLFPPKPKDTLMI |
| | | SRTPEVTCVVVDVSHEDPEVKENWYVDGVEVHNAKIKPREEQYASTYRVVS |
| | | VLIVLHQDWINGKEYKCKVSNKALPAPIEKTISKAKGQPREPQVYTIPPSR |
| | | KKLTKNQVKLTCLVKGFYPSDIAVEWESNGQPENNYKTIPPVLDCDGSFFL |
| | | YSKLTVDKSRWQQGNVFSCSVMHEALHNHYTQKSLSLSPGK* |
| 341 | HC_B | DIQLIQSPSSLSASVGDRVTITCRASQSVDFHGISFLHWYQQKPGKAPKLL |
| | | IYAASSLQSGVPSRESGSGSGTDFILTISSLQPEDFATYYCQQSYRSPRTF |
| | | GQGIKVEIKGGGGSGGGGSGGGGSGGGGSEVQLVESGGGLVQPGGSLRLSC |
| | | AASGFSLSTSGVGVSWIRQAPGKGLEWIGEIYHSGSTYYNPSLKSRVTISR |
| | | DNSKNTLYLQLNSLRAEDTAVYYCARYEYGYYAFDYWGQGTLVTVSSEPKS |
| | | SDKTHTCPPCPAPELLGGPSVFLEPPKPKDTLMISRTPEVTCVVVDVSHED |
| | | PEVKENWYVDGVEVHNAKTKPREEQYASTYRVVSVLTVLHQDWLNGKEYKC |
| | | KVSNKALPAPIEKTISKAKGQPREPQVYTDPPSRDELIKNQVSITCLVDGF |
| | | YPSDIAVEWESNGQPENCYKTTPPVLDSDGSFFLYSKLTVDKSRWQQGNVE |
| | | SCSVMHEALHNHYTQDSLSLSPGK* |

TY28660

| | | |
|---|---|---|
| 331 | LC | DIQMTQSPSSLSASVGDRVTITCKASQDVSIGVAWYQQKPGKAPKLLIYSA |
| | | SYRYTGVPSRFSGSGSGTDFTLTISSLQPEDFATYYCQQYYTYPYTEGQGT |
| | | KVEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VIKSENRGEC* |
| 332 | HC_A | EVQLVESGGGLVQPGGSLRLSCAASGFTFTDYTMDWVRQAPGKGLEWVADV |
| | | NPNSGGSIYNQRFKGRFTLSVDRSKNTLYLQMNSLRAEDTAVYYGARNLGP |
| | | SFYFDYWGQGTLVTVSSASTKGPSVFPLAPSSKSTSGGTAALGCLVKDYFP |
| | | EPVTVSWNSGALTSGVHTTPAVLQSSGLYSLSSVVTVPSSSIGTQTYICNV |
| | | NHKPSNTKVDKKVEPKSCDKTHTCPPCPAPELLGGPSVFLEPPKPKDILMI |
| | | SRTPEVTCVVVDVSHEDPEVKENWYVDGVEVHNAKIKPREEQYASTYRVVS |
| | | VLIVLHQDWINGKEYKCKVSNKALPAPIEKTISKAKGQPREPQVYTLPPSR |
| | | KKLIKNQVKLTCLVKGFYPSDIAVEWESNGQPENNYKTTPPVLDCDGSFEL |
| | | YSKLTVDKSRWQQGNVFSCSVMHEALHNHYTQKSESLSPGK* |
| 342 | HC_B | DIQLTQSPSSLSASVGDRVTITCRASQSVDEHGKSFLHWYQQKPGKAPKLL |
| | | IYAASSLQSGVPSRFSGSGSGTDFTLTISSLQPEDFATYYCQQSYRSPRTF |
| | | GQGTKVEIKGGGGSGGGGSGGGGSGGGGSEVQLVESGGGLVQPGGSLRLSC |
| | | AASGFSISTSGVGVSWIRQAPGKGLEWIGEIYHSGSTYYSPSLKSRVTISR |
| | | DNSKNTLYLQINSLRAEDTAVYYCARYEYYSYAFDYWGQGTLVTVSSEPKS |
| | | SDKTHTCPPCPAPELLGGPSVFLFPPKPKDTLMISRTPEVTCVVVDVSHED |
| | | PEVKFNWYVDGVEVHNAKTKPREEQYASTYRVVSVLTVLHQDWINGKEYKC |
| | | KVSNKALPAPIEKTISKAKGQPREPQVYTDPPSRDELTKNQVSLTCLVDGE |
| | | YPSDIAVEWESNGQPENCYKTTPPVLDSDGSIFLYSKLTVDKSRWQQGNVF |
| | | SCSVMHEALHNHYTQDSLSLSPGK* |

TY28661

| | | |
|---|---|---|
| 331 | LC | DIQMTQSPSSLSASVGDRVTITCKASQDVSIGVAWYQQKPGKAPKLLIYSA |
| | | SYRYTGVPSRESGSGSGTDETLTISSLQPEDFATYYCQQYYTYPYTEGQGT |
| | | KVEIKRTVAAPSVFIFPPSDEQLKSGTASVVCLINNFYPREAKVQWKVDNA |
| | | LQSGNSQESVTEQDSKDSTYSLSSTLTLSKADYEKHKVYACEVTHQGLSSP |
| | | VTKSFNRGEC* |
| 332 | HC_A | EVQLVESGGGLVQPGGSLRLSCAASGETETDYTMDWVRQAPGKGLEWVADV |
| | | NPNSGGSIYNQRFKGRFTLSVDRSKNTLYLQMNSLRAEDTAVYYCARNIGP |
| | | SFYFDYWGQGTLVTVSSASTKGPSVFPLAPSSKSTSGGTAALGCLVKDYFP |
| | | EPVTVSWNSGALTSGVHTTPAVLQSSGLYSLSSVVTVPSSSLGTQTYICNV |
| | | NHKPSNTKVDKKVEPKSCDKTHTCPPCPAPELLGGPSVELFPPKPKDTLMI |
| | | SRTPEVTCVVVDVSHEDPEVKFNWYVDGVEVHNAKTKPREBQYASTYRVVS |
| | | VLIVLHQDWINGKEYKCKVSNKALPAPIEKTISKAKGQPREPQVYTIPPSR |
| | | KKLTKNQVKLTCLVKGFYPSDIAVEWESNGQPENNYKTIPPVLDCDGSFFL |
| | | YSKLTVDKSRWQQGNVFSCSVMHEALHNHYTQKSLSLSPGK* |
| 343 | HC. B | DIQLTQSPSSLSASVGDRVTITCRASQSVDFYGISFLHWYQQKPGKAPKLL |
| | | IYAASSLESGVPSRFSGSGSGTDFTLTISSLQPEDFATYYCQQSYRTPLTF |
| | | GQGTKVEIKGGGGSGGGGSGGGGSGGGGSEVQLVESGGGLVQPGGSLRLSC |
| | | AASGFSLSTGGVGVSWIRQAPGKGLEWIGSIYHSGSTYYSPSLKSRVTISR |
| | | DNSKNTLYLQLNSLRAEDTAVYYCARYEYDYYAFDYWGQGTLVTVSSEPKS |
| | | SDKTHTCPPCPAPELLGGPSVFLFPPKPKDTIMISRTPEVTCVVVDVSHED |
| | | PEVKFNWYVDGVEVHNAKTKPREEQYASTYRVVSVLIVLHQDNINGKEYKC |
| | | KVSNKALPAPIEKTISKAKGQPREPQVYTDPPSRDELTKNQVSLICLVDGE |
| | | YPSDIAVEWESNGQPENCYKITPPVLDSDGSFFLYSKLTVDKSRWQQGNVF |
| | | SCSVMHEALHNHYTQDSLSLSPGK* |

HER2XCD28 Bispecific Antibodies (VH-VL)
TY29109 (WT)

| | | |
|---|---|---|
| 331 | LC | DIQMTQSPSSLSASVGDRVTITCKASQDVSIGVAWYQQKPGKAPKLLIYSA |
| | | SYRYTGVPSRFSGSGSGTDETLTISSLQPEDEATYYCQQYYTYPYTEGQGT |
| | | KVEIKRIVAAPSVFIFPPSDEQLKSGTASVVCLINNFYPREAKVQWKVDNA |
| | | LQSGNSQESVTEQDSKDSTYSLSSTLTISKADYEKHKVYACEVTHQGLSSP |
| | | VIKSENRGEC* |
| 332 | HC_A | EVQLVESGGGLVQPGGSLRLSCAASGFTFTDYTMDWVRQAPGKGLEWVADV |
| | | NPNSGGSIYNQRFKGRFTLSVDRSKNTLYLQMNSLRAEDTAVYYCARNLGP |
| | | SFYFDYWGQGTLVTVSSASTKGPSVFPLAPSSKSTSGGTAALGCLVKDYEP |
| | | EPVTVSWNSGALTSGVHTEPAVLQSSGLYSLSSVVTVPSSSIGTQTYICNV |
| | | NHKPSNTKVDKKVEPKSCDKTHTCPPCPAPELLGGPSVFLEPPKPKDTIMI |
| | | SRTPEVTCVVVDVSHEDPEVKENWYVDGVEVHNAKIKPREEQYASTYRVVS |
| | | VLIVLHQDWINGKEYKCKVSNKALPAPIEKTISKAKGQPREPQVYTLPPSR |
| | | KKLTKNQVKLICLVKGFYPSDIAVEWESNGQPENNYKTTPPVLDCDGSEFL |
| | | YSKLTVDKSRWQQGNVFSCSVMHEALHNHYTQKSLSLSPGK* |
| 327 | HC_B | EVQLVESGGGLVQPGSSLRLSCAASGFSLSTGGVGVSWIRQAPGKGLEWIG |
| | | EIYHSGSTYYSPSLKSRVTISRDNSKNILYLQLNSLRAEDTAVYYCARYEY |
| | | YYYDEDYWGQGTLVTVSSGGGGSGGGGSGGGGSGGGGSDIQLTQSPSSLSA |
| | | SVGDRVTITCRASQSVDFHGISELHWYQQKPGKAPKLLIYAASSLQSGVPS |
| | | RFSGSGSGTDFILTISSLQPEDFATYYCQQSYRSPRTFGQGTKVEIKEPKS |
| | | SDKTHTCPPCPAPELLGGPSVFLFPPKPKDTLMISRTPEVTCVVVDVSHED |
| | | PEVKENWYVDGVEVHNAKIKPREEQYASTYRVVSVLTVLHQDWINGKEYKC |
| | | KVSNKALPAPIEKTISKAKGQPREPQVYTDPPSRDELIKNQVSLTCLVDGF |
| | | YPSDIAVEWESNGQPENCYKTIPPVLDSDGSFFLYSKLIVDKSRWQQGNVE |
| | | SCSVMHEALHNHYTQDSLSLSPGK* |

TY29306

| | | |
|---|---|---|
| 331 | LC | DIQMTQSPSSLSASVGDRVTITCKASQDVSIGVAWYQQKPGKAPKLLIYSA |
| | | SYRYTGVPSRESGSGSGIDFTLTISSLQPEDFATYYCQQYYTYPYTEGQGT |
| | | KVEIKRIVAAPSVFIFPPSDEQLKSGTASVVCLINNEYPREAKVQWKVDNA |
| | | LQSGNSQESVTEQDSKDSTYSLSSTLTLSKADYEKHKVYACEVTHQGLSSP |
| | | VIKSENRGEC* |
| 332 | HC_A | EVQLVESGGGLVQPGGSLRLSCAASGFTFTDYTMDWVRQAPGKGLEWVADV |
| | | NPNSGGSIYNQRFKGRFTLSVDRSKNTLYLQMNSLRAEDTAVYYCARNLGP |
| | | SFYEDYWGQGTLVTVSSASTKGPSVFPLAPSSKSTSGGTAALGCLVKDYFP |
| | | EPVTVSWNSGALTSGVHTEPAVLQSSGLYSLSSVVTVPSSSIGTQTYICNV |
| | | NHKPSNTKVDKKVEPKSCDKTHTCPPCPAPELLGGPSVFLEPPKPKDTLMI |
| | | SRTPEVTCVVVDVSHEDPEVKFNWYVDGVEVHNAKIKPREEQYASTYRVVS |
| | | VLIVLHQDWINGKEYKCKVSNKALPAPIEKTISKAKGQPREPQVYTIPPSR |
| | | KKLTKNQVKLTCLVKGFYPSDIAVEWESNGQPENNYKTTPPVLDCDGSFFL |
| | | YSKLTVDKSRWQQGNVFSCSVMHEALHNHYTQKSLSLSPGK* |
| 344 | HC_B | EVQLVESGGGLVQPGGSLRLSCAASGESLSTGGVGVSWIRQAPGKGLEWIG |
| | | SIYHSGSTYYNPSLKSRVTISRDNSKNTLYLQLNSLRAEDTAVYYCARYEY |
| | | DYYAFDYWGQGTLVTVSSGGGGSGGGGSGGGGSGGGGSDIQLTQSPSSLSA |
| | | SVGDRVTITCRASQSVDEYGKSFLHWYQQKPGKAPKLLIYAASSLESGVPS |
| | | RFSGSGSGIDFTLTISSLQPEDFATYYCQQSYRSPPTFGQGTKVEIKEPKS |
| | | SDKTHTCPPCPAPELLGGPSVFLFPPKPKDTLMISRTPEVTCVVVDVSHED |
| | | PEVKENWYVDGVEVHNAKIKPREEQYASTYRVVSVLIVLHQDWINGKEYKC |
| | | KVSNKALPAPIEKTISKAKGQPREPQVYTDPPSRDELTKNQVSLTCLVDGF |
| | | YPSDIAVEWESNGQPENCYKITPPVLDSDGSFFLYSKLTVDKSRWQQGNVE |
| | | SCSVMHEALHNHYTQDSLSLSPGK* |

TY29307

| | | |
|---|---|---|
| 331 | LC | DIQMTQSPSSLSASVGDRVTITCKASQDVSIGVAWYQQKPGKAPKLLIYSA |
| | | SYRYTGVPSRFSGSGSGIDFILTISSLQPEDFATYYCQQYYIYPYTFGQGT |
| | | KVEIKRTVAAPSVFIFPPSDEQLKSGTASVVCLINNFYPREAKVQWKVDNA |
| | | LQSGNSQESVTEQDSKDSTYSLSSTLTLSKADYEKHKVYACEVTHQGLSSP |
| | | VIKSENRGEC* |
| 332 | HC_A | EVQLVESGGGLVQPGSSLRISCAASGFIFTDYTMDWVRQAPGKGLEWVADV |
| | | NPNSGGSIYNQRFKGRFTLSVDRSKNTLYLQMNSLRAEDTAVYYCARNLGP |
| | | SFYEDYWGQGTLVTVSSASTKGPSVFPLAPSSKSTSGGTAALGCLVKDYFP |
| | | EPVTVSWNSGALTSGVHTTPAVLQSSGLYSLSSVVTVPSSSIGTQTYICNV |
| | | NHKPSNTKVDKKVEPKSCDKTHTCPPCPAPELLGGPSVELFPPKPKDTLMI |
| | | SRTPEVTCVVVDVSHEDPEVKENWYVDGVEVHNAKIKPREEQYASTYRVVS |
| | | VITVLHQDWINGKEYKCKVSNKALPAPIEKTISKAKGQPREPQVYTLPPSR |
| | | KKLTKNQVKLTCLVKGFYPSDIAVEWESNGQPENNYKTIPPVLDCDGSFEL |
| | | YSKLTVDKSRWQQGNVESCSVMHEALHNHYTQKSLSLSPGK* |
| 345 | HC_B | EVQLVESGGGLVQPGGSLRISCAASGFSLSIGGVGVSWIRQAPGKGLEWIG |
| | | SIYHSGSTYYNPSLKSRVTISRDNSKNILYLQLNSLRAEDTAVYYCARYEY |
| | | GYYALDYWGQGTLVTVSSGGGGSGGGGSGGGGSGGGGSDIQLTQSPSSLSA |
| | | SVGDRVTITGRASQSVDFYGISELHWYQQKPGKAPKLLIYAASSLQSGVPS |
| | | RFSGSGSGTDFTLTISSLQPEDFATYYCQQSYRTPPTFGQGIKVEIKEPKS |
| | | SDKTHTCPPCPAPELLGGPSVFLFPPKPKDTIMISRTPEVTCVVVDVSHED |
| | | PEVKFNWYVDGVEVHNAKIKPREEQYASTYRVVSVLIVLHQDWINGKEYKC |
| | | KVSNKALPAPIEKTISKAKGQPREPQVYTDPPSRDELTKNQVSLTCLVDGF |
| | | YPSDIAVEWESNGQPENCYKTTPPVLDSDGSFFLYSKLTVDKSRWQQGNVE |
| | | SCSVMHEALHNHYTQDSLSLSPGK* |

TY29308

| | | |
|---|---|---|
| 331 | LC | DIQMTQSPSSLSASVGDRVTITCKASQDVSIGVAWYQQKPGKAPKLLIYSA |
| | | SYRYTGVPSRESGSGSGTDFTLTISSLQPEDFATYYCQQYYTYPYTEGQGT |
| | | KVEIKRTVAAPSVFIFPPSDEQLKSGTASVVCLINNEYPREAKVQWKVDNA |
| | | LQSGNSQESVTEQDSKDSTYSLSSTLILSKADYEKHKVYACEVTHQGLSSP |
| | | VIKSENRGEC* |
| 332 | HC_A | EVQLVESGGGLVQPGGSLRLSCAASGFTFTDYTMDWVRQAPGKGLEWVADV |
| | | NPNSGGSIYNQRFKGRFTLSVDRSKNTLYLQMNSLRAEDTAVYYCARNLGP |
| | | SFYFDYWGQGTLVTVSSASTKGPSVFPLAPSSKSTSGGTAALGCLVKDYFP |
| | | EPVTVSWNSGALISGVHTFPAVLQSSGLYSLSSVVTVPSSSIGTQTYICNV |
| | | NHKPSNTKVDKKVEPKSCDKTHTCPPCPAPELLGGPSVFLEPPKPKDTLMI |
| | | SRTREVTCVVVDVSHEDPEVKENWYVDGVEVHNAKIKPREEQYASTYRVVS |
| | | VLTVLHQDWINGKEYKCKVSNKALPAPIEKTISKAKGQPREPQVYTLPPSR |
| | | KKLTKNQVKLTCLVKGFYPSDIAVEWESNGQPENNYKITPPVLDCDGSFEL |
| | | YSKLTVDKSRWQQGNVFSCSVMHEALHNHYTQKSLSLSPGK* |
| 346 | HC_B | EVQLVESGGGLVQPGGSLRLSCAASGFSLSTSGVGVSWIRQAPGKGLEWIG |
| | | SIYHSGSTYYNPSLKSRVTISRDNSKNTLYLQLNSLRAEDTAVYYCARYEY |
| | | GYYAFDYWGQGTLVTVSSGGGGSGGGGSGGGGSGGGGSDIQLTQSPSSLSA |
| | | SVGDRVTITCRASQSVDFYGKSELHWYQQKPGKAPKLLIYAASSLQSGVPS |
| | | RESGSGSGIDETLTISSLQPEDFATYYCQQSYRTPLIEGQGTKVEIKEPKS |
| | | SDKTHTCPPCPAPELLGGPSVFLEPPKPKDTLMISRTPEVTCVVVDVSHED |
| | | PEVKFNWYVDGVEVHNAKIKPREEQYASTYRVVSVLIVLHQDWINGKEYKC |
| | | KVSNKALPAPIEKTISKAKGQPREPQVYTDPPSRDELTKNQVSLTCLVDGE |
| | | YPSDIAVEWESNGQPENCYKTTPPVLDSDGSFFLYSKLTVDKSRWQQGNVF |
| | | SCSVMHEALHNHYTQDSLSLSPGK* |

TY29309

| | | |
|---|---|---|
| 331 | LC | DIQMTQSPSSLSASVGDRVTITCKAS@DVSIGVAWYQQKPGKAPKLLIYSA |
| | | SYRYTGVPSRESGSGSGTDFILTISSLQPEDFATYYCQQYYTYPYTEGQGT |
| | | KVEIKRTVAAPSVFIFPPSDEQLKSGTASVVCLINNFYPREAKVQWKVDNA |
| | | LQSGNSQESVTEQDSKDSTYSLSSTLILSKADYEKHKVYACEVTHQGLSSP |
| | | VIKSENRGEC* |
| 332 | HC_A | EVQLVESGGGLVQPGGSIRLSCAASGFTFTDYTMDWVRQAPGKGLEWVADV |
| | | NPNSGGSIYNQRFKGRETLSVDRSKNTLYLQMNSLRAEDTAVYYCARNLGP |
| | | SEYEDYWGQGILVTVSSASTKGPSVIPLAPSSKSTSGGTAALGCLVKDYFP |
| | | EPVTVSWNSGALTSGVHTFPAVLQSSGLYSLSSVVTVPSSSIGTQTYICNV |
| | | NHKPSNIKVDKKVEPKSCDKTHTCPPCPAPELLGGPSVELFPPKPKDTIMI |
| | | SRTPEVTCVVVDVSHEDPEVKENWYVDGVEVHNAKTKPREEQYASTYRVVS |
| | | VLTVLHQDWINGKEYKCKVSNKALPAPIEKTISKAKGQPREPQVYTLPPSR |
| | | KKLIKNQVKLTCLVKGFYPSDIAVEWESNGQPENNYKITPPVLDCDGSFFL |
| | | YSKLTVDKSRWQQGNVESCSVMHEALHNHYTQKSLSLSPGK* |
| 347 | HC_B | EVQLVESGGGLVQPGGSLRLSCAASGESLSTSGVGVSWIRQAPGKGLEWIG |
| | | SIYHSGSTYYNPSLKSRVTISRDNSKNILYLQLNSLRAEDTAVYYCARYEY |
| | | DYYAFDYWGQGTLVIVSSGGGGSGGGGSGGGGSGGGGSDIQLTQSPSSLSA |
| | | SVGDRVTITCRASQSVDFYGISELHWYQQKPGKAPKLLIYAASSLQSGVPS |
| | | RESGSGSGTDFTLTISSLQPEDFATYYCQQSYRTPRIFGQGTKVEIKEPKS |
| | | SDKTHTCPPCPAPELLGGPSVFLEPPKPKDTIMISRTPEVTCVVVDVSHED |
| | | PEVKFNWYVDGVEVHNAKTKPREEQYASTYRVVSVLIVLHQDWINGKEYKC |
| | | KVSNKALPAPIEKTISKAKGQPREPQVYTDPPSRDELIKNQVSLTCLVDGE |
| | | YPSDIAVEWESNGQPENCYKTTPPVLDSDGSEFLYSKLTVDKSRWQQGNVE |
| | | SCSVMHEALHNHYTQDSLSLSPGK* |

TY29310

| | | |
|---|---|---|
| 331 | LC | DIQMTQSPSSLSASVGDRVTITCKASQDVSIGVAWYQQKPGKAPKLLIYSA |
| | | SYRYTGVPSRFSGSGSGIDFILTISSLQPEDFATYYCQQYYTYPYTEGQGT |
| | | KVEIKRIVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VIKSENRGEC* |
| 332 | HC. A | EVQLVESGGGLVQPGGSLRLSCAASGFTFTDYTMDWVRQAPGKGLEWVADV |
| | | NPNSGGSIYNQRFKGRFTLSVDRSKNTLYLQMNSLRAEDTAVYYCARNLGP |
| | | SFYFDYWGQGTLVTVSSASTKGPSVFPLAPSSKSTSGGTAALGCLVKDYEP |
| | | EPVTVSWNSGALTSGVHTEPAVLQSSGLYSLSSVVTVPSSSIGTQTYICNV |
| | | NHKPSNTKVDKKVEPKSCDKTHTCPPCPAPELLGGPSVFLFPPKPKDTLMI |
| | | SRIPEVICVVVDVSHEDPEVKENWYVDGVEVHNAKTKPREEQYASTYRVVS |
| | | VLIVLHQDWLNGKEYKCKVSNKALPAPIEKTISKAKGQPREPQVYTLPPSR |
| | | KKLTKNQVKLTCLVKGFYPSDIAVEWESNGQPENNYKTTPPVLDCDGSFFL |
| | | YSKLTVDKSRWQQGNVFSCSVMHEALHNHYTQKSLSLSPGK* |
| 348 | HC_B | EVQLVESGGGLVQPGGSLRLSCAASGESLSTSGVGVGWIRQAPGKGLEWIG |
| | | EIYHSGSTNYNPSLKSRVTISRDNSKNTLYLQINSLRAEDTAVYYCARYEY |
| | | GYYALDYWGQGTLVTVSSGGGGSGGGGSGGGGSGGGGSDIQLTQSPSSLSA |
| | | SVGDRVTITCRASQSVDFYGKSFLHWYQQKPGKAPKLLIYAASSLQSGVPS |
| | | RESGSGSGTDFTLTISSLQPEDFATYYCQQSYRTPRTFGQGTKVEIKEPKS |
| | | SDKTHTCPPCPAPELLGGPSVFLFPPKPKDTIMISRTPEVTCVVVDVSHED |
| | | PEVKENWYVDGVEVHNAKTKPREEQYASTYRVVSVLTVLHQDWLNGKEYKC |
| | | KVSNKALPAPIEKTISKAKGQPREPQVYTDPPSRDELIKNQVSLTCLVDGE |
| | | YPSDIAVEWESNGQPENCYKTIPPVLDSDGSEFLYSKLTVDKSRWQQGNVE |
| | | SCSVMHEALHNHYTQDSLSLSPGK* |

TY29311

| | | |
|---|---|---|
| 331 | LC | DIQMTQSPSSLSASVGDRVTITCKASQDVSIGVAWYQQKPGKAPKLLIYSA |
| | | SYRYIGVPSRESGSGSGIDFILTISSLQPEDFATYYCQQYYTYPYTEGQGT |
| | | KVEIKRIVAAPSVEIFPPSDEQLKSGTASVVCLINNFYPREAKVQWKVDNA |
| | | LQSGNSQESVTEQDSKDSTYSLSSTLTLSKADYEKHKVYACEVTHQGLSSP |
| | | VTKSENRGEC* |
| 332 | HC_A | EVQLVESGGGLVQPGGSLRLSCAASGETFTDYTMDWVRQAPGKGLEWVADV |
| | | NPNSGGSIYNQRFKGRETLSVDRSKNTLYLQMNSLRAEDTAVYYCARNIGP |
| | | STYFDYWGQGTLVIVSSASTKGPSVEPLAPSSKSTSGGTAALGCLVKDYFP |
| | | EPVTVSWNSGALTSGVHIFPAVLQSSGLYSLSSVVTVPSSSIGTQTYICNV |
| | | NHKPSNTKVDKKVEPKSCDKTHTCPPCPAPELLGGPSVFLFPPKPKDTLMI |
| | | SRTPEVICVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPSR |
| | | KKLTKNQVKLTCLVKGFYPSDIAVEWESNGQPENNYKTTPPVLDCDGSEFL |
| | | YSKLTVDKSRWQQGNVFSCSVMHEALHNHYTQKSLSLSPGK* |
| 349 | HC_B | EVQLVESGGGLVQPGGSIRLSCAASGESLSTGGVGVGWIRQAPGKGLEWIG |
| | | EIYHSGSTYYNPSLKSRVTISRDNSKNTLYLQLNSLRAEDTAVYYCARYEY |
| | | GYYAFDYWGQGTLVTVSSGGGGSGGGGSGGGGSGGGGSDIQLTQSPSSLSA |
| | | SVGDRVTITCRASQSVDFHGISFLHWYQQKPGKAPKLLIYAASSLESGVPS. |
| | | RESGSGSGTDFTLTISSLQPEDFATYYCQQSYRIPRTFGQGTKVEIKEPKS |
| | | SDKTHTCPPCPAPELLGGPSVFLFPPKPKDTLMISRTPEVTCVVVDVSHED |
| | | PEVKFNWYVDGVEVHNAKIKPREEQYASTYRVVSVLTVLHQDWLNGKEYKC |
| | | KVSNKALPAPIEKTISKAKGQPREPQVYTDPPSRDELTKNQVSLTCLVDGF |
| | | YPSDIAVEWESNGQPENCYKTIPPVLDSDGSFFLYSKLTVDKSRWQQGNVE |
| | | SCSVMHEALHNHYTQDSLSLSPGK* |

TY29312

| | | |
|---|---|---|
| 331 | LC | DIQMTQSPSSLSASVGDRVTITCKASQDVSIGVAWYQQKPGKAPKLLIYSA |
| | | SYRYTGVPSRESGSGSGIDETLTISSLQPEDFATYYCQQYYTYPYTEGQGT |
| | | KVEIKRIVAAPSVFIFPPSDEQLKSGTASVVCLUNNFYPREAKVQWKVDNA |
| | | LQSGNSQESVTEQDSKDSTYSLSSTLTLSKADYEKHKVYACEVTHQGLSSP |
| | | VTKSFNRGEC* |
| 332 | HC_A | EVQLVESGGGLVQPGGSIRLSCAASGFTFTDYTMDWVRQAPGKGLEWVADV |
| | | NPNSGGSIYNQRFKGRFTLSVDRSKNTLYLQMNSLRAEDTAVYYCARNLGP |
| | | SEYEDYWGQGTLVTVSSASTKGPSVFPLAPSSKSTSGGTAALGCLVKDYFP |
| | | EPVTVSWNSGALISGVHTEPAVLQSSGLYSLSSVVTVPSSSLGTQTYICNV |
| | | NHKPSNTKVDKKVEPKSCDKTHTCPPCPAPELLGGPSVFLEPPKPKDTLMI |
| | | SRTPEVTCVVVDVSHEDPEVKENWYVDGVEVHNAKTKPREEQYASTYRVVS |
| | | VLIVLHQDWINGKEYKCKVSNKALPAPIEKTISKAKGQPREPQVYTLPPSR |
| | | KKLTKNQVKLICLVKGFYPSDIAVEWESNGQPENNYKTTPPVLDCDGSFFL |
| | | YSKLTVDKSRWQQGNVESCSVMHEALHINHYTQKSLSLSPGK* |
| 350 | HC_B | EVQLVESGGGLVQPGGSLRLSCAASGFSLSTSGVGVGWIRQAPGKGLEWIG |
| | | SIYHSGSTYYNPSLKSRVTISRDNSKNTLYLQLNSLRAEDTAVYYCARYEY |
| | | DYYDFDYWGQGTLVTVSSGGGGSGGGGSGGGGSGGGGSDIQLTQSPSSLSA |
| | | SVGDRVTITCRASQSVDFYGKSFLHWYQQKPGKAPKLLIYAASSLESGVPS |
| | | RESGSGSGTDETLTISSLQPEDFATYYCQQSYRSPLIEGQGTKVEIKEPKS |
| | | SDKTHTCPPCPAPELLGGPSVFLFPPKPKDTLMISRTPEVTCVVVDVSHED |
| | | PEVKENWYVDGVEVHNAKIKPREEQYASTYRVVSVLIVLHQDWINGKEYKC |
| | | KVSNKALPAPIEKTISKAKGQPREPQVYIDEPSRDELIKNQVSLTCLVDGE |
| | | YPSDIAVEWESNGQPENCYKTTPPVLDSDGSFFLYSKLTVDKSRWQQGNVE |
| | | SCSVMHEALHNHYTQDSLSLSPGK* |

TY29313

| | | |
|---|---|---|
| 331 | LC | DIQMTQSPSSLSASVGDRVTITCKASQDVSIGVAWYQQKPGKAPKLLIYSA |
| | | SYRYTGVPSRESGSGSGIDFTLTISSLQPEDFATYYCQQYYTYPYTEGQGT |
| | | KVEIKRTVAAPSVFIEPPSDEQLKSGIASVVCLINNFYPREAKVQWKVDNA |
| | | LQSGNSQESVTEQDSKDSTYSLSSTLILSKADYEKHKVYACEVTHQGLSSP |
| | | VIKSFNRGEC* |
| 332 | HC_A | EVQLVESGGGLVQPGGSLRLSCAASGETFTDYTMDWVRQAPGKGLEWVADV |
| | | NPNSGGSTYNQRFKGRETLSVDRSKNTLYLQMNSLRAEDTAVYYCARNLGP |
| | | SFYFDYWGQGTLVTVSSASTKGPSVTPLAPSSKSTSGGTAALGCLVKDYFP |
| | | EPVTVSWNSGALTSGVHTFPAVLQSSGLYSLSSVVIVPSSSIGTQTYICNV |
| | | NHKPSNTKVDKKVEPKSCDKTHTCPPCPAPELLGGPSVFLFPPKPKDTLMI |
| | | SRTPEVTCVVVDVSHEDPEVKENWYVDGVEVHNAKTKPREEQYASTYRVVS |
| | | VLTVLHQDWINGKEYKCKVSNKALPAPIEKTISKAKGQPREPQVYTLPPSR |
| | | KKLTKNQVKLICLVKGFYPSDIAVEWESNGQPENNYKTTPPVLDCDGSFFL |
| | | YSKLTVDKSRWQQGNVFSCSVMHEALHNHYTQKSLSLSPGK* |
| 351 | HC_B | EVQLVESGGGLVQPGGSLRLSCAASGFSISTSGVGVSWIRQAPGKGLEWIG |
| | | EIYHSGSTYYNPSLKSRVTISRDNSKNTLYLQLNSLRAEDTAVYYCARYEY |
| | | GYYAEDYWGQGTLVTVSSGGGGSGGGGSGGGGSGGGGSDIQLTQSPSSLSA |
| | | SVGDRVTITGRASQSVDFHGISFLHWYQQKPGKAPKLLIYAASSLQSGVPS |
| | | RESGSGSGIDETLTISSLQPEDFATYYCQQSYRSPRIEGQGTKVEIKEPKS |
| | | SDKTHTCPPCPAPELLGGPSVFLFPPKPKDTLMISRTPEVTCVVVDVSHED |
| | | PEVKENWYVDGVEVHNAKIKPREEQYASTYRVVSVLIVLHQDWLNGKEYKC |
| | | KVSNKALPAPIEKTISKAKGQPREPQVYTDPPSRDELTKNQVSLTCLVDGE |
| | | YPSDIAVEWESNGQPENCYKTIPPVLDSDGSFFLYSKLTVEKSRWQQGNVE |
| | | SCSVMHEALHNHYTQDSLSLSPGK* |

TY29314

| | | |
|---|---|---|
| 331 | LC | DIQMTQSPSSLSASVGDRVTITCKASQDVSIGVAWYQQKPGKAPKLLIYSA |
| | | SYRYTGVPSRFSGSGSGIDETLTISSLQPEDFATYYCQQYYTYPYTFGQGT |
| | | KVEIKRTVAAPSVFIFPPSDEQLKSGTASVVCLINNFYPREAKVQWKVDNA |
| | | LQSGNSQESVTEQDSKDSTYSISSTLILSKADYEKHKVYACEVTHQGLSSP |
| | | VTKSFNRGEC* |
| 332 | HC_A | EVQLVESGGGLVQPGGSLRLSCAASGFTETDYTMDWVRQAPGKGLEWVADV |
| | | NPNSGGSIYNQRFKGRETLSVDRSKNILYLQMNSLRAEDTAVYYCARNLGP |
| | | SFYFDYWGQGTLVTVSSASTKGPSVFPLAPSSKSTSGGTAALGCLVKDYFP |
| | | EPVTVSWNSGALTSGVHTYPAVLQSSGLYSLSSVVTVPSSSLGIQTYICNV |
| | | NHKPSNTKVDKKVEPKSCDKTHTCPPCPAPELLGGPSVFLFPPKPKDTLMI |
| | | SRTPEVTCVVVDVSHEDPEVKENNYVDGVEVHNAKIKPREEQYASTYRVVS |
| | | VLTVLHQDWLNGKEYKCKVSNKALPAPIEKTISKAKGQPREPQVYTIPPSR |
| | | KKLTKNQVKLTCLVKGFYPSDIAVEWESNGQPENNYKTIPPVLDCDGSFFL |
| | | YSKLTVDKSRWQQGNVFSCSVMHEALHNHYTQKSLSLSPGK* |
| 352 | HC_B | EVQLVESGGGEVQPGGSLRISCAASGFSLSTSGVGVSWIRQAPGKGLEWIG |
| | | ETYHSGSTYYSPSLKSRVTISRDNSKNILYLQLNSLRAEDTAVYYCARYEY |
| | | YSYAFDYWGQGTLVTVSSGGGGSGGGGSGGGGSGGGGSDIQLTQSPSSLSA |
| | | SVGDRVTITCRASQSVDEHGKSELHWYQQKPGKAPKLLIYAASSLQSGVPS |
| | | RFSGSGSGTDFTLTISSLQPEDFATYYCQQSYRSPRTFGQGTKVEIKEPKS |
| | | SDKTHTCPPCPAPELLGGPSVFLFPPKPKDTLMISRTPEVTCVVVDVSHED |
| | | PEVKINWYVDGVEVHNAKTKPREEQYASTYRVVSVLTVLHQDWINGKEYKC |
| | | KVSNKALPAPIEKTISKAKGQPREPQVYTDPPSRDELTKNQVSLTCLVDGE |
| | | YPSDIAVEWESNGQPENCYKTTPPVLDSDGSEFLYSKLTVDKSRWQQGNVF |
| | | SCSVMHEALHNHYTQDSLSLSPGK* |

TY29315

| | | |
|---|---|---|
| 331 | LC | DIQMTQSPSSLSASVGDRVTITCKASQDVSIGVAWYQQKPGKAPKLLIYSA |
| | | SYRYTGVPSRESGSGSGTDETLTISSLQPEDFATYYCQQYYIYPYTEGQGT |
| | | KVEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VIKSENRGEC* |
| 332 | HC_A | EVQLVESGGGLVQPGGSLRLSCAASGFTFTDYTMDWVRQAPGKGLEWVADV |
| | | NPNSGGSIYNQRFKGRFTLSVDRSKNTLYLQMNSLRAEDTAVYYCARNLGP |
| | | SFYEDYWGQGILVTVSSASTKGPSVFPLAPSSKSTSGGTAALGCLVKDYFP |
| | | EPVTVSWNSGALTSGVHTTPAVLQSSGLYSLSSVVTVPSSSIGTQTYICNV |
| | | NHKPSNTKVDKKVEPKSCDKTHTCPPCPAPELLGGPSVFLEPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPSR |
| | | KKLTKNQVKLTCLVKGFYPSDIAVEWESNGQPENNYKITPPVLDCDGSFFL |
| | | YSKLTVDKSRWQQGNVFSCSVMHEALHNHYTQKSLSLSPGK* |
| 353 | HC_B | EVQLVESGGGLVQPGGSLRLSCAASGESLSTGGVGVSWIRQAPGKGLEWIS |
| | | SIYHSGSTYYSPSLKSRVTISRDNSKNTLYLQINSLRAEDTAVYYCARYEY |
| | | DYYAFDYWGQGTLVTVSSGGGGSGGGGSGGGGSGGGGSDIQLTQSPSSLSA |
| | | SVGDRVTITGRASQSVDFYGISFLHWYQQKPGKAPKLLIYAASSLESGVPS |
| | | RESGSGSGTDETLTISSLQPEDFATYYCQQSYRTPLTFGQGTKVEIKEPKS |
| | | SDKTHTCPPCPAPELLGGPSVFLFPPKPKDTLMISRTPEVTCVVVDVSHED |
| | | PEVKFNWYVDGVEVHNAKIKPREEQYASTYRVVSVLTVLHQDWINGKEYKC |
| | | KVSNKALPAPIEKTISKAKGQPREPQVYIDPPSRDELTKNQVSLTCLVDGE |
| | | YPSDIAVEWESNGQPENCYKITPPVLDSDGSFFLYSKLTVDKSRWQQGNVE |
| | | SCSVMHEALHNHYTQDSLSLSPGK* |

B7H3XCD28 Bispecific Antibodies (VH-VL)
TY29021 (WT)

| | | |
|---|---|---|
| 354 | LC | DIQLTQSPSSLSASVGDRVTITCRASQSIRKYLNWYQQKPGKAPKLLIYAA |
| | | STLQSGVPSRESGSGSGIDETLTISSLQPEDFATYFCSQGTHVPFTFGQGT |
| | | KVEIKRIVAAPSVFIFPPSDEQLKSGTASVVCLINNFYPREAKVQWKVDNA |
| | | LQSGNSQESVTEQDSKDSTYSLSSTLTLSKADYEKHKVYACEVTHQGLSSP |
| | | VIKSENRGEC* |
| 355 | HC_A | EVQLVESGGGLVQPGGSLRISCAASGFTESGYGIHWVRQAPGKGLEWIGWI |
| | | SPSGGDTKYAQKFQGRVTISRDNSKNTLYLQLNSLRAEDTAVYYCARGGTV |
| | | VYFDYWGQGTLVTVSSASTKGPSVFPLAPSSKSTSGGTAALGCLVKDYFPE |
| | | PVTVSWNSGALISGVHTEPAVLQSSGLYSLSSVVTVPSSSLGTQTYICNVN |
| | | HKPSNTKVDKKVEPKSCDKTHTCPPCPAPELLGGPSVFLFPPKPKDTLMIS |
| | | RTPEVTCVVVDVSHEDPEVKENWYVDGVEVHNAKTKPREEQYASTYRVVSV |
| | | ITVLHQDWLNGKEYKCKVSNKALPAPIEKTISKAKGQPREPQVYTLPPSRK |
| | | KLTKNQVKLTCLVKGFYPSDIAVEWESNGQPENNYKTTPPVLDCDGSFFLY |
| | | SKLTVDKSRWQQGNVFSCSVMHEALHNHYTQKSLSLSPGK* |
| 327 | HC_B | EVQLVESGGGLVQPGGSIRLSCAASGESLSIGGVGVSWIRQAPGKGLEWIG |
| | | EIYHSGSTYYSPSLKSRVTISRDNSKNTLYLQLNSLRAEDTAVYYCARYEY |
| | | YYYDEDYWGQGTLVTVSSGGGGSGGGGSGGGGSGGGGSDIQLTQSPSSLSA |
| | | SVGDRVTITCRASQSVDFHGISELHWYQQKPGKAPKLLIYAASSLQSGVPS |
| | | RFSGSGSGTDFTLTISSLQPEDFATYYCQQSYRSPRIFGQGTKVEIKEPKS |
| | | SDKTHTCPPCPAPELLGGPSVFLEPPKPKDTIMISRTPEVTCVVVDVSHED |
| | | PEVKENWYVDGVEVHNAKIKPREEQYASTYRVVSVLIVLHQDWINGKEYKC |
| | | KVSNKALPAPIEKTISKAKGQPREPQVYTDPPSRDELTKNQVSLICLVDGF |
| | | YPSDIAVEWESNGQPENCYKTTPPVLDSDGSFFLYSKLTVEKSRWQQGNVE |
| | | SCSVMHEALHNHYTQDSLSLSPGK* |

TY30120

| | | |
|---|---|---|
| 354 | LC | DIQLTQSPSSLSASVGDRVTITCRASQSIRKYLNWYQQKPGKAPKLLIYAA |
| | | STLQSGVPSRESGSGSGTDFTLTISSLQPEDFATYFCSQGTHVPFTFGQGT |
| | | KVEIKRIVAAPSVEIFPPSDEQLKSGTASVVCLINNFYPREAKVQWKVDNA |
| | | LQSGNSQESVTEQDSKDSTYSLSSTLTLSKADYEKHKVYACEVTHQGLSSP |
| | | VIKSENRGEC* |
| 355 | HC_A | EVQLVESGGGLVQPGGSIRLSCAASGFTESGYGIHWVRQAPGKGLEWIGWI |
| | | SPSGGDTKYAQKTQGRVTISRDNSKNTLYLQINSLRAEDTAVYYCARGGTV |
| | | VYFDYWGQGTLVTVSSASTKGPSVFPLAPSSKSTSGGTAALGCLVKDYFPE |
| | | PVTVSWNSGALTSGVHTFPAVLQSSGLYSLSSVVTVPSSSIGTQTYICNVN |
| | | HKPSNTKVDKKVEPKSCDKTHTCPPCPAPELLGGPSVELFPPKPKDTIMIS |
| | | RTPEVTCVVVDVSHEDPEVKENWYVDGVEVHNAKTKPREEQYASTYRVVSV |
| | | LIVLHQDWINGKEYKCKVSNKALPAPIEKTISKAKGQPREPQVYTLPPSRK |
| | | KLIKNQVKLTCLVKGFYPSDIAVEWESNGQPENNYKTTPPVLDCDGSFFLY |
| | | SKLTVDKSRWQQGNVFSCSVMHEALHNHYTQKSLSLSPGK* |
| 356 | HC_B | EVQLVESGGGLVQPGGSLRLSCAASGFSLSTSGVGVSWIRQAPGKGLEWIG |
| | | ETYHSGSTYYSPSLKSRVTISRDNSKNTLYLQLNSERAEDTAVYYCARYGY |
| | | YYYDFDYWGQGTLVTVSSGGGGSGGGGSGGGGSGGGGSDIQLTQSPSSLSA |
| | | SVGDRVTITCRASQSVDFHGISFLHWYQQKPGKAPKLLIYAASSLQSGVPS |
| | | RFSGSGSGTDFTLTISSLQPEDFATYYCQQSYRSPRIEGQGTKVEIKEPKS |
| | | SDKTHTCPPCPAPELLGGPSVFLFPPKPKDTIMISRIPEVTCVVVDVSHED |
| | | PEVKENWYVDGVEVHNAKTKPREEQYASTYRVVSVLIVLHQDWINGKEYKC |
| | | KVSNKALPAPIEKTISKAKGQPREPQVYTDPPSRDELTKNQVSLICLVDGE |
| | | YPSDIAVEWESNGQPENCYKTTPPVLDSDGSFFLYSKLTVDKSRWQQGNVE |
| | | SCSVMHEALUNHYTQDSLSLSPGK* |

TY30121

| | | |
|---|---|---|
| 354 | LC | DIQLTQSPSSLSASVGDRVTITCRASQSIRKYLNWYQQKPGKAPKLLIYAA |
| | | STLQSGVPSRESGSGSGTDFTLTISSLQPEDFATYFCSQGTHVPFTFGQGT |
| | | KVEIKRIVAAPSVFIFPPSDEQLKSGTASVVCLINNEYPREAKVQWKVDNA |
| | | LQSGNSQESVTEQDSKDSTYSLSSTLTLSKADYEKHKVYACEVTHQGLSSP |
| | | VIKSENRGEC* |
| 355 | HC | EVQLVESGGGLVQPGGSLRLSCAASGFTESGYGIHWVRQAPGKGLEWIGWI |
| | | SPSGGDTKYAQKFQGRVTISRDNSKNTLYLQINSLRAEDTAVYYCARGGTV |
| | | VYFDYWGQGTLVTVSSASTKGPSVFPLAPSSKSTSGGTAALGCLVKDYFRE |
| | | PVTVSWNSGALTSGVHTFPAVLQSSGLYSLSSVVTVPSSSIGTQTYICNVN |
| | | HKPSNTKVDKKVEPKSCDKTHTCPPCPAPELLGGPSVFLEPPKPKDTLMIS |
| | | RIPEVTCVVVDVSHEDPEVKENWYVDGVEVHNAKTKPREEQYASTYRVVSV |
| | | LTVLHQDWLNGKEYKCKVSNKALPAPIEKTISKAKGQPREPQVYTLPPSRK |
| | | KLTKNQVKLTCLVKGEYPSDIAVEWESNGQPENNYKTIPPVLDCDGSEFLY |
| | | SKLTVDKSRWQQGNVFSCSVMHEALHNHYTQKSLSLSPGK* |
| 357 | HC_B | EVQLVESGGGLVQPGGSLRLSCAASGFSLSTSGVGVGWIRQAPGKGLEWIG |
| | | EIYHSGSTYYNPSLKSRVTISRDNSKNTLYLQLNSLRAEDTAVYYCARYKY |
| | | DYYDFDYWGQGTLVTVSSGGGGSGGGGSGGGGSGGGGSDIQLTQSPSSLSA |
| | | SVGDRVTITGRASQSVDFHGISFLHWYQQKPGKAPKLLIYAASSLQSGVPS |
| | | RFSGSGSGTDETLTISSLQPEDFATYYCQQSYRSPRTFGQGTKVEIKEPKS |
| | | SDKTHTCPPCPAPELLGGPSVELFPPKPKDTIMISRTPEVTCVVVDVSHED |
| | | PEVKENWYVDGVEVHNAKTKPREEQYASTYRVVSVLTVLHQDWLNGKEYKC |
| | | KVSNKALPAPIEKTISKAKGQPREPQVYTDPPSRDELTKNQVSLTCLVDGF |
| | | YPSDIAVEWESNGQPENCYKTTPPVEDSDGSEFLYSKLTVDKSRWQQGNVF |
| | | SCSVMHEALHNHYTQDSLSLSPGK* |

TY30123

| | | |
|---|---|---|
| 354 | LC | DIQLTQSPSSLSASVGDRVTITCRASQSIRKYLNWYQQKPGKAPKLLIYAA |
| | | STLQSGVPSRFSGSGSGTDFLTISSLQPEDEATYFCSQGTHIVPFTFGQGT |
| | | KVEIKRTVAAPSVFIFPPSDEQLKSGTASVVCLINNFYPREAKVQWKVDNA |
| | | LQSGNSQESVTEQDSKDSTYSLSSTLILSKADYEKHKVYACEVTHQGLSSP |
| | | VTKSENRGEC* |
| 355 | HC_A | EVQLVESGGGLVQPGGSLRISCAASGFTFSGYGIHWVRQAPGKGLEWIGWI |
| | | SPSGGDIKYAQKFQGRVTISRDNSKNTLYLQLNSLRAEDTAVYYCARGGTV |
| | | VYFDYWGQGTLVTVSSASTKGPSVFPLAPSSKSTSGGTAALGCLVKDYFPE |
| | | PVTVSWNSGALTSGVHTEPAVLQSSGLYSLSSVVTVPSSSIGTQTYICNVN |
| | | HKPSNTKVDKKVEPKSCDKTHTCPPCPAPELLGGPSVELEPPKPKDTLMIS |
| | | RTPEVTCVVVDVSHEDPEVKFNWYVDGVEVHNAKTKPREEQYASTYRVVSV |
| | | LTVLHQDWLNGKEYKCKVSNKALPAPIEKTISKAKGQPREPQVYTLPPSRK |
| | | KLTKNQVKLTCLVKGFYPSDIAVEWESNGQPENNYKITPPVLDCDGSFFLY |
| | | SKLTVDKSRWQQGNVFSCSVMHEALHINHYTQKSLSLSPGK* |
| 358 | HC_B | EVQLVESGGGLVQPGGSLRLSCAASGFSLSTGGVGVGWIRQAPGKGLEWIG |
| | | ETYHSGSTYYSPSLKSRVTISRDNSKNTLYLQLNSLRAEDTAVYYCARYGY |
| | | YYYDFDYWGQGTLVTVSSGGGGSGGGGSGGGGSGGGGSDIQLTQSPSSLSA |
| | | SVGDRVTITCRASQSVDFHGISELHWYQQKPGKAPKLLIYAASSLQSGVPS |
| | | RFSGSGSGTDFILTISSLQPEDFATYYCQQSYRSPRTFGQGIKVEIKEPKS |
| | | SDKTHTCPPCPAPELLGGPSVFLFPPKPKDTLMISRTPEVTCVVVDVSHED |
| | | PEVKFNWYVDGVEVHNAKIKPREEQYASTYRVVSVLTVLHQDWLNGKEYKC |
| | | KVSNKALPAPIEKTISKAKGQPREPQVYTDPPSRDELTKNQVSLTCLVDGF |
| | | YPSDIAVEWESNGQPENCYKTTPPVLDSDGSEFLYSKLTVDKSRWQQGNVE |
| | | SCSVMHEALHNHYTQDSLSLSPGK* |

1. An antigen-binding protein, or an antigen-binding fragment thereof, comprising an CD28-binding portion, wherein the CD28-binding portion binds human CD28 and is cross-reactive with cynomolgus monkey and mouse CD28.

2. The antigen-binding protein or fragment thereof of claim 1, wherein the CD28-binding portion binds to a CD28 epitope comprising amino acid residues 51-122 of human CD28 (SEQ ID NO: 1).

3. The antigen-binding protein or fragment thereof of claim 2, wherein the CD28 epitope comprises amino acid residues 51, 52, 54, 55, 98-101, 110-111, 113-114, and 118-122 of SEQ ID NO: 1.

4. The antigen-binding protein or fragment thereof of any one of claims 1-3, wherein the CD28-binding portion comprises an antibody heavy chain variable domain (V_{H}) and an antibody light chain variable domain (V_{L}), and wherein the V_{H} and V_{L} comprise heavy chain complementarity-determining regions (CDRs) 1-3 and light chain CDR1-3 set forth in
SEQ ID NOs: 5-10, respectively,
SEQ ID NOs: 15, 6, 16, 17-19, respectively,
SEQ ID NOs: 24, 6, 25, 26-28, respectively,
SEQ ID NOs: 33, 6, 35-38, respectively,
SEQ ID NOs: 43, 6, 44, 45, 9, and 46, respectively,
SEQ ID NOs: 33, 51-53, 300, and 10, respectively,
SEQ ID NOs: 24, 58, 59, 60, 300, and 61, respectively,
SEQ ID NOs: 66-69, 300 and 70, respectively,
SEQ ID NOs: 24, 6, 75, 76, 18, and 28, respectively,
SEQ ID NOs: 24, 58, 81, 82, 27, and 83, respectively,
SEQ ID NOs: 88-91, 300, and 70, respectively,
SEQ ID NOs: 24, 96-98, 9, and 70, respectively,
SEQ ID NOs: 103-106, 18, and 83, respectively,
SEQ ID NOs: 111, 6, 112, 113, 18, and 114, respectively,
SEQ ID NOs: 15, 6, 119, 120, 9, and 121, respectively,
SEQ ID NOs: 126, 67, 127, 128, 18, and 129, respectively,
SEQ ID NOs: 134, 6, 135, 136, 27, and 83, respectively,
SEQ ID NOs: 43, 58, 141, 142, 300, and 143, respectively,
SEQ ID NOs: 148, 6, 149, 150, 300, and 83, respectively,
SEQ ID NOs: 15, 155, 16, 156, 27, and 70, respectively,
SEQ ID NOs: 161, 6, 162, 163, 300, and 164, respectively,
SEQ ID NOs: 5, 96, 307, 76, 308, and 46, respectively,
SEQ ID NOs: 5, 96, 309, 310, 9, and 311, respectively,
SEQ ID NOs: 43, 96, 312, 76, 9, and 28, respectively,
SEQ ID NOs: 43, 96, 307, 310, 9, and 313, respectively,
SEQ ID NOs: 314, 315, 309, 76, 9, and 313, respectively,
SEQ ID NOs: 33, 316, 312, 8, 308, and 313, respectively,
SEQ ID NOs: 314, 96, 317, 76, 308, and 318, respectively,
SEQ ID NOs: 43, 316, 312, 8, 9, and 10, respectively,
SEQ ID NOs: 43, 6, 319, 320, 9, and 10, respectively,
SEQ ID NOs: 5, 67, 307, 310, 308, and 28, respectively,
SEQ ID NOs: 43, 6, 321, 8, 9, and 10, respectively,
SEQ ID NOs: 314, 316, 322, 8, 9, and 10, respectively,
SEQ ID NOs: 33, 6, 321, 8, 9, and 10, respectively,
SEQ ID NOs: 5, 6, 317, 8, 9, and 10, respectively,
SEQ ID NOs: 5, 6, 323, 8, 9, and 10, respectively, or
SEQ ID NOs: 5, 6, 324, 8, 9, and 10, respectively.

5. An antigen-binding protein or antigen-binding fragment thereof, comprising a CD28-binding portion that binds human CD28, wherein the CD28-binding portion comprises an antibody heavy chain variable domain (V_{H}) and an antibody light chain variable domain (V_{L}), and wherein the V_{H} and V_{L} comprise heavy chain complementarity-determining regions (CDRs) 1-3 and light chain CDR1-3 set forth in
SEQ ID NOs: 5-10, respectively,
SEQ ID NOs: 15, 6, 16, 17-19, respectively,
SEQ ID NOs: 24, 6, 25, 26-28, respectively,
SEQ ID NOs: 33, 6, 35-38, respectively,
SEQ ID NOs: 43, 6, 44, 45, 9, and 46, respectively,
SEQ ID NOs: 33, 51-53, 300, and 10, respectively,
SEQ ID NOs: 24, 58, 59, 60, 300, and 61, respectively,
SEQ ID NOs: 66-69, 300 and 70, respectively,
SEQ ID NOs: 24, 6, 75, 76, 18, and 28, respectively,
SEQ ID NOs: 24, 58, 81, 82, 27, and 83, respectively,
SEQ ID NOs: 88-91, 300, and 70, respectively,
SEQ ID NOs: 24, 96-98, 9, and 70, respectively,
SEQ ID NOs: 103-106, 18, and 83, respectively,
SEQ ID NOs: 111, 6, 112, 113, 18, and 114, respectively,
SEQ ID NOs: 15, 6, 119, 120, 9, and 121, respectively,
SEQ ID NOs: 126, 67, 127, 128, 18, and 129, respectively,
SEQ ID NOs: 134, 6, 135, 136, 27, and 83, respectively,
SEQ ID NOs: 43, 58, 141, 142, 300, and 143, respectively,
SEQ ID NOs: 148, 6, 149, 150, 300, and 83, respectively,
SEQ ID NOs: 15, 155, 16, 156, 27, and 70, respectively, or
SEQ ID NOs: 161, 6, 162, 163, 300, and 164, respectively
SEQ ID NOs: 5, 96, 307, 76, 308, and 46, respectively,
SEQ ID NOs: 5, 96, 309, 310, 9, and 311, respectively,
SEQ ID NOs: 43, 96, 312, 76, 9, and 28, respectively,
SEQ ID NOs: 43, 96, 307, 310, 9, and 313, respectively,
SEQ ID NOs: 314, 315, 309, 76, 9, and 313, respectively,
SEQ ID NOs: 33, 316, 312, 8, 308, and 313, respectively,
SEQ ID NOs: 314, 96, 317, 76, 308, and 318, respectively,
SEQ ID NOs: 43, 316, 312, 8, 9, and 10, respectively,
SEQ ID NOs: 43, 6, 319, 320, 9, and 10, respectively,
SEQ ID NOs: 5, 67, 307, 310, 308, and 28, respectively,
SEQ ID NOs: 43, 6, 321, 8, 9, and 10, respectively,
SEQ ID NOs: 314, 316, 322, 8, 9, and 10, respectively,
SEQ ID NOs: 33, 6, 321, 8, 9, and 10, respectively,
SEQ ID NOs: 5, 6, 317, 8, 9, and 10, respectively,
SEQ ID NOs: 5, 6, 323, 8, 9, and 10, respectively, or
SEQ ID NOs: 5, 6, 324, 8, 9, and 10, respectively.

6. The antigen-binding protein or fragment thereof of claim 4 or 5, wherein the CD28-binding portion comprises V_{H} and V_{L} set forth in
SEQ ID NOs: 11 and 12, respectively,
SEQ ID NOs: 20 and 21, respectively,
SEQ ID NOs: 29 and 30, respectively,
SEQ ID NOs: 39 and 40, respectively,
SEQ ID NOs: 47 and 48, respectively,
SEQ ID NOs: 54 and 55, respectively,
SEQ ID NOs: 62 and 63, respectively,
SEQ ID NOs: 71 and 72, respectively,
SEQ ID NOs: 77 and 78, respectively,
SEQ ID NOs: 84 and 85, respectively,
SEQ ID NOs: 92 and 93, respectively,
SEQ ID NOs: 99 and 100, respectively,
SEQ ID NOs: 107 and 108, respectively,
SEQ ID NOs: 115 and 116, respectively,
SEQ ID NOs: 122 and 123, respectively,
SEQ ID NOs: 130 and 131, respectively,
SEQ ID NOs: 137 and 138 respectively,
SEQ ID NOs: 144 and 145, respectively,
SEQ ID NOs: 151 and 152, respectively,
SEQ ID NOs: 157 and 158, respectively,
SEQ ID NOs: 165 and 166, respectively,
SEQ ID NOs: 362 and 363, respectively,
SEQ ID NOs: 364 and 365, respectively,
SEQ ID NOs: 366 and 367, respectively,
SEQ ID NOs: 368 and 369, respectively,
SEQ ID NOs: 370 and 371, respectively,
SEQ ID NOs: 372 and 373, respectively,
SEQ ID NOs: 374 and 375, respectively,
SEQ ID NOs: 376 and 12, respectively,
SEQ ID NOs: 377 and 378, respectively,
SEQ ID NOs: 379 and 380, respectively,
SEQ ID NOs: 381 and 12, respectively,
SEQ ID NOs: 382 and 12, respectively,
SEQ ID NOs: 383 and 12, respectively,
SEQ ID NOs: 384 and 12, respectively,
SEQ ID NOs: 385 and 12, respectively, or
SEQ ID NOs: 386 and 12, respectively.

7. The antigen-binding protein of claim 4 or 5, comprising an antibody heavy chain and antibody light chain set forth in
SEQ ID NOs: 13 and 14, respectively,
SEQ ID NOs: 22 and 23, respectively,
SEQ ID NOs: 31 and 32, respectively,
SEQ ID NOs: 41 and 42, respectively,
SEQ ID NOs: 49 and 50, respectively,
SEQ ID NOs: 56 and 57, respectively,
SEQ ID NOs: 64 and 65, respectively
SEQ ID NOs: 73 and 74, respectively,
SEQ ID NOs: 79 and 80, respectively,
SEQ ID NOs: 86 and 87, respectively,
SEQ ID NOs: 94 and 95, respectively,
SEQ ID NOs: 101 and 102, respectively,
SEQ ID NOs: 109 and 110, respectively,
SEQ ID NOs: 117 and 118, respectively,
SEQ ID NOs: 124 and 125, respectively,
SEQ ID NOs: 141 and 142, respectively,
SEQ ID NOs: 132 and 133, respectively,
SEQ ID NOs: 139 and 140, respectively,
SEQ ID NOs: 146 and 147, respectively,
SEQ ID NOs: 153 and 154, respectively,
SEQ ID NOs: 159 and 160, respectively, or
SEQ ID NOs: 167 and 168, respectively.

8. The antigen-binding protein or fragment thereof of any one of claims 1-7, wherein the antigen-binding protein or fragment thereof does not have superagonist activity.

9. The antigen-binding protein or fragment thereof of any one of claims 1-8, further comprising a second antigen-binding portion targeting a tumor-associated antigen (TAA).

10. The antigen-binding protein or fragment thereof of claim 9, wherein the TAA is HER2, B7-H3, or TROP-2.

11. The antigen-binding protein or fragment thereof of claim 10, comprising a HER2-binding portion comprising
HCDR1-3 and LCDR1-3 set forth in SEQ ID NOs: 262-264 and 258-260, respectively, or
V_{H} and V_{L} set forth in SEQ ID NOs: 265 and 261, respectively.

12. The antigen-binding protein or fragment thereof of claim 10, comprising a B7-H3-binding portion comprising
HCDR1-3 and LCDR1-3 set forth in SEQ ID NOs: 290-292 and 287, 18, and 288, respectively, or
V_{H} and V_{L} set forth in SEQ ID NOs: 293 and 289, respectively.

13. The antigen-binding protein or fragment thereof of claim 10, comprising a TROP-2-binding portion comprising
HCDR1-3 and LCDR1-3 set forth in SEQ ID NOs: 280-282 and 277, 259, and 278, respectively, or
V_{H} and V_{L} set forth in SEQ ID NOs: 283 and 279, respectively.

14. The antigen-binding protein or fragment thereof of any one of claims 1-13, wherein either or both of the CD28-binding portion and the TAA-binding portion are a single chain Fv (scFv), Fv, scFab, or Fab.

15. The antigen-binding protein or fragment thereof of claim 11, wherein the HER2-binding portion comprises an antibody light chain and an antibody heavy chain comprising SEQ ID NOs: 169 and 170, respectively, and wherein the CD28-binding portion comprises a heavy chain comprising SEQ ID NO: 171 or a scFv fusion polypeptide comprising SEQ ID NO: 266.

16. The antigen-binding protein or fragment thereof of claim 11, wherein the HER2-binding portion comprises an antibody light chain and an antibody heavy chain comprising SEQ ID NOs: 169 and 170, respectively, and wherein the CD28-binding portion comprises a heavy chain comprising SEQ ID NO: 172 or a scFv fusion polypeptide comprising SEQ ID NO: 266.

17. The antigen-binding protein or fragment thereof of claim 11, wherein the HER2-binding portion comprises an antibody light chain and an antibody heavy chain comprising SEQ ID NOs: 271 and 276, respectively, and wherein the CD28-binding portion comprises a heavy chain comprising SEQ ID NO: 171 or a scFv fusion polypeptide comprising SEQ ID NO: 266.

18. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 327.

19. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 333.

20. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 334.

21. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 335.

22. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 336.

23. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 337.

24. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 338.

25. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 339.

26. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 340.

27. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 341.

28. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 342.

29. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 343.

30. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 344.

31. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 345.

32. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 346.

33. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 347.

34. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 348.

35. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 349.

36. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 350.

37. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 351.

38. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 352.

39. The antigen-binding protein or fragment thereof of claim 10, wherein the HER2-binding portion comprises an antibody light chain and an antibody heavy chain comprising SEQ ID NOs: 331 and 332, respectively, and wherein the CD28-binding portion comprises a heavy chain comprising SEQ ID NO: 353.

40. The antigen-binding protein or fragment thereof of claim 12, wherein the B7-H3-binding portion comprises an antibody light chain and an antibody heavy chain comprising SEQ ID NOs: 175 and 176, respectively, and wherein the CD28-binding portion comprises a heavy chain comprising SEQ ID NO: 171 or a scFv fusion polypeptide comprising SEQ ID NO: 266.

41. The antigen-binding protein or fragment thereof of claim 10, wherein the B7-H3-binding portion comprises an antibody light chain and an antibody heavy chain comprising SEQ ID NOs: 354 and 355, respectively, and wherein the CD28-binding portion comprises a heavy chain comprising SEQ ID NO: 327.

42. The antigen-binding protein or fragment thereof of claim 10, wherein the B7-H3-binding portion comprises an antibody light chain and an antibody heavy chain comprising SEQ ID NOs: 354 and 355, respectively, and wherein the CD28-binding portion comprises a heavy chain comprising SEQ ID NO: 356.

43. The antigen-binding protein or fragment thereof of claim 10, wherein the B7-H3-binding portion comprises an antibody light chain and an antibody heavy chain comprising SEQ ID NOs: 354 and 355, respectively, and wherein the CD28-binding portion comprises a heavy chain comprising SEQ ID NO: 357.

44. The antigen-binding protein or fragment thereof of claim 10, wherein the B7-H3-binding portion comprises an antibody light chain and an antibody heavy chain comprising SEQ ID NOs: 354 and 355, respectively, and wherein the CD28-binding portion comprises a heavy chain comprising SEQ ID NO: 358.

45. The antigen-binding protein or fragment thereof of claim 13, wherein the TROP2-binding portion comprises an antibody light chain and an antibody heavy chain comprising SEQ ID NOs: 173 and 174, respectively, and wherein the CD28-binding portion comprises a heavy chain comprising SEQ ID NO: 171 or a scFv fusion polypeptide comprising SEQ ID NO: 266.

46. The antigen-binding protein or fragment thereof of any one of claims 1-45, further comprising another antigen-binding portion targeting an immune checkpoint inhibitor.

47. The antigen-binding protein of claim 46, wherein the immune checkpoint inhibitor is PD-L1.

48. The antigen-binding protein or fragment thereof of claim 47, wherein the PD-L1-binding portion comprises an antibody light chain and an antibody heavy chain comprising SEQ ID NOs: 325 and 326, respectively, and wherein the CD28-binding portion comprises a heavy chain comprising SEQ ID NO: 327.

49. The antigen-binding protein or fragment thereof of claim 47, wherein the PD-L1-binding portion comprises an antibody light chain and an antibody heavy chain comprising SEQ ID NOs: 325 and 326, respectively, and wherein the CD28-binding portion comprises a heavy chain comprising SEQ ID NO: 328.

50. The antigen-binding protein or fragment thereof of claim 47, wherein the PD-L1-binding portion comprises an antibody light chain and an antibody heavy chain comprising SEQ ID NOs: 325 and 326, respectively, and wherein the CD28-binding portion comprises a heavy chain comprising SEQ ID NO: 329.

51. The antigen-binding protein or fragment thereof of claim 47, wherein the PD-L1-binding portion comprises an antibody light chain and an antibody heavy chain comprising SEQ ID NOs: 325 and 326, respectively, and wherein the CD28-binding portion comprises a heavy chain comprising SEQ ID NO: 330.

52. The antigen-binding protein or fragment thereof of any one of claims 1-51, further comprising an Fc region.

53. The antigen-binding protein or fragment thereof of claim 52, wherein the Fc region is of the human IgG1 subclass.

54. The antigen-binding protein or fragment thereof of claim 52, wherein the Fc region is of the human IgG4 subclass.

55. The antigen-binding protein or fragment thereof of any one of claims 52-54, wherein the Fc region has reduced or no effector function, reduced or no antibody-dependent cell cytotoxicity (ADCC) effect, and/or reduced or no crosslinking effects.

56. The antigen-binding protein or fragment thereof of any one of claims 1-55, comprising a first CH3 domain and a second CH3 domain, wherein:
i) the first CH3 domain comprises a cysteine (C) residue at position 390 and the second CH3 domain comprises a cysteine residue at position 400, or the first CH3 domain comprises a cysteine residue at position 400 and the second CH3 domain comprises a cysteine residue at position 390; or
ii) the first CH3 domain comprises a cysteine residue at position 392 and the second CH3 domain comprises a cysteine residue at position 397, or the first CH3 domain comprises a cysteine residue at position 397 and the second CH3 domain comprises a cysteine residue at position 392; or
iii) the first CH3 domain comprises a cysteine residue at position 392 and the second CH3 domain comprises a cysteine residue at position 400, or the first CH3 domain comprises a cysteine residue at position 400 and the second CH3 domain comprises a cysteine residue at position 392; and wherein the amino acid residue numbering is based on Eu numbering.

57. The antigen-binding protein or fragment thereof of claim 56, wherein:
i) the first CH3 domain further comprises a positively charged residue at position 357 and the second CH3 domain further comprises a negatively charged residue at position 351, or the first CH3 domain further comprises a negatively charged residue at position 351 and the second CH3 domain further comprises a positively charged residue at position 357; or
ii) the first CH3 domain further comprises a positively charged residue at position 411 and the second CH3 domain further comprises a negatively charged residue at position 370, or the first CH3 domain further comprises a negatively charged residue at position 370 and the second CH3 domain further comprises a positively charged residue at position 411; or
iii) the first CH3 domain further comprises a positively charged residue at position 364 and the second CH3 domain further comprises a negatively charged residue at position 370, or the first CH3 domain further comprises a negatively charged residue at position 370 and the second CH3 domain further comprises a positively charged residue at position 364; or a combination of i) and ii), or a combination of i) and iii); and wherein the amino acid residue numbering is based on Eu numbering.

58. The antigen-binding protein or fragment thereof of claim 57, wherein the first CH3 domain comprises D/E356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D/E356K, E357K, S364K and S400C substitutions (Eu numbering).

59. The antigen-binding protein or fragment thereof of claim 58, wherein the CH3 domain further comprises an N297A substitution (Eu numbering).

60. The antigen-binding protein or fragment thereof of any one of claims 1-60, further comprising at least one masking peptide, wherein the masking peptide (MP) is linked to an N-terminus of the V_{L}, wherein the MP comprises, from N-terminus to C-terminus, a masking unit (MU) and a linkage unit (LU) with or without cleavage sites.

61. The antigen-binding protein or fragment thereof of claim 60, and wherein the MU comprises a sequence selected from the group consisting of SEQ ID NOs: 173-206.

62. The antigen-binding protein or fragment thereof of claim 61, wherein the MP further comprises an N-terminal unit (NU) linked to the N-terminus of the MU.

63. The antigen-binding protein or fragment thereof of claim 62, wherein the N-terminal unit is about 1-10 amino acid residues long.

64. The antigen-binding protein or fragment thereof of claim 63, wherein the N-terminal unit comprises E or EVGSY.

65. The antigen-binding protein or fragment thereof of any one of claims 60-64, wherein the LU comprises a cleavage site.

66. The antigen-binding protein or fragment thereof of claim 65, wherein the first cleavage site is a protease cleavage site for a protease selected from the group consisting of urokinase-type plasminogen activator/uPA, matrix metalloproteinase-1/MMP-1, MMP-2, MMP-3, MMP-8, MMP-9, MMP-14, Tobacco Etch Virus protease/TEV protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE.

67. The antigen-binding protein or fragment thereof of any one of claims 60-66, wherein at least one masking peptide is linked to the B7-H3-, HER2-, or TROP2-binding portion or CD28-binding portion, optionally wherein the antigen-binding protein or fragment thereof comprises two masking peptides that bind respectively the B7-H3-, HER2-, or TROP2-binding portion and the CD28-binding portion.

68. The antigen-binding protein or fragment thereof of any one of claims 1-67, further comprising a conjugated therapeutic moiety, optionally wherein the therapeutic moiety is a radioactive moiety or a cytotoxic moiety.

69. A pharmaceutical composition comprising the antigen-binding protein or fragment thereof of any one of claims 1-68 and a pharmaceutically acceptable carrier.

70. A nucleic acid molecule or nucleic acid molecule (s) encoding the antigen-binding protein or fragment thereof of any one of claims 1-68.

71. A host cell comprising nucleotide sequence (s) claim 70.

72. A method of producing an antigen-binding protein or antigen-binding fragment thereof, comprising:
culturing the host cell of claim 71 under conditions that allow expression of the antigen-binding protein or fragment thereof, and isolating the antigen-binding protein or fragment thereof from the culture.

73. A method of treating cancer in a patient in need thereof, comprising administering to the patient a therapeutically effective amount of the antigen-binding protein or fragment thereof of any one of claims 1-68 or the pharmaceutical composition of claim 69.

74. The method of claim 73, further comprising administering to the patient another anti-cancer therapeutic.

75. The method of claim 74, wherein the additional anti-cancer therapeutic is a bispecific antibody targeting CD3 and a tumor antigen, optionally wherein the tumor antigen is the same as or different from the TAA.

76. The antigen-binding protein or fragment thereof of claim 75, wherein the TAA is B7-H3, HER2, or TROP2.

77. The method of claim 74, wherein the additional anti-cancer therapeutics is an immune checkpoint inhibitor, optionally an anti-PD-1, anti-CTLA-4, or anti-PD-L1 antibody.

78. The method of any one of claims 73-77, wherein the patient has a solid tumor or a hematological malignancy, optionally selected from breast cancer, gastric cancer, lung cancer, ovarian cancer, kidney cancer, pancreatic cancer, and colon cancer.

79. Use of the antigen-binding protein or fragment thereof of any one of claims 1-68 for the manufacture of a medicament in treating cancer, optionally in a method of any one of claims 73-78.

80. The antigen-binding protein or fragment thereof of any one of claims 1-68, or the pharmaceutical composition of claim 69, for use in treating cancer, optionally in a method of any one of claims 73-78.


SEQUENCE LISTING (361 sequences)

SEQ ID NO: 1 - protein, 220 aa, Homo sapiens
  REGION 1..220: note = misc_feature - Human CD28
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 220 aa, Macaca mulatta
  REGION 1..220: note = misc_feature - Macaca mulatta CD28
MLRLLLALNLLPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWALVVVGGVLACYSLLVTVAFCIFWMRSKRSRLLHSDYMNMTPRRPGPTRKHYQPYAPPRDFAAYRS

SEQ ID NO: 3 - protein, 220 aa, Macaca fascicularis; identical to SEQ ID NO: 2
  REGION 1..220: note = misc_feature - Macaca fascicularis CD28

SEQ ID NO: 4 - protein, 218 aa, Mus musculus
  REGION 1..218: note = misc_feature - Mouse CD28
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 5 - protein, 7 aa, synthetic construct
  REGION 1..7: note = Synthetic: HCDR1/HCDR1-B
TGGVGVS

SEQ ID NO: 6 - protein, 16 aa, synthetic construct
  REGION 1..16: note = Synthetic: HCDR2/HCDR2-B
EIYHSGSTYYSPSLKS

SEQ ID NO: 7 - protein, 10 aa, synthetic construct
  REGION 1..10: note = Synthetic: HCDR3/HCDR3-B
YEYYYYDFDY

SEQ ID NO: 8 - protein, 15 aa, synthetic construct
  REGION 1..15: note = Synthetic: LCDR1/LCDR1-B
RASQSVDFHGISFLH

SEQ ID NO: 9 - protein, 7 aa, synthetic construct
  REGION 1..7: note = Synthetic: LCDR2 / LCDR2-B
AASSLQS

SEQ ID NO: 10 - protein, 9 aa, synthetic construct
  REGION 1..9: note = Synthetic: LCDR3 / LCDR3-B
QQSYRSPRT

SEQ ID NO: 11 - protein, 120 aa, synthetic construct
  REGION 1..120: note = Synthetic: VH
EVQLVESGGGLVQPGGSLRLSCAASGFSLSTGGVGVSWIRQAPGKGLEWIGEIYHSGSTYYSPSLKSRVTISRDNSKNTLYLQLNSLRAEDTAVYYCARYEYYYYDFDYWGQGTLVTVSS

SEQ ID NO: 12 - protein, 111 aa, synthetic construct
  REGION 1..111: note = Synthetic: VL
DIQLTQSPSSLSASVGDRVTITCRASQSVDFHGISFLHWYQQKPGKAPKLLIYAASSLQSGVPSRFSGSGSGTDFTLTISSLQPEDFATYYCQQSYRSPRTFGQGTKVEIK

SEQ ID NO: 13 - protein, 447 aa, synthetic construct
  REGION 1..447: note = Synthetic: HC
EVQLVESGGGLVQPGGSLRLSCAASGFSLSTGGVGVSWIRQAPGKGLEWIGEIYHSGSTYYSPSLKSRVTISRDNSKNTLYLQLNSLRAEDTAVYYCARYEYYYYDFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 218 aa, synthetic construct
  REGION 1..218: note = Synthetic: LC
DIQLTQSPSSLSASVGDRVTITCRASQSVDFHGISFLHWYQQKPGKAPKLLIYAASSLQSGVPSRFSGSGSGTDFTLTISSLQPEDFATYYCQQSYRSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 6 aa, synthetic construct
  REGION 1..6: note = Synthetic: HCDR1
SGHHWS

SEQ ID NO: 16 - protein, 9 aa, synthetic construct
  REGION 1..9: note = Synthetic: HCDR3
KDDYYAFDY

SEQ ID NO: 17 - protein, 15 aa, synthetic construct
  REGION 1..15: note = Synthetic: LCDR1
RASESVDFYGISFLH

SEQ ID NO: 18 - protein, 7 aa, synthetic construct
  REGION 1..7: note = Synthetic: LCDR2 / LCDR2-A
AASTLQS

SEQ ID NO: 19 - protein, 9 aa, synthetic construct
  REGION 1..9: note = Synthetic: LCDR3
QQSYRTPIT

SEQ ID NO: 20 - protein, 118 aa, synthetic construct
  REGION 1..118: note = Synthetic: VH
EVQLVESGGGLVQPGGSLRLSCAASGYSITSGHHWSWIRQAPGKGLEWIGEIYHSGSTYYSPSLKSRVTISRDNSKNTLYLQLNSLRAEDTAVYYCARKDDYYAFDYWGQGTLVTVSS

SEQ ID NO: 21 - protein, 111 aa, synthetic construct
  REGION 1..111: note = Synthetic: VL
DIQLTQSPSSLSASVGDRVTITCRASESVDFYGISFLHWYQQKPGKAPKLLIYAASTLQSGVPSRFSGSGSGTDFTLTISSLQPEDFATYYCQQSYRTPITFGQGTKVEIK

SEQ ID NO: 22 - protein, 445 aa, synthetic construct
  REGION 1..445: note = Synthetic: HC
EVQLVESGGGLVQPGGSLRLSCAASGYSITSGHHWSWIRQAPGKGLEWIGEIYHSGSTYYSPSLKSRVTISRDNSKNTLYLQLNSLRAEDTAVYYCARKDDYYAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 218 aa, synthetic construct
  REGION 1..218: note = Synthetic: LC
DIQLTQSPSSLSASVGDRVTITCRASESVDFYGISFLHWYQQKPGKAPKLLIYAASTLQSGVPSRFSGSGSGTDFTLTISSLQPEDFATYYCQQSYRTPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 6 aa, synthetic construct
  REGION 1..6: note = Synthetic: HCDR1
SGYHWG

SEQ ID NO: 25 - protein, 9 aa, synthetic construct
  REGION 1..9: note = Synthetic: HCDR3
RRGDGYFDF

SEQ ID NO: 26 - protein, 15 aa, synthetic construct
  REGION 1..15: note = Synthetic: LCDR1
RASQSVDFLGISFLH

SEQ ID NO: 27 - protein, 7 aa, synthetic construct
  REGION 1..7: note = Synthetic: LCDR2
DASNRAT

SEQ ID NO: 28 - protein, 9 aa, synthetic construct
  REGION 1..9: note = Synthetic: LCDR3
QQSYRTPLT

SEQ ID NO: 29 - protein, 118 aa, synthetic construct
  REGION 1..118: note = Synthetic: VH
EVQLVESGGGLVQPGGSLRLSCAASGYSITSGYHWGWIRQAPGKGLEWIGEIYHSGSTYYSPSLKSRVTISRDNSKNTLYLQLNSLRAEDTAVYYCARRRGDGYFDFWGQGTLVTVSS

SEQ ID NO: 30 - protein, 111 aa, synthetic construct
  REGION 1..111: note = Synthetic: VL
DIQLTQSPSSLSASVGDRVTITCRASQSVDFLGISFLHWYQQKPGKAPKLLIYDASNRATGIPSRFSGSGSGTDFTLTISSLQPEDFATYYCQQSYRTPLTFGQGTKVEIK

SEQ ID NO: 31 - protein, 445 aa, synthetic construct
  REGION 1..445: note = Synthetic: HC
EVQLVESGGGLVQPGGSLRLSCAASGYSITSGYHWGWIRQAPGKGLEWIGEIYHSGSTYYSPSLKSRVTISRDNSKNTLYLQLNSLRAEDTAVYYCARRRGDGYFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 218 aa, synthetic construct
  REGION 1..218: note = Synthetic: LC
DIQLTQSPSSLSASVGDRVTITCRASQSVDFLGISFLHWYQQKPGKAPKLLIYDASNRATGIPSRFSGSGSGTDFTLTISSLQPEDFATYYCQQSYRTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 7 aa, synthetic construct
  REGION 1..7: note = Synthetic: HCDR1
TGGVGVG

SEQ ID NO: 34 - protein, 5 aa, synthetic construct
  REGION 1..5: note = Synthetic: HCDR1-B
TYAIN

SEQ ID NO: 35 - protein, 10 aa, synthetic construct
  REGION 1..10: note = Synthetic: HCDR3
YGYSYYALDY

SEQ ID NO: 36 - protein, 10 aa, synthetic construct
  REGION 1..10: note = Synthetic: LCDR1
SASSSVGYVQ

SEQ ID NO: 37 - protein, 7 aa, synthetic construct
  REGION 1..7: note = Synthetic: LCDR2
DASNLET

SEQ ID NO: 38 - protein, 9 aa, synthetic construct
  REGION 1..9: note = Synthetic: LCDR3
QQSYTSPRT

SEQ ID NO: 39 - protein, 120 aa, synthetic construct
  REGION 1..120: note = Synthetic: VH
EVQLVESGGGLVQPGGSLRLSCAASGFSLSTGGVGVGWIRQAPGKGLEWIGEIYHSGSTYYSPSLKSRVTISRDNSKNTLYLQLNSLRAEDTAVYYCARYGYSYYALDYWGQGTLVTVSS

SEQ ID NO: 40 - protein, 106 aa, synthetic construct
  REGION 1..106: note = Synthetic: VL
DIQLTQSPSSLSASVGDRVTITCSASSSVGYVQWYQQKPGKAPKLLIYDASNLETGVPSRFSGSGSGTDFTLTISSLQPEDFATYYCQQSYTSPRTFGQGTKVEIK

SEQ ID NO: 41 - protein, 447 aa, synthetic construct
  REGION 1..447: note = Synthetic: HC
EVQLVESGGGLVQPGGSLRLSCAASGFSLSTGGVGVGWIRQAPGKGLEWIGEIYHSGSTYYSPSLKSRVTISRDNSKNTLYLQLNSLRAEDTAVYYCARYGYSYYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 213 aa, synthetic construct
  REGION 1..213: note = Synthetic: LC
DIQLTQSPSSLSASVGDRVTITCSASSSVGYVQWYQQKPGKAPKLLIYDASNLETGVPSRFSGSGSGTDFTLTISSLQPEDFATYYCQQSYTSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 7 aa, synthetic construct
  REGION 1..7: note = Synthetic: HCDR1
TSGVGVS

SEQ ID NO: 44 - protein, 10 aa, synthetic construct
  REGION 1..10: note = Synthetic: HCDR3
KGYYYYAFDY

SEQ ID NO: 45 - protein, 11 aa, synthetic construct
  REGION 1..11: note = Synthetic: LCDR1
RASQDIGSFLA

SEQ ID NO: 46 - protein, 9 aa, synthetic construct
  REGION 1..9: note = Synthetic: LCDR3
QQSYRSPPT

SEQ ID NO: 47 - protein, 120 aa, synthetic construct
  REGION 1..120: note = Synthetic: VH
EVQLVESGGGLVQPGGSLRLSCAASGFSLSTSGVGVSWIRQAPGKGLEWIGEIYHSGSTYYSPSLKSRVTISRDNSKNTLYLQLNSLRAEDTAVYYCARKGYYYYAFDYWGPGTLVTVSS

SEQ ID NO: 48 - protein, 107 aa, synthetic construct
  REGION 1..107: note = Synthetic: VL
DIQLTQSPSSLSASVGDRVTITCRASQDIGSFLAWYQQKPGKAPKLLIYAASSLQSGVPSRFSGSGSGTDFTLTISSLQPEDFATYYCQQSYRSPPTFGQGTKVEIK

SEQ ID NO: 49 - protein, 447 aa, synthetic construct
  REGION 1..447: note = Synthetic: HC
EVQLVESGGGLVQPGGSLRLSCAASGFSLSTSGVGVSWIRQAPGKGLEWIGEIYHSGSTYYSPSLKSRVTISRDNSKNTLYLQLNSLRAEDTAVYYCARKGYYYYAFDYWGPGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, synthetic construct
  REGION 1..214: note = Synthetic: LC
DIQLTQSPSSLSASVGDRVTITCRASQDIGSFLAWYQQKPGKAPKLLIYAASSLQSGVPSRFSGSGSGTDFTLTISSLQPEDFATYYCQQSYRSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 16 aa, synthetic construct
  REGION 1..16: note = Synthetic: HCDR2
EIYHSGSTYYPPSLKS

SEQ ID NO: 52 - protein, 10 aa, synthetic construct
  REGION 1..10: note = Synthetic: HCDR3
YEYSSYGLDY

SEQ ID NO: 53 - protein, 11 aa, synthetic construct
  REGION 1..11: note = Synthetic: LCDR1
RASQDIRKFLA

SEQ ID NO: 54 - protein, 120 aa, synthetic construct
  REGION 1..120: note = Synthetic: VH
EVQLVESGGGLVQPGGSLRLSCAASGFSLSTGGVGVGWIRQAPGKGLEWIGEIYHSGSTYYPPSLKSRVTISRDNSKNTLYLQLNSLRAEDTAVYYCARYEYSSYGLDYWGQGTLVTVSS

SEQ ID NO: 55 - protein, 107 aa, synthetic construct
  REGION 1..107: note = Synthetic: VL
DIQLTQSPSSLSASVGDRVTITCRASQDIRKFLAWYQQKPGKAPKLLIYDASSLESGVPSRFSGSGSGTDFTLTISSLQPEDFATYYCQQSYRSPRTFGQGTKVEIK

SEQ ID NO: 56 - protein, 447 aa, synthetic construct
  REGION 1..447: note = Synthetic: HC
EVQLVESGGGLVQPGGSLRLSCAASGFSLSTGGVGVGWIRQAPGKGLEWIGEIYHSGSTYYPPSLKSRVTISRDNSKNTLYLQLNSLRAEDTAVYYCARYEYSSYGLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 214 aa, synthetic construct
  REGION 1..214: note = Synthetic: LC
DIQLTQSPSSLSASVGDRVTITCRASQDIRKFLAWYQQKPGKAPKLLIYDASSLESGVPSRFSGSGSGTDFTLTISSLQPEDFATYYCQQSYRSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 16 aa, synthetic construct
  REGION 1..16: note = Synthetic: HCDR2
AIYHSGSTYYSPSLKS

SEQ ID NO: 59 - protein, 10 aa, synthetic construct
  REGION 1..10: note = Synthetic: HCDR3
SGLVYYAFDY

SEQ ID NO: 60 - protein, 11 aa, synthetic construct
  REGION 1..11: note = Synthetic: LCDR1
RASQGIGSYLA

SEQ ID NO: 61 - protein, 9 aa, synthetic construct
  REGION 1..9: note = Synthetic: LCDR3
QQYSTSPLT

SEQ ID NO: 62 - protein, 119 aa, synthetic construct
  REGION 1..119: note = Synthetic: VH
QVQLVQSGAEVKKPGSSVKVSCKASGYSISSGYHWGWIRQAPGQGLEWIGAIYHSGSTYYSPSLKSRVTITADKSTSTAYMELSSLRSEDTAVYYCARSGLVYYAFDYWGQGTLVTVSS

SEQ ID NO: 63 - protein, 107 aa, synthetic construct
  REGION 1..107: note = Synthetic: VL
DIQLTQSPSSLSASVGDRVTITCRASQGIGSYLAWYQQKPGKAPKLLIYDASSLESGVPSRFSGSGSGTDFTLTISSLQPEDFATYYCQQYSTSPLTFGQGTKVEIK

SEQ ID NO: 64 - protein, 446 aa, synthetic construct
  REGION 1..446: note = Synthetic: HC
QVQLVQSGAEVKKPGSSVKVSCKASGYSISSGYHWGWIRQAPGQGLEWIGAIYHSGSTYYSPSLKSRVTITADKSTSTAYMELSSLRSEDTAVYYCARSGLVYYAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 214 aa, synthetic construct
  REGION 1..214: note = Synthetic: LC
DIQLTQSPSSLSASVGDRVTITCRASQGIGSYLAWYQQKPGKAPKLLIYDASSLESGVPSRFSGSGSGTDFTLTISSLQPEDFATYYCQQYSTSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 5 aa, synthetic construct
  REGION 1..5: note = Synthetic: HCDR1
SYAIH

SEQ ID NO: 67 - protein, 16 aa, synthetic construct
  REGION 1..16: note = Synthetic: HCDR2
SIYHSGSTYYSPSLKS

SEQ ID NO: 68 - protein, 10 aa, synthetic construct
  REGION 1..10: note = Synthetic: HCDR3
DVYGSYALDV

SEQ ID NO: 69 - protein, 15 aa, synthetic construct
  REGION 1..15: note = Synthetic: LCDR1
RASQSVDFDGISFLH

SEQ ID NO: 70 - protein, 9 aa, synthetic construct
  REGION 1..9: note = Synthetic: LCDR3
QQSYRSPIT

SEQ ID NO: 71 - protein, 118 aa, synthetic construct
  REGION 1..118: note = Synthetic: VH
QVQLVQSGAEVKKPGSSVKVSCKASGFTFSSYAIHWVRQAPGQGLEWIGSIYHSGSTYYSPSLKSRVTITADKSTSTAYMELSSLRSEDTAVYYCARDVYGSYALDVWGQGTLVTVSS

SEQ ID NO: 72 - protein, 111 aa, synthetic construct
  REGION 1..111: note = Synthetic: VL
DIQLTQSPSSLSASVGDRVTITCRASQSVDFDGISFLHWYQQKPGKAPKLLIYDASSLESGVPSRFSGSGSGTDFTLTISSLQPEDFATYYCQQSYRSPITFGQGTKVEIK

SEQ ID NO: 73 - protein, 445 aa, synthetic construct
  REGION 1..445: note = Synthetic: HC
QVQLVQSGAEVKKPGSSVKVSCKASGFTFSSYAIHWVRQAPGQGLEWIGSIYHSGSTYYSPSLKSRVTITADKSTSTAYMELSSLRSEDTAVYYCARDVYGSYALDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 218 aa, synthetic construct
  REGION 1..218: note = Synthetic: LC
DIQLTQSPSSLSASVGDRVTITCRASQSVDFDGISFLHWYQQKPGKAPKLLIYDASSLESGVPSRFSGSGSGTDFTLTISSLQPEDFATYYCQQSYRSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 9 aa, synthetic construct
  REGION 1..9: note = Synthetic: HCDR3
KDYYYALDY

SEQ ID NO: 76 - protein, 15 aa, synthetic construct
  REGION 1..15: note = Synthetic: LCDR1
RASQSVDFYGKSFLH

SEQ ID NO: 77 - protein, 118 aa, synthetic construct
  REGION 1..118: note = Synthetic: VH
EVQLVESGGGLVQPGGSLRLSCAASGYSISSGYHWGWIRQAPGKGLEWIGEIYHSGSTYYSPSLKSRVTISRDNSKNTLYLQLNSLRAEDTAVYYCARKDYYYALDYWGQGTLVTVSS

SEQ ID NO: 78 - protein, 111 aa, synthetic construct
  REGION 1..111: note = Synthetic: VL
DIQLTQSPSSLSASVGDRVTITCRASQSVDFYGKSFLHWYQQKPGKAPKLLIYAASTLQSGVPSRFSGSGSGTDFTLTISSLQPEDFATYYCQQSYRTPLTFGQGTKVEIK

SEQ ID NO: 79 - protein, 445 aa, synthetic construct
  REGION 1..445: note = Synthetic: HC
EVQLVESGGGLVQPGGSLRLSCAASGYSISSGYHWGWIRQAPGKGLEWIGEIYHSGSTYYSPSLKSRVTISRDNSKNTLYLQLNSLRAEDTAVYYCARKDYYYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 218 aa, synthetic construct
  REGION 1..218: note = Synthetic: LC
DIQLTQSPSSLSASVGDRVTITCRASQSVDFYGKSFLHWYQQKPGKAPKLLIYAASTLQSGVPSRFSGSGSGTDFTLTISSLQPEDFATYYCQQSYRTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 10 aa, synthetic construct
  REGION 1..10: note = Synthetic: HCDR3
GPLSYYAFDY

SEQ ID NO: 82 - protein, 11 aa, synthetic construct
  REGION 1..11: note = Synthetic: LCDR1
RASQSVSRYLA

SEQ ID NO: 83 - protein, 9 aa, synthetic construct
  REGION 1..9: note = Synthetic: LCDR3
QQYYRSPIT

SEQ ID NO: 84 - protein, 119 aa, synthetic construct
  REGION 1..119: note = Synthetic: VH
QVQLVQSGAEVKKPGSSVKVSCKASGYSITSGYHWGWIRQAPGQGLEWIGAIYHSGSTYYSPSLKSRVTITADKSTSTAYMELSSLRSEDTAVYYCARGPLSYYAFDYWGQGTLVTVSS

SEQ ID NO: 85 - protein, 107 aa, synthetic construct
  REGION 1..107: note = Synthetic: VL
DIQLTQSPSSLSASVGDRVTITCRASQSVSRYLAWYQQKPGKAPKLLIYDASNRATGIPSRFSGSGSGTDFTLTISSLQPEDFATYYCQQYYRSPITFGQGTKVEIK

SEQ ID NO: 86 - protein, 446 aa, synthetic construct
  REGION 1..446: note = Synthetic: HC
QVQLVQSGAEVKKPGSSVKVSCKASGYSITSGYHWGWIRQAPGQGLEWIGAIYHSGSTYYSPSLKSRVTITADKSTSTAYMELSSLRSEDTAVYYCARGPLSYYAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 214 aa, synthetic construct
  REGION 1..214: note = Synthetic: LC
DIQLTQSPSSLSASVGDRVTITCRASQSVSRYLAWYQQKPGKAPKLLIYDASNRATGIPSRFSGSGSGTDFTLTISSLQPEDFATYYCQQYYRSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 7 aa, synthetic construct
  REGION 1..7: note = Synthetic: HCDR1
TGGVAVA

SEQ ID NO: 89 - protein, 16 aa, synthetic construct
  REGION 1..16: note = Synthetic: HCDR2
QIYYSGSTYYNPSLKS

SEQ ID NO: 90 - protein, 9 aa, synthetic construct
  REGION 1..9: note = Synthetic: HCDR3
SSAHYYFDV

SEQ ID NO: 91 - protein, 15 aa, synthetic construct
  REGION 1..15: note = Synthetic: LCDR1
RASESVDFQGISFLD

SEQ ID NO: 92 - protein, 119 aa, synthetic construct
  REGION 1..119: note = Synthetic: VH
QVQLVQSGAEVKKPGSSVKVSCKASGFSLSTGGVAVAWIRQAPGQGLEWIGQIYYSGSTYYNPSLKSRVTITADKSTSTAYMELSSLRSEDTAVYYCARSSAHYYFDVWGQGTLVTVSS

SEQ ID NO: 93 - protein, 111 aa, synthetic construct
  REGION 1..111: note = Synthetic: VL
DIQLTQSPSSLSASVGDRVTITCRASESVDFQGISFLDWYQQKPGKAPKLLIYDASSLESGVPSRFSGSGSGTDFTLTISSLQPEDFATYYCQQSYRSPITFGQGTKVEIK

SEQ ID NO: 94 - protein, 446 aa, synthetic construct
  REGION 1..446: note = Synthetic: HC
QVQLVQSGAEVKKPGSSVKVSCKASGFSLSTGGVAVAWIRQAPGQGLEWIGQIYYSGSTYYNPSLKSRVTITADKSTSTAYMELSSLRSEDTAVYYCARSSAHYYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 95 - protein, 218 aa, synthetic construct
  REGION 1..218: note = Synthetic: LC
DIQLTQSPSSLSASVGDRVTITCRASESVDFQGISFLDWYQQKPGKAPKLLIYDASSLESGVPSRFSGSGSGTDFTLTISSLQPEDFATYYCQQSYRSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 16 aa, synthetic construct
  REGION 1..16: note = Synthetic: HCDR2
SIYHSGSTYYNPSLKS

SEQ ID NO: 97 - protein, 10 aa, synthetic construct
  REGION 1..10: note = Synthetic: HCDR3
SGLRYYAFDV

SEQ ID NO: 98 - protein, 11 aa, synthetic construct
  REGION 1..11: note = Synthetic: LCDR1
AASQDVSTAVA

SEQ ID NO: 99 - protein, 119 aa, synthetic construct
  REGION 1..119: note = Synthetic: VH
QVQLVQSGAEVKKPGSSVKVSCKASGYSISSGYHWGWIRQAPGQGLEWIGSIYHSGSTYYNPSLKSRVTITADKSTSTAYMELSSLRSEDTAVYYCARSGLRYYAFDVWGQGTLVTVSS

SEQ ID NO: 100 - protein, 107 aa, synthetic construct
  REGION 1..107: note = Synthetic: VL
DIQLTQSPSSLSASVGDRVTITCAASQDVSTAVAWYQQKPGKAPKLLIYAASSLQSGVPSRFSGSGSGTDFTLTISSLQPEDFATYYCQQSYRSPITFGQGTKVEIK

SEQ ID NO: 101 - protein, 446 aa, synthetic construct
  REGION 1..446: note = Synthetic: HC
QVQLVQSGAEVKKPGSSVKVSCKASGYSISSGYHWGWIRQAPGQGLEWIGSIYHSGSTYYNPSLKSRVTITADKSTSTAYMELSSLRSEDTAVYYCARSGLRYYAFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 214 aa, synthetic construct
  REGION 1..214: note = Synthetic: LC
DIQLTQSPSSLSASVGDRVTITCAASQDVSTAVAWYQQKPGKAPKLLIYAASSLQSGVPSRFSGSGSGTDFTLTISSLQPEDFATYYCQQSYRSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 5 aa, synthetic construct
  REGION 1..5: note = Synthetic: HCDR1
GYAIH

SEQ ID NO: 104 - protein, 16 aa, synthetic construct
  REGION 1..16: note = Synthetic: HCDR2
QIYHSGSTYYNPSLKS

SEQ ID NO: 105 - protein, 10 aa, synthetic construct
  REGION 1..10: note = Synthetic: HCDR3
SGYGGYDFDY

SEQ ID NO: 106 - protein, 12 aa, synthetic construct
  REGION 1..12: note = Synthetic: LCDR1
RASQSVPRRYLA

SEQ ID NO: 107 - protein, 118 aa, synthetic construct
  REGION 1..118: note = Synthetic: VH
QVQLVQSGAEVKKPGSSVKVSCKASGFTFSGYAIHWVRQAPGQGLEWIGQIYHSGSTYYNPSLKSRVTITADKSTSTAYMELSSLRSEDTAVYYCARSGYGGYDFDYWGQGTLVTVSS

SEQ ID NO: 108 - protein, 108 aa, synthetic construct
  REGION 1..108: note = Synthetic: VL
DIQLTQSPSSLSASVGDRVTITCRASQSVPRRYLAWYQQKPGKAPKLLIYAASTLQSGVPSRFSGSGSGTDFTLTISSLQPEDFATYYCQQYYRSPITFGQGTKVEIK

SEQ ID NO: 109 - protein, 445 aa, synthetic construct
  REGION 1..445: note = Synthetic: HC
QVQLVQSGAEVKKPGSSVKVSCKASGFTFSGYAIHWVRQAPGQGLEWIGQIYHSGSTYYNPSLKSRVTITADKSTSTAYMELSSLRSEDTAVYYCARSGYGGYDFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 215 aa, synthetic construct
  REGION 1..215: note = Synthetic: LC
DIQLTQSPSSLSASVGDRVTITCRASQSVPRRYLAWYQQKPGKAPKLLIYAASTLQSGVPSRFSGSGSGTDFTLTISSLQPEDFATYYCQQYYRSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 6 aa, synthetic construct
  REGION 1..6: note = Synthetic: HCDR1
SGHHWG

SEQ ID NO: 112 - protein, 10 aa, synthetic construct
  REGION 1..10: note = Synthetic: HCDR3
RELGYAYFDY

SEQ ID NO: 113 - protein, 11 aa, synthetic construct
  REGION 1..11: note = Synthetic: LCDR1
RASQDIRKYLA

SEQ ID NO: 114 - protein, 9 aa, synthetic construct
  REGION 1..9: note = Synthetic: LCDR3
QQYYRTPLT

SEQ ID NO: 115 - protein, 119 aa, synthetic construct
  REGION 1..119: note = Synthetic: VH
EVQLVESGGGLVQPGGSLRLSCAASGYSISSGHHWGWIRQAPGKGLEWIGEIYHSGSTYYSPSLKSRVTISRDNSKNTLYLQLNSLRAEDTAVYYCARRELGYAYFDYWGQGTLVTVSS

SEQ ID NO: 116 - protein, 107 aa, synthetic construct
  REGION 1..107: note = Synthetic: VL
EIVLTQSPATLSLSPGERATLSCRASQDIRKYLAWYQQKPGQAPRLLIYAASTLQSGVPARFSGSGSGTDFTLTISSLEPEDFAVYYCQQYYRTPLTFGQGTKVEIK

SEQ ID NO: 117 - protein, 446 aa, synthetic construct
  REGION 1..446: note = Synthetic: HC
EVQLVESGGGLVQPGGSLRLSCAASGYSISSGHHWGWIRQAPGKGLEWIGEIYHSGSTYYSPSLKSRVTISRDNSKNTLYLQLNSLRAEDTAVYYCARRELGYAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 214 aa, synthetic construct
  REGION 1..214: note = Synthetic: LC
EIVLTQSPATLSLSPGERATLSCRASQDIRKYLAWYQQKPGQAPRLLIYAASTLQSGVPARFSGSGSGTDFTLTISSLEPEDFAVYYCQQYYRTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 10 aa, synthetic construct
  REGION 1..10: note = Synthetic: HCDR3
RDFGASYFDY

SEQ ID NO: 120 - protein, 11 aa, synthetic construct
  REGION 1..11: note = Synthetic: LCDR1
RASQGVGPWLA

SEQ ID NO: 121 - protein, 8 aa, synthetic construct
  REGION 1..8: note = Synthetic: LCDR3
QQYYSYST

SEQ ID NO: 122 - protein, 119 aa, synthetic construct
  REGION 1..119: note = Synthetic: VH
EVQLVESGGGLVQPGGSLRLSCAASGYSISSGHHWSWIRQAPGKGLEWIGEIYHSGSTYYSPSLKSRVTISRDNSKNTLYLQLNSLRAEDTAVYYCARRDFGASYFDYWGQGTLVTVSS

SEQ ID NO: 123 - protein, 106 aa, synthetic construct
  REGION 1..106: note = Synthetic: VL
DIQLTQSPSSLSASVGDRVTITCRASQGVGPWLAWYQQKPGKAPKLLIYAASSLQSGVPSRFSGSGSGTDFTLTISSLQPEDFATYYCQQYYSYSTFGQGTKVEIK

SEQ ID NO: 124 - protein, 446 aa, synthetic construct
  REGION 1..446: note = Synthetic: HC
EVQLVESGGGLVQPGGSLRLSCAASGYSISSGHHWSWIRQAPGKGLEWIGEIYHSGSTYYSPSLKSRVTISRDNSKNTLYLQLNSLRAEDTAVYYCARRDFGASYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 125 - protein, 213 aa, synthetic construct
  REGION 1..213: note = Synthetic: LC
DIQLTQSPSSLSASVGDRVTITCRASQGVGPWLAWYQQKPGKAPKLLIYAASSLQSGVPSRFSGSGSGTDFTLTISSLQPEDFATYYCQQYYSYSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 7 aa, synthetic construct
  REGION 1..7: note = Synthetic: HCDR1
TGGVGVT

SEQ ID NO: 127 - protein, 10 aa, synthetic construct
  REGION 1..10: note = Synthetic: HCDR3
YDYGYYSFDV

SEQ ID NO: 128 - protein, 15 aa, synthetic construct
  REGION 1..15: note = Synthetic: LCDR1
RASQSVDFDAFSFLA

SEQ ID NO: 129 - protein, 8 aa, synthetic construct
  REGION 1..8: note = Synthetic: LCDR3
QQSYSAST

SEQ ID NO: 130 - protein, 120 aa, synthetic construct
  REGION 1..120: note = Synthetic: VH
QVQLVQSGAEVKKPGSSVKVSCKASGFSLSTGGVGVTWIRQAPGQGLEWIGSIYHSGSTYYSPSLKSRVTITADKSTSTAYMELSSLRSEDTAVYYCARYDYGYYSFDVWGQGTLVTVSS

SEQ ID NO: 131 - protein, 110 aa, synthetic construct
  REGION 1..110: note = Synthetic: VL
DIQLTQSPSSLSASVGDRVTITCRASQSVDFDAFSFLAWYQQKPGKAPKLLIYAASTLQSGVPSRFSGSGSGTDFTLTISSLQPEDFATYYCQQSYSASTFGQGTKVEIK

SEQ ID NO: 132 - protein, 447 aa, synthetic construct
  REGION 1..447: note = Synthetic: HC
QVQLVQSGAEVKKPGSSVKVSCKASGFSLSTGGVGVTWIRQAPGQGLEWIGSIYHSGSTYYSPSLKSRVTITADKSTSTAYMELSSLRSEDTAVYYCARYDYGYYSFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 133 - protein, 217 aa, synthetic construct
  REGION 1..217: note = Synthetic: LC
DIQLTQSPSSLSASVGDRVTITCRASQSVDFDAFSFLAWYQQKPGKAPKLLIYAASTLQSGVPSRFSGSGSGTDFTLTISSLQPEDFATYYCQQSYSASTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 6 aa, synthetic construct
  REGION 1..6: note = Synthetic: HCDR1
SGHHWT

SEQ ID NO: 135 - protein, 10 aa, synthetic construct
  REGION 1..10: note = Synthetic: HCDR3
RDYGSSYFDY

SEQ ID NO: 136 - protein, 12 aa, synthetic construct
  REGION 1..12: note = Synthetic: LCDR1
RASQSVSRSFLA

SEQ ID NO: 137 - protein, 119 aa, synthetic construct
  REGION 1..119: note = Synthetic: VH
EVQLVESGGGLVQPGGSLRLSCAASGYSISSGHHWTWIRQAPGKGLEWIGEIYHSGSTYYSPSLKSRVTISRDNSKNTLYLQLNSLRAEDTAVYYCARRDYGSSYFDYWGQGTLVTVSS

SEQ ID NO: 138 - protein, 108 aa, synthetic construct
  REGION 1..108: note = Synthetic: VL
DIQLTQSPSSLSASVGDRVTITCRASQSVSRSFLAWYQQKPGKAPKLLIYDASNRATGIPSRFSGSGSGTDFTLTISSLQPEDFATYYCQQYYRSPITFGQGTKVEIK

SEQ ID NO: 139 - protein, 446 aa, synthetic construct
  REGION 1..446: note = Synthetic: HC
EVQLVESGGGLVQPGGSLRLSCAASGYSISSGHHWTWIRQAPGKGLEWIGEIYHSGSTYYSPSLKSRVTISRDNSKNTLYLQLNSLRAEDTAVYYCARRDYGSSYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 140 - protein, 215 aa, synthetic construct
  REGION 1..215: note = Synthetic: LC
DIQLTQSPSSLSASVGDRVTITCRASQSVSRSFLAWYQQKPGKAPKLLIYDASNRATGIPSRFSGSGSGTDFTLTISSLQPEDFATYYCQQYYRSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 10 aa, synthetic construct
  REGION 1..10: note = Synthetic: HCDR3
YGYSYYAFDY

SEQ ID NO: 142 - protein, 11 aa, synthetic construct
  REGION 1..11: note = Synthetic: LCDR1
RASQTIGSFLN

SEQ ID NO: 143 - protein, 8 aa, synthetic construct
  REGION 1..8: note = Synthetic: LCDR3
QQSYSYQT

SEQ ID NO: 144 - protein, 120 aa, synthetic construct
  REGION 1..120: note = Synthetic: VH
EVQLVESGGGLVQPGGSLRLSCAASGFSLSTSGVGVSWIRQAPGKGLEWIGAIYHSGSTYYSPSLKSRVTISRDNSKNTLYLQLNSLRAEDTAVYYCARYGYSYYAFDYWGQGTLVTVSS

SEQ ID NO: 145 - protein, 106 aa, synthetic construct
  REGION 1..106: note = Synthetic: VL
DIQLTQSPSSLSASVGDRVTITCRASQTIGSFLNWYQQKPGKAPKLLIYDASSLESGVPSRFSGSGSGTDFTLTISSLQPEDFATYYCQQSYSYQTFGQGTKVEIK

SEQ ID NO: 146 - protein, 447 aa, synthetic construct
  REGION 1..447: note = Synthetic: HC
EVQLVESGGGLVQPGGSLRLSCAASGFSLSTSGVGVSWIRQAPGKGLEWIGAIYHSGSTYYSPSLKSRVTISRDNSKNTLYLQLNSLRAEDTAVYYCARYGYSYYAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 147 - protein, 213 aa, synthetic construct
  REGION 1..213: note = Synthetic: LC
DIQLTQSPSSLSASVGDRVTITCRASQTIGSFLNWYQQKPGKAPKLLIYDASSLESGVPSRFSGSGSGTDFTLTISSLQPEDFATYYCQQSYSYQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 5 aa, synthetic construct
  REGION 1..5: note = Synthetic: HCDR1
NYGIH

SEQ ID NO: 149 - protein, 10 aa, synthetic construct
  REGION 1..10: note = Synthetic: HCDR3
GPLSYYALDY

SEQ ID NO: 150 - protein, 11 aa, synthetic construct
  REGION 1..11: note = Synthetic: LCDR1
RASQSIRRFLN

SEQ ID NO: 151 - protein, 118 aa, synthetic construct
  REGION 1..118: note = Synthetic: VH
QVQLVQSGAEVKKPGSSVKVSCKASGYTFTNYGIHWVRQAPGQGLEWIGEIYHSGSTYYSPSLKSRVTITADKSTSTAYMELSSLRSEDTAVYYCARGPLSYYALDYWGQGTLVTVSS

SEQ ID NO: 152 - protein, 107 aa, synthetic construct
  REGION 1..107: note = Synthetic: VL
DIQLTQSPSSLSASVGDRVTITCRASQSIRRFLNWYQQKPGKAPKLLIYDASSLESGVPSRFSGSGSGTDFTLTISSLQPEDFATYYCQQYYRSPITFGQGTKVEIK

SEQ ID NO: 153 - protein, 445 aa, synthetic construct
  REGION 1..445: note = Synthetic: HC
QVQLVQSGAEVKKPGSSVKVSCKASGYTFTNYGIHWVRQAPGQGLEWIGEIYHSGSTYYSPSLKSRVTITADKSTSTAYMELSSLRSEDTAVYYCARGPLSYYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 154 - protein, 214 aa, synthetic construct
  REGION 1..214: note = Synthetic: LC
DIQLTQSPSSLSASVGDRVTITCRASQSIRRFLNWYQQKPGKAPKLLIYDASSLESGVPSRFSGSGSGTDFTLTISSLQPEDFATYYCQQYYRSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 16 aa, synthetic construct
  REGION 1..16: note = Synthetic: HCDR2
DIYHSGSTYYNPSLKS

SEQ ID NO: 156 - protein, 15 aa, synthetic construct
  REGION 1..15: note = Synthetic: LCDR1
RAPQSVDFHGKSFLH

SEQ ID NO: 157 - protein, 118 aa, synthetic construct
  REGION 1..118: note = Synthetic: VH
EVQLVESGGGLVQPGGSLRLSCAASGYSITSGHHWSWIRQAPGKGLEWIGDIYHSGSTYYNPSLKSRVTISRDNSKNTLYLQLNSLRAEDTAVYYCARKDDYYAFDYWGQGTLVTVSS

SEQ ID NO: 158 - protein, 111 aa, synthetic construct
  REGION 1..111: note = Synthetic: VL
DIQLTQSPSSLSASVGDRVTITCRAPQSVDFHGKSFLHWYQQKPGKAPKLLIYDASNRATGIPSRFSGSGSGTDFTLTISSLQPEDFATYYCQQSYRSPITFGQGTKVEIK

SEQ ID NO: 159 - protein, 445 aa, synthetic construct
  REGION 1..445: note = Synthetic: HC
EVQLVESGGGLVQPGGSLRLSCAASGYSITSGHHWSWIRQAPGKGLEWIGDIYHSGSTYYNPSLKSRVTISRDNSKNTLYLQLNSLRAEDTAVYYCARKDDYYAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 160 - protein, 218 aa, synthetic construct
  REGION 1..218: note = Synthetic: LC
DIQLTQSPSSLSASVGDRVTITCRAPQSVDFHGKSFLHWYQQKPGKAPKLLIYDASNRATGIPSRFSGSGSGTDFTLTISSLQPEDFATYYCQQSYRSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 7 aa, synthetic construct
  REGION 1..7: note = Synthetic: HCDR1
TSGVAVG

SEQ ID NO: 162 - protein, 10 aa, synthetic construct
  REGION 1..10: note = Synthetic: HCDR3
HGYHYYALDY

SEQ ID NO: 163 - protein, 11 aa, synthetic construct
  REGION 1..11: note = Synthetic: LCDR1
PASQDVGTAVA

SEQ ID NO: 164 - protein, 9 aa, synthetic construct
  REGION 1..9: note = Synthetic: LCDR3
QQYYSSPPT

SEQ ID NO: 165 - protein, 120 aa, synthetic construct
  REGION 1..120: note = Synthetic: VH
EVQLVESGGGLVQPGGSLRLSCAASGFSLSTSGVAVGWIRQAPGKGLEWIGEIYHSGSTYYSPSLKSRVTISRDNSKNTLYLQLNSLRAEDTAVYYCARHGYHYYALDYWGQGTLVTVSS

SEQ ID NO: 166 - protein, 107 aa, synthetic construct
  REGION 1..107: note = Synthetic: VL
DIQLTQSPSSLSASVGDRVTITCPASQDVGTAVAWYQQKPGKAPKLLIYDASSLESGVPSRFSGSGSGTDFTLTISSLQPEDFATYYCQQYYSSPPTFGQGTKVEIK

SEQ ID NO: 167 - protein, 447 aa, synthetic construct
  REGION 1..447: note = Synthetic: HC
EVQLVESGGGLVQPGGSLRLSCAASGFSLSTSGVAVGWIRQAPGKGLEWIGEIYHSGSTYYSPSLKSRVTISRDNSKNTLYLQLNSLRAEDTAVYYCARHGYHYYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 168 - protein, 214 aa, synthetic construct
  REGION 1..214: note = Synthetic: LC
DIQLTQSPSSLSASVGDRVTITCPASQDVGTAVAWYQQKPGKAPKLLIYDASSLESGVPSRFSGSGSGTDFTLTISSLQPEDFATYYCQQYYSSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 214 aa, synthetic construct
  REGION 1..214: note = Synthetic: LC
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 449 aa, synthetic construct
  REGION 1..449: note = Synthetic: HC-A
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKKLTKNQVKLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 171 - protein, 483 aa, synthetic construct
  REGION 1..483: note = Synthetic: HC-B
DIQLTQSPSSLSASVGDRVTITCRASQSVDFHGISFLHWYQQKPGKAPKLLIYAASSLQSGVPSRFSGSGSGTDFTLTISSLQPEDFATYYCQQSYRSPRTFGQGTKVEIKGGGGSGGGGSGGGGSGGGGSEVQLVESGGGLVQPGGSLRLSCAASGFSLSTGGVGVSWIRQAPGKGLEWIGEIYHSGSTYYSPSLKSRVTISRDNSKNTLYLQLNSLRAEDTAVYYCARYEYYYYDFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 172 - protein, 483 aa, synthetic construct
  REGION 1..483: note = Synthetic: HC-B
DIQLTQSPSSLSASVGDRVTITCRASQSVDFYGKSFLHWYQQKPGKAPKLLIYAASSLQSGVPSRFSGSGSGTDFTLTISSLQPEDFATYYCQQSYRSPLTFGQGTKVEIKGGGGSGGGGSGGGGSGGGGSEVQLVESGGGLVQPGGSLRLSCAASGFSLSTSGVGVSWIRQAPGKGLEWIGEIYHSGSTYYSPSLKSRVTISRDNSKNTLYLQLNSLRVEDTAVYYCARDEYDYYAFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 173 - protein, 214 aa, synthetic construct
  REGION 1..214: note = Synthetic: LC
DIQLTQSPSSLSASVGDRVTITCRASQDVSIAVAWYQQKPGKAPKLLIYSASYRYTGVPARFSGSGSGTDFTLTISSLQPEDFATYYCQQHYITPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 451 aa, synthetic construct
  REGION 1..451: note = Synthetic: HC-A
QVQLLQSGSELKKPGASVKVSCKASGYTFTNYGINWVRQAPGQGLEWLGWIHTYTGEPTYTDDFKGRFVFSLDTSVSTAYLQISSLKADDTAVYYCARGGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKKLTKNQVKLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 175 - protein, 214 aa, synthetic construct
  REGION 1..214: note = Synthetic: LC
DIQLTQSPSSLSASVGDRVTITCRASQSIRKYLNWYQQKPGKAPKLLIYAASTLQSGVPSRFSGSGSGTDFTLTISSLQPEDFATYFCSQGTHVPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 448 aa, synthetic construct
  REGION 1..448: note = Synthetic: HC-A
EVQLVESGGGLVQPGGSLRLSCAASGFTFSGYGIHWVRQAPGKGLEWIGWISPSGGDTKYAQKFQGRVTISRDNSKNTLYLQLNSLRAEDTAVYYCARGGTVVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKKLTKNQVKLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 177 - protein, 486 aa, synthetic construct
  REGION 1..486: note = Synthetic: HC-B
QAVVTQEPSLTVSPGGTVTLTCGSSTGAVTTSNYPNWVQQKPGQAPRGLIGGTNKRAPGVPARFSGSLLGGKAALTLSGAQPEDEAEYYCALWYSNLWVFGGGTKLTVLGGGGSGGGGSGGGGSGGGGSEVQLVESGGGLVQPGGSLRLSCAASGFTFNTYAINWVRQAPGKGLEWVGRIRSKYNNYATYYAESVKGRFTISRDDSKNTLYLQINSLRAEDTAVYYCVRHGNFGTSYVSWFAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 178 - protein, 16 aa, synthetic construct
  REGION 1..16: note = Synthetic: Masking Unit (MU)
ASDLYVCAHDPYFCHS

SEQ ID NO: 179 - protein, 16 aa, synthetic construct
  REGION 1..16: note = Synthetic: Masking Unit (MU)
PNVFDTCYVDPYSCTI

SEQ ID NO: 180 - protein, 16 aa, synthetic construct
  REGION 1..16: note = Synthetic: Masking Unit (MU)
YAAAPSCPPANYYCAD

SEQ ID NO: 181 - protein, 16 aa, synthetic construct
  REGION 1..16: note = Synthetic: Masking Unit (MU)
HHADPLCFAQHSACNT

SEQ ID NO: 182 - protein, 16 aa, synthetic construct
  REGION 1..16: note = Synthetic: Masking Unit (MU)
YDTHDYCLIHYHSCDT

SEQ ID NO: 183 - protein, 16 aa, synthetic construct
  REGION 1..16: note = Synthetic: Masking Unit (MU)
ADFAPYCYADPYYCHD

SEQ ID NO: 184 - protein, 16 aa, synthetic construct
  REGION 1..16: note = Synthetic: Masking Unit (MU)
DIASDSCYYHPNHCDT

SEQ ID NO: 185 - protein, 16 aa, synthetic construct
  REGION 1..16: note = Synthetic: Masking Unit (MU)
AATYSDCYSDPYACHD

SEQ ID NO: 186 - protein, 16 aa, synthetic construct
  REGION 1..16: note = Synthetic: Masking Unit (MU)
PTYDHDCAVYNNFCNA

SEQ ID NO: 187 - protein, 16 aa, synthetic construct
  REGION 1..16: note = Synthetic: Masking Unit (MU)
HNHALTCYTDPYACHS

SEQ ID NO: 188 - protein, 16 aa, synthetic construct
  REGION 1..16: note = Synthetic: Masking Unit (MU)
AAFSPACYTDPYSCHA

SEQ ID NO: 189 - protein, 16 aa, synthetic construct
  REGION 1..16: note = Synthetic: Masking Unit (MU)
TPHPDACFVDPYHCHT

SEQ ID NO: 190 - protein, 16 aa, synthetic construct
  REGION 1..16: note = Synthetic: Masking Unit (MU)
APSDAYCYADPYACHT

SEQ ID NO: 191 - protein, 16 aa, synthetic construct
  REGION 1..16: note = Synthetic: Masking Unit (MU)
AVDDDPCYTDPYQCTA

SEQ ID NO: 192 - protein, 16 aa, synthetic construct
  REGION 1..16: note = Synthetic: Masking Unit (MU)
THSDAVCAPDHAYCLY

SEQ ID NO: 193 - protein, 16 aa, synthetic construct
  REGION 1..16: note = Synthetic: Masking Unit (MU)
PYIYVDCYAPPFFCAY

SEQ ID NO: 194 - protein, 14 aa, synthetic construct
  REGION 1..14: note = Synthetic: Masking Unit (MU)
YFCAEGVPIVLCIG

SEQ ID NO: 195 - protein, 14 aa, synthetic construct
  REGION 1..14: note = Synthetic: Masking Unit (MU)
DYCEGPPAFFYCAK

SEQ ID NO: 196 - protein, 14 aa, synthetic construct
  REGION 1..14: note = Synthetic: Masking Unit (MU)
FPCEPLADPFLCRQ

SEQ ID NO: 197 - protein, 14 aa, synthetic construct
  REGION 1..14: note = Synthetic: Masking Unit (MU)
LVCEDVPFPAACRA

SEQ ID NO: 198 - protein, 14 aa, synthetic construct
  REGION 1..14: note = Synthetic: Masking Unit (MU)
ALCEFYAFPYFCAR

SEQ ID NO: 199 - protein, 14 aa, synthetic construct
  REGION 1..14: note = Synthetic: Masking Unit (MU)
IVCKYFDDPFLCRR

SEQ ID NO: 200 - protein, 14 aa, synthetic construct
  REGION 1..14: note = Synthetic: Masking Unit (MU)
YFCPVRVPLAVCLR

SEQ ID NO: 201 - protein, 14 aa, synthetic construct
  REGION 1..14: note = Synthetic: Masking Unit (MU)
PSCPDYVPFPVCAQ

SEQ ID NO: 202 - protein, 14 aa, synthetic construct
  REGION 1..14: note = Synthetic: Masking Unit (MU)
VACAKAFAYVYCLV

SEQ ID NO: 203 - protein, 14 aa, synthetic construct
  REGION 1..14: note = Synthetic: Masking Unit (MU)
VLCGDSVPFLLCEP

SEQ ID NO: 204 - protein, 14 aa, synthetic construct
  REGION 1..14: note = Synthetic: Masking Unit (MU)
ILCPEGVPVVFCVL

SEQ ID NO: 205 - protein, 14 aa, synthetic construct
  REGION 1..14: note = Synthetic: Masking Unit (MU)
SDCGLGVPLLVCLK

SEQ ID NO: 206 - protein, 14 aa, synthetic construct
  REGION 1..14: note = Synthetic: Masking Unit (MU)
TDCGIPFPVDDCLS

SEQ ID NO: 207 - protein, 16 aa, synthetic construct
  REGION 1..16: note = Synthetic: Masking Unit (MU)
HIPDHYCAADHHYCYY

SEQ ID NO: 208 - protein, 16 aa, synthetic construct
  REGION 1..16: note = Synthetic: Masking Unit (MU)
AATYSDCYSDPYACHD

SEQ ID NO: 209 - protein, 13 aa, synthetic construct
  REGION 1..13: note = Synthetic: Masking Unit (MU)
YSDCYSDPYACHD

SEQ ID NO: 210 - protein, 16 aa, synthetic construct
  REGION 1..16: note = Synthetic: Masking Unit (MU)
AAFSPACYTDPYSCHA

SEQ ID NO: 211 - protein, 13 aa, synthetic construct
  REGION 1..13: note = Synthetic: Masking Unit (MU)
SPACYTDPYSCHA

SEQ ID NO: 212 - protein, 5 aa, synthetic construct
  REGION 1..5: note = Synthetic: N-terminal Unit
EVGSY

SEQ ID NO: 213 - protein, 13 aa, synthetic construct
  REGION 1..13: note = Synthetic: Linkage Unit (LU)
GGGPLGLAGSGGS

SEQ ID NO: 214 - protein, 14 aa, synthetic construct
  REGION 1..14: note = Synthetic: Linkage Unit (LU)
SGGGPLGLAGSGGS

SEQ ID NO: 215 - protein, 34 aa, synthetic construct
  REGION 1..34: note = Synthetic: Masking+Cleavage Peptide Sequence
EVGSYASDLYVCAHDPYFCHSGGGPLGLAGSGGS

SEQ ID NO: 216 - protein, 34 aa, synthetic construct
  REGION 1..34: note = Synthetic: Masking+Cleavage Peptide Sequence
EVGSYPNVFDTCYVDPYSCTIGGGPLGLAGSGGS

SEQ ID NO: 217 - protein, 34 aa, synthetic construct
  REGION 1..34: note = Synthetic: Masking+Cleavage Peptide Sequence
EVGSYYAAAPSCPPANYYCADGGGPLGLAGSGGS

SEQ ID NO: 218 - protein, 34 aa, synthetic construct
  REGION 1..34: note = Synthetic: Masking+Cleavage Peptide Sequence
EVGSYHHADPLCFAQHSACNTGGGPLGLAGSGGS

SEQ ID NO: 219 - protein, 34 aa, synthetic construct
  REGION 1..34: note = Synthetic: Masking+Cleavage Peptide Sequence
EVGSYYDTHDYCLIHYHSCDTGGGPLGLAGSGGS

SEQ ID NO: 220 - protein, 34 aa, synthetic construct
  REGION 1..34: note = Synthetic: Masking+Cleavage Peptide Sequence
EVGSYADFAPYCYADPYYCHDGGGPLGLAGSGGS

SEQ ID NO: 221 - protein, 34 aa, synthetic construct
  REGION 1..34: note = Synthetic: Masking+Cleavage Peptide Sequence
EVGSYDIASDSCYYHPNHCDTGGGPLGLAGSGGS

SEQ ID NO: 222 - protein, 34 aa, synthetic construct
  REGION 1..34: note = Synthetic: Masking+Cleavage Peptide Sequence
EVGSYAATYSDCYSDPYACHDGGGPLGLAGSGGS

SEQ ID NO: 223 - protein, 34 aa, synthetic construct
  REGION 1..34: note = Synthetic: Masking+Cleavage Peptide Sequence
EVGSYPTYDHDCAVYNNFCNAGGGPLGLAGSGGS

SEQ ID NO: 224 - protein, 34 aa, synthetic construct
  REGION 1..34: note = Synthetic: Masking+Cleavage Peptide Sequence
EVGSYHNHALTCYTDPYACHSGGGPLGLAGSGGS

SEQ ID NO: 225 - protein, 34 aa, synthetic construct
  REGION 1..34: note = Synthetic: Masking+Cleavage Peptide Sequence
EVGSYAAFSPACYTDPYSCHAGGGPLGLAGSGGS

SEQ ID NO: 226 - protein, 34 aa, synthetic construct
  REGION 1..34: note = Synthetic: Masking+Cleavage Peptide Sequence
EVGSYTPHPDACFVDPYHCHTGGGPLGLAGSGGS

SEQ ID NO: 227 - protein, 34 aa, synthetic construct
  REGION 1..34: note = Synthetic: Masking+Cleavage Peptide Sequence
EVGSYAPSDAYCYADPYACHTGGGPLGLAGSGGS

SEQ ID NO: 228 - protein, 34 aa, synthetic construct
  REGION 1..34: note = Synthetic: Masking+Cleavage Peptide Sequence
EVGSYAVDDDPCYTDPYQCTAGGGPLGLAGSGGS

SEQ ID NO: 229 - protein, 34 aa, synthetic construct
  REGION 1..34: note = Synthetic: Masking+Cleavage Peptide Sequence
EVGSYTHSDAVCAPDHAYCLYGGGPLGLAGSGGS

SEQ ID NO: 230 - protein, 34 aa, synthetic construct
  REGION 1..34: note = Synthetic: Masking+Cleavage Peptide Sequence
EVGSYPYIYVDCYAPPFFCAYGGGPLGLAGSGGS

SEQ ID NO: 231 - protein, 32 aa, synthetic construct
  REGION 1..32: note = Synthetic: Masking+Cleavage Peptide Sequence
EVGSYYFCAEGVPIVLCIGGGGPLGLAGSGGS

SEQ ID NO: 232 - protein, 32 aa, synthetic construct
  REGION 1..32: note = Synthetic: Masking+Cleavage Peptide Sequence
EVGSYDYCEGPPAFFYCAKGGGPLGLAGSGGS

SEQ ID NO: 233 - protein, 32 aa, synthetic construct
  REGION 1..32: note = Synthetic: Masking+Cleavage Peptide Sequence
EVGSYFPCEPLADPFLCRQGGGPLGLAGSGGS

SEQ ID NO: 234 - protein, 32 aa, synthetic construct
  REGION 1..32: note = Synthetic: Masking+Cleavage Peptide Sequence
EVGSYLVCEDVPFPAACRAGGGPLGLAGSGGS

SEQ ID NO: 235 - protein, 32 aa, synthetic construct
  REGION 1..32: note = Synthetic: Masking+Cleavage Peptide Sequence
EVGSYALCEFYAFPYFCARGGGPLGLAGSGGS

SEQ ID NO: 236 - protein, 32 aa, synthetic construct
  REGION 1..32: note = Synthetic: Masking+Cleavage Peptide Sequence
EVGSYIVCKYFDDPFLCRRGGGPLGLAGSGGS

SEQ ID NO: 237 - protein, 32 aa, synthetic construct
  REGION 1..32: note = Synthetic: Masking+Cleavage Peptide Sequence
EVGSYYFCPVRVPLAVCLRGGGPLGLAGSGGS

SEQ ID NO: 238 - protein, 32 aa, synthetic construct
  REGION 1..32: note = Synthetic: Masking+Cleavage Peptide Sequence
EVGSYPSCPDYVPFPVCAQGGGPLGLAGSGGS

SEQ ID NO: 239 - protein, 32 aa, synthetic construct
  REGION 1..32: note = Synthetic: Masking+Cleavage Peptide Sequence
EVGSYVACAKAFAYVYCLVGGGPLGLAGSGGS

SEQ ID NO: 240 - protein, 32 aa, synthetic construct
  REGION 1..32: note = Synthetic: Masking+Cleavage Peptide Sequence
EVGSYVLCGDSVPFLLCEPGGGPLGLAGSGGS

SEQ ID NO: 241 - protein, 32 aa, synthetic construct
  REGION 1..32: note = Synthetic: Masking+Cleavage Peptide Sequence
EVGSYILCPEGVPVVFCVLGGGPLGLAGSGGS

SEQ ID NO: 242 - protein, 32 aa, synthetic construct
  REGION 1..32: note = Synthetic: Masking+Cleavage Peptide Sequence
EVGSYSDCGLGVPLLVCLKGGGPLGLAGSGGS

SEQ ID NO: 243 - protein, 32 aa, synthetic construct
  REGION 1..32: note = Synthetic: Masking+Cleavage Peptide Sequence
EVGSYTDCGIPFPVDDCLSGGGPLGLAGSGGS

SEQ ID NO: 244 - protein, 34 aa, synthetic construct
  REGION 1..34: note = Synthetic: Masking+Cleavage Peptide Sequence
EVGSYHIPDHYCAADHHYCYYGGGPLGLAGSGGS

SEQ ID NO: 245 - protein, 31 aa, synthetic construct
  REGION 1..31: note = Synthetic: Masking+Cleavage Peptide Sequence
EAATYSDCYSDPYACHDSGGGPLGLAGSGGS

SEQ ID NO: 246 - protein, 28 aa, synthetic construct
  REGION 1..28: note = Synthetic: Masking+Cleavage Peptide Sequence
EYSDCYSDPYACHDSGGGPLGLAGSGGS

SEQ ID NO: 247 - protein, 30 aa, synthetic construct
  REGION 1..30: note = Synthetic: Masking+Cleavage Peptide Sequence
EAAFSPACYTDPYSCHAGGGPLGLAGSGGS

SEQ ID NO: 248 - protein, 27 aa, synthetic construct
  REGION 1..27: note = Synthetic: Masking+Cleavage Peptide Sequence
ESPACYTDPYSCHAGGGPLGLAGSGGS

SEQ ID NO: 249 - protein, 5 aa, synthetic construct
  REGION 1..5: note = Synthetic: Flexible Linker
GGGGS

SEQ ID NO: 250 - protein, 5 aa, synthetic construct
  REGION 1..5: note = Synthetic: Flexible Linker
GGGGT

SEQ ID NO: 251 - protein, 4 aa, synthetic construct
  REGION 1..4: note = Synthetic: Flexible Linker
SGGS

SEQ ID NO: 252 - protein, 4 aa, synthetic construct
  REGION 1..4: note = Synthetic: Flexible Linker
GGSG

SEQ ID NO: 253 - protein, 5 aa, synthetic construct
  REGION 1..5: note = Synthetic: Flexible Linker
GGSGG

SEQ ID NO: 254 - protein, 5 aa, synthetic construct
  REGION 1..5: note = Synthetic: Flexible Linker
GSGSG

SEQ ID NO: 255 - protein, 5 aa, synthetic construct
  REGION 1..5: note = Synthetic: Flexible Linker
GSGGG

SEQ ID NO: 256 - protein, 5 aa, synthetic construct
  REGION 1..5: note = Synthetic: Flexible Linker
GGGSG

SEQ ID NO: 257 - protein, 5 aa, synthetic construct
  REGION 1..5: note = Synthetic: Flexible Linker
GSSSG

SEQ ID NO: 258 - protein, 11 aa, synthetic construct
  REGION 1..11: note = Synthetic: LCDR1
KASQDVSIGVA

SEQ ID NO: 259 - protein, 7 aa, synthetic construct
  REGION 1..7: note = Synthetic: LCDR2 / LCDR2-A
SASYRYT

SEQ ID NO: 260 - protein, 9 aa, synthetic construct
  REGION 1..9: note = Synthetic: LCDR3
QQYYIYPYT

SEQ ID NO: 261 - protein, 107 aa, synthetic construct [sequence omitted]

SEQ ID NO: 262 - protein, 5 aa, synthetic construct
  REGION 1..5: note = Synthetic: HCDR1-A
DYTMD

SEQ ID NO: 263 - protein, 17 aa, synthetic construct
  REGION 1..17: note = Synthetic: HCDR2-A
DVNPNSGGSIYNQRFKG

SEQ ID NO: 264 - protein, 10 aa, synthetic construct
  REGION 1..10: note = Synthetic: HCDR3-A
NLGPSFYFDY

SEQ ID NO: 265 - protein, 119 aa, synthetic construct [sequence omitted]

SEQ ID NO: 266 - protein, 251 aa, synthetic construct
  REGION 1..251: note = Synthetic: scFv-B
DIQLTQSPSSLSASVGDRVTITCRASQSVDFHGISFLHWYQQKPGKAPKLLIYAASSLQSGVPSRFSGSGSGTDFTLTISSLQPEDFATYYCQQSYRSPRTFGQGTKVEIKGGGGSGGGGSGGGGSGGGGSEVQLVESGGGLVQPGGSLRLSCAASGFSLSTGGVGVSWIRQAPGKGLEWIGEIYHSGSTYYSPSLKSRVTISRDNSKNTLYLQLNSLRAEDTAVYYCARYEYYYYDFDYWGQGTLVTVSS

SEQ ID NO: 267 - protein, 11 aa, synthetic construct
  REGION 1..11: note = Synthetic: LCDR1
RASQDVNTAVA

SEQ ID NO: 268 - protein, 7 aa, synthetic construct
  REGION 1..7: note = Synthetic: LCDR2
SASFLYS

SEQ ID NO: 269 - protein, 9 aa, synthetic construct
  REGION 1..9: note = Synthetic: LCDR3
QQHYTTPPT

SEQ ID NO: 270 - protein, 107 aa, synthetic construct [sequence omitted]

SEQ ID NO: 271 - protein, 214 aa, synthetic construct
  REGION 1..214: note = Synthetic: LC
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 272 - protein, 5 aa, synthetic construct
  REGION 1..5: note = Synthetic: HCDR1-A
DTYIH

SEQ ID NO: 273 - protein, 17 aa, synthetic construct
  REGION 1..17: note = Synthetic: HCDR2-A
RIYPTNGYTRYADSVKG

SEQ ID NO: 274 - protein, 11 aa, synthetic construct
  REGION 1..11: note = Synthetic: HCDR3-A
WGGDGFYAMDY

SEQ ID NO: 275 - protein, 120 aa, synthetic construct [sequence omitted]

SEQ ID NO: 276 - protein, 450 aa, synthetic construct
  REGION 1..450: note = Synthetic: HC-A
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKKLTKNQVKLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 277 - protein, 11 aa, synthetic construct
  REGION 1..11: note = Synthetic: LCDR1-A
RASQDVSIAVA

SEQ ID NO: 278 - protein, 9 aa, synthetic construct
  REGION 1..9: note = Synthetic: LCDR3-A
QQHYITPLT

SEQ ID NO: 279 - protein, 107 aa, synthetic construct
  REGION 1..107: note = Synthetic: VL-A
DIQLTQSPSSLSASVGDRVTITCRASQDVSIAVAWYQQKPGKAPKLLIYSASYRYTGVPARFSGSGSGTDFTLTISSLQPEDFATYYCQQHYITPLTFGQGTKVEIK

SEQ ID NO: 280 - protein, 5 aa, synthetic construct
  REGION 1..5: note = Synthetic: HCDR1-A
NYGIN

SEQ ID NO: 281 - protein, 17 aa, synthetic construct
  REGION 1..17: note = Synthetic: HCDR2-A
WIHTYTGEPTYTDDFKG

SEQ ID NO: 282 - protein, 12 aa, synthetic construct
  REGION 1..12: note = Synthetic: HCDR3-A
GGFGSSYWYFDV

SEQ ID NO: 283 - protein, 121 aa, synthetic construct
  REGION 1..121: note = Synthetic: VH-A
QVQLLQSGSELKKPGASVKVSCKASGYTFTNYGINWVRQAPGQGLEWLGWIHTYTGEPTYTDDFKGRFVFSLDTSVSTAYLQISSLKADDTAVYYCARGGFGSSYWYFDVWGQGTLVTVSS

SEQ ID NO: 284-286 - skipped (3 entries)

SEQ ID NO: 287 - protein, 11 aa, synthetic construct
  REGION 1..11: note = Synthetic: LCDR1-A
RASQSIRKYLN

SEQ ID NO: 288 - protein, 8 aa, synthetic construct
  REGION 1..8: note = Synthetic: LCDR3-A
QGTHVPFT

SEQ ID NO: 289 - protein, 107 aa, synthetic construct
  REGION 1..107: note = Synthetic: VL-A
DIQLTQSPSSLSASVGDRVTITCRASQSIRKYLNWYQQKPGKAPKLLIYAASTLQSGVPSRFSGSGSGTDFTLTISSLQPEDFATYFCSQGTHVPFTFGQGTKVEIK

SEQ ID NO: 290 - protein, 5 aa, synthetic construct
  REGION 1..5: note = Synthetic: HCDR1-A
GYGIH

SEQ ID NO: 291 - protein, 17 aa, synthetic construct
  REGION 1..17: note = Synthetic: HCDR2-A
WISPSGGDTKYAQKFQG

SEQ ID NO: 292 - protein, 9 aa, synthetic construct
  REGION 1..9: note = Synthetic: HCDR3-A
GGTVVYFDY

SEQ ID NO: 293 - protein, 118 aa, synthetic construct
  REGION 1..118: note = Synthetic: VH-A
EVQLVESGGGLVQPGGSLRLSCAASGFTFSGYGIHWVRQAPGKGLEWIGWISPSGGDTKYAQKFQGRVTISRDNSKNTLYLQLNSLRAEDTAVYYCARGGTVVYFDYWGQGTLVTVSS

SEQ ID NO: 294 - protein, 19 aa, synthetic construct
  REGION 1..19: note = Synthetic: HCDR2-B
RIRSKYNNYATYYAESVKG

SEQ ID NO: 295 - protein, 14 aa, synthetic construct
  REGION 1..14: note = Synthetic: HCDR3-B
HGNFGTSYVSWFAY

SEQ ID NO: 296 - protein, 14 aa, synthetic construct
  REGION 1..14: note = Synthetic: LCDR1-B
GSSTGAVTTSNYPN

SEQ ID NO: 297 - protein, 7 aa, synthetic construct
  REGION 1..7: note = Synthetic: LCDR2-B
GTNKRAP

SEQ ID NO: 298 - protein, 9 aa, synthetic construct
  REGION 1..9: note = Synthetic: LCDR3-B
ALWYSNLWV

SEQ ID NO: 299 - protein, 254 aa, synthetic construct
  REGION 1..254: note = Synthetic: scFv-B
QAVVTQEPSLTVSPGGTVTLTCGSSTGAVTTSNYPNWVQQKPGQAPRGLIGGTNKRAPGVPARFSGSLLGGKAALTLSGAQPEDEAEYYCALWYSNLWVFGGGTKLTVLGGGGSGGGGSGGGGSGGGGSEVQLVESGGGLVQPGGSLRLSCAASGFTFNTYAINWVRQAPGKGLEWVGRIRSKYNNYATYYAESVKGRFTISRDDSKNTLYLQINSLRAEDTAVYYCVRHGNFGTSYVSWFAYWGQGTLVTVSS

SEQ ID NO: 300 - protein, 7 aa, synthetic construct
  REGION 1..7: note = Synthetic: LCDR2
DASSLES

SEQ ID NO: 301 - protein, 34 aa, synthetic construct; identical to SEQ ID NO: 222
  REGION 1..34: note = Synthetic: Masking+cleavage peptide sequence

SEQ ID NO: 302 - protein, 31 aa, synthetic construct; identical to SEQ ID NO: 245
  REGION 1..31: note = Synthetic: Masking+cleavage peptide sequence

SEQ ID NO: 303 - protein, 28 aa, synthetic construct
  REGION 1..28: note = Synthetic: Masking+cleavage peptide sequence
EYSDCYSDPYACHDSGGGPLGLAGSGGS

SEQ ID NO: 304 - protein, 34 aa, synthetic construct; identical to SEQ ID NO: 225
  REGION 1..34: note = Synthetic: Masking+cleavage peptide sequence

SEQ ID NO: 305 - protein, 30 aa, synthetic construct; identical to SEQ ID NO: 247
  REGION 1..30: note = Synthetic: Masking+cleavage peptide sequence

SEQ ID NO: 306 - protein, 27 aa, synthetic construct
  REGION 1..27: note = Synthetic: Masking+cleavage peptide sequence
ESPACYTDPYSCHAGGGPLGLAGSGGS

SEQ ID NO: 307 - protein, 10 aa, synthetic construct
  REGION 1..10: note = Synthetic: HCDR3
YEYDYYAFDY

SEQ ID NO: 308 - protein, 7 aa, synthetic construct
  REGION 1..7: note = Synthetic: LCDR2
AASSLES

SEQ ID NO: 309 - protein, 10 aa, synthetic construct
  REGION 1..10: note = Synthetic: HCDR3
YEYGYYALDY

SEQ ID NO: 310 - protein, 15 aa, synthetic construct
  REGION 1..15: note = Synthetic: LCDR1
RASQSVDFYGISFLH

SEQ ID NO: 311 - protein, 9 aa, synthetic construct
  REGION 1..9: note = Synthetic: LCDR3
QQSYRTPPT

SEQ ID NO: 312 - protein, 10 aa, synthetic construct
  REGION 1..10: note = Synthetic: HCDR3
YEYGYYAFDY

SEQ ID NO: 313 - protein, 9 aa, synthetic construct
  REGION 1..9: note = Synthetic: LCDR3
QQSYRTPRT

SEQ ID NO: 314 - protein, 7 aa, synthetic construct
  REGION 1..7: note = Synthetic: HCDR1
TSGVGVG

SEQ ID NO: 315 - protein, 16 aa, synthetic construct
  REGION 1..16: note = Synthetic: HCDR2
EIYHSGSTNYNPSLKS

SEQ ID NO: 316 - protein, 16 aa, synthetic construct
  REGION 1..16: note = Synthetic: HCDR2
EIYHSGSTYYNPSLKS

SEQ ID NO: 317 - protein, 10 aa, synthetic construct
  REGION 1..10: note = Synthetic: HCDR3
YEYDYYDFDY

SEQ ID NO: 318 - protein, 9 aa, synthetic construct
  REGION 1..9: note = Synthetic: LCDR3
QQSYRSPLT

SEQ ID NO: 319 - protein, 10 aa, synthetic construct
  REGION 1..10: note = Synthetic: HCDR3
YEYYSYAFDY

SEQ ID NO: 320 - protein, 15 aa, synthetic construct
  REGION 1..15: note = Synthetic: LCDR1
RASQSVDFHGKSFLH

SEQ ID NO: 321 - protein, 10 aa, synthetic construct
  REGION 1..10: note = Synthetic: HCDR3
YGYYYYDFDY

SEQ ID NO: 322 - protein, 10 aa, synthetic construct
  REGION 1..10: note = Synthetic: HCDR3
YKYDYYDFDY

SEQ ID NO: 323 - protein, 10 aa, synthetic construct
  REGION 1..10: note = Synthetic: HCDR3
YEYYEYDFDY

SEQ ID NO: 324 - protein, 10 aa, synthetic construct
  REGION 1..10: note = Synthetic: HCDR3
YEYYHYDFDY

SEQ ID NO: 325 - protein, 216 aa, synthetic construct
  REGION 1..216: note = Synthetic: LC
DIQLTQSPSSLSASVGDRVTITCRASQSIPSFLNWYQQKPGKAPKLLIYAASSLQSGVPSRFSGSGSGTDFTLTISSLQPEDFATYYCQHYISWPRQFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 326 - protein, 448 aa, synthetic construct
  REGION 1..448: note = Synthetic: HC_A
EVQLVESGGGLVQPGGSLRLSCAASGYSISSGYYWGWIRQAPGKGLEWIGIIYPSGGGTNYAQKFQGRVTISRDNSKNTLYLQLNSLRAEDTAVYYCARGGGLGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKKLTKNQVKLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 327 - protein, 483 aa, synthetic construct
  REGION 1..483: note = Synthetic: HC_B
EVQLVESGGGLVQPGGSLRLSCAASGFSLSTGGVGVSWIRQAPGKGLEWIGEIYHSGSTYYSPSLKSRVTISRDNSKNTLYLQLNSLRAEDTAVYYCARYEYYYYDFDYWGQGTLVTVSSGGGGSGGGGSGGGGSGGGGSDIQLTQSPSSLSASVGDRVTITCRASQSVDFHGISFLHWYQQKPGKAPKLLIYAASSLQSGVPSRFSGSGSGTDFTLTISSLQPEDFATYYCQQSYRSPRTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 328 - protein, 483 aa, synthetic construct
  REGION 1..483: note = Synthetic: HC_B
EVQLVESGGGLVQPGGSLRLSCAASGFSLSTGGVGVSWIRQAPGKGLEWIGEIYHSGSTYYSPSLKSRVTISRDNSKNTLYLQLNSLRAEDTAVYYCARYEYDYYDFDYWGQGTLVTVSSGGGGSGGGGSGGGGSGGGGSDIQLTQSPSSLSASVGDRVTITCRASQSVDFHGISFLHWYQQKPGKAPKLLIYAASSLQSGVPSRFSGSGSGTDFTLTISSLQPEDFATYYCQQSYRSPRTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 329 - protein, 483 aa, synthetic construct
  REGION 1..483: note = Synthetic: HC_B
EVQLVESGGGLVQPGGSLRLSCAASGFSLSTGGVGVSWIRQAPGKGLEWIGEIYHSGSTYYSPSLKSRVTISRDNSKNTLYLQLNSLRAEDTAVYYCARYEYYEYDFDYWGQGTLVTVSSGGGGSGGGGSGGGGSGGGGSDIQLTQSPSSLSASVGDRVTITCRASQSVDFHGISFLHWYQQKPGKAPKLLIYAASSLQSGVPSRFSGSGSGTDFTLTISSLQPEDFATYYCQQSYRSPRTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 330 - protein, 483 aa, synthetic construct
  REGION 1..483: note = Synthetic: HC_B
EVQLVESGGGLVQPGGSLRLSCAASGFSLSTGGVGVSWIRQAPGKGLEWIGEIYHSGSTYYSPSLKSRVTISRDNSKNTLYLQLNSLRAEDTAVYYCARYEYYHYDFDYWGQGTLVTVSSGGGGSGGGGSGGGGSGGGGSDIQLTQSPSSLSASVGDRVTITCRASQSVDFHGISFLHWYQQKPGKAPKLLIYAASSLQSGVPSRFSGSGSGTDFTLTISSLQPEDFATYYCQQSYRSPRTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 331 - protein, 214 aa, synthetic construct; identical to SEQ ID NO: 169
  REGION 1..214: note = Synthetic: LC

SEQ ID NO: 332 - protein, 449 aa, synthetic construct; identical to SEQ ID NO: 170
  REGION 1..449: note = Synthetic: HC_A

SEQ ID NO: 333 - protein, 483 aa, synthetic construct; identical to SEQ ID NO: 171
  REGION 1..483: note = Synthetic: HC_B

SEQ ID NO: 334 - protein, 483 aa, synthetic construct
  REGION 1..483: note = Synthetic: HC_B
DIQLTQSPSSLSASVGDRVTITCRASQSVDFYGKSFLHWYQQKPGKAPKLLIYAASSLESGVPSRFSGSGSGTDFTLTISSLQPEDFATYYCQQSYRSPPTFGQGTKVEIKGGGGSGGGGSGGGGSGGGGSEVQLVESGGGLVQPGGSLRLSCAASGFSLSTGGVGVSWIRQAPGKGLEWIGSIYHSGSTYYNPSLKSRVTISRDNSKNTLYLQLNSLRAEDTAVYYCARYEYDYYAFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 335 - protein, 483 aa, synthetic construct
  REGION 1..483: note = Synthetic: HC_B
DIQLTQSPSSLSASVGDRVTITCRASQSVDFYGISFLHWYQQKPGKAPKLLIYAASSLQSGVPSRFSGSGSGTDFTLTISSLQPEDFATYYCQQSYRTPPTFGQGTKVEIKGGGGSGGGGSGGGGSGGGGSEVQLVESGGGLVQPGGSLRLSCAASGFSLSTGGVGVSWIRQAPGKGLEWIGSIYHSGSTYYNPSLKSRVTISRDNSKNTLYLQLNSLRAEDTAVYYCARYEYGYYALDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 336 - protein, 483 aa, synthetic construct
  REGION 1..483: note = Synthetic: HC_B
DIQLTQSPSSLSASVGDRVTITCRASQSVDFYGKSFLHWYQQKPGKAPKLLIYAASSLQSGVPSRFSGSGSGTDFTLTISSLQPEDFATYYCQQSYRTPLTFGQGTKVEIKGGGGSGGGGSGGGGSGGGGSEVQLVESGGGLVQPGGSLRLSCAASGFSLSTSGVGVSWIRQAPGKGLEWIGSIYHSGSTYYNPSLKSRVTISRDNSKNTLYLQLNSLRAEDTAVYYCARYEYGYYAFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 337 - protein, 483 aa, synthetic construct
  REGION 1..483: note = Synthetic: HC_B
DIQLTQSPSSLSASVGDRVTITCRASQSVDFYGISFLHWYQQKPGKAPKLLIYAASSLQSGVPSRFSGSGSGTDFTLTISSLQPEDFATYYCQQSYRTPRTFGQGTKVEIKGGGGSGGGGSGGGGSGGGGSEVQLVESGGGLVQPGGSLRLSCAASGFSLSTSGVGVSWIRQAPGKGLEWIGSIYHSGSTYYNPSLKSRVTISRDNSKNTLYLQLNSLRAEDTAVYYCARYEYDYYAFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 338 - protein, 483 aa, synthetic construct
  REGION 1..483: note = Synthetic: HC_B
DIQLTQSPSSLSASVGDRVTITCRASQSVDFYGKSFLHWYQQKPGKAPKLLIYAASSLQSGVPSRFSGSGSGTDFTLTISSLQPEDFATYYCQQSYRTPRTFGQGTKVEIKGGGGSGGGGSGGGGSGGGGSEVQLVESGGGLVQPGGSLRLSCAASGFSLSTSGVGVGWIRQAPGKGLEWIGEIYHSGSTNYNPSLKSRVTISRDNSKNTLYLQLNSLRAEDTAVYYCARYEYGYYALDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 339 - protein, 483 aa, synthetic construct
  REGION 1..483: note = Synthetic: HC_B
DIQLTQSPSSLSASVGDRVTITCRASQSVDFHGISFLHWYQQKPGKAPKLLIYAASSLESGVPSRFSGSGSGTDFTLTISSLQPEDFATYYCQQSYRTPRTFGQGTKVEIKGGGGSGGGGSGGGGSGGGGSEVQLVESGGGLVQPGGSLRLSCAASGFSLSTGGVGVGWIRQAPGKGLEWIGEIYHSGSTYYNPSLKSRVTISRDNSKNTLYLQLNSLRAEDTAVYYCARYEYGYYAFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 340 - protein, 483 aa, synthetic construct
  REGION 1..483: note = Synthetic: HC_B
DIQLTQSPSSLSASVGDRVTITCRASQSVDFYGKSFLHWYQQKPGKAPKLLIYAASSLESGVPSRFSGSGSGTDFTLTISSLQPEDFATYYCQQSYRSPLTFGQGTKVEIKGGGGSGGGGSGGGGSGGGGSEVQLVESGGGLVQPGGSLRLSCAASGFSLSTSGVGVGWIRQAPGKGLEWIGSIYHSGSTYYNPSLKSRVTISRDNSKNTLYLQLNSLRAEDTAVYYCARYEYDYYDFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 341 - protein, 483 aa, synthetic construct
  REGION 1..483: note = Synthetic: HC_B
DIQLTQSPSSLSASVGDRVTITCRASQSVDFHGISFLHWYQQKPGKAPKLLIYAASSLQSGVPSRFSGSGSGTDFTLTISSLQPEDFATYYCQQSYRSPRTFGQGTKVEIKGGGGSGGGGSGGGGSGGGGSEVQLVESGGGLVQPGGSLRLSCAASGFSLSTSGVGVSWIRQAPGKGLEWIGEIYHSGSTYYNPSLKSRVTISRDNSKNTLYLQLNSLRAEDTAVYYCARYEYGYYAFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 342 - protein, 483 aa, synthetic construct
  REGION 1..483: note = Synthetic: HC_B
DIQLTQSPSSLSASVGDRVTITCRASQSVDFHGKSFLHWYQQKPGKAPKLLIYAASSLQSGVPSRFSGSGSGTDFTLTISSLQPEDFATYYCQQSYRSPRTFGQGTKVEIKGGGGSGGGGSGGGGSGGGGSEVQLVESGGGLVQPGGSLRLSCAASGFSLSTSGVGVSWIRQAPGKGLEWIGEIYHSGSTYYSPSLKSRVTISRDNSKNTLYLQLNSLRAEDTAVYYCARYEYYSYAFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 343 - protein, 483 aa, synthetic construct
  REGION 1..483: note = Synthetic: HC_B
DIQLTQSPSSLSASVGDRVTITCRASQSVDFYGISFLHWYQQKPGKAPKLLIYAASSLESGVPSRFSGSGSGTDFTLTISSLQPEDFATYYCQQSYRTPLTFGQGTKVEIKGGGGSGGGGSGGGGSGGGGSEVQLVESGGGLVQPGGSLRLSCAASGFSLSTGGVGVSWIRQAPGKGLEWIGSIYHSGSTYYSPSLKSRVTISRDNSKNTLYLQLNSLRAEDTAVYYCARYEYDYYAFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 344 - protein, 483 aa, synthetic construct
  REGION 1..483: note = Synthetic: HC_B
EVQLVESGGGLVQPGGSLRLSCAASGFSLSTGGVGVSWIRQAPGKGLEWIGSIYHSGSTYYNPSLKSRVTISRDNSKNTLYLQLNSLRAEDTAVYYCARYEYDYYAFDYWGQGTLVTVSSGGGGSGGGGSGGGGSGGGGSDIQLTQSPSSLSASVGDRVTITCRASQSVDFYGKSFLHWYQQKPGKAPKLLIYAASSLESGVPSRFSGSGSGTDFTLTISSLQPEDFATYYCQQSYRSPPTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 345 - protein, 483 aa, synthetic construct
  REGION 1..483: note = Synthetic: HC_B
EVQLVESGGGLVQPGGSLRLSCAASGFSLSTGGVGVSWIRQAPGKGLEWIGSIYHSGSTYYNPSLKSRVTISRDNSKNTLYLQLNSLRAEDTAVYYCARYEYGYYALDYWGQGTLVTVSSGGGGSGGGGSGGGGSGGGGSDIQLTQSPSSLSASVGDRVTITCRASQSVDFYGISFLHWYQQKPGKAPKLLIYAASSLQSGVPSRFSGSGSGTDFTLTISSLQPEDFATYYCQQSYRTPPTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 346 - protein, 483 aa, synthetic construct
  REGION 1..483: note = Synthetic: HC_B
EVQLVESGGGLVQPGGSLRLSCAASGFSLSTSGVGVSWIRQAPGKGLEWIGSIYHSGSTYYNPSLKSRVTISRDNSKNTLYLQLNSLRAEDTAVYYCARYEYGYYAFDYWGQGTLVTVSSGGGGSGGGGSGGGGSGGGGSDIQLTQSPSSLSASVGDRVTITCRASQSVDFYGKSFLHWYQQKPGKAPKLLIYAASSLQSGVPSRFSGSGSGTDFTLTISSLQPEDFATYYCQQSYRTPLTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 347 - protein, 483 aa, synthetic construct
  REGION 1..483: note = Synthetic: HC_B
EVQLVESGGGLVQPGGSLRLSCAASGFSLSTSGVGVSWIRQAPGKGLEWIGSIYHSGSTYYNPSLKSRVTISRDNSKNTLYLQLNSLRAEDTAVYYCARYEYDYYAFDYWGQGTLVTVSSGGGGSGGGGSGGGGSGGGGSDIQLTQSPSSLSASVGDRVTITCRASQSVDFYGISFLHWYQQKPGKAPKLLIYAASSLQSGVPSRFSGSGSGTDFTLTISSLQPEDFATYYCQQSYRTPRTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 348 - protein, 483 aa, synthetic construct
  REGION 1..483: note = Synthetic: HC_B
EVQLVESGGGLVQPGGSLRLSCAASGFSLSTSGVGVGWIRQAPGKGLEWIGEIYHSGSTNYNPSLKSRVTISRDNSKNTLYLQLNSLRAEDTAVYYCARYEYGYYALDYWGQGTLVTVSSGGGGSGGGGSGGGGSGGGGSDIQLTQSPSSLSASVGDRVTITCRASQSVDFYGKSFLHWYQQKPGKAPKLLIYAASSLQSGVPSRFSGSGSGTDFTLTISSLQPEDFATYYCQQSYRTPRTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 349 - protein, 483 aa, synthetic construct
  REGION 1..483: note = Synthetic: HC_B
EVQLVESGGGLVQPGGSLRLSCAASGFSLSTGGVGVGWIRQAPGKGLEWIGEIYHSGSTYYNPSLKSRVTISRDNSKNTLYLQLNSLRAEDTAVYYCARYEYGYYAFDYWGQGTLVTVSSGGGGSGGGGSGGGGSGGGGSDIQLTQSPSSLSASVGDRVTITCRASQSVDFHGISFLHWYQQKPGKAPKLLIYAASSLESGVPSRFSGSGSGTDFTLTISSLQPEDFATYYCQQSYRTPRTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 350 - protein, 483 aa, synthetic construct
  REGION 1..483: note = Synthetic: HC_B
EVQLVESGGGLVQPGGSLRLSCAASGFSLSTSGVGVGWIRQAPGKGLEWIGSIYHSGSTYYNPSLKSRVTISRDNSKNTLYLQLNSLRAEDTAVYYCARYEYDYYDFDYWGQGTLVTVSSGGGGSGGGGSGGGGSGGGGSDIQLTQSPSSLSASVGDRVTITCRASQSVDFYGKSFLHWYQQKPGKAPKLLIYAASSLESGVPSRFSGSGSGTDFTLTISSLQPEDFATYYCQQSYRSPLTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 351 - protein, 483 aa, synthetic construct
  REGION 1..483: note = Synthetic: HC_B
EVQLVESGGGLVQPGGSLRLSCAASGFSLSTSGVGVSWIRQAPGKGLEWIGEIYHSGSTYYNPSLKSRVTISRDNSKNTLYLQLNSLRAEDTAVYYCARYEYGYYAFDYWGQGTLVTVSSGGGGSGGGGSGGGGSGGGGSDIQLTQSPSSLSASVGDRVTITCRASQSVDFHGISFLHWYQQKPGKAPKLLIYAASSLQSGVPSRFSGSGSGTDFTLTISSLQPEDFATYYCQQSYRSPRTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 352 - protein, 483 aa, synthetic construct
  REGION 1..483: note = Synthetic: HC_B
EVQLVESGGGLVQPGGSLRLSCAASGFSLSTSGVGVSWIRQAPGKGLEWIGEIYHSGSTYYSPSLKSRVTISRDNSKNTLYLQLNSLRAEDTAVYYCARYEYYSYAFDYWGQGTLVTVSSGGGGSGGGGSGGGGSGGGGSDIQLTQSPSSLSASVGDRVTITCRASQSVDFHGKSFLHWYQQKPGKAPKLLIYAASSLQSGVPSRFSGSGSGTDFTLTISSLQPEDFATYYCQQSYRSPRTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 353 - protein, 483 aa, synthetic construct
  REGION 1..483: note = Synthetic: HC_B
EVQLVESGGGLVQPGGSLRLSCAASGFSLSTGGVGVSWIRQAPGKGLEWIGSIYHSGSTYYSPSLKSRVTISRDNSKNTLYLQLNSLRAEDTAVYYCARYEYDYYAFDYWGQGTLVTVSSGGGGSGGGGSGGGGSGGGGSDIQLTQSPSSLSASVGDRVTITCRASQSVDFYGISFLHWYQQKPGKAPKLLIYAASSLESGVPSRFSGSGSGTDFTLTISSLQPEDFATYYCQQSYRTPLTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 354 - protein, 214 aa, synthetic construct; identical to SEQ ID NO: 175
  REGION 1..214: note = Synthetic: LC

SEQ ID NO: 355 - protein, 448 aa, synthetic construct; identical to SEQ ID NO: 176
  REGION 1..448: note = Synthetic: HC_A

SEQ ID NO: 356 - protein, 483 aa, synthetic construct
  REGION 1..483: note = Synthetic: HC_B
EVQLVESGGGLVQPGGSLRLSCAASGFSLSTSGVGVSWIRQAPGKGLEWIGEIYHSGSTYYSPSLKSRVTISRDNSKNTLYLQLNSLRAEDTAVYYCARYGYYYYDFDYWGQGTLVTVSSGGGGSGGGGSGGGGSGGGGSDIQLTQSPSSLSASVGDRVTITCRASQSVDFHGISFLHWYQQKPGKAPKLLIYAASSLQSGVPSRFSGSGSGTDFTLTISSLQPEDFATYYCQQSYRSPRTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 357 - protein, 483 aa, synthetic construct
  REGION 1..483: note = Synthetic: HC_B
EVQLVESGGGLVQPGGSLRLSCAASGFSLSTSGVGVGWIRQAPGKGLEWIGEIYHSGSTYYNPSLKSRVTISRDNSKNTLYLQLNSLRAEDTAVYYCARYKYDYYDFDYWGQGTLVTVSSGGGGSGGGGSGGGGSGGGGSDIQLTQSPSSLSASVGDRVTITCRASQSVDFHGISFLHWYQQKPGKAPKLLIYAASSLQSGVPSRFSGSGSGTDFTLTISSLQPEDFATYYCQQSYRSPRTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 358 - protein, 483 aa, synthetic construct
  REGION 1..483: note = Synthetic: HC_B
EVQLVESGGGLVQPGGSLRLSCAASGFSLSTGGVGVGWIRQAPGKGLEWIGEIYHSGSTYYSPSLKSRVTISRDNSKNTLYLQLNSLRAEDTAVYYCARYGYYYYDFDYWGQGTLVTVSSGGGGSGGGGSGGGGSGGGGSDIQLTQSPSSLSASVGDRVTITCRASQSVDFHGISFLHWYQQKPGKAPKLLIYAASSLQSGVPSRFSGSGSGTDFTLTISSLQPEDFATYYCQQSYRSPRTFGQGTKVEIKEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 359 - protein, 108 aa, Homo sapiens
  REGION 1..108: note = misc_feature - sp|P10747|CD28_HUMAN
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNE

SEQ ID NO: 360 - protein, 108 aa, Mus musculus
  REGION 1..108: note = misc_feature - sp|P31041|CD28_MOUSE
NKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNE

SEQ ID NO: 361 - protein, 108 aa, synthetic construct; identical to SEQ ID NO: 359
  REGION 1..108: note = Synthetic: TY24865